SEQUENCE LISTING (81 sequences)

SEQ ID NO: 1 - DNA, 981 bp, Lactobacillus reuteri
atgaaagggagactgtttattatgtctaagaatcatcaaaaagttgttcttgttggtgacggtgctgtcggttcaagttacgccttcgcaatggcacaacaaggaattgctgaagaatttgctatcgttgatattattaaggaacggactgaaggggacgcaatggaccttgaagacgctactgccttcaccgctcctaagaacatctactcagcagattacgacacttgcaaagacgctgatttagttgttattactgctggtgctccacaaaagcctggtgaaactcgtcttcaattagttgataagaacttgaagatcattaagtccgttgtagaaccaatcgttaagtctggttttgacggaatcttcttagttgcagctaacccagttgacatccttacctatgctgttcaaaagctttctggcttccctaagaacaaggttgttggttcaggtacttcacttgattccgcacgtcttcgggttgctcttggtaagaagcttgatgttgaccctcgtgatgttattgctaacattatgggtgaacatggtgattccgaatttgctgcttactcaagtgctactgttggtggcaagccattacttgacatcgctaaggatgaaggtatttcagaagatgaattacttaagattgaagatgatgttcgtaacaaggcatacgaaatcattaaccgcaaaggtgctaccttctatggtgttgccactgctttaatgcggatttcaaaggctattcttcgtgatgaaaattctgttctccctatcggtgccccaatgaacggtgaatacggacttaacgacctttacatcggtactccagctgttgttaatgcttctggtgttgcaaaggttattgaagttccacttaacgaccgtgaaaagaaggctatggccgactctgctaagcaattggaagaagttgctaagaacggtatggcaaagttacaaggtaacaactaa

SEQ ID NO: 2 - protein, 326 aa, Lactobacillus reuteri
MKGRLFIMSKNHQKVVLVGDGAVGSSYAFAMAQQGIAEEFAIVDIIKERTEGDAMDLEDATAFTAPKNIYSADYDTCKDADLVVITAGAPQKPGETRLQLVDKNLKIIKSVVEPIVKSGFDGIFLVAANPVDILTYAVQKLSGFPKNKVVGSGTSLDSARLRVALGKKLDVDPRDVIANIMGEHGDSEFAAYSSATVGGKPLLDIAKDEGISEDELLKIEDDVRNKAYEIINRKGATFYGVATALMRISKAILRDENSVLPIGAPMNGEYGLNDLYIGTPAVVNASGVAKVIEVPLNDREKKAMADSAKQLEEVAKNGMAKLQGNN

SEQ ID NO: 3 - DNA, 930 bp, Lactobacillus plantarum
atggataagaagcaacgcaaagtcgtaattgttggtgatggctcggtgggttcatcatttgccttttcattggtccaaaattgcgccctagatgaactcgttatcgttgacttggttaaaacgcacgcagagggggacgttaaggatttggaagatgttgccgcctttacgaatgcgaccaacattcataccggtgaatatgcggatgcgcgtgatgctgacatcgttgtcattacggctggtgtgcctcgtaagcctggtgagagtcgtttagatttgattaaccgcaatacgaagattctggaatccatcgtcaaaccagtggttgcgagtggttttaatggttgcttcgttatctcaagtaatcccgtcgatattttgacttcgatgacgcaacgtttatccggttttccacggcatcgggtcattggtaccgggacttccttggatacggcgcggttacgggtcgccttggctcagaagttgaatgttgccactactgcagttgatgctgcggtacttggagaacatggtgatagttccatcgttaattttgatgaaattatgatcaatgctcagcccttaaagacggtcacaacggtcgatgatcagttcaaagctgaaatcgagcaagctgttcgtggtaaaggtggtcaaatcattagtcagaagggggccacgttctatggggtcgccgttagtttgatgcaaatctgccgagcaattttgaacgatgaaaatgctgagttgattgtctccgccgctttgtctggtcaatatggcgttaacgatttgtacttggggtcacccgccattattaaccgcaacgggctccaaaaagtgatcgaagctgagctatcagatgatgagcgtgcccggatgcaacatttcgcagccaagatgctgaccatgatgaatgtggcatcataa

SEQ ID NO: 4 - protein, 309 aa, Lactobacillus plantarum
MDKKQRKVVIVGDGSVGSSFAFSLVQNCALDELVIVDLVKTHAEGDVKDLEDVAAFTNATNIHTGEYADARDADIVVITAGVPRKPGESRLDLINRNTKILESIVKPVVASGFNGCFVISSNPVDILTSMTQRLSGFPRHRVIGTGTSLDTARLRVALAQKLNVATTAVDAAVLGEHGDSSIVNFDEIMINAQPLKTVTTVDDQFKAEIEQAVRGKGGQIISQKGATFYGVAVSLMQICRAILNDENAELIVSAALSGQYGVNDLYLGSPAIINRNGLQKVIEAELSDDERARMQHFAAKMLTMMNVAS

SEQ ID NO: 5 - DNA, 1551 bp, Lactobacillus brevis
ttgaatattgaaaaagcattctttcctgccatcaagtcttttcgacaggataaattggaaaggaagcacaatattatggcacaaaatgctgtagcattaaaacattatcaaatgtatattaacggagaatttaaagattcagagtcaggtaatatgttgacggtactgaatccttcaacgggagaagcaatttctactgttccaagtgcaacccgtgaagaaacaaaagaagcaattgatgcggcttatgaagctgaaaaatcatggaagaaggttccggctgctactcgtggccaatatcttcatgatgttgtgacagaagttcgtaagcaaaaggatcacttaattgaaatgcttcaagaggagcagggaaagattaagtccttggcaacaactgaaatcttgttctcggctgattactttgattatatggctagtgctgctcggacatacgaaggcgaaatccttcaatccgacaacaaaaatgaaaacattatgattgctaagcaaccaattggtgtcgctgccgggatcttaccatggaacttcccattcttcttgattgcccgtaaaatgggtccggctttggtaacaggtaataccattgttatgaagccaagttcggatacacctaacttggcacttgaatttgctaagatctgtgacaaagttggtattccaaagggtgttgtcaacattgtttcaggaccgggttcggtcgttggtgatgagttaactcgaaacaagaagatcggtattatcagtttgaccggttctgttgaatcaggcaagcgcattatggaaggtgctgccactcatatggccaaggtatcacttgaattaggtggtaaagcaccagccattgtttgcaaggatgccgatattgatctcgccgtacaatcaattattgactctcgagtcgataacaatggtcaactttgcaacaactgtgaacgggtttacgttcaagaagatgttgcggacgaatttatcaagaagttgtccaaaaagatgtctgagattacggttggcaacccaatgaaagatgagaagatcggtatgggaccattgattaatcaagcagctttggataaggttgccggaatggttgatcgtgctgttaaagccggtggcaagatcacggccggtggtcacaagctgaatattgaaaatggattctattacgaaccaactgtcattaccaacgttaaacaggattccgaaattgttcaagacgaaatctttggaccggttcttcctgtattaacctttaagacattggatgatgccattaaaatggctaatgacagtgactttggattgacttcatcaatctttactgagaatttggataacgcaatgcgtgccgcaaatgaacttgaagacggtgaaacctatatcaaccgcttcaattttgaagctatgaatggttctcactccggctggaaagaatccggtattggtggagacgatggtcgccatggtattgaagaatacttgaatacccacgtcatctacctgcagggtcatcctgaaaaggctaatggttaa

SEQ ID NO: 6 - protein, 516 aa, Lactobacillus brevis
MNIEKAFFPAIKSFRQDKLERKHNIMAQNAVALKHYQMYINGEFKDSESGNMLTVLNPSTGEAISTVPSATREETKEAIDAAYEAEKSWKKVPAATRGQYLHDVVTEVRKQKDHLIEMLQEEQGKIKSLATTEILFSADYFDYMASAARTYEGEILQSDNKNENIMIAKQPIGVAAGILPWNFPFFLIARKMGPALVTGNTIVMKPSSDTPNLALEFAKICDKVGIPKGVVNIVSGPGSVVGDELTRNKKIGIISLTGSVESGKRIMEGAATHMAKVSLELGGKAPAIVCKDADIDLAVQSIIDSRVDNNGQLCNNCERVYVQEDVADEFIKKLSKKMSEITVGNPMKDEKIGMGPLINQAALDKVAGMVDRAVKAGGKITAGGHKLNIENGFYYEPTVITNVKQDSEIVQDEIFGPVLPVLTFKTLDDAIKMANDSDFGLTSSIFTENLDNAMRAANELEDGETYINRFNFEAMNGSHSGWKESGIGGDDGRHGIEEYLNTHVIYLQGHPEKANG

SEQ ID NO: 7 - DNA, 1440 bp, Escherichia coli
atgtcagtacccgttcaacatcctatgtatattgatggacagtttgttacctggcgtggagacgcatggattgatgtggtaaaccctgcaacagaggctgtcatttcccgcattcccgatggtcaggccgaggatgtccgtaaggcaatcgatgcagcagaacgtgcacaaccagaatgggaagcgttgcctgctattgaacgcgccagttggttgcgcaaaatctccgccgggatccgcaaacgcgccagcgaaatcagtgcgctgattgttgaagaagggggcaagatccagcagctggctgaagtcgaagtggcttttactgcggactatatcgattacatggcggagtgggcacggcgttatgaaggcgagattattcaaagcgatcgtccaggagaaaatattcttctgtttaaacgtgcgcttggcgtgactaccggcattctgccgtggaacttcccgttcttcctcattgcccgcaaaatggcccccgctcttttgaccggtaataccatcgtcattaaacccagtgaatttacgccaaacaatgcgattgcattcgccaaaattgtcgatgaaataggccttccgcgcggcgtgtttaaccttgttctggggcgtggtgaaaccgttgggcaagaactggcgggtaacccaaaggtcgcaatggtcagtatgacaggcagcgtctctgcaggtgaaaagattatggcgactgcggcgaaaaacatcaccaaagtgtgcctggaattggggggtaaagcaccggctatcgtaatggacgatgccgatcttgaactggcagtcaaagccatcgttgattcacgcgttattaatagtgggcaagtgtgtaactgtgcagaacgtgtttatgtacagaaaggcatttatgatcagtttgtcaatcggctgggtgaagcgttgcaggcggttcaatttggtaaccccgctgaacgcaacgacattgcgatggggccgttgattaacgccgcggcgctggaaagggtcgagcaaaaagtggcgcgcgcagtagaagaaggggcgagagtggcgttgggtggcaaagcggtagaggggaaaggatattattatccgccgacattgctgctggatgttcgccaggaaatgtcgattatgcatgaggaaacctttggcccggttctgccggtggtcgcatttgatacgctggaagaggctatctcaatggcgaatgatagtgattacggcctgacctcatcaatctatacccaaaatctgaacgtcgcgatgaaagccattaacgggctgaagtttggtgaaacttacatcaaccgtgaaaacttcgaagctatgcaaggcttccacgccggatggcgtaaatctggtattggcggcgcagatggtaaacatggcctgcatgaatatctgcagacccaggtggtttatttacagtcttaa

SEQ ID NO: 8 - protein, 479 aa, Escherichia coli
MSVPVQHPMYIDGQFVTWRGDAWIDVVNPATEAVISRIPDGQAEDVRKAIDAAERAQPEWEALPAIERASWLRKISAGIRKRASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEALQAVQFGNPAERNDIAMGPLINAAALERVEQKVARAVEEGARVALGGKAVEGKGYYYPPTLLLDVRQEMSIMHEETFGPVLPVVAFDTLEEAISMANDSDYGLTSSIYTQNLNVAMKAINGLKFGETYINRENFEAMQGFHAGWRKSGIGGADGKHGLHEYLQTQVVYLQS

SEQ ID NO: 9 - DNA, 1152 bp, Escherichia coli
atgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 10 - protein, 383 aa, Escherichia coli
MMANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 11 - DNA, 1149 bp, Citrobacter koseri
atggcgaacagaatgatcctgaacgaaacggcatggtttggccggggggcggtggcggcgttaaccgatgaggttcaacgccggggctatcgcaaggcgctaattgtcagtgacaaaacgctggtgcagtgcggtgtggtcagaaacgtcacggataagatggatgccgcagggctggcatgggcgatctacgatggcgtgatcccaaatccaaccatcggcgtggtgaaagaggggctgaacgtatttcaacaaagcggggcagactacctgattgccatcggcggcggctccccgcaggatacctgtaaggcgatcggcattatcagcaataaccctgagtttgctgacgtgcgcagtctggaaggtctgtcgctgacccgttatccgagcgtgccggtgatggcgatcccgaccactgcgggaaccgcagcggaagtgaccattaactatgtgatcaccgatgaagaaaagcggcgtaaatttgtttgtgtcgatccgcatgatatcccgcaggtcgcgtttattgatgccgacatgatggacggcatgccgcccgcgctgaaggcggcaaccggggtggatgcgttaacccacgcgatcgaaggatacatcacccgtgccgcgtgggcgttgacggatgcgctgcatattaaggccattgagatcatcgccggggcattgcgtggttcggttgcgggcgagcgtgaagcgggcgaagcgatggcgctggggcagtacgttgccgggatgggcttttccaacgtggggcttgggctggtgcacgggatggcgcatccgctgggcgcgttttataacactccgcacggcgttgctaatgcgattcttctgccgcatatcatgcgctacaacgcggaatataccggtgagaagttccgtgatatcgcgcgtgcgatgggcgagaaggtcgaagggataagtttgagcgaggcgcgtaatgccgccgtggaagcggtatttaccctcaaccgggacgtcggtattccgttgcatttacgcgatgtcggcgtgcgtaaagaggatattccggcgctggcgcaggcggcatttgacgatgtttgcaccggtggtaatccgcgtgaagcgagtattgccgatatcgtcgagttgtaccataccgcctggtaa

SEQ ID NO: 12 - protein, 382 aa, Citrobacter koseri
MANRMILNETAWFGRGAVAALTDEVQRRGYRKALIVSDKTLVQCGVVRNVTDKMDAAGLAWAIYDGVIPNPTIGVVKEGLNVFQQSGADYLIAIGGGSPQDTCKAIGIISNNPEFADVRSLEGLSLTRYPSVPVMAIPTTAGTAAEVTINYVITDEEKRRKFVCVDPHDIPQVAFIDADMMDGMPPALKAATGVDALTHAIEGYITRAAWALTDALHIKAIEIIAGALRGSVAGEREAGEAMALGQYVAGMGFSNVGLGLVHGMAHPLGAFYNTPHGVANAILLPHIMRYNAEYTGEKFRDIARAMGEKVEGISLSEARNAAVEAVFTLNRDVGIPLHLRDVGVRKEDIPALAQAAFDDVCTGGNPREASIADIVELYHTAW

SEQ ID NO: 13 - DNA, 675 bp, Klebsiella oxytoca
atggaaattaatgaaaaattgctgcgccagataattgaagacgtgctcagcgagatgaagggcagcgataaaccggtctcgtttaatgcgccggcggcctccgcggcgccccaggccacgccgcccgccggcgacggcttcctgacggaagtgggcgaagcgcgtcagggaacccagcaggacgaagtgattatcgccgtcggcccggctttcggcctggcgcagaccgtcaatatcgtcggcatcccgcataagagcattttgcgcgaagtcattgccggtattgaagaagaaggcattaaggcgcgcgtgattcgctgctttaaatcctccgacgtggccttcgtcgccgttgaaggtaatcgcctgagcggctccggcatctctatcggcatccagtcgaaaggcaccacggtgatccaccagcaggggctgccgccgctctctaacctggagctgttcccgcaggcgccgctgctgaccctggaaacctatcgccagatcggcaaaaacgccgcccgctatgcgaaacgcgaatcgccgcagccggtcccgacgctgaatgaccagatggcgcggccgaagtaccaggcgaaatcggccattttgcacattaaagagaccaagtacgtggtgacgggcaaaaacccgcaggaactgcgcgtggcgctttga

SEQ ID NO: 14 - protein, 224 aa, Klebsiella oxytoca
MEINEKLLRQIIEDVLSEMKGSDKPVSFNAPAASAAPQATPPAGDGFLTEVGEARQGTQQDEVIIAVGPAFGLAQTVNIVGIPHKSILREVIAGIEEEGIKARVIRCFKSSDVAFVAVEGNRLSGSGISIGIQSKGTTVIHQQGLPPLSNLELFPQAPLLTLETYRQIGKNAARYAKRESPQPVPTLNDQMARPKYQAKSAILHIKETKYVVTGKNPQELRVAL

SEQ ID NO: 15 - DNA, 522 bp, Klebsiella oxytoca
atgaataccgacgcaattgaatcgatggtacgcgacgtattgagccgcatgaacagcctgcagggcgaggcgcctgcggcggctccggcggctggcggcgcgtcccgtagcgccagggtcagcgactacccgctggcgaacaagcacccggaatgggtgaaaaccgccaccaataaaacgctggacgactttacgctggaaaacgtgctgagcaataaagtcaccgcccaggatatgcgtattaccccggaaaccctgcgcttacaggcttctattgccaaagacgcgggccgcgaccggctggcgatgaacttcgagcgcgccgccgagctgaccgcggtaccggacgatcgcattcttgaaatctacaacgccctccgcccctatcgctcgacgaaagaggagctgctggcgatcgccgacgatctcgaaagccgctatcaggcgaagatttgcgccgctttcgttcgcgaagcggccacgctgtacgtcgagcgtaaaaaactcaaaggcgacgattaa

SEQ ID NO: 16 - protein, 173 aa, Klebsiella oxytoca
MNTDAIESMVRDVLSRMNSLQGEAPAAAPAAGGASRSARVSDYPLANKHPEWVKTATNKTLDDFTLENVLSNKVTAQDMRITPETLRLQASIAKDAGRDRLAMNFERAAELTAVPDDRILEIYNALRPYRSTKEELLAIADDLESRYQAKICAAFVREAATLYVERKKLKGDD

SEQ ID NO: 17 - DNA, 711 bp, Lactobacillus reuteri
atggctgatattgatgaaaacttattacgtaaaatcgttaaagaagttttaagcgaaactaatcaaatcgatactaagattgactttgataaaagtaatgatagtactgcaacagcaactcaagaggtgcaacaaccaaatagtaaagctgttccagaaaagaaacttgactggttccaaccagttggagaagcaaaacctggatattctaaggatgaagttgtaattgcagtcggtcctgcattcgcaactgttcttgataagacagaaactggtattcctcataaagaagtgcttcgtcaagttattgctggtattgaagaagaagggcttaaggcgcgggtagttaaagtttaccggagttcagatgtagcattctgtgctgtccaaggtgatcacctttctggttcaggaattgctattggtatccaatcaaaagggacgacagttattcaccaaaaggatcaagaccctcttggtaaccttgagttattcccacaagcgccagtacttactcccgaaacttatcgtgcaattggtaagaatgccgctatgtatgctaagggtgaatctccagaaccagttccagctaaaaacgatcaacttgctcgtattcactatcaagctatttcagcaattatgcatattcgtgaaactcaccaagttgttgttggtaagcctgaagaagaaattaaggttacgtttgattaa

SEQ ID NO: 18 - protein, 236 aa, Lactobacillus reuteri
MADIDENLLRKIVKEVLSETNQIDTKIDFDKSNDSTATATQEVQQPNSKAVPEKKLDWFQPVGEAKPGYSKDEVVIAVGPAFATVLDKTETGIPHKEVLRQVIAGIEEEGLKARVVKVYRSSDVAFCAVQGDHLSGSGIAIGIQSKGTTVIHQKDQDPLGNLELFPQAPVLTPETYRAIGKNAAMYAKGESPEPVPAKNDQLARIHYQAISAIMHIRETHQVVVGKPEEEIKVTFD

SEQ ID NO: 19 - DNA, 516 bp, Lactobacillus reuteri
atgagtgaagttgatgatttagtagcaaagatcatggcacagatgggaaatagctcatcttccgatagttcaacaagtgctacttcaacaaataacggtaaggaaatgacagcagatgactatcctctttaccaaaagcaccgtgatttagtaaagacaccatcaggaaagaaacttgatgatattactttacaaaaggttgtaaatgatcaagttaatccaaaagaattacggattactccagaagctctaaaacttcaaggtgagatcgcagcaaacgctggtcggccagcaattcaaaagaacttacaacgggcagctgaattaacacgtgttccagacgaacgtgttttgcaaatgtatgatgcattacggccattccgttcaacgaagcaagaattactagatattgctaatgaactccgtgataaatatcatgcagaagtatgtgcagcttggtttgaagaagctgcagattactatgaaagtcgaaagaagctcaagggtgataactag

SEQ ID NO: 20 - protein, 171 aa, Lactobacillus reuteri
MSEVDDLVAKIMAQMGNSSSSDSSTSATSTNNGKEMTADDYPLYQKHRDLVKTPSGKKLDDITLQKVVNDQVNPKELRITPEALKLQGEIAANAGRPAIQKNLQRAAELTRVPDERVLQMYDALRPFRSTKQELLDIANELRDKYHAEVCAAWFEEAADYYESRKKLKGDN

SEQ ID NO: 21 - DNA, 1011 bp, Escherichia coli
atgaaggctgcagttgttacgaaggatcatcatgttgacgttacgtataaaacactgcgctcactgaaacatggcgaagccctgctgaaaatggagtgttgtggtgtatgtcataccgatcttcatgttaagaatggcgattttggtgacaaaaccggcgtaattctgggccatgaaggcatcggtgtggtggcagaagtgggtccaggtgtcacctcattaaaaccaggcgatcgtgccagcgtggcgtggttctacgaaggatgcggtcattgcgaatactgtaacagtggtaacgaaacgctctgccgttcagttaaaaatgccggatacagcgttgatggcgggatggcggaagagtgcatcgtggtcgccgattacgcggtaaaagtgccagatggtctggactcggcggcggccagcagcattacctgtgcgggagtcaccacctacaaagccgttaagctgtcaaaaattcgtccagggcagtggattgctatctacggtcttggcggtctgggtaacctcgccctgcaatacgcgaagaatgtctttaacgccaaagtgatcgccattgatgtcaatgatgagcagttaaaactggcaaccgaaatgggcgcagatttagcgattaactcacacaccgaagacgccgccaaaattgtgcaggagaaaactggtggcgctcacgctgcggtggtaacagcggtagctaaagctgcgtttaactcggcagttgatgctgtccgtgcaggcggtcgtgttgtggctgtcggtctaccgccggagtctatgagcctggatatcccacgtcttgtgctggatggtattgaagtggtcggttcgctggtcggcacgcgccaggatttaactgaagccttccagtttgccgccgaaggtaaagtggtgccgaaagtcgccctgcgtccgttagcggacatcaacaccatctttactgagatggaagaaggcaaaatccgtggccgcatggtgattgatttccgtcactaa

SEQ ID NO: 22 - protein, 336 aa, Escherichia coli
MKAAVVTKDHHVDVTYKTLRSLKHGEALLKMECCGVCHTDLHVKNGDFGDKTGVILGHEGIGVVAEVGPGVTSLKPGDRASVAWFYEGCGHCEYCNSGNETLCRSVKNAGYSVDGGMAEECIVVADYAVKVPDGLDSAAASSITCAGVTTYKAVKLSKIRPGQWIAIYGLGGLGNLALQYAKNVFNAKVIAIDVNDEQLKLATEMGADLAINSHTEDAAKIVQEKTGGAHAAVVTAVAKAAFNSAVDAVRAGGRVVAVGLPPESMSLDIPRLVLDGIEVVGSLVGTRQDLTEAFQFAAEGKVVPKVALRPLADINTIFTEMEEGKIRGRMVIDFRH

SEQ ID NO: 23 - DNA, 1131 bp, Propionibacterium freudenreichii
atgaagcaattccaactggccacttctctcgcctgcgggcctgacgcactcaccgcgctggacaggctcggcggacgccgcgtcttggtcatcacggacgccttcatggcctcctcggcactgatggacaccgtgcgcggccacctcggcagcgccgagatcacggtcttcgaccaggtgcagcccaatcccgacgtccaggccgtcgcgcgcggcctgcgggccttccttgactgcgcccccgaggccctgctggccctgggcgggggatcgccgatcgacaccgccaaggcggtgcgcaagattgccctcgagcagggccagccactgtcggcgggcttctatgtggtgcccaccacctcgggaaccgggtcggaggtcagctcgttcgcggtggtcaccgatcccgagcacgacgccaagctccccatgacctcacccgacatggtggcagatgtggccatcctcgaccctgacgcggtgcgcacctgcccgcccacgctcaccgccgactccgggatggacgcgctcagccacgcggtggaggcctatgttgcgctggaccacaacgacatcacggacgcactggccgagaaggcgctgcgactgatcagcgcgaacctggtggccagcttccgcgacgggaacgaccttgcggccagggaacaccagcagaatgcggcgacgatggccggcatcgccttcgagaactccggactgggcatcgtgcacgggttgtcccatgccatcggcggcagcttccatgtggcgcacggcaggctcaacggcatcctgatgccgcatgtgatcggcttcaacgccggcgagctgggcttcggggctgccacgttgtcgcccatcgccgagcgctatgcacagctcgcggcggccatcggcatcgacgcggccacccgacgcggactggtgacggggctcgtcgacttcatcaccgccatacgtcgtgaccttgacatgccggcctcgctcaccgacgccggcgtcgaccgcgccgccttccgggctgccattccacagctgtcccagaccgcattgcgcgacttctgcacatcgggaaacccccggccggtgaccgctcatgagctcgccgggctgttggcccacgccctctag

SEQ ID NO: 24 - protein, 376 aa, Propionibacterium freudenreichii
MKQFQLATSLACGPDALTALDRLGGRRVLVITDAFMASSALMDTVRGHLGSAEITVFDQVQPNPDVQAVARGLRAFLDCAPEALLALGGGSPIDTAKAVRKIALEQGQPLSAGFYVVPTTSGTGSEVSSFAVVTDPEHDAKLPMTSPDMVADVAILDPDAVRTCPPTLTADSGMDALSHAVEAYVALDHNDITDALAEKALRLISANLVASFRDGNDLAAREHQQNAATMAGIAFENSGLGIVHGLSHAIGGSFHVAHGRLNGILMPHVIGFNAGELGFGAATLSPIAERYAQLAAAIGIDAATRRGLVTGLVDFITAIRRDLDMPASLTDAGVDRAAFRAAIPQLSQTALRDFCTSGNPRPVTAHELAGLLAHAL

SEQ ID NO: 25 - DNA, 1551 bp, Lactobacillus buchneri
ttgaatattgaaaaagcattctttcctgccatcaagtcttttcgacaggataaattggaaaggaagcacaatattatggcacaaaatgctgtagcattaaaacattatcaaatgtatattaacggagaatttaaagattcagagtcaggtaatatgttgacggtactaaatccttcaacgggagaagcaatttctactgttccaagtgcaacccgtgaagaaacaaaagaagcaattgatgcggcttatgaagctgaaaaatcatggaagaaggttccggctgctactcgtggccaatatcttcatgatgttgctacagaagttcgtaagcaaaaggatcacttaattgaaatgcttcaagaggaacaggggaagattaagtccttagcaacaactgaaatcttgttctcggctgattactttgattatatggctagtgctgctcggacatacgaaggcgaaatccttcaatccgacaacaaaaatgaaaacattatgattgctaagcaaccaattggtgtcgctgctgggatcttaccatggaacttcccattcttcttgattgcccgtaaaatgggtccggctttggtaacaggtaataccattgttatgaagccaagttcggatacacctaacttggcacttgaatttgctaatatctgtgacaaagttggtattccaaagggtgttgtcaacattgtttcaggaccgggttcggtcgttggtgatgagttaactcgaaacaagaagatcggtattatcagtttgaccggttctgttgaatcaggcaagcgcattatggaaggtgctgccactcatatggccaaggtatcacttgaattaggtggtaaagcaccagccattgtttgcaaggatgccgatattgatctcgccgtacaatcaattattgactctcgagtcgataacaatggtcaactttgcaacaactgtgaacgggtttacgttcaagaagatgttgcggacgaatttatcaagaagttgtccaaaaagatgtctgagattacggttggcaacccaatgaaagatgagaagatcggtatgggaccattgattaatcaagcagctttggataaggttgccggaatggttgatcgtgctgttaaagccggtggcaagattacggccggtggtcacaagctgaatattgaaaatggattctattacgaaccaactgtcattaccaacgttaaacaggattccgaaattgttcaagacgaaatctttggaccagttcttcctgtattaacctttaagacattggatgatgccattgaaatggctaatgacagtgactttggattgacttcatcaatctttactgagaatttggataacgcaatgcgtgccgcaaatgaacttgaagacggtgaaacctatatcaaccgcttcaattttgaagctatgaatggttctcactccggctggaaagaatccggtattggtggagacgatggtcgtcatggtattgaagaatacttgaatacccacgtcatctacctgcagggtcatcctgaaaaggctaatggttaa

SEQ ID NO: 26 - protein, 516 aa, Lactobacillus buchneri
MNIEKAFFPAIKSFRQDKLERKHNIMAQNAVALKHYQMYINGEFKDSESGNMLTVLNPSTGEAISTVPSATREETKEAIDAAYEAEKSWKKVPAATRGQYLHDVATEVRKQKDHLIEMLQEEQGKIKSLATTEILFSADYFDYMASAARTYEGEILQSDNKNENIMIAKQPIGVAAGILPWNFPFFLIARKMGPALVTGNTIVMKPSSDTPNLALEFANICDKVGIPKGVVNIVSGPGSVVGDELTRNKKIGIISLTGSVESGKRIMEGAATHMAKVSLELGGKAPAIVCKDADIDLAVQSIIDSRVDNNGQLCNNCERVYVQEDVADEFIKKLSKKMSEITVGNPMKDEKIGMGPLINQAALDKVAGMVDRAVKAGGKITAGGHKLNIENGFYYEPTVITNVKQDSEIVQDEIFGPVLPVLTFKTLDDAIEMANDSDFGLTSSIFTENLDNAMRAANELEDGETYINRFNFEAMNGSHSGWKESGIGGDDGRHGIEEYLNTHVIYLQGHPEKANG

SEQ ID NO: 27 - DNA, 1170 bp, Lactobacillus buchneri
atgaaagaaaactttgatttcttaatgccgagtgtcaacttctttggtaaaggggttatcaagaaaattggcgatcgtgcaaaaatgttaaatatgaaaaagccattgattgtgactgacaagtttttgcggtcaatggacaatggaccggttgctcaaacattagcgtcacttgaagcagttggcgttccttataccatctatgatggtgttgaacctaatccaaaaattcgaaatattcaagaagcaaagaaagtttatttagataatgattgtgacagtattatcactgttggtggtggttcttcacatgatgccggaaaagggaccggtatcattttgacaaacggtgatgacatcaccaagcttgccggaatcgaaacgctgcaaaatccattgccaccattgatggctgttaatacaaccgccgggaccggttcggagctgacccgtcattgtgtgatcacaaatgaaaaaactaaattgaaatttgttgttgtgtcatggcgaaatattccattggtatcatttaatgatccaatgttaatgttggatgttccggcatcattgacggctgcaacagggatggatacatttgttcaggcaattgaaccttatgtttcaaccaaccgaaatgaaattacggatggtcaatgcgttcaagcaattaaattggttgaagaaagtcttcgtgaagctgttgctaatggtcacaacattgaagcccgcaccaaaatggttgaagctgaaatgttgggtggtatggcttttaacaatgctgatttgggatatgttcatgcaatggcccatcaattgggcggtcaatatgatgcgccacacggtgtttgctgtgcaatgttacttccaattgttgaaaagtataatattgttgccgcaccggaaagatttgcacaattagctaaaattatgggtgaaaatacggacggattatccacaagggatgctgccgacttatccatcaaagcaatgcgccaaatgtgtgatgatgtcaagattcctcgaagcattaaagcaattggtgctaaaccggaagactttgaattgatggctgagaatgcgcttaaagacggtaacgccatttcaaatcctcgtaaaggaaccaaagaagaaattgttcaattattccaggaagcatatgatcaaaaataa

SEQ ID NO: 28 - protein, 389 aa, Lactobacillus buchneri
MKENFDFLMPSVNFFGKGVIKKIGDRAKMLNMKKPLIVTDKFLRSMDNGPVAQTLASLEAVGVPYTIYDGVEPNPKIRNIQEAKKVYLDNDCDSIITVGGGSSHDAGKGTGIILTNGDDITKLAGIETLQNPLPPLMAVNTTAGTGSELTRHCVITNEKTKLKFVVVSWRNIPLVSFNDPMLMLDVPASLTAATGMDTFVQAIEPYVSTNRNEITDGQCVQAIKLVEESLREAVANGHNIEARTKMVEAEMLGGMAFNNADLGYVHAMAHQLGGQYDAPHGVCCAMLLPIVEKYNIVAAPERFAQLAKIMGENTDGLSTRDAADLSIKAMRQMCDDVKIPRSIKAIGAKPEDFELMAENALKDGNAISNPRKGTKEEIVQLFQEAYDQK

SEQ ID NO: 29 - DNA, 1464 bp, Lactobacillus casei
atggctgaattgcagtcaacgcactatcagatgtatatcaatggcaagttcatggattcggcatcaggcgatgttgaaaccgtgatcgatccttccacaggtaagaaattgtggaccgcaccggcagcgacaattgaggaagcccgcgcagccgtggatgctgcttgggcggctaagaatgcttggcggcgcgtgcctgctccaacgcgtggtgcgtacttgcgcaagcttgctactttagtgcgtgagcatgaagatgaactggtgaagattctgatgttggagcaaggaaaaattcatgccttagcgaaaaccgaaattgagttttcggcggattattttgactatatggccaacgccgcacgcgtttattccggcgatgtgatgcaatctgacaatgaaaacgaaaatattatcattgcccgtcagccaatcggtgttgcggttggtattattgcttggaacttcccgttcttcctgattgctcggaaaatggggccagctttggtaaccggcaataccatcgtgatcaaaccaagttcggacaccccaggactggcgttggcatttgcaaaactggttgatgaagtcggtttgccggcaggggtagccaactttgtgagtggtcgcgggtccatcattggggatgatttggcgaagaaccccaaagtcggcattatcagcctgactgggtccaccgcgtccggtaagaaggtcatggcagctgcggcgaatcatgtggcaaagatgtcattggaacttggcgggaaggcaccggcaattgttgctaaggatgctgatatcgatttagctgtgcaggccatttttgactcgcgaattgataacaatgggcaaatttgtaacaacgctgaacggatttatgtgcaagaagatgttgcggatgagtttattaagaaaatgacggcaaagatggcggcggtgaaagtcggtaatccgaacactgatccagatgccgaaatgggtccgttgatcaatcaaggcgcccttgataaagtggacggtttggttcagcgggcggttgaagcaggcggccagattcaaacaggtggtcacccagctgacattgacggtggtttcttctacgaaccaactgtgattaccaatgtcgcgcaggattccgagattgtgcaaaatgagatcttcggaccggtgttaccgattttgacattcaagagcattccagaagcaatcgaaatggcgaatgacacggtatatggcctgaccagctccgtttatacgcgtgatctggaaaccgcttcgtatgttgctaacgagattgagtttggcgagacctacattaaccgattcaactttgaaggcatccaaggcttccatgctggttggaaacaatctggtgtcggaggcgccgatggtattcacggtgttgaagaatttaccaacacacatgtcatttatttacagcgtcacccagaaagcttaaaa

SEQ ID NO: 30 - protein, 488 aa, Lactobacillus casei
MAELQSTHYQMYINGKFMDSASGDVETVIDPSTGKKLWTAPAATIEEARAAVDAAWAAKNAWRRVPAPTRGAYLRKLATLVREHEDELVKILMLEQGKIHALAKTEIEFSADYFDYMANAARVYSGDVMQSDNENENIIIARQPIGVAVGIIAWNFPFFLIARKMGPALVTGNTIVIKPSSDTPGLALAFAKLVDEVGLPAGVANFVSGRGSIIGDDLAKNPKVGIISLTGSTASGKKVMAAAANHVAKMSLELGGKAPAIVAKDADIDLAVQAIFDSRIDNNGQICNNAERIYVQEDVADEFIKKMTAKMAAVKVGNPNTDPDAEMGPLINQGALDKVDGLVQRAVEAGGQIQTGGHPADIDGGFFYEPTVITNVAQDSEIVQNEIFGPVLPILTFKSIPEAIEMANDTVYGLTSSVYTRDLETASYVANEIEFGETYINRFNFEGIQGFHAGWKQSGVGGADGIHGVEEFTNTHVIYLQRHPESLK

SEQ ID NO: 31 - DNA, 1548 bp, Lactobacillus hilgardii
ttgaatattgaaaaagcattttttcctgccatcaagtcttttcgacaggataaattggaaaggaagcacaatattatggcacaaaatgctgtagcattaaaacattatcaaatgtacattaacggagaatttaaagattcagagtcaggtaatatgttgacggtactgaatccttcaacgggagaagcaatttctactgttccaagtgcaacccgtgaagaaacaaaagaagcaattgatgcggcttatgaagctgaaaaatcatggaagaaggttccggctgctactcgtggccaatatcttcacgatgttgcaacagaagttcgtaagcaaaaggatcacttaattgaaatgcttcaagaggagcagggaaagattaagtccttagcgacaactgaaatcttgttctcggctgactactttgattatatggctagtgccgctcggacatacgaaggcgaaatccttcaatccgacaacaaaaatgaaaacattatgattgctaagcaaccaattggtgtcgctgccgggatcttaccatggaacttcccattcttcttgattgcccgtaaaatgggtccggctttggtaacaggtaataccattgttatgaagccaagttcggatacacctaacttggcacttgaatttgctaagatctgtgacaaagttggtattccaaagggtgttgtcaacattgtttcaggaccgggttcggtcgttggtgatgagttaactcgaaacaagaagatcggtattatcagtttgaccggttctgttgaatcaggcaagcgcattatggaaggtgctgccactcatatggccaaggtatcacttgaattaggtggtaaagcaccagccattgtttgcaaggatgccgatattgatctcgccgtacaatcaattattgactctcgagtcgataacaatggtcaactttgcaacaactgtgaacgggtttacgttcaagaagatgttgcggacgaatttatcaagaagttgtccaaaaagatgtctgagattacggttggcaacccaatgaaagatgagaagatcggtatgggaccattgattaatcaagcagctttggataaggttgccggaatggttgatcgtgctgttaaagccggtggcaagatcacggccggtggtcacaagctgaatattgaaaatggattctattacgaaccaactgtcattaccaacgttaaacaggattccgaaattgttcaagacgaaatctttggaccggttcttcctgtattaacctttaagacattggatgatgccattgaaatggctaatgacagtgactttggattgacttcatcaatctttactgagaatttggataacgcaatgcgtgccgcaaatgaacttgaagacggtgaaacctatatcaaccgcttcaattttgaagctatgaatggttctcactccggctggaaagaatccggtattggtggagacgatggtcgtcatggtattgaagaatacttgaatacccacgtcatctacctgcagggtcatcctgaaaaggctaatggt

SEQ ID NO: 32 - protein, 516 aa, Lactobacillus hilgardii
MNIEKAFFPAIKSFRQDKLERKHNIMAQNAVALKHYQMYINGEFKDSESGNMLTVLNPSTGEAISTVPSATREETKEAIDAAYEAEKSWKKVPAATRGQYLHDVATEVRKQKDHLIEMLQEEQGKIKSLATTEILFSADYFDYMASAARTYEGEILQSDNKNENIMIAKQPIGVAAGILPWNFPFFLIARKMGPALVTGNTIVMKPSSDTPNLALEFAKICDKVGIPKGVVNIVSGPGSVVGDELTRNKKIGIISLTGSVESGKRIMEGAATHMAKVSLELGGKAPAIVCKDADIDLAVQSIIDSRVDNNGQLCNNCERVYVQEDVADEFIKKLSKKMSEITVGNPMKDEKIGMGPLINQAALDKVAGMVDRAVKAGGKITAGGHKLNIENGFYYEPTVITNVKQDSEIVQDEIFGPVLPVLTFKTLDDAIEMANDSDFGLTSSIFTENLDNAMRAANELEDGETYINRFNFEAMNGSHSGWKESGIGGDDGRHGIEEYLNTHVIYLQGHPEKANG

SEQ ID NO: 33 - DNA, 972 bp, Lactobacillus helveticus
atggcaagagaggaaaaacctcgtaaagttattttagtcggtgatggtgctgtaggttctacctttgcattttcaatggtacaacaaggtatcgctgaagaattaggtattatcgatatcgctaaggaacacgttgaaggtgacgcaatcgatttagctgacgcaactccttggacttctccaaagaacatttacgcagctgactacccagattgtaaggatgctgacttagttgttattactgctggtgctccacaaaagccaggcgaaactcgtcttgatcttgttaacaagaacttgaagattttatcatcaatcgttgaaccagttgttgaatcaggttttgaaggtattttcttagtagttgctaacccagttgatatcttaactcacgcaacttggagaatgtcaggcttccctaaggatcgtgttatcggttcaggtacttcacttgatactggtcgtcttcaaaaagttattggtaaaatggaaaacgttgacccaagttcagttaatgcatacatgcttggtgaacacggtgatactgaattcccagcatggagctacaacaatgttgctggcgtaaaggttgctgactgggttaaggctcacaacatgcctgaatctaagcttgaagacatccaccaagaagttaaggacatggcttacgacattattaacaagaaaggtgctaccttctacggtatcggtactgcttcagcaatgatcgctaaggctatcttgaacgatgaacaccgtgtacttccactttcagtaccaatggatggtgaatatggtttacacgatcttcacatcggtactcctgcagttgttggccgcaagggtcttgaacaagttatcgaaatgccattaagcgataaggaacaagaattaatgactgcttcagcagatcaattaaagaaggttatggacaaggccttcaaagaaactggcgttaaggttcgtcaataa

SEQ ID NO: 34 - protein, 323 aa, Lactobacillus helveticus
MAREEKPRKVILVGDGAVGSTFAFSMVQQGIAEELGIIDIAKEHVEGDAIDLADATPWTSPKNIYAADYPDCKDADLVVITAGAPQKPGETRLDLVNKNLKILSSIVEPVVESGFEGIFLVVANPVDILTHATWRMSGFPKDRVIGSGTSLDTGRLQKVIGKMENVDPSSVNAYMLGEHGDTEFPAWSYNNVAGVKVADWVKAHNMPESKLEDIHQEVKDMAYDIINKKGATFYGIGTASAMIAKAILNDEHRVLPLSVPMDGEYGLHDLHIGTPAVVGRKGLEQVIEMPLSDKEQELMTASADQLKKVMDKAFKETGVKVRQ

SEQ ID NO: 35 - DNA, 957 bp, Bacillus megaterium
atgaaaacacaatttacaccaaaaacacgaaaagttgccgttatcggaactggttttgttggctcaagctacgctttttcaatggtgaatcaaggtattgccaatgaattagtgttaatcgatatgaacaaagaaaaagcagaaggtgaagcacgtgatatcaatcatggaatgccatttgccacaccgatgaaaatctgggctggagattataaagactgtgctgacgctgatttagcagttattacagcgggcgctaatcaagctccaggggaaacacgcttagatctagttgaaaaaaacgttaaaattttcgaatgcattgtaaaagatattatgaacagcggatttgacggcatcattttagtggcaacaaatccagttgatattctcgcacacgttacacaaaaagtatcaggattaccaaacggacgggtaattggttcaggaacgattcttgacacagctcgcttccgctacttgttaagcgactatttcgaagtagattctcgcaacgtccacgcttatattatgggggaacatggagatacggaatttcctgtttggagccacgcgcaaattggcggtgtgaagctcgaacattttatcaatactgccgctattgaaaaagaaccggatatgcagcatctattcgaacaaacccgcgatgcggcttaccatattattaatcgaaaaggagcgacttattacggaattgcaatggggcttgtacgcattaccaaggctattttagatgatgaaaattctattttaacagtatctgctttattagaaggacaatacggtatttctgatgtgtatatcggcgtaccagctatcattaataaaaacggcgtgcgtcaaattattgaattgaatttaactcctcacgaacagcagcagctcgagcactctgctagcattcttaagcaaactcgcgacagagcttttgtgtaa

SEQ ID NO: 36 - protein, 318 aa, Bacillus megaterium
MKTQFTPKTRKVAVIGTGFVGSSYAFSMVNQGIANELVLIDMNKEKAEGEARDINHGMPFATPMKIWAGDYKDCADADLAVITAGANQAPGETRLDLVEKNVKIFECIVKDIMNSGFDGIILVATNPVDILAHVTQKVSGLPNGRVIGSGTILDTARFRYLLSDYFEVDSRNVHAYIMGEHGDTEFPVWSHAQIGGVKLEHFINTAAIEKEPDMQHLFEQTRDAAYHIINRKGATYYGIAMGLVRITKAILDDENSILTVSALLEGQYGISDVYIGVPAIINKNGVRQIIELNLTPHEQQQLEHSASILKQTRDRAFV

SEQ ID NO: 37 - DNA, 1179 bp, Clostridium acetobutylicum
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 38 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 39 - DNA, 702 bp, Bacillus subtilis
atgggaaaaggaaaagtgatggactcttatcaagatgctgctgcactgatcaaggacggagacaccctcatcgcaggaggcttcggcctgtgcggcattccggaacaattaattctcgcgatcagggacagcggcgtaaaaaacttgaccgttgtcagtaataactgcggtgttgatgattgggggttaggcctcttgcttgcaaaccggcagatcaaaaaaatggtcgcatcctatgtgggagaaaacaaaacctttgaacggcagtttttaagcggggagctggaagtcgaattggtgccccaaggaacgctcgcagaaagaattcgcgcgggaggggcgggcattccggcgttttacacacctgcgggcgtcggaacatcagtagccgagggaaaagaacataagacatttgacggacgcacctatcttctggagaaagggattacagggaacgccgccatcgtaaaggcctggaaagccgatcctctgggcaacctgatgttcagaaagacggcgagaaatttcaatccgctcgctgcaatggcaggcaaagtgacgattgcagaggctgaagaaatcgtcgatgccggagagcttgatccggatcaaattcatacgccgggtatttttgtgcagcacgtgctgctcggcgggattcatgaaaaacggattgaacgccgcaccgtccggcaagcatga

SEQ ID NO: 40 - protein, 233 aa, Bacillus subtilis
MGKGKVMDSYQDAAALIKDGDTLIAGGFGLCGIPEQLILAIRDSGVKNLTVVSNNCGVDDWGLGLLLANRQIKKMVASYVGENKTFERQFLSGELEVELVPQGTLAERIRAGGAGIPAFYTPAGVGTSVAEGKEHKTFDGRTYLLEKGITGNAAIVKAWKADPLGNLMFRKTARNFNPLAAMAGKVTIAEAEEIVDAGELDPDQIHTPGIFVQHVLLGGIHEKRIERRTVRQA

SEQ ID NO: 41 - DNA, 651 bp, Bacillus subtilis
gtgaaggaagcgagaaaacgaatggtcaaacgggctgtacaagaaatcaaggacggcatgaatgtgaatctcgggattggaatgccgacgcttgtcgcaaatgagatacccgatggcgttcacgtcatgcttcagtcggaaaacggcttgctcggaattggcccctatcctctggaaggaacggaagacgcggatttgatcaatgcgggaaaggaaacgatcactgaagtgacaggcgcctcttattttgacagcgctgagtcattcgcgatgataagaggcgggcatatcgatttagctattctcggcggaatggaggtttcggagcagggggatttggccaattggatgatcccgggcaaaatggtaaaagggatgggcggcgccatggatctcgtcaacggggcgaaacgaatcgttgtcatcatggagcacgtcaataagcatggtgaatcaaaggtgaaaaaaacatgctcccttccgctgacaggccagaaagtcgtacacaggctgattacggatttggctgtatttgattttgtgaacggccgcatgacactgacggagcttcaggatggtgtcacaattgaagaggtttatgaaaaaacagaagctgatttcgctgtaagccagtctgtactcaattcttaa

SEQ ID NO: 42 - protein, 216 aa, Bacillus subtilis
  MISC_FEATURE (1)..(1): X=XAA ANY AMINO ACID
XKEARKRMVKRAVQEIKDGMNVNLGIGMPTLVANEIPDGVHVMLQSENGLLGIGPYPLEGTEDADLINAGKETITEVTGASYFDSAESFAMIRGGHIDLAILGGMEVSEQGDLANWMIPGKMVKGMGGAMDLVNGAKRIVVIMEHVNKHGESKVKKTCSLPLTGQKVVHRLITDLAVFDFVNGRMTLTELQDGVTIEEVYEKTEADFAVSQSVLNS

SEQ ID NO: 43 - DNA, 714 bp, Bacillus mojavensis
atgggaaaagtgctgtcatcgagtaaggaagccgcggaactcattcgggagggggatacactgatcgcgggcggattcggcctgtgcggaattcccgagcagctcattctggcgataagggataaaggggtaaaagatttaaccgtcgtcagcaataattgcggagttgatgattgggggctcggtctgctgctggcaaacaagcaaatcaaaaaaatgatcgcttcctacgtcggagaaaacaaaatttttgaaaagcaatttttaagcggagaattggaagtggaattggttccccaagggaccctcgctgaaagaatccgagccggaggagcgggtataccgggattttacacagccacaggcgtcggaacatctatcgctgacgggaaagagcataaaacctttgacggacgcacttatgtgttagaaaaagggattactggggatgtcgccattgtaaaagcatggaaagcggacaccatggggaatttagtttttcggaaaacggcaagaaatttcaatccggttgccgccatggcgggaaagatcacaattgccgaggcagaagaaattgttgaggcgggagagctcgatcccgaccacatacacacgcctggtatttacgtacagcatgttgtgctcggcacacatgaaaagcggattgaaaaacgaactgttcagcaagcggagggaaaggaggcggcacaatga

SEQ ID NO: 44 - protein, 237 aa, Bacillus mojavensis
MGKVLSSSKEAAELIREGDTLIAGGFGLCGIPEQLILAIRDKGVKDLTVVSNNCGVDDWGLGLLLANKQIKKMIASYVGENKIFEKQFLSGELEVELVPQGTLAERIRAGGAGIPGFYTATGVGTSIADGKEHKTFDGRTYVLEKGITGDVAIVKAWKADTMGNLVFRKTARNFNPVAAMAGKITIAEAEEIVEAGELDPDHIHTPGIYVQHVVLGTHEKRIEKRTVQQAEGKEAAQ

SEQ ID NO: 45 - DNA, 657 bp, Bacillus mojavensis
atgaaggaagccagaaaacgaatggtcaaacgtgctgtaaaggaaataaaagacggtatgaacgtcaatcttgggatagggatgccgacacttgtggcaaatgaaataccggagggcgttcatgtgatgcttcaatcagaaaacggcttgcttgggatcggcccgtatccgctggacggaacggaagacccggatctgatcaatgcggggaaagaaacgatcaccgccgtaacaggcgcatcctattttgacagcgcagaatcctttgcgatgatacgaggcggtcatatcgacctggctatcctcgggggcatggaggtttctgagcaaggggatttggcgaactggatgatcccggggaaaatggtgaagggaatgggcggcgctatggatttggtcaacggggctaagcgaatcgttgtcatcatggagcacgtcaataaacatggggaatcgaaggtgaaaaaacaatgctccctcccgctgacaggacagaaagtcgttcatcggctgatcactgatttagctgtttttgattttgataacggccatatgacactgactgagctccaggacggcgtcacgctggaagaggtatatgagaaaactgaagctgacttcgccgtaagccagtcagtcatccggcaaaaatcttaa

SEQ ID NO: 46 - protein, 218 aa, Bacillus mojavensis
MKEARKRMVKRAVKEIKDGMNVNLGIGMPTLVANEIPEGVHVMLQSENGLLGIGPYPLDGTEDPDLINAGKETITAVTGASYFDSAESFAMIRGGHIDLAILGGMEVSEQGDLANWMIPGKMVKGMGGAMDLVNGAKRIVVIMEHVNKHGESKVKKQCSLPLTGQKVVHRLITDLAVFDFDNGHMTLTELQDGVTLEEVYEKTEADFAVSQSVIRQKS

SEQ ID NO: 47 - DNA, 741 bp, Clostridium beijerinckii
atgttagaaagtgaagtatctaaacaaattacaactccacttgctgctccagcgtttcctagaggaccatataggtttcacaatagagaatatctaaacattatttatcgaactgatttagatgctcttcgaaaaatagtaccagagccacttgaattagatagagcatatgttagatttgaaatgatggctatgcctgatacaaccggactaggctcatatacagaatgtggtcaagctattccagtaaaatataatggtgttaagggtgactacttgcatatgatgtatctagataatgaacctgctattgctgttggaagagaaagtagcgcttatccaaaaaagcttggctatccaaagctatttgttgattcagatactttagttgggacacttaaatatggtacattaccagtagctactgcaacaatgggatataagcacgagcctctagatcttaaagaagcctatgctcaaattgcaagacccaattttatgctaaaaatcattcaaggttacgatggtaagccaagaatttgtgaactaatatgtgcagaaaatactgatataactattcacggtgcttggactggaagtgcacgtctacaattatttagccatgcactagctcctcttgctgatttacctgtattagagattgtatcagcatctcatatcctcacagatttaactcttggaacacctaaggttgtacatgattatctttcagtaaaataa

SEQ ID NO: 48 - protein, 246 aa, Clostridium beijerinckii
MLESEVSKQITTPLAAPAFPRGPYRFHNREYLNIIYRTDLDALRKIVPEPLELDRAYVRFEMMAMPDTTGLGSYTECGQAIPVKYNGVKGDYLHMMYLDNEPAIAVGRESSAYPKKLGYPKLFVDSDTLVGTLKYGTLPVATATMGYKHEPLDLKEAYAQIARPNFMLKIIQGYDGKPRICELICAENTDITIHGAWTGSARLQLFSHALAPLADLPVLEIVSASHILTDLTLGTPKVVHDYLSVK

SEQ ID NO: 49 - DNA, 1056 bp, Clostridium beijerinckii
atgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagacaagccaaaagacttaattaaagcagtagttatattataa

SEQ ID NO: 50 - protein, 351 aa, Clostridium beijerinckii
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

SEQ ID NO: 51 - DNA, 1059 bp, Thermoanaerobacter ethanolicus
atgaaaggttttgcaatgctcagtatcggtaaagtcggttggattgaaaaagaaaagcctactcccggcccttttgacgctatcgtaagacctctagctgtggccccttgcacttcggacgttcataccgtttttgaaggtgctattggcgaaagacataacatgatactcggtcacgaagctgtaggtgaagtagttgaagtaggtagtgaggtaaaagattttaaacctggtgatcgcgttgttgtaccagctattacccctgattggcgaacctctgaagtacaaagaggatatcaccaacactctggtggaatgctggcaggctggaaattttcgaatataaaagatggtgtttttggtgaattttttcatgtgaacgatgctgatatgaatttagcacatctgcctaaggaaattccattggaagctgcagttatgattcccgatatgatgactactggctttcacggagccgaactggcagaaatagaattaggtgcaacggtagcggttttgggtattggtccagtaggtcttatggcagtcgctggtgccaaattgcggggtgctggaagaattattgcagtaggcagtagacctgtttgtgtagatgctgcaaaatactatggagctactgatattgtaaactataaaaatggtcctatcgaaagccagattatggatttaacggaaggcaaaggtgttgatgctgccatcatcgctggaggaaatgctgacattatggctacagcagttaagattgttaaaccaggcggcaccatcgctaatgtaaattattttggcgaaggagatgttctgcctgttcctcgtcttgaatggggttgcggcatggctcataaagctataaaaggcggtttatgccctggtggacgtctaagaatggaaagactgattgaccttgttttttataagcgtgtcgatccttccaaactcgtcactcatgtttttcaaggatttgataatattgaaaaagctctaatgctgatgaaagataaaccaaaagacctaatcaaacctgttgtaatattagcataa

SEQ ID NO: 52 - protein, 352 aa, Thermoanaerobacter ethanolicus
MKGFAMLSIGKVGWIEKEKPTPGPFDAIVRPLAVAPCTSDVHTVFEGAIGERHNMILGHEAVGEVVEVGSEVKDFKPGDRVVVPAITPDWRTSEVQRGYHQHSGGMLAGWKFSNIKDGVFGEFFHVNDADMNLAHLPKEIPLEAAVMIPDMMTTGFHGAELAEIELGATVAVLGIGPVGLMAVAGAKLRGAGRIIAVGSRPVCVDAAKYYGATDIVNYKNGPIESQIMDLTEGKGVDAAIIAGGNADIMATAVKIVKPGGTIANVNYFGEGDVLPVPRLEWGCGMAHKAIKGGLCPGGRLRMERLIDLVFYKRVDPSKLVTHVFQGFDNIEKALMLMKDKPKDLIKPVVILA

SEQ ID NO: 53 - DNA, 1146 bp, Salmonella enterica
atgagctttatgttggcactgccaaaaattagcctgcatggcgctggagcgattgccgatatggtgaacctcgtggcgaataagcaatggggcaaagcgctgatcgttaccgatggtcaactggtaaagctgggcctgctggatagcctgtttagcgcactggatgaacatcaaatgtcttaccacctgtttgatgaggtgttcccaaatccgacagaagagctggtgcagaaaggttttgcggcgtatcagagcgcagaatgtgactacattatcgccttcggcggcggcagcccgatcgataccgcaaaagcggtgaaaatcctcaccgccaaccctggcccttccaccgcctactccggcgtcggcaaagtgaaaaacgccggcgtaccgctggtagcaattaatactacggcaggtaccgcggccgagatgaccagcaacgcggtgattatcgactctgcccgtaaggtcaaagaggtgatcatcgacccgaatatcatcccggatattgccgtggatgacgccagcgtcatgctggaaatcccagcgtctgtgaccgccgcaaccggcatggatgccctgacccatgcggtggaagcctatgtctccgttggcgcgcatccgctcaccgacgctaacgcgctggaagcgattcgcttaatcaatgtgtggctaccgaaagcggttgacgatggccataacctcgaagcccgtgagcaaatggcattcggccagtacctggcgggtatggcgttcaacagcgccggtttggggctggttcacgcgctggcgcaccagccgggcgcaacccacaatctgccgcacggcgtctgcaacgctatcctgctgccaattgtcgagaactttaaccgtccgaacgccgtcgcgcgttttgcccgcatcgcacaggcgatgggcgtggagacgcgcggtatgtctgacgaagccgcaagtcaggaagcgattaacgctatccgtacgctgagcaaacgcgtcggcattccagaaggtttcagcaagctgggcgtcacaaaagaagatatcgaaggctggctggataaagccctcgccgacccgtgcgcgccgtgtaacccgcgcaccgccagccgcgatgaagttcgcgagctgtacctggaggcgtta

SEQ ID NO: 54 - protein, 382 aa, Salmonella enterica
MSFMLALPKISLHGAGAIADMVNLVANKQWGKALIVTDGQLVKLGLLDSLFSALDEHQMSYHLFDEVFPNPTEELVQKGFAAYQSAECDYIIAFGGGSPIDTAKAVKILTANPGPSTAYSGVGKVKNAGVPLVAINTTAGTAAEMTSNAVIIDSARKVKEVIIDPNIIPDIAVDDASVMLEIPASVTAATGMDALTHAVEAYVSVGAHPLTDANALEAIRLINVWLPKAVDDGHNLEAREQMAFGQYLAGMAFNSAGLGLVHALAHQPGATHNLPHGVCNAILLPIVENFNRPNAVARFARIAQAMGVETRGMSDEAASQEAINAIRTLSKRVGIPEGFSKLGVTKEDIEGWLDKALADPCAPCNPRTASRDEVRELYLEAL

SEQ ID NO: 55 - DNA, 1146 bp, Clostridium butyricum
atggtaaatagaattgtattaaatgaaacatcatatcatggagcaggggctataaatgaaatagtaactgaggtaaaagacagaggattaaacaaagcgtttttatgttcagatccagatttggttaagtttggggtaactagaaagattacatctgtactagaaagcgcaggtctaaactatgaattatattcaaatataaaacctaatccaacaattgaaaacgttaaaagtggggtagcagcttacaaaaactctggtgcagattatataattgctgttggaggaggctcatcaatggatacagctaaggctgttggaataataataaacaatccagaatttggagatgtagttagtttagaaggtgtagctccaactaaaaataaatgtgtgccaattattgctgttccaacaacagctggtacagccgcagaagtcactattaattatgtcatcacagatgttgagaaaaatagaaaatttgtatgtgtagatacacatgatataccagtagtagcaattattgatcctgagatgatggctagtatgccaagaggattaacagcagcaacaggaatggatgctttaactcatgctatagaaggatttacaactagtgcagcatgggaaatgacagatatgtttcatttaaaggctattgaattaatttctaaaaatttaagaggtgctgtagataatactttagaaggaagagagggaatggcactcggtcaatatattgctggaatgggattttcaaatgtaggtcttggaatagtacattctatggctcattcacttggagcggtatatgatacaccacacggtgttgcaaatgcaataatacttccaactgttatgaaatataatgaagaatcaactggagataaatataaatacattgcagcagctatgggagtggagaatactgaaaaaatgactgttgaagagtatagaaaagcagctatagatgcagttaagaagctttcaaaggatgttggaattccagaaaatttatcacaaatagtaaaagaggaggatataagatttttatcagaatcagcttatgcagatgcatgtagacctggaaatccaaaggaaacaaatgtgaatgatatatcagaattatataaatcattaata

SEQ ID NO: 56 - protein, 382 aa, Clostridium butyricum
MVNRIVLNETSYHGAGAINEIVTEVKDRGLNKAFLCSDPDLVKFGVTRKITSVLESAGLNYELYSNIKPNPTIENVKSGVAAYKNSGADYIIAVGGGSSMDTAKAVGIIINNPEFGDVVSLEGVAPTKNKCVPIIAVPTTAGTAAEVTINYVITDVEKNRKFVCVDTHDIPVVAIIDPEMMASMPRGLTAATGMDALTHAIEGFTTSAAWEMTDMFHLKAIELISKNLRGAVDNTLEGREGMALGQYIAGMGFSNVGLGIVHSMAHSLGAVYDTPHGVANAIILPTVMKYNEESTGDKYKYIAAAMGVENTEKMTVEEYRKAAIDAVKKLSKDVGIPENLSQIVKEEDIRFLSESAYADACRPGNPKETNVNDISELYKSLI

SEQ ID NO: 57 - DNA, 738 bp, Lactobacillus diolivorans
atgaaggatactgatacgccaatttcatttggcaagaaggattcttctgcaaaccacgaatctaatcaacttgcattgcatggagacagctggtttaagtcgggtgatgatccggtttcaaattcatcatctgcgaccgcaacagcaagtgcacccagccagtccacatcaggaaatggtgatattaagagccttgattggttcaagcatgttggggttgcaaaaccaggattatcaaaagatgaagttgttattggggttgcacctgcatttgctgaagtgctaactaagacaatgactaaaatccctcataaagatgtcttacgtcaaattattgccggaattgaagaggaaggtctaaaagcccgggttgttaaagtttaccggacatcagatgtttcatttattgctgatgaggttgataaactttcaggttccggaatcgcagttgctgttcaatcaaaaggaactgctattattcaccaaaaggatcaggaacctctaaataacttggaattgttcccacaggcaccagtgattgatttgccaacttatcgggcaatcggtaagaacgcagctcaatatgccaagggattatctccagacccggttccaacagttaatgatcaaatggctcgtgttcaatacactgccttatcatcattgatgcatattgctgaaacgaaacgggtggttgttggtaagcctgccgaagaaattcaagtaacctttaattga

SEQ ID NO: 58 - protein, 245 aa, Lactobacillus diolivorans
MKDTDTPISFGKKDSSANHESNQLALHGDSWFKSGDDPVSNSSSATATASAPSQSTSGNGDIKSLDWFKHVGVAKPGLSKDEVVIGVAPAFAEVLTKTMTKIPHKDVLRQIIAGIEEEGLKARVVKVYRTSDVSFIADEVDKLSGSGIAVAVQSKGTAIIHQKDQEPLNNLELFPQAPVIDLPTYRAIGKNAAQYAKGLSPDPVPTVNDQMARVQYTALSSLMHIAETKRVVVGKPAEEIQVTFN

SEQ ID NO: 59 - DNA, 543 bp, Lactobacillus diolivorans
atgagcgaagttgatgaattagtttctaaaattctgagtcaactaaataatggtgatagctcacagtcaaccggaacaaccactgcttcatcggtaacaccgacaagtagtcctgcaaattccggtaagacgtttgataagggcgattatccactattcagaaagcacccagatgatgttaaaacgccaacgggcaaagcagtaagtgatattacacttgataacgttgttagcggcaaggttgattctaaagatttacgaattaccgcaaatactttgagacgccaaggcgaaattgctgccagtgccggccgtccagctattcaacgaaactttcaacgagccgctgaactaactaagattccggatgacaaagtgctatctttctatcatgcattgcgaccattccgatcaagtaaacaggacttactggatattgctaagcaactccgtgacacctatcacgcaccagtttgtgcaaattggtttgaagaagctgccggtaattacgaaatcagcaagaagttgaagggtgataactag

SEQ ID NO: 60 - protein, 180 aa, Lactobacillus diolivorans
MSEVDELVSKILSQLNNGDSSQSTGTTTASSVTPTSSPANSGKTFDKGDYPLFRKHPDDVKTPTGKAVSDITLDNVVSGKVDSKDLRITANTLRRQGEIAASAGRPAIQRNFQRAAELTKIPDDKVLSFYHALRPFRSSKQDLLDIAKQLRDTYHAPVCANWFEEAAGNYEISKKLKGDN

SEQ ID NO: 61 - DNA, 1122 bp, Lactobacillus reuteri
atggaaaaatatagtatgccaacccggatttattcgggaacagatagtttgaaagaactagagacacttaataatgaacgtattttattagtctgtgattctttcttgcctggtagtgataccttaaaagaaattgagagtcacattaaggataataataagtgtgaaattttctctgatgttgtccccgatcctccactagataagattatggaaggggttcaacaattccttaaacttaaaccaacaattgtgattggtatcggtggcggatcagctttggatactggtaagggaattcgtttctttggtgaaaagttgggcaagtgcaagatcaatgaatatattgctattccaacaacgagtggtactggttcagaagttacgaatactgcggttatttctgatacgaaagaacatcgtaaaattcctattttggaagattatttgacacctgattgtgctttactagatcctaaactagttatgactgctcctaagagtgtaactgcatattcaggaatggatgttttaacacatgcacttgaatctttggttgctaaggatgcaaatttattcacagttgcattatcagaagaagcaattgatgccgttattaaacatttagttgagtgttatcgtcacggcgataatgtggatgctcgtaagattgttcatgaagcatcaaatattgccggaactgcatttaatattgctggattagggatttgccactcaattgcgcatcaattgggagctaatttccacgttccccatggtttagcaaatacaatgctcttgccatatgttatcgcatataatgctgaacatagtgaagaggcattgcataagtttgcaattgctgctaagaaagctggaattgctgctcctggagtaggcgatcgtcttgcagtaaagcgactaattgctaaaattagggaaatggcacgacaaatgaattgtccaatgactcttcaagcattcggtgttgatcctgctaaagctgaagaattagctgatactgttgttgcaaatgcgaagaaagatgcaacattccctggcaatccagttgttccttcagataatgatctgaagatggtttacgaagcaataattcgttaa

SEQ ID NO: 62 - protein, 373 aa, Lactobacillus reuteri
MEKYSMPTRIYSGTDSLKELETLNNERILLVCDSFLPGSDTLKEIESHIKDNNKCEIFSDVVPDPPLDKIMEGVQQFLKLKPTIVIGIGGGSALDTGKGIRFFGEKLGKCKINEYIAIPTTSGTGSEVTNTAVISDTKEHRKIPILEDYLTPDCALLDPKLVMTAPKSVTAYSGMDVLTHALESLVAKDANLFTVALSEEAIDAVIKHLVECYRHGDNVDARKIVHEASNIAGTAFNIAGLGICHSIAHQLGANFHVPHGLANTMLLPYVIAYNAEHSEEALHKFAIAAKKAGIAAPGVGDRLAVKRLIAKIREMARQMNCPMTLQAFGVDPAKAEELADTVVANAKKDATFPGNPVVPSDNDLKMVYEAIIR

SEQ ID NO: 63 - DNA, 29 bp, Escherichia coli
gaagatctatggtgcaaaacctttcgcgg

SEQ ID NO: 64 - DNA, 27 bp, Escherichia coli
aaaagtactcaaccaagtcattctgag

SEQ ID NO: 65 - DNA, 2637 bp, Lactobacillus reuteri
atgcctgctaacaacaagaaacaagttgaaaagaaagaattaactgctgaagaaaaaaagcaaaacgcccaaaagctagttgacgatttaatgactaagagtcaagctgcttttgaaaagttacgttactattcacaagaacaagttgacaagatttgtcaggcaatggctctcgctgccgaagaacaccacatggacttagctgttgatgctgctaacgaaactggtcgtggggttgctgaagataaggctatcaagaacatctacgcaagtgaatacatttggaacaacatccgtcacgataagactgttggtattatcgaagacaatgatgaagaccaaactatcaaaattgctgatccacttggtgtcattgccggaattgttccagttactaaccctacttcaacaacgatcttcaaatcaatcattagtgctaagacacggaatacaatcatcttttctttccaccgtcaagcaatgaagtcatctatcaagactgcaaagattctccaagaagctgctgaaaaagccggtgcgccaaagaacatgattcaatggctccctgaaagtacccgcgaaaacactaccgcattactccaacaccctaatactgctactattttagcaaccggtggtccttcattagttaaggctgcctacagttctggtaaccctgctcttggtgttggtcctggtaacggtcctgcttacatcgaaaaaactgccaacatcgaacgttctgtttacgacatcgttctttctaagacattcgataacggtatgatttgtgccactgaaaactcagttgttgttgatgaagaaatctacgacaaggttaaagaagaattccaaaagtggaactgttacttcttgaagccaaacgaaattgataaatttactgatggctttattgacccagatcgtcatcaagttcgtggtccaatcgctggtcgttcagctaatgctattgctgacatgtgtggtattaaagtacctgacaacactaaggttatcattgctgaatacgaaggtgttggtgacaagtacccactttcagctgaaaagctttcaccagtattaacaatgtacaaggcaacctctcacgaaaatgcctttgatatctgtgctcaattattacactacggtggtgaaggtcacactgctgctattcacacccttgatgatgatttagctactaagtacggtcttgaaatgcgtgcttcacggatcattgttaactccccatctggtattggtggtattggtaacatctacaacaacatgactccatcccttactttaggtactggttcatacggtagtaactcaatttctcacaacgttactgattgggacctcttaaacatcaaaacaattgcaaagcggcgtgaaaaccgtcaatgggttaagattcccccaaaagtatactttcaacgcaactcactaaaagaattgcaagatattccaaacattaaccgggcattcatcgttactggtcctggaatgagcaagcgtggttacgttcaacgtgttatcgatcaattgcgtcaacgccaaaacaacactgctttcttagtatttgatgacgttgaagaagatccatcaacaaacactgttgaaaaaggtgttgccatgatgaatgacttcaaacctgatacaattattgctcttggtggtggttcaccaatggatgctgctaaggctatgtggatgttctatgagcacccagaaacttcatggtatggggttatgcaaaagtaccttgatattcggaagcgtgcttaccaaatcaagaagcctactaagtctcaacttattggtatccctactacatcaggtactggttcagaagttactccatttgcggttattaccgattcaaaaactcatgttaagtacccacttgctgactacgccttaacaccaaacattgcaatcgttgactcacaattcgttgaaactgttccagcaaaaactactgcttggactggactagatgttttatgtcacgctactgaatcatatgtttctgttatggcaactgactacactcgtggttggtcactacaaaccatcaagggtgttatggaaaaccttcctaagtcagttcaaggtgataagttagctcgtcgtaagatgcacgacttctcaacaatggccgggatggcatttggtcaagccttcttaggaattaaccactcccttgcccacaagatgggtggagcattcggtcttcctcacggtttgcttatcgctattgcaatgccacaagtaattcgctttaacgcaaaacgtccacaaaagcttgctctctggcctcactatgagacttaccatgcaactaaggactacgctgacattgcacggttcattggtttgaaaggcaacactgatgaagaattagctgaagcatatgctaagaaagttatcgaacttgctcacgaatgtggtgttaagcttagtcttaaggacaatggtgttacacgtgaagaatttgataaggcggttgacgatcttgctcgcttagcttacgaagatcaatgtactactactaacccagttgaaccacttgttagccaactcaaggaattacttgaacgttgctacgatggtactggcgttgaagaaaaataa

SEQ ID NO: 66 - protein, 878 aa, Lactobacillus reuteri
MPANNKKQVEKKELTAEEKKQNAQKLVDDLMTKSQAAFEKLRYYSQEQVDKICQAMALAAEEHHMDLAVDAANETGRGVAEDKAIKNIYASEYIWNNIRHDKTVGIIEDNDEDQTIKIADPLGVIAGIVPVTNPTSTTIFKSIISAKTRNTIIFSFHRQAMKSSIKTAKILQEAAEKAGAPKNMIQWLPESTRENTTALLQHPNTATILATGGPSLVKAAYSSGNPALGVGPGNGPAYIEKTANIERSVYDIVLSKTFDNGMICATENSVVVDEEIYDKVKEEFQKWNCYFLKPNEIDKFTDGFIDPDRHQVRGPIAGRSANAIADMCGIKVPDNTKVIIAEYEGVGDKYPLSAEKLSPVLTMYKATSHENAFDICAQLLHYGGEGHTAAIHTLDDDLATKYGLEMRASRIIVNSPSGIGGIGNIYNNMTPSLTLGTGSYGSNSISHNVTDWDLLNIKTIAKRRENRQWVKIPPKVYFQRNSLKELQDIPNINRAFIVTGPGMSKRGYVQRVIDQLRQRQNNTAFLVFDDVEEDPSTNTVEKGVAMMNDFKPDTIIALGGGSPMDAAKAMWMFYEHPETSWYGVMQKYLDIRKRAYQIKKPTKSQLIGIPTTSGTGSEVTPFAVITDSKTHVKYPLADYALTPNIAIVDSQFVETVPAKTTAWTGLDVLCHATESYVSVMATDYTRGWSLQTIKGVMENLPKSVQGDKLARRKMHDFSTMAGMAFGQAFLGINHSLAHKMGGAFGLPHGLLIAIAMPQVIRFNAKRPQKLALWPHYETYHATKDYADIARFIGLKGNTDEELAEAYAKKVIELAHECGVKLSLKDNGVTREEFDKAVDDLARLAYEDQCTTTNPVEPLVSQLKELLERCYDGTGVEEK

SEQ ID NO: 67 - DNA, 1434 bp, Lactobacillus reuteri
atgcagattaatgatattgaaagtgctgtacgcaaaattcttgccgaagaactagataatgccagctcttcaagtgcaaacgttgcagctactactgataatggtcatcgcggaattttcactaatgtcaatgatgcaattgctgctgcaaaagctgctcaagaaatatatcgggataagccaattgctgttcgccaacaagtgattgatgccattaaggaaggattccgcccatatattgaaaaaatggctaaagatatcaaagaagaaacaggaatgggaacagtagaggccaaaattgctaagttaaacaatgccttgtacaacactcctggtcccgagattcttgaaccagttgtagaaaacggtgacggtgggatggttatgtatgaacggttaccatatggtgttattggtgcggttggcccaagtacaaacccttcagaaactgtaattgctaatgcgatcatgatgcttgccggtggtaatactctttactttggtgctcaccctggcgcaaagaatgttactcgctggacaattgaaaagatgaacgattttattgcagatgcaacaggccttcataatttagttgtaagtattgaaacaccaacaattgaatcagttcaacaaatgatgaagcaccccgacattgcaatgttagcagtaactggtggcccagctgttgttcaccaagcaatgaccagtggtaagaaagcggttggtgctggtcctggtaatcctcctgcaatggttgatgctactgctgatattgatttagctgctcataatatcattacatctgcttcatttgataatgatattttatgtactgctgaaaaggaagtagttgcagaaagtagcattaaagatgaattaattcgtaagatgcaagatgaaggtgcctttgtagttaaccgtgaacaagccgataaattagctgatatgtgtatccaagaaaatggtgctcctgatcgtaaatttgttggtaaggatgcaacttatatcttagaccaagctaatattccttacacaggccacccagttgaaattatttgtgaacttcctaaggaacatccattagtaatgactgaaatgttaatgccaattttaccagttgtttcttgtccaacatttgatgatgttttgaagactgctgttgaagttgaaaaaggtaaccatcacacagctactattcattccaataaccttaagcatattaataatgctgctcaccggatgcaatgttcaatctttgttgttaatggcccatcctatgttggtacaggtgttgcagataatggagctcactcaggtgcttcagcattaacaattgctacgccaactggtgaaggaacatgtactgcacgaacatttactcgtcgggttcgtttgaactcaccacaaggattctcagtacgtaactggtattaa

SEQ ID NO: 68 - protein, 477 aa, Lactobacillus reuteri
MQINDIESAVRKILAEELDNASSSSANVAATTDNGHRGIFTNVNDAIAAAKAAQEIYRDKPIAVRQQVIDAIKEGFRPYIEKMAKDIKEETGMGTVEAKIAKLNNALYNTPGPEILEPVVENGDGGMVMYERLPYGVIGAVGPSTNPSETVIANAIMMLAGGNTLYFGAHPGAKNVTRWTIEKMNDFIADATGLHNLVVSIETPTIESVQQMMKHPDIAMLAVTGGPAVVHQAMTSGKKAVGAGPGNPPAMVDATADIDLAAHNIITSASFDNDILCTAEKEVVAESSIKDELIRKMQDEGAFVVNREQADKLADMCIQENGAPDRKFVGKDATYILDQANIPYTGHPVEIICELPKEHPLVMTEMLMPILPVVSCPTFDDVLKTAVEVEKGNHHTATIHSNNLKHINNAAHRMQCSIFVVNGPSYVGTGVADNGAHSGASALTIATPTGEGTCTARTFTRRVRLNSPQGFSVRNWY

SEQ ID NO: 69 - DNA, 90 bp, Artificial Sequence
  ARTIFICIAL DNA PRIMER
caagaaacaagttgaaaagaaagaattaactgctgaagaaaagctttaaaacgcccaaaagctagttgacgatttaatgactaagagtca

SEQ ID NO: 70 - DNA, 21 bp, Artificial Sequence
  ARTIFICIAL DNA PRIMER
agggtgttggagtaatgcggt

SEQ ID NO: 71 - DNA, 30 bp, Artificial Sequence
  ARTIFICIAL DNA PRIMER
agaaagaattaactgctgaagaaaagcttt

SEQ ID NO: 72 - DNA, 30 bp, Artificial Sequence
  ARTIFICIAL DNA PRIMER
tgaatgatagtgattatgacgttaaagatc

SEQ ID NO: 73 - protein, 19 aa, Artificial Sequence
  ARTIFICIAL PRIMER
MAGNSSNFIHKIKQIFTHR

SEQ ID NO: 74 - DNA, 90 bp, Artificial Sequence
  ARTIFICIAL DNA PRIMER
gtagtagctgcaacgtttgcacttgaagagctggcattatcctaagcttcggcaagaattttgcgtacagcactttcaatatcattaatc

SEQ ID NO: 75 - DNA, 26 bp, Artificial Sequence
  ARTIFICIAL DNA PRIMER
acaactaaattatgaaggcctgttgc

SEQ ID NO: 76 - DNA, 24 bp, Artificial Sequence
  ARTIFICIAL DNA PRIMER
cgcaaaattcttgccgaagcttag

SEQ ID NO: 77 - DNA, 30 bp, Artificial Sequence
  ARTIFICIAL DNA PRIMER
ataatgcttctaaaaatctatttgatcggc

SEQ ID NO: 78 - DNA, 100 bp, Artificial Sequence
  ARTIFICIAL DNA PRIMER
gtgcgaagcttgtcgactcgagagcgctatagttgttgacagaatggacatactatgatatattgttgctatagcgcgagactattacaaggagatttta

SEQ ID NO: 79 - DNA, 32 bp, Artificial Sequence
  ARTIFICIAL DNA PRIMER
gtgcgaagcttccgactggaaagcgggcagtg

SEQ ID NO: 80 - DNA, 47 bp, Artificial Sequence
  ARTIFICIAL DNA PRIMER
cggggtttagttgttgacagggaggcttcgttgtgataagatggtag

SEQ ID NO: 81 - DNA, 47 bp, Artificial Sequence
  ARTIFICIAL DNA PRIMER
agcgctatagttgttgacagaatggacatactatgatatattgttgc

(19) United States
(12) Patent Application Publication
(10) Pub. No.: US 2014/0134691 A1
(43) Pub. Date: May 15, 2014

(54) Microorganisms for n-Propanol Production

(71) Applicant: Novozymes A/S, Bagsvaerd (DK)

(72) Inventors: Peter Olsen, Copenhagen (DK); Lars Christensen, Alleroed (DK); Steen Joergensen, Alleroed (DK); Torsten Regueira, Copenhagen (DK); Bjarke Christensen, Kgs. Lyngby (DK); Brian Kobmann, Herlev (DK); Thomas Grotkjaer, Frederiksberg (DK)

(73) Assignee: Novozymes A/S, Bagsvaerd (DK)

(21) Appl. No.: 14/118,856

(22) PCT Filed: May 25, 2012
(86) PCT No.: PCT/EP2012/059909

§ 371 Date: Jan. 27, 2014

Related U.S. Application Data
(60) Provisional application No. 61/490,989, filed on May 27, 2011.
(60) Provisional application No. 61/490,995, filed on May 27, 2011.

(51) Int. Cl. C12P 7/04; C12P 5/02

(52) U.S. Cl. CPC C12P 7/04; C12P 5/026
USPC 435/157; 435/252.3; 435/252.33; 435/167

(57) ABSTRACT

Described herein are host cells comprising lactate dehydrogenase activity, lactaldehyde dehydrogenase activity, lactaldehyde reductase activity, propanediol dehydratase activity, and/or n-propanol dehydrogenase activity, wherein the cells are capable of producing n-propanol. Also described are methods of producing n-propanol comprising (a) cultivating the host cells having lactate dehydrogenase activity, lactaldehyde dehydrogenase activity, lactaldehyde reductase activity, propanediol dehydratase activity, and/or n-propanol dehydrogenase activity in a medium under suitable conditions to produce n-propanol; and (b) recovering the n-propanol. Methods of producing polypropylene from the recombinant n-propanol are also provided.

### CROSS REFERENCE TO RELATED APPLICATIONS

This application claims priority benefit of U.S. Provisional Application No. 61/490,989, filed May 27, 2011, and U.S. Provisional Application No. 61/490,995, filed May 27, 2011, the entire contents of which are incorporated herein by reference.

### REFERENCE TO A SEQUENCE LISTING

This application contains a Sequence Listing in computer readable form, which is incorporated herein by reference.

### BACKGROUND

Concerns related to future supply of oil have prompted research in the area of renewable energy and renewable sources of other raw materials. Biofuels, such as ethanol and bioplastics (e.g., particularly polylactic acid) are examples of products that can be made directly from agricultural sources using microorganisms. Additional desired products may then be derived using non-enzymatic chemical conversions, e.g., dehydration of ethanol to ethylene.

Polymerization of ethylene provides polyethylene, a type of plastic with a wide range of useful applications. Ethylene is traditionally produced by refined non-renewable fossil fuels, but dehydration of biologically-derived ethanol to ethylene offers an alternative route to ethylene from renewable carbon sources, i.e., ethanol from fermentation of fermentable sugars. This process has been utilized for the production of “Green Polyethylene” that—save for minute differences in the carbon isotope distribution—is identical to polyethylene produced from oil.

Similarly, isopropanol and n-propanol can be dehydrated to propylene, which in turn can be polymerized to polypropylene. As with polyethylene, using biologically-derived starting material (i.e., isopropanol or n-propanol) would result in “Green Polypropylene.” However, unlike polyethylene, the production of the polyethylene starting material from renewable sources has proved challenging. Proposed efforts at propanol production have been reported in WO 2009/049274, WO 2009/103026, WO 2009/131286, WO 2010/071697, WO 2011/031897, WO 2011/029166, and WO 2011/022651. It is clear that the successful development of a process for the biological production of propanol requires careful selection of enzymes in the metabolic pathways as well as an efficient overall metabolic engineering strategy.

It would be advantageous in the art to improve n-propanol production, as a result of genetic engineering using recombinant DNA techniques.

### SUMMARY

Described herein are recombinant host cells comprising lactate dehydrogenase activity, lactaldehyde dehydrogenase activity, lactaldehyde reductase activity, propanediol dehydratase activity, and/or n-propanol dehydrogenase activity, wherein the host cell produces (or is capable of producing) an increased amount of n-propanol. In one aspect, the recombinant host cells comprises one or more (e.g., two, several) heterologous polynucleotides encoding a lactate dehydrogenase, lactaldehyde dehydrogenase, lactaldehyde reductase, propanediol dehydratase, and/or n-propanol dehydrogenase, wherein the host cell produces (or is capable of producing) and/or secretes (or is capable of secreting) a greater amount of n-propanol compared to the host cell without the heterologous polynucleotides when cultivated under the same conditions. The host cell may further comprise one or more heterologous polynucleotides encoding a thiolase; a CoA-transferase (e.g., a succinyl-CoA:acetoacetate transferase); an acetoacetate decarboxylase; and/or an isopropanol dehydrogenase, wherein the host cell produces (or is capable of producing) both n-propanol and isopropanol. In some aspects, the host cell is a lactobacillus host cell.

Also described are methods of using recombinant host cells for the production of n-propanol. In one aspect, a method of producing n-propanol, comprises: (a) cultivating a recombinant host cell (e.g., a lactobacillus host cell) having lactate dehydrogenase activity, lactaldehyde dehydrogenase activity, lactaldehyde reductase activity, propanediol dehydratase activity, and/or n-propanol dehydrogenase activity in a medium under suitable conditions to produce the n-propanol; and (b) recovering the n-propanol. In some aspects, the recombinant host cell comprises one or more (e.g., two, several) heterologous polynucleotides encoding a lactate dehydrogenase, lactaldehyde dehydrogenase, lactaldehyde reductase, propanediol dehydratase, and/or n-propanol dehydrogenase. In another aspect, a method of producing n-propanol comprises: (a) transforming into a host cell (e.g., a lactobacillus host cell) one or more heterologous polynucleotides encoding a lactate dehydrogenase, lactaldehyde dehydrogenase, lactaldehyde reductase, propanediol dehydratase, and/or n-propanol dehydrogenase described herein; (b) cultivating the transformed organism in a medium under suitable conditions to produce the n-propanol; and (c) recovering the n-propanol. In some aspects of the methods, the host cell further comprises one or more heterologous polynucleotides encoding a thiolase; a CoA-transferase (e.g., a succinyl-CoA:acetoacetate transferase); an acetoacetate decarboxylase; and/or an isopropanol dehydrogenase, and produces both n-propanol and isopropanol. In some aspects of the methods, the host cell is a lactobacillus host cell.

### BRIEF DESCRIPTION OF THE FIGURES

FIG. 1 shows a metabolic pathway from glucose to n-propanol.

FIG. 2 shows a metabolic pathway from glucose to n-propanol and isopropanol.

FIG. 3 show the DNA sequence and the deduced amino acid sequence of a Lactobacillus reuteri lactate dehydrogenase gene (SEQ ID NOs: 1 and 2, respectively).

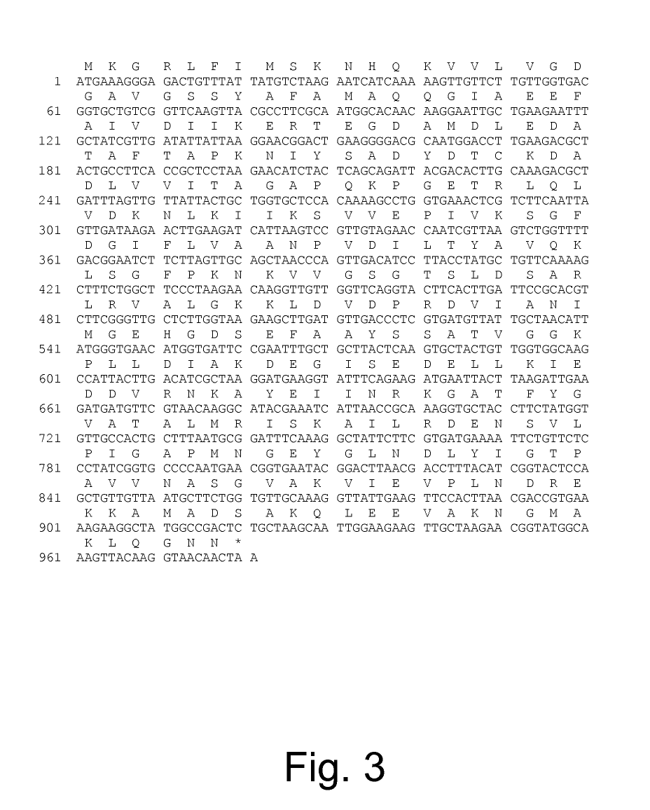

FIG. 4 show the DNA sequence and the deduced amino acid sequence of a Lactobacillus plantarum lactate dehydrogenase gene (SEQ ID NOs: 3 and 4, respectively).

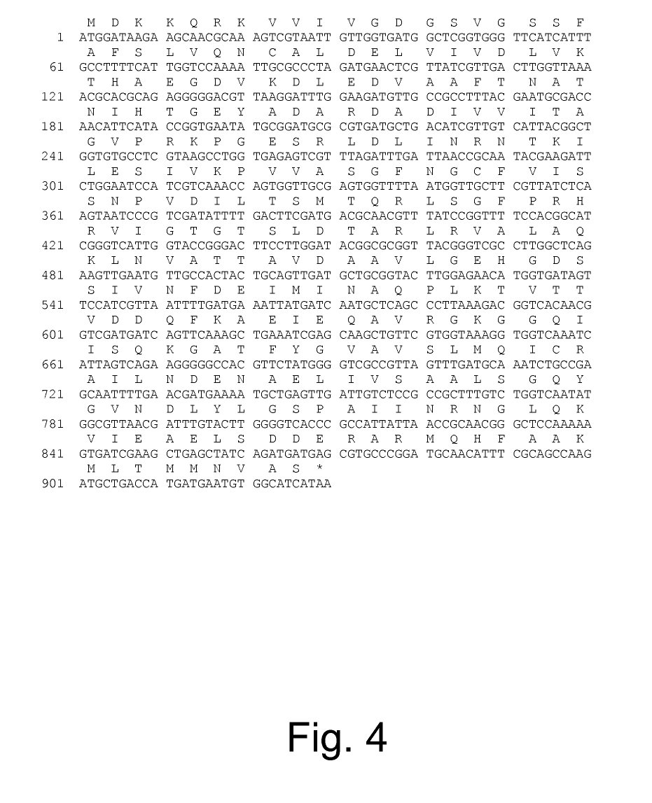

FIG. 5 show the DNA sequence and the deduced amino acid sequence of a Lactobacillus brevis lactaldehyde dehydrogenase gene (SEQ ID NOs: 5 and 6, respectively).

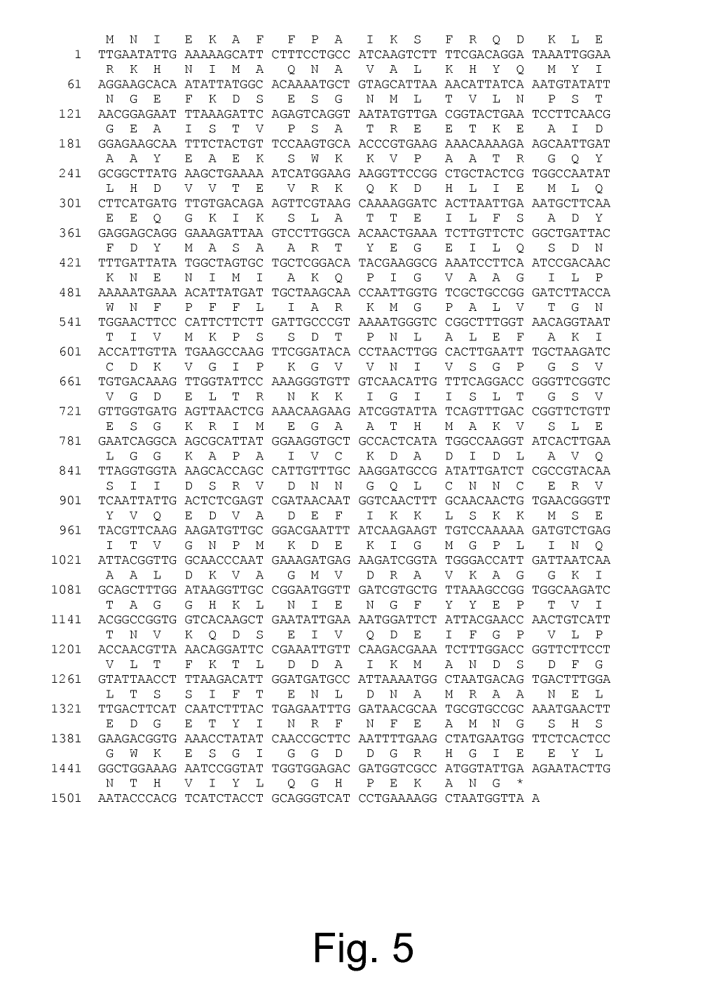

FIG. 6 show the DNA sequence and the deduced amino acid sequence of an Escherichia coli lactaldehyde dehydrogenase gene (SEQ ID NOs: 7 and 8, respectively).

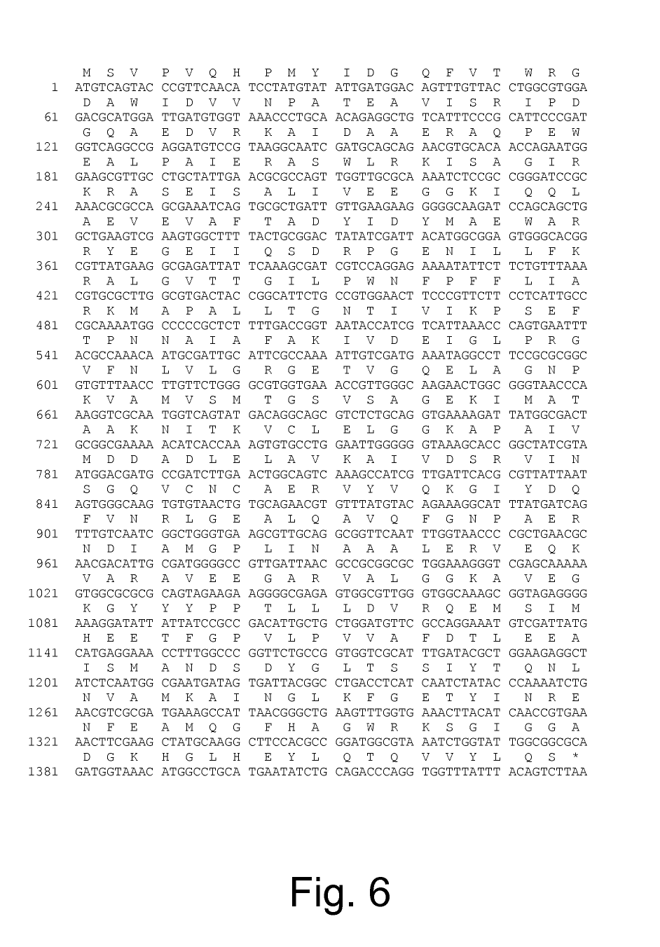

FIG. 7 show the DNA sequence and the deduced amino acid sequence of an Escherichia coli lactaldehyde reductase gene (SEQ ID NOs: 9 and 10, respectively).

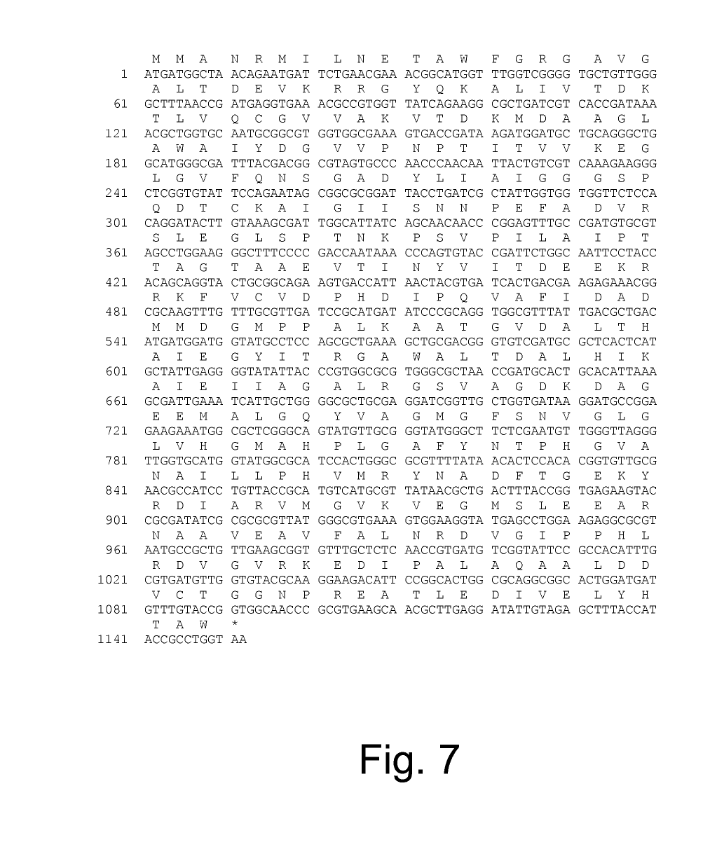

FIG. 8 show the DNA sequence and the deduced amino acid sequence of an Citrobacter koseri lactaldehyde reductase gene (SEQ ID NOs: 11 and 12, respectively).

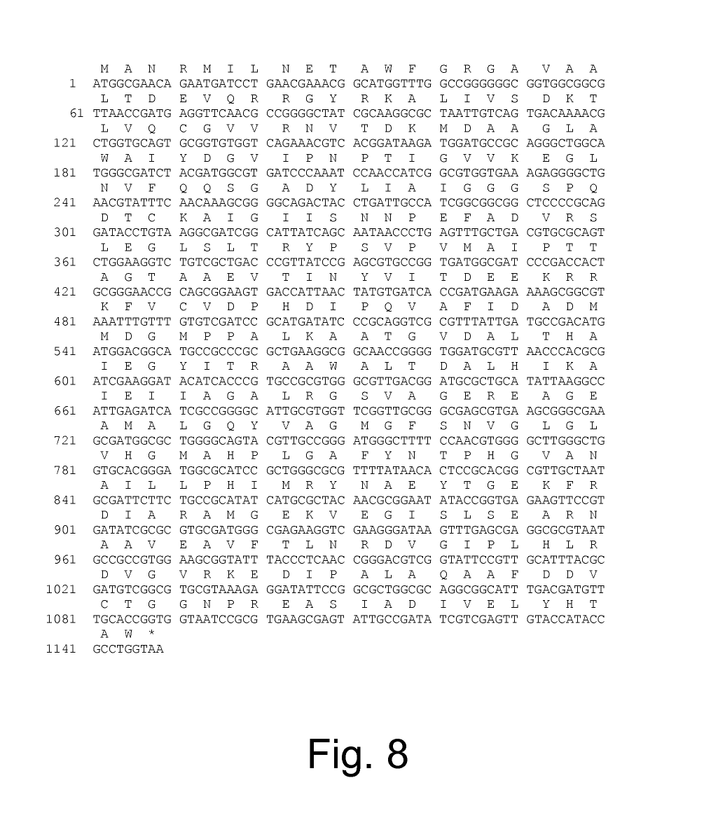

FIG. 9 shows the DNA sequence and deduced amino acid sequence of the Klebsiella oxytoca propanediol dehydratase beta subunit gene (SEQ ID NOs: 13 and 14, respectively).

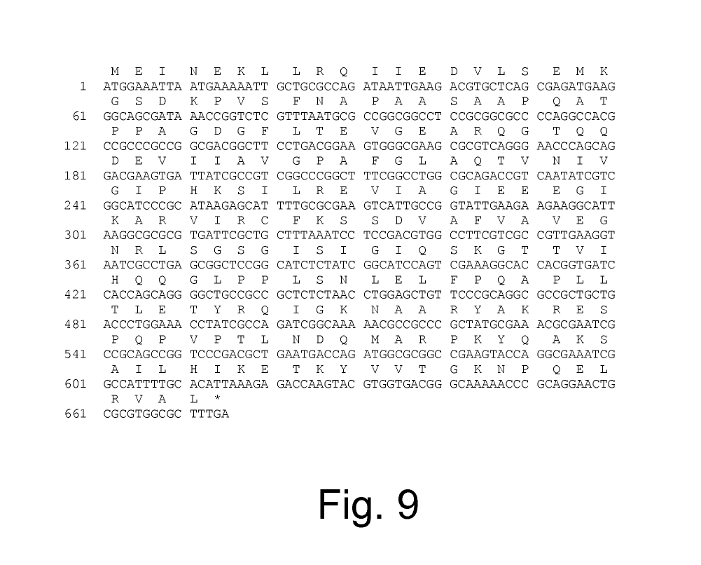

FIG. 10 shows the DNA sequence and deduced amino acid sequence of the Klebsiella oxytoca propanediol dehydratase gamma subunit gene (SEQ ID NOs: 15 and 16, respectively).

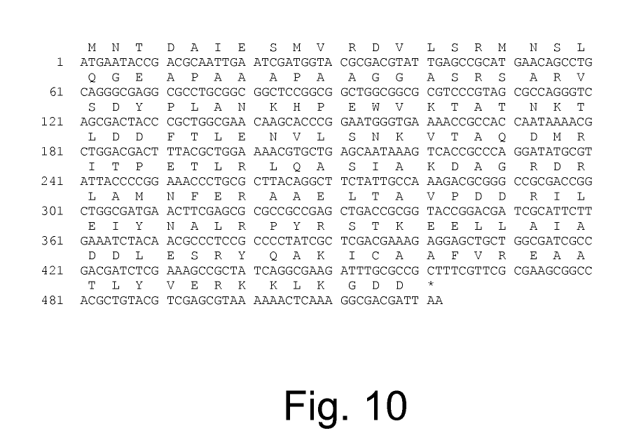

FIG. 11 shows the DNA sequence and deduced amino acid sequence of the Lactobacillus reuteri propanediol dehydratase medium subunit gene (SEQ ID NOs: 17 and 18, respectively).

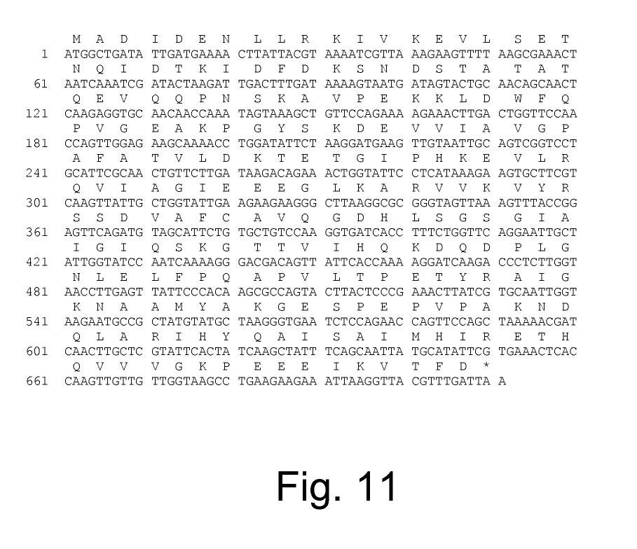

FIG. 12 shows the DNA sequence and deduced amino acid sequence of the Lactobacillus reuteri propanediol dehydratase small subunit gene (SEQ ID NOs: 19 and 20, respectively).

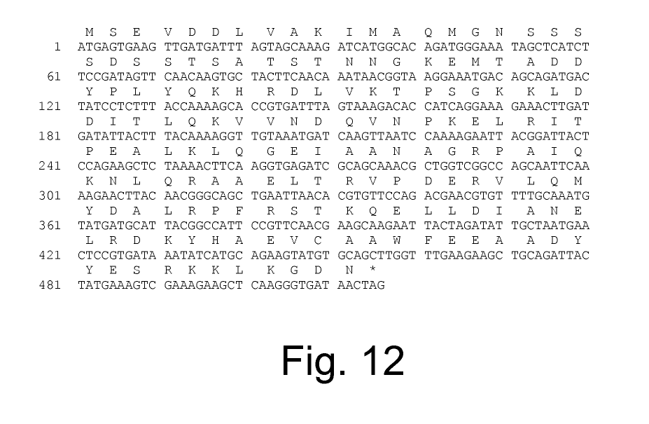

FIG. 13 show the DNA sequence and the deduced amino acid sequence of an Lactobacillus buchneri lactaldehyde dehydrogenase gene (SEQ ID NOs: 25 and 26, respectively).

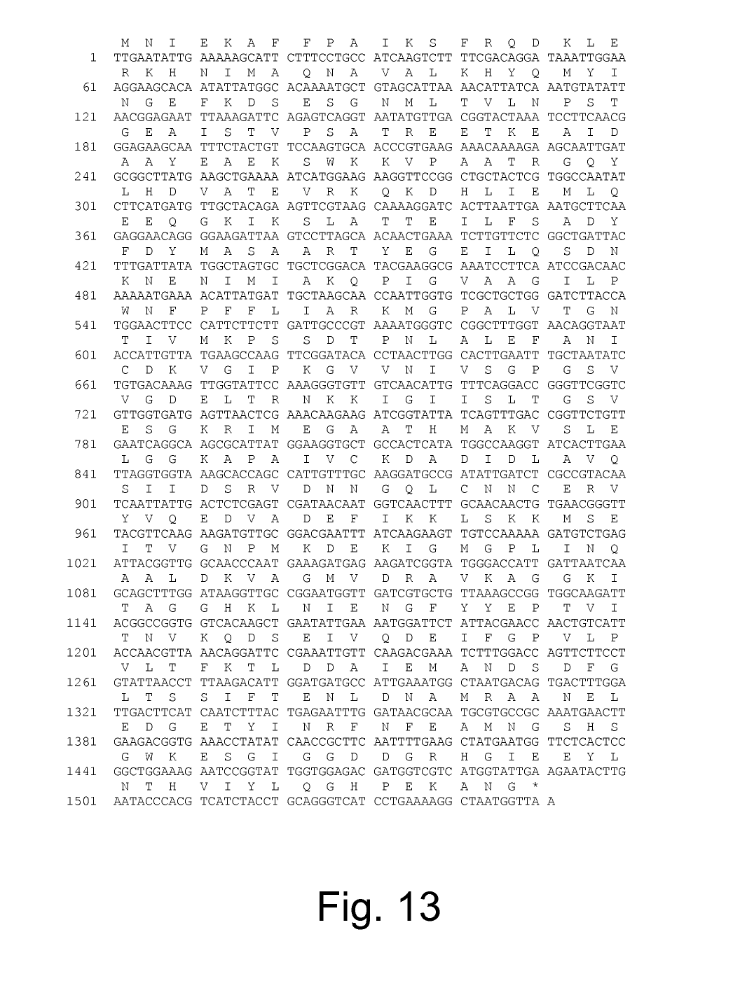

FIG. 14 show the DNA sequence and the deduced amino acid sequence of an Lactobacillus buchneri lactaldehyde reductase gene (SEQ ID NOs: 27 and 28, respectively).

FIG. 15 shows a restriction map of pTRGU88.

FIG. 16 shows a restriction map of pTRGU130.

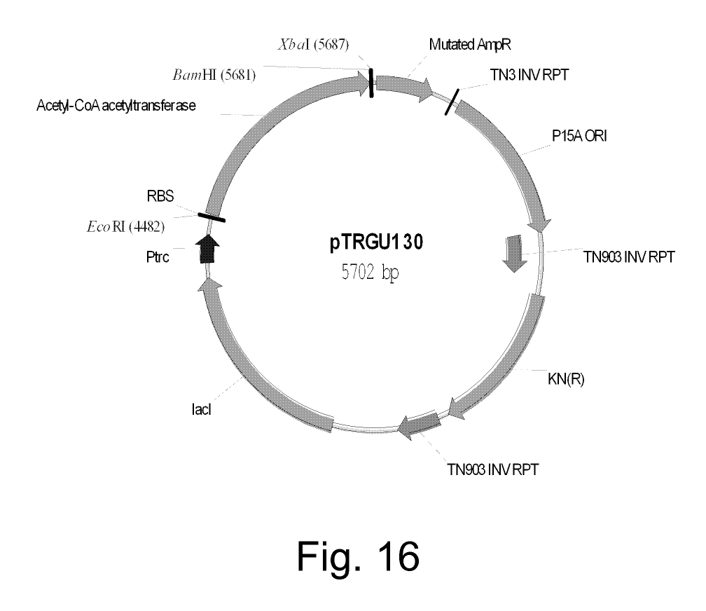

FIG. 17 shows a restriction map of pTRGU152.

FIG. 18 shows a restriction map of pJP042.

FIG. 19 shows a restriction map of pBKQ332.

FIG. 20 shows a restriction map of pBKQ405.

FIG. 21 shows a restriction map of pBKQ175.

FIG. 22 shows a restriction map of pBKQ178.

FIG. 23 shows a restriction map of pBKQ186.

FIG. 24 shows a restriction map of pBKQ156.

### DEFINITIONS

Lactate dehydrogenase: The term “lactate dehydrogenase” is defined herein as an enzyme that catalyzes the reversible conversion of pyruvate to lactate (e.g., EC 1.1.1.27 or EC 1.1.1.28). The lactate dehydrogenase may be a L-lactate dehydrogenase or a D-lactate dehydrogenase. The lactate dehydrogenase may be monomeric or in the form of a protein complex comprising two or more subunits (e.g., two heteromeric subunits) under cellular conditions. Lactate dehydrogenase activity can be determined from cell-free extracts as described in the art, e.g., as described in Q. Jin et al., 2009, J Biotechnol 144(2): 160-164. For example, lactate dehydrogenase activity may be determined in 100 mM Tris-HCl (pH 8.0) solution including 15 mM NAD⁺ and 100 mM lithium salts of L-lactate (Sigma-Aldrich Co., St. Louis, Mo., USA), by monitoring the optical density at 342 nm.

Lactaldehyde dehydrogenase: The term “lactaldehyde dehydrogenase” is defined herein as an enzyme that catalyzes the reversible conversion of L-lactate to L-lactaldehyde (e.g., EC 1.2.1.22). The lactaldehyde dehydrogenase may be monomeric or in the form of a protein complex comprising two or more subunits (e.g., two heteromeric subunits) under cellular conditions. Lactaldehyde dehydrogenase activity can be determined from cell-free extracts as described in the art, e.g., as described in J. Rodriguiz-Zavala et al., 2009, Protein Science 15(6): 1387-1396. For example, lactaldehyde dehydrogenase activity may be determined in a buffer containing 100 mM H₂NaPO₄ (pH 7.5), 100 mM NaCl, 20 mM 2-mercaptoethanol, and 2 mM NAD⁺ by the addition of L-lactaldehyde and following the increase in fluorescence due to NADH formation (340 nm for the excitation and recording the emission at 460 nm).

Lactaldehyde reductase: The term “lactaldehyde reductase” is defined herein as a an enzyme that catalyzes the reversible conversion of L-lactaldehyde to L-1,2-propandiol (e.g., EC 1.1.1.55 or 1.1.1.77). The lactaldehyde reductase may be monomeric or in the form of a protein complex comprising two or more subunits (e.g., two heteromeric subunits) under cellular conditions. Lactaldehyde reductase activity can be determined from cell-free extracts as described in the art, e.g., as described in A. Boronat and J. Aguilar, 1979, J. Bacteriol. 140, 320-326. For example, lactaldehyde reductase activity may be determined spectrophotometrically at 25° C. in 100 mM sodium phosphate buffer (pH 7.0) containing 2.5 mM L-lactaldehyde and 0.125 mM NADH by measuring the NADH loss at 340 nm.

Propanediol dehydratase: The term “propanediol dehydratase” is defined herein as a an enzyme that catalyzes the reversible conversion of L-1,2-propandiol to Propanal (e.g., EC 4.2.1.28). The propanediol dehydratase may be monomeric or in the form of a protein complex comprising two or more subunits (e.g., two heteromeric subunits) under cellular conditions. Propanediol dehydratase activity can be determined from cell-free extracts as described in the art, e.g., as described in D. Sriramulul et al., 2008, J. Bacteriol. 190: 4559-4567. For example, propanediol dehydratase activity may be determined using the “3-methyl-2-benzothiazolinone hydrazone method” (T. Toraya et al., 1977, J. Biol. Chem. 252: 963-970) wherein the assay mixture contains propanediol dehydratase, 0.2 M 1,2-propanediol, 0.05 M KCl, 0.035 M potassium phosphate buffer (pH 8.0), and 15 μM adenosylcobalamin, in a total volume of 1.0 mL. After incubation at 37° C. for 10 min, the enzymatic reaction is terminated by adding 1 mL of 0.1 M potassium citrate buffer (pH 3.6) and 0.5 mL of 0.1% MBTH hydrochloride. After 15 min at 37° C., 1 mL of water is added and the amount of propionaldehyde is determined from the absorbance at 305 nm.

n-Propanol dehydrogenase: The term “n-propanol dehydrogenase” is defined herein as any alcohol dehydrogenase (e.g., EC 1.1.1.1) that catalyzes the reduction of propanal to n-propanol. The n-propanol dehydrogenase may be monomeric or in the form of a protein complex comprising two or more subunits (e.g., two heteromeric subunits) under cellular conditions. n-Propanol dehydrogenase activity can be determined from cell-free extracts as described in the art, e.g., as described in C. Drewke and M. Ciriacy, 1988, Biochemica et Biophysica Acta, 950:54-60. For example, n-propanol dehydrogenase activity may be measured spectrophotometrically following the kinetics of NAD⁺ reduction of NADH oxidation at pH 8.3.

Thiolase: The term “thiolase” is defined herein as an acyltransferase that catalyzes the chemical reaction of two molecules of acetyl-CoA to acetoacetyl-CoA and CoA (e.g., EC 2.3.1.9). The thiolase may be monomeric or in the form of a protein complex comprising two or more subunits (e.g., two heteromeric subunits) under cellular conditions. Thiolase activity may be determined from cell-free extracts as described in the art, e.g., as described in D. P. Wiesenborn et al., 1988, Appl. Environ. Microbiol. 54:2717-2722. For example, thiolase activity may be measured spectrophotometrically by monitoring the condensation reaction coupled to the oxidation of NADH using 3-hydroxyacyl-CoA dehydrogenase in 100 mM Tris hydrochloride (pH 7.4), 1.0 mM acetyl-CoA, 0.2 mM NADH, 1 mM dithiothreitol, and 2 U of 3-hydroxyacyl-CoA dehydrogenase. After equilibration of the cuvette contents at 30° C. for 2 min, the reaction is initiated by the addition of about 125 ng of thiolase in 10 μL. The absorbance decrease at 340 nm due to oxidation of NADH is measured, and an extinction coefficient of 6.22 mM⁻¹ cm⁻¹ used.

CoA-transferase: As used herein, the term “CoA-transferase” is defined as any enzyme that catalyzes the removal of coenzyme A from acetoacetyl-CoA to generate acetoacetate. In some aspects, the CoA-transferase is an acetoacetyl-CoA:acetate/butyrate CoA transferase of EC 2.8.3.9. In some aspects, the CoA-transferase is an acetoacetyl-CoA hydrolase of EC 3.1.2.11. In some aspects, the CoA-transferase is an acetoacetyl-CoA transferase that converts acetoacetyl-CoA and acetate to acetoacetate and acetyl-CoA.

In some aspects, the CoA-transferase is a succinyl-CoA:acetoacetate transferase. As used herein, “succinyl-CoA:acetoacetate transferase” is an acetotransferase that catalyzes the chemical reaction of acetoacetyl-CoA and succinate to acetoacetate and succinyl-CoA (EC 2.8.3.5). The succinyl-CoA:acetoacetate transferase may be in the form of a protein complex comprising two or more subunits (e.g., two heteromeric subunits) as described herein. Succinyl-CoA:acetoacetate transferase activity may be determined from cell-free extracts as described in the art, e.g., as described in L. Stols et al., 1989, Protein Expression and Purification 53:396-403. For example, succinyl-CoA:acetoacetate transferase activity may be measured spectrophotometrically by monitoring the formation of the enolate anion of acetoacetyl-CoA, wherein absorbance is measured at 310 nm/30° C. over 4 minutes in an assay buffer of 67 mM lithium acetoacetate, 300 μM succinyl-CoA, and 15 mM MgCl₂ in 50 mM Tris, pH 9.1.

Acetoacetate decarboxylase: The term “acetoacetate decarboxylase” is defined herein as an enzyme that catalyzes the chemical reaction of acetoacetate to carbon dioxide and acetone (e.g., EC 4.1.1.4). The acetoacetate decarboxylase may be monomeric or in the form of a protein complex comprising two or more subunits (e.g., two heteromeric subunits) under cellular conditions. Acetoacetate decarboxylase activity may be determined from cell-free extracts as described in the art, e.g., as described in D.J. Petersen, et al., 1990, Appl. Environ. Microbiol. 56, 3491-3498. For example, acetoacetate decarboxylase activity may be measured spectrophotometrically by monitoring the depletion of acetoacetate at 270 nm in 5 nM acetoacetate, 0.1 M KPO₄, pH 5.9 at 26° C.

Isopropanol dehydrogenase: The term “isopropanol dehydrogenase” is defined herein as any suitable oxidoreductase that catalyzes the reduction of acetone to isopropanol (e.g., any suitable enzyme of EC1.1.1.1 or EC 1.1.1.80). The isopropanol dehydrogenase may be monomeric or in the form of a protein complex comprising two or more subunits (e.g., two heteromeric subunits) under cellular conditions. Acetoacetate decarboxylase activity may be determined spectrophotometrically from cell-free extracts as described in the art, e.g., by decrease in absorbance at 340 nm in an assay containing 200 μM NADPH and 10 mM acetone in 25 mM potassium phosphate, pH 7.2 at 25° C.

Pyruvate decarboxylase: The term “pyruvate decarboxylase” is defined herein as an enzyme that catalyzes the decarboxylation of pyruvic acid to acetaldehyde (e.g., EC 4.1.1.1). Pyruvate decarboxylase activity may be determined from cell-free extracts as described in the art, e.g., by J. Wang et al., 2001, Biochemistry, 40:1755-1763. For example, pyruvate decarboxylase activity may be measured spectrophotometrically by monitoring the aldehyde dehydrogenase-coupled depletion of NADH at 340 nm at 25° C., pH 6.0.

Propionaldehyde dehydrogenase: The term “propionaldehyde dehydrogenase” is defined herein as an enzyme that catalyzes the conversion of propanal to propionyl-CoA. Propionaldehyde dehydrogenase activity may be determined from cell-free extracts as described in the art, e.g., by N. Hosoi et al., 1979, J. Ferment. Technol., 57:418-427. For example, propionaldehyde dehydrogenase activity may be measured spectrophotometrically by monitoring the reduction of NAD+ by an increase in absorbance at 340 nm at 30° C. using a 3 mL solution containing 100 μmol propionaldehyde, 3 μmol NAD+, 0.3 μmol CoA, 30 μmol GSH, 100 μg bovine serum albumin, 120 μmol veronal-HCl buffer (pH 8.6).

Heterologous polynucleotide: The term “heterologous polynucleotide” is defined herein as a polynucleotide that is not native to the host cell; a native polynucleotide in which one or more (e.g., several) structural modifications have been made to the coding region; a native polynucleotide whose expression is quantitatively altered as a result of manipulation of the DNA by recombinant DNA techniques, e.g., a different (foreign) promoter linked to the polynucleotide; or a native polynucleotide whose expression is quantitatively altered by the introduction of one or more extra copies of the polynucleotide into the host cell.

Isolated/purified: The terms “isolated” and “purified” mean a polypeptide or polynucleotide that is removed from at least one component with which it is naturally associated. For example, a polypeptide may be at least 1% pure, e.g., at least 5% pure, at least 10% pure, at least 20% pure, at least 40% pure, at least 60% pure, at least 80% pure, at least 90% pure, at least 93% pure, at least 95% pure, at least 97%, at least 98% pure, or at least 99% pure, as determined by SDS-PAGE and a polynucleotide may be at least 1% pure, e.g., at least 5% pure, at least 10% pure, at least 20% pure, at least 40% pure, at least 60% pure, at least 80% pure, at least 90%, at least 93% pure, at least 95% pure, at least 97%, at least 98% pure, or at least 99% pure, as determined by agarose electrophoresis.

Coding sequence: The term “coding sequence” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Mature polypeptide sequence: The term “mature polypeptide sequence” means the portion of the referenced polypeptide sequence after any post-translational sequence modifications (such as N-terminal processing and/or C-terminal truncation). The mature polypeptide sequence may be predicted, e.g., based on the SignalP program (Nielsen et al., 1997, Protein Engineering 10: 1-6) or the InterProScan program (The European Bioinformatics Institute). In some instances, the mature polypeptide sequence may be identical to the entire referenced polypeptide sequence. It is known in the art that a host cell may produce a mixture of two of more different mature polypeptide sequences (i.e., with a different C-terminal and/or N-terminal amino acid) expressed by the same polynucleotide.

Mature polypeptide coding sequence: The term “mature polypeptide coding sequence” means the portion of the referenced polynucleotide sequence that encodes a mature polypeptide sequence. The mature polypeptide coding sequence may be predicted, e.g., based on the SignalP program (supra) or the InterProScan program (supra). In some instances, the mature polypeptide coding sequence may be identical to the entire referenced polynucleotide sequence.

Fragment: The term “fragment” means a polypeptide having one or more (e.g., several) amino acids deleted from the amino and/or carboxyl terminus of a referenced polypeptide sequence. In one aspect, the fragment has lactate dehydrogenase, lactaldehyde dehydrogenase, lactaldehyde reductase, propanediol dehydratase, or n-propanol dehydrogenase activity. In another aspect, the number of amino acid residues in the fragment is at least 75%, e.g., at least 80%, 85%, 90%, or 95% of the number of amino acid residues in SEQ ID NOs: 2, 4, 6, 8, 10, 12, 14, 16, 18, 20, 22, or 24.

Subsequence: The term “subsequence” means a polynucleotide having one or more (e.g., several) nucleotides deleted from the 5′ and/or 3′ end of the referenced nucleotide sequence. In one aspect, the subsequence encodes a fragment having lactate dehydrogenase, lactaldehyde dehydrogenase, lactaldehyde reductase, propanediol dehydratase, or n-propanol dehydrogenase activity. In another aspect, the number of nucleotides residues in the subsequence is at least 75%, e.g., at least 80%, 85%, 90%, or 95% of the number of nucleotide residues in SEQ ID NOs: 1, 3, 5, 7, 9, 11, 13, 15, 17, or 19.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Nucleic acid construct: The term “nucleic acid construct” means a polynucleotide comprises one or more (e.g., several) control sequences. The polynucleotide may be single-stranded or double-stranded, and may be isolated from a naturally occurring gene, modified to contain segments of nucleic acids in a manner that would not otherwise exist in nature, or synthetic.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Operably linked: The term “operably linked” means a configuration in which a control sequence is placed at an appropriate position relative to the coding sequence of a polynucleotide such that the control sequence directs the expression of the coding sequence.

Expression vector: The term “expression vector” means a linear or circular DNA molecule that comprises a polynucleotide encoding a polypeptide and is operably linked to control sequences, wherein the control sequences provide for expression of the polynucleotide encoding the polypeptide. At a minimum, the expression vector comprises a promoter sequence, and transcriptional and translational stop signal sequences.

Host cell: The term “host cell” means any cell type that is susceptible to transformation, transfection, transduction, and the like with a nucleic acid construct or expression vector comprising one or more (e.g., two, several) polynucleotides described herein (e.g., one or more polynucleotides encoding a lactate dehydrogenase, lactaldehyde dehydrogenase, lactaldehyde reductase, propanediol dehydratase, or n-propanol dehydrogenase). The term “host cell” encompasses any progeny of a parent cell that is not identical to the parent cell due to mutations that occur during replication.

Disruption: The term “disruption” means that a promoter, coding region, and/or terminator of a polynucleotide encoding a polypeptide having enzyme activity within a host cell is partially or entirely modified (such as by modification, deletion, insertion, and/or substitution of one or more nucleotides) resulting in the absence or decrease of said enzyme activity of the host cell. The absence or decrease of enzyme activity can be measured directly by techniques known in the art (such as cell-free extract measurements referenced herein); or by the absence or decrease of corresponding mRNA (e.g., at least 50% decrease, at least 60% decrease, at least 70% decrease, at least 80% decrease, or at least 90% decrease); the absence or decrease in the amount of corresponding polypeptide having enzyme activity (e.g., at least 50% decrease, at least 60% decrease, at least 70% decrease, at least 80% decrease, or at least 90% decrease); or the absence or decrease of the specific activity of the corresponding polypeptide having enzyme activity (e.g., at least 50% decrease, at least 60% decrease, at least 70% decrease, at least 80% decrease, or at least 90% decrease) if present. Disruptions of a particular gene of interest can be generated by methods known in the art, e.g., by directed homologous recombination (see Methods in Yeast Genetics (1997 edition), Adams, Gottschling, Kaiser, and Stems, Cold Spring Harbor Press (1998)); or by RNAi or antisense technology.

Volumetric productivity: The term “volumetric productivity” refers to the amount of referenced product produced (e.g., the amount of n-propanol produced) per volume of the system used (e.g., the total volume of media and contents therein) per unit of time.

Fermentable medium: The term “fermentable medium” refers to a medium comprising one or more (e.g., several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as n-propanol. In some instances, the fermentation medium is derived from a natural source, such as sugar cane, starch, or cellulose, and may be the result of pretreating the source by enzymatic hydrolysis (saccharification).

Reference to “about” a value or parameter herein includes aspects that are directed to that value or parameter per se. For example, description referring to “about X” includes the aspect “X”. When used in combination with measured values, “about” includes a range that encompasses at least the uncertainty associated with the method of measuring the particular value, and can include a range of plus or minus two standard deviations around the stated value.

As used herein and in the appended claims, the singular forms “a,” “or,” and “the” include plural referents unless the context clearly dictates otherwise. It is understood that the aspects described herein include “consisting” and/or “consisting essentially of” aspects.

Unless defined otherwise or clearly indicated by context, all technical and scientific terms used herein have the same meaning as commonly understood by one of ordinary skill in the art.

### DETAILED DESCRIPTION

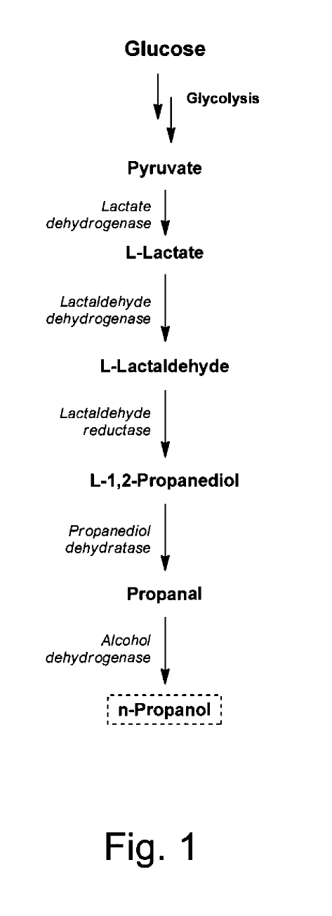

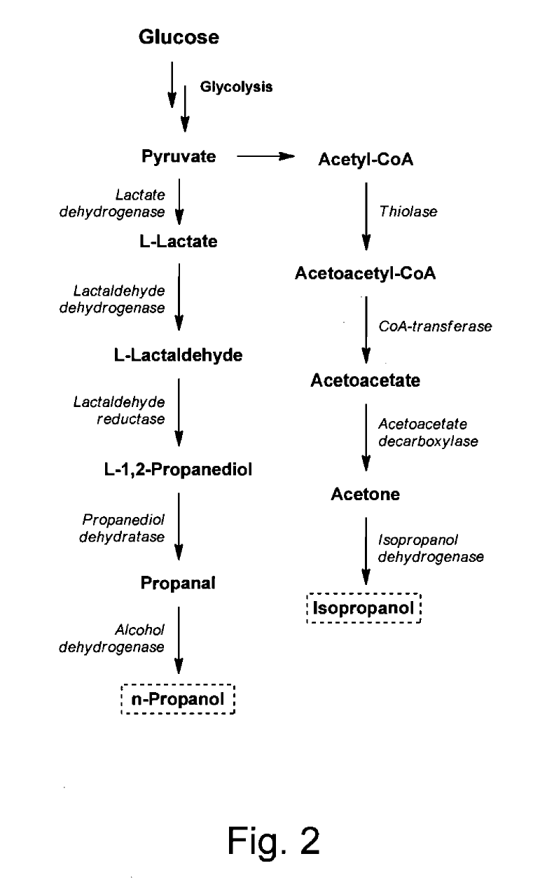

Described herein, inter alia, is the increased expression of specific genes in a recombinant host cell to enhance the production of n-propanol. The host cell may comprise lactate dehydrogenase activity, lactaldehyde dehydrogenase activity, lactaldehyde reductase activity, propanediol dehydratase activity, and/or n-propanol dehydrogenase activity to produce n-propanol, e.g., as depicted in FIG. 1. The host cell may further comprise thiolase activity, CoA-transferase activity (e.g., succinyl-CoA:acetoacetate transferase activity), acetoacetate decarboxylase activity, and/or isopropanol dehydrogenase activity, to produce both n-propanol and isopropanol, e.g., as depicted in FIG. 2.

The lactate dehydrogenase, lactaldehyde dehydrogenase, lactaldehyde reductase, propanediol dehydratase, and n-propanol dehydrogenase transformations shown in FIGS. 1 and 2; and the thiolase, CoA-transferase, acetoacetate decarboxylase, and isopropanol dehydrogenase transformations shown in FIG. 2, may result from activities of heterologous polynucleotides encoding each polypeptide, or from a combination of activities of endogenous gene expression supplemented with one or more heterologous polynucleotides described herein. Any suitable lactate dehydrogenase, lactaldehyde dehydrogenase, lactaldehyde reductase, propanediol dehydratase, n-propanol dehydrogenase, thiolase, CoA-transferase, acetoacetate decarboxylase, and/or isopropanol dehydrogenase may be overexpressed under culture conditions to increase the titers of n-propanol (and optionally isopropanol).

### Lactate Dehydrogenases and Polynucleotides Encoding Lactate Dehydrogenases

In some aspects of the recombinant host cells and methods described herein, the host cells have lactate dehydrogenase activity. The lactate dehydrogenase can be any lactate dehydrogenase that is suitable for the host cells and their methods of use described herein, such as a naturally occurring lactate dehydrogenase or a variant thereof that retains lactate dehydrogenase activity. In one aspect, the lactate dehydrogenase is present in the cytosol of the host cells. In some aspects, the host cell comprises one or more (e.g., two, several) heterologous polynucleotides that encode a lactate dehydrogenase.

In some aspects, the host cells comprising the one or more (e.g., two, several) heterologous polynucleotides that encode a lactate dehydrogenase have an increased level of lactate dehydrogenase activity compared to the host cells without the one or more polynucleotides that encode a lactate dehydrogenase, when cultivated under the same conditions. In some aspects, the host cells comprising the one or more heterologous polynucleotides that encode a lactate dehydrogenase have an increased level of lactate dehydrogenase activity of at least 5%, e.g., at least 10%, at least 15%, at least 20%, at least 25%, at least 50%, at least 100%, at least 150%, at least 200%, at least 300%, or at 500% compared to the host cells without the one or more polynucleotides that encode a lactate dehydrogenase, when cultivated under the same conditions.

In some aspects, the host cell comprises a heterologous polynucleotide that encodes a lactate dehydrogenase. In one aspect, the heterologous polynucleotide that encodes a lactate dehydrogenase is selected from: (a) a polynucleotide that encodes a lactate dehydrogenase having at least 65% sequence identity to SEQ ID NO: 2, 4, or the mature polypeptide sequence thereof; (b) a polynucleotide that hybridizes under low stringency conditions with SEQ ID NO: 1, 3, the mature polypeptide coding sequence thereof, or the full-length complementary strand of the foregoing; and (c) a polynucleotide that has at least 65% sequence identity to SEQ ID NO: 1, 3, or the mature polypeptide coding sequence thereof. As can be appreciated by one of skill in the art, the heterologous polynucleotide that encodes a lactate dehydrogenase may qualify under more than one of the selections (a), (b) and (c) noted above.

In one aspect, the heterologous polynucleotide encodes a lactate dehydrogenase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 2, 4, or the mature polypeptide sequence thereof. In one aspect, lactate dehydrogenase sequence differs by no more than ten amino acids, e.g., by no more than five amino acids, by no more than four amino acids, by no more than three amino acids, by no more than two amino acids, or by one amino acid from SEQ ID NO: 2, 4, or the mature polypeptide sequence thereof.

In one aspect, the lactate dehydrogenase comprises or consists of the amino acid sequence of SEQ ID NO: 2 or 4, the mature polypeptide sequence of SEQ ID NO: 2 or 4, an allelic variant thereof, or a fragment of the foregoing having lactate dehydrogenase activity. In another aspect, the lactate dehydrogenase comprises or consists of the amino acid sequence of SEQ ID NO: 2 or 4. In another aspect, the lactate dehydrogenase comprises or consists of the mature polypeptide sequence of SEQ ID NO: 2 or 4. In another aspect, the lactate dehydrogenase comprises or consists of amino acids 1 to 326 of SEQ ID NO: 2 or amino acids 1 to 309 of SEQ ID NO: 4.

In one aspect, the heterologous polynucleotide encodes a lactate dehydrogenase having an amino acid substitution, deletion, and/or insertion at one or more (e.g., two, several) positions of SEQ ID NO: 2, 4, or the mature polypeptide sequence thereof. An amino acid substitution means that an amino acid corresponding to a position of the referenced sequence is different; an amino acid deletion means that an amino acid corresponding to a position of the referenced sequence is not present; and an amino acid insertion means that an amino acid is present that is not present at a corresponding position of the referenced sequence. In some of these aspects, the total number of amino acid substitutions, deletions and/or insertions of SEQ ID NO: 2, 4, or the mature polypeptide sequence thereof, is not more than 10, e.g., not more than 9, 8, 7, 6, 5, 4, 3, 2, or 1.

The amino acid changes are generally of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the lactate dehydrogenase, alter the substrate specificity, change the pH optimum, and the like.

Essential amino acids in a lactate dehydrogenase can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for lactate dehydrogenase activity to identify amino acid residues that are critical to the activity of the molecule. See also, Hilton et al., 1996, J. Biol. Chem. 271: 4699-4708. The active site of the lactate dehydrogenase or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with other lactate dehydrogenase that are related to the referenced lactate dehydrogenase.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active lactate dehydrogenases can be recovered from the host cells and rapidly sequenced using standard methods in the art. These methods allow the rapid determination of the importance of individual amino acid residues in a polypeptide.

In one aspect, the heterologous polynucleotide that encodes a lactate dehydrogenase hybridizes under at least low stringency conditions, e.g., medium stringency conditions, medium-high stringency conditions, high stringency conditions, or very high stringency conditions with (i) SEQ ID NO: 1, 3, the mature polypeptide coding sequence thereof, or the full-length complementary of the foregoing (see, e.g., J. Sambrook, E. F. Fritsch, and T. Maniatus, 1989, Molecular Cloning, A Laboratory Manual, 2d edition, Cold Spring Harbor, New York).

In one aspect, the heterologous polynucleotide that encodes a lactate dehydrogenase has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 1, 3, or the mature polypeptide coding sequence thereof.

In one aspect, the heterologous polynucleotide that encodes a lactate dehydrogenase comprises the sequence of SEQ ID NO: 1, 3, or the mature polypeptide coding sequence thereof. In one aspect, the heterologous polynucleotide that encodes a lactate dehydrogenase comprises the sequence of SEQ ID NO: 1 or 3. In one aspect, the heterologous polynucleotide that encodes a lactate dehydrogenase comprises the mature polypeptide coding sequence of SEQ ID NO: 1 or 3. In one aspect, the mature polypeptide coding sequence is nucleotides 1 to 981 of SEQ ID NO: 1 or nucleotides 1 to 930 of SEQ ID NO: 3. In one aspect, the heterologous polynucleotide that encodes a lactate dehydrogenase comprises a subsequence of SEQ ID NO: 1, 3, or the mature polypeptide coding sequence thereof, wherein the subsequence encodes a polypeptide having lactate dehydrogenase activity. In one aspect, the number of nucleotides residues in the subsequence is at least 75%, e.g., at least 80%, 85%, 90%, or 95% of the number of nucleotide residues in SEQ ID NO: 1 or 3.

In one aspect, the heterologous polynucleotide encodes a fragment of SEQ ID NO: 2, 4, or the mature polypeptide sequence thereof, wherein the fragment has lactate dehydrogenase activity. In one aspect, the number of amino acid residues in the fragment is at least 75%, e.g., at least 80%, 85%, 90%, or 95% of the number of amino acid residues in SEQ ID NO: 2 or 4.

The lactate dehydrogenase may be a fused polypeptide or cleavable fusion polypeptide in which another polypeptide is fused at the N-terminus or the C-terminus of the lactate dehydrogenase. A fused polypeptide may be produced by fusing a polynucleotide encoding another polypeptide to a polynucleotide encoding the lactate dehydrogenase. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Techniques used to isolate or clone a polynucleotide—such as a polynucleotide encoding a lactate dehydrogenase—as well as any other polypeptide used in any of the aspects mentioned herein, are known in the art and include isolation from genomic DNA, preparation from cDNA, or a combination thereof. The cloning of the polynucleotides from such genomic DNA can be effected, e.g., by using the well known polymerase chain reaction (PCR) or antibody screening of expression libraries to detect cloned DNA fragments with shares structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain of Aspergillus, or another or related organism, and thus, for example, may be an allelic or species variant of the polypeptide encoding region of the nucleotide sequence.

The polynucleotide of SEQ ID NO: 1, 3, or a subsequence thereof; as well as the amino acid sequence of SEQ ID NO: 2, 4, or a fragment thereof; may be used to design nucleic acid probes to identify and clone a lactate dehydrogenase from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, e.g., at least 14 nucleotides, at least 25 nucleotides, at least 35 nucleotides, at least 70 nucleotides in lengths. The probes may be longer, e.g., at least 100 nucleotides, at least 200 nucleotides, at least 300 nucleotides, at least 400 nucleotides, at least 500 nucleotides in lengths. Even longer probes may be used, e.g., at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin).

A genomic DNA or cDNA library prepared from such other strains may be screened for DNA that hybridizes with the probes described above and encodes a polypeptide having lactate dehydrogenase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1, 3, or a subsequence thereof, the carrier material may be used in a Southern blot.

For purposes of the probes described above, hybridization indicates that the polynucleotide hybridizes to a labeled nucleic acid probe corresponding to SEQ ID NO: 1 or 3, the mature polypeptide coding sequence of SEQ ID NO: 1 or 3, or the full-length complementary strand thereof, or a subsequence of the foregoing; under very low to very high stringency conditions. Molecules to which the nucleic acid probe hybridizes under these conditions can be detected using, for example, X-ray film.

In one aspect, the nucleic acid probe is SEQ ID NO: 1 or 3. In another aspect, the nucleic acid probe is the mature polypeptide coding sequence of SEQ ID NO: 1 or 3. In another aspect, the nucleic acid probe is a polynucleotide that encodes the polypeptide of SEQ ID NO: 2, 4, or a fragment thereof.

For long probes of at least 100 nucleotides in length, very low to very high stringency conditions are defined as prehybridization and hybridization at 42° C. in 5×SSPE, 0.3% SDS, 200 micrograms/mL sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), at 50° C. (low stringency), at 55° C. (medium stringency), at 60° C. (medium-high stringency), at 65° C. (high stringency), and at 70° C. (very high stringency).

For short probes of about 15 nucleotides to about 70 nucleotides in length, stringency conditions are defined as prehybridization and hybridization at about 5° C. to about 10° C. below the calculated Tₘ using the calculation according to Bolton and McCarthy (1962, Proc. Natl. Acad. Sci. USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per mL following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

Polynucleotides encoding the lactate dehydrogenase described herein may be obtained from a microorganism of any genus. As used herein, the term “obtained from” in connection with a given source shall mean that the polypeptide encoded by a polynucleotide is produced by the source or by a cell in which the polynucleotide from the source has been inserted.

The lactate dehydrogenase may be a bacterial lactate dehydrogenase. For example, the lactate dehydrogenase may be a Gram-positive bacterial lactate dehydrogenase such as a Bacillus, Streptococcus, Streptomyces, Staphylococcus, Enterococcus, Lactobacillus, Lactococcus, Clostridium, Geobacillus, or Oceanobacillus lactate dehydrogenase, or a Gram-negative bacterial lactate dehydrogenase such as an E. coli, Pseudomonas, Salmonella, Campylobacter, Helicobacter, Flavobacterium, Fusobacterium, Ilyobacter, Neisseria, or Ureaplasma lactate dehydrogenase.

In one aspect, the lactate dehydrogenase is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis lactate dehydrogenase.

In another aspect, the lactate dehydrogenase is a Streptococcus equisimilis, Streptococcus pyogenes, Streptococcus uberis, or Streptococcus equi subsp. Zooepidemicus lactate dehydrogenase. In another aspect, the lactate dehydrogenase is a Streptomyces achromogenes, Streptomyces avermitilis, Streptomyces coelicolor, Streptomyces griseus, or Streptomyces lividans lactate dehydrogenase.

In one aspect, the lactate dehydrogenase is a Lactobacillus lactate dehydrogenase, such as a Lactobacillus reuteri lactate dehydrogenase of SEQ ID No: 2 or a Lactobacillus plantarum lactate dehydrogenase of SEQ ID No: 4.

The lactate dehydrogenase may be a fungal lactate dehydrogenase. In one aspect, the fungal lactate dehydrogenase is a yeast lactate dehydrogenase such as a Candida, Kluyveromyces, Pichia, Saccharomyces, Schizosaccharomyces, or Yarrowia lactate dehydrogenase.

In another aspect, the fungal lactate dehydrogenase is a filamentous fungal lactate dehydrogenase such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria lactate dehydrogenase.

In another aspect, the lactate dehydrogenase is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis lactate dehydrogenase.

In another aspect, the lactate dehydrogenase is an Acremonium cellulolyticus, Aspergillus aculeatus, Aspergillus awamori, Aspergillus flavus, Aspergillus fumigatus, Aspergillus foetidus, Aspergillus japonicus, Aspergillus nidulans, Aspergillus niger, Aspergillus oryzae, Aspergillus sojae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, or Trichoderma viride lactate dehydrogenase.

It will be understood that for the aforementioned species, both the perfect and imperfect states, and other taxonomic equivalents, e.g., anamorphs, are encompassed regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

Other lactate dehydrogenase candidates that can be used with the host cells and methods of use described herein include, but are not limited to L-lactate dehydrogenases genes obtained from Lactobacillus helveticus, Lactobacillus casei, Bacillus megaterium, Pediococcus acidilactici, Rhizopus oryzae and bovine sources such as Bos taurus, and D-lactate dehydrogenase genes obtained from Lactobacillus helveticus, Lactobacillus johnsonii, Lactobacillus bulgaricus, Lactobacillus delbrueckii, Lactobacillus plantarum, Lactobacillus pentosus and P. acidilactici. For example, the lactate dehydrogenase may be the Lactobacillus helveticus lactate dehydrogenase of SEQ ID NO: 34 (encoded by the polynucleotide sequence of SEQ ID NO: 33) or the Bacillus megaterium lactate dehydrogenase of SEQ ID NO: 36 (encoded by the polynucleotide sequence of SEQ ID NO: 35). Any aspect described herein related to sequence identity, hybridization, amino acid modifications (e.g., substitutions, deletions, and/or insertions), fragments or subsequences thereof is embraced for the lactate dehydrogenases above.

In some aspects, the lactate dehydrogenase has at least 20%, e.g., at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or at least 100% of the lactate dehydrogenase activity of the mature polypeptide sequence of SEQ ID NO: 2 or 4 under the same conditions.

The lactate dehydrogenase may also be identified and obtained from other sources including microorganisms isolated from nature (e.g., soil, composts, water, silage, etc.) or DNA samples obtained directly from natural materials (e.g., soil, composts, water, silage, etc.) using the above-mentioned probes. Techniques for isolating microorganisms and DNA directly from natural habitats are well known in the art. The polynucleotide encoding a lactate dehydrogenase may then be derived by similarly screening a genomic or cDNA library of another microorganism or mixed DNA sample. Once a polynucleotide encoding a lactate dehydrogenase has been detected with suitable probe(s) as described herein, the sequence may be isolated or cloned by utilizing techniques that are known to those of ordinary skill in the art (see, e.g., J. Sambrook, E. F. Fritsch, and T. Maniatus, 1989, Molecular Cloning, A Laboratory Manual, 2d edition, Cold Spring Harbor, New York).

### Lactaldehyde Dehydrogenases and Polynucleotides Encoding Lactaldehyde Dehydrogenases

In some aspects of the recombinant host cells and methods of use thereof, the host cells have lactaldehyde dehydrogenase activity. The lactaldehyde dehydrogenase can be any lactaldehyde dehydrogenase that is suitable for the host cells and their methods of use described herein, such as a naturally occurring lactaldehyde dehydrogenase or a variant thereof that retains lactaldehyde dehydrogenase activity. In one aspect, the lactaldehyde dehydrogenase is present in the cytosol of the host cells. In some aspects, the host cells comprise one or more (e.g., two, several) heterologous polynucleotides that encode a lactaldehyde dehydrogenase.

In some aspects, the host cells comprising the one or more (e.g., two, several) heterologous polynucleotides that encode a lactaldehyde dehydrogenase have an increased level of lactaldehyde dehydrogenase activity compared to the host cells without the one or more polynucleotides that encode a lactaldehyde dehydrogenase, when cultivated under the same conditions. In some aspects, the host cells comprising the one or more heterologous polynucleotides that encode a lactaldehyde dehydrogenase have an increased level of lactaldehyde dehydrogenase activity of at least 5%, e.g., at least 10%, at least 15%, at least 20%, at least 25%, at least 50%, at least 100%, at least 150%, at least 200%, at least 300%, or at 500% compared to the host cells without the one or more polynucleotides that encode a lactaldehyde dehydrogenase, when cultivated under the same conditions.

In some aspects, the host cell comprises a heterologous polynucleotide that encodes a lactaldehyde dehydrogenase. In one aspect, the heterologous polynucleotide that encodes a lactaldehyde dehydrogenase is selected from: (a) a polynucleotide that encodes a lactaldehyde dehydrogenase having at least 65% sequence identity to SEQ ID NO: 6, 8, 26, or the mature polypeptide sequence thereof; (b) a polynucleotide that hybridizes under low stringency conditions with SEQ ID NO: 5, 7, 25, the mature polypeptide coding sequence thereof, or the full-length complementary strand of the foregoing; and (c) a polynucleotide that has at least 65% sequence identity to SEQ ID NO: 5, 7, 25, or the mature polypeptide coding sequence thereof. As can be appreciated by one of skill in the art, in some instances the heterologous polynucleotide that encodes a lactaldehyde dehydrogenase may qualify under more than one of the selections (a), (b) and (c) noted above.

In one aspect, the polynucleotide encodes a lactaldehyde dehydrogenase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 6, 8, 26, or the mature polypeptide sequence thereof. In one aspect, the lactaldehyde dehydrogenase sequence differs by no more than ten amino acids, e.g., by no more than five amino acids, by no more than four amino acids, by no more than three amino acids, by no more than two amino acids, or by one amino acid from SEQ ID NO: 6, 8, 26, or the mature polypeptide sequence thereof.

In one aspect, the lactaldehyde dehydrogenase comprises or consists of the amino acid sequence of SEQ ID NO: 6 or 8, the mature polypeptide sequence of SEQ ID NO: 6 or 8, an allelic variant thereof, or a fragment of the foregoing having lactaldehyde dehydrogenase activity. In another aspect, the lactaldehyde dehydrogenase comprises or consists of the amino acid sequence of SEQ ID NO: 6, 8, or 26. In another aspect, the lactaldehyde dehydrogenase comprises or consists of the mature polypeptide sequence of SEQ ID NO: 6, 8, or 26. In another aspect, the lactaldehyde dehydrogenase comprises or consists of amino acids 1 to 516 of SEQ ID NO: 6, amino acids 1 to 479 of SEQ ID NO: 8, or amino acids 1 to 516 of SEQ ID NO: 26.

For example, in one aspect, the heterologous polynucleotide encodes a lactaldehyde dehydrogenase having an amino acid substitution, deletion, and/or insertion of one or more (e.g., two, several) amino acids of SEQ ID NO: 6, 8, 26, or the mature polypeptide sequence thereof, as described supra. In some aspects, the total number of amino acid substitutions, deletions and/or insertions of SEQ ID NO: 6, 8, 26, or the mature polypeptide sequence thereof, is not more than 10, e.g., not more than 9, 8, 7, 6, 5, 4, 3, 2, or 1.

In one aspect, the heterologous polynucleotide that encodes a lactaldehyde dehydrogenase hybridizes under at least low stringency conditions, e.g., medium stringency conditions, medium-high stringency conditions, high stringency conditions, or very high stringency conditions with SEQ ID NO: 5, 7, 25, the mature polypeptide coding sequence thereof, or the full-length complementary strand of the foregoing (see, e.g., J. Sambrook, E. F. Fritsch, and T. Maniatus, 1989, supra).

In one aspect, the heterologous polynucleotide that encodes a lactaldehyde dehydrogenase has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 5, 7, 25, or the mature polypeptide coding sequence thereof.

In one aspect, the heterologous polynucleotide that encodes a lactaldehyde dehydrogenase comprises the sequence of SEQ ID NO: 5, 7, 25, or the mature polypeptide coding sequence thereof. In one aspect, the heterologous polynucleotide that encodes the lactaldehyde dehydrogenase comprises the sequence of SEQ ID NO: 5, 7, or 25. In one aspect, the heterologous polynucleotide that encodes the lactaldehyde dehydrogenase comprises the mature polypeptide coding sequence of SEQ ID NO: 5, 7, or 25. In one aspect, the mature polypeptide coding sequence is nucleotides 1 to 1551 of SEQ ID NO: 5, nucleotides 1 to 1440 of SEQ ID NO: 7, or nucleotides 1 to 1551 of SEQ ID NO: 25. In one aspect, the heterologous polynucleotide comprises a subsequence of SEQ ID NO: 5, 7, 25, or the mature polypeptide coding sequence thereof, wherein the subsequence encodes a polypeptide having lactaldehyde dehydrogenase activity. In one aspect, the number of nucleotides residues in the subsequence is at least 75%, e.g., at least 80%, 85%, 90%, or 95% of the number of nucleotide residues in SEQ ID NO: 5, 7, or 25.

In one aspect, the heterologous polynucleotide encodes a fragment of SEQ ID NO: 6, 8, 26, or the mature polypeptide sequence thereof, wherein the fragment has lactaldehyde dehydrogenase activity. In one aspect, the number of amino acid residues in the fragment is at least 75%, e.g., at least 80%, 85%, 90%, or 95% of the number of amino acid residues in SEQ ID NO: 6, 8, or 26.

The lactaldehyde dehydrogenase may also be an allelic variant or artificial variant of a lactaldehyde dehydrogenase.

The lactaldehyde dehydrogenase can also include fused polypeptides or cleavable fusion polypeptides, as described supra.

Techniques used to isolate or clone a polynucleotide encoding a lactaldehyde dehydrogenase are described supra.

The polynucleotide sequence of SEQ ID NO: 5, 7, 25, or a subsequence thereof; as well as the amino acid sequence of SEQ ID NO: 6, 8, 26, or a fragment thereof; may be used to design nucleic acid probes to identify and clone DNA encoding a lactaldehyde dehydrogenase from strains of different genera or species, as described supra. A genomic DNA or cDNA library prepared from such other organisms may be screened for DNA that hybridizes with the probes described above and encodes a lactaldehyde dehydrogenase, as described supra.

In one aspect, the nucleic acid probe is SEQ ID NO: 5, 7, or 25. In another aspect, the nucleic acid probe is the mature polypeptide coding sequence of SEQ ID NO: 5, 7, or 25. In another aspect, the nucleic acid probe is a polynucleotide sequence that encodes SEQ ID NO: 6, 8, 26, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

Polynucleotides encoding the lactaldehyde dehydrogenase may be obtained from microorganisms of any genus. In one aspect, the lactaldehyde dehydrogenase may be a bacterial, a yeast, or a filamentous fungal lactaldehyde dehydrogenase obtained from the microorganisms described herein. In another aspect, the lactaldehyde dehydrogenase is a Lactobacillus lactaldehyde dehydrogenase, such as the Lactobacillus brevis lactaldehyde dehydrogenase of SEQ ID NO: 6, or the Lactobacillus buchneri lactaldehyde dehydrogenase of SEQ ID NO: 26. In another aspect, the lactaldehyde dehydrogenase is an Escherichia lactaldehyde dehydrogenase, such as the Escherichia coli lactaldehyde dehydrogenase of SEQ ID NO: 8.

Other lactaldehyde dehydrogenase candidates that can be used with the host cells and methods of use described herein include, but are not limited to, the Lactobacillus casei lactaldehyde dehydrogenase of SEQ ID NO: 30 (encoded by the polynucleotide sequence of SEQ ID NO: 29; UNIPROT:D8GC01), and the Lactobacillus hilgardii lactaldehyde dehydrogenase of SEQ ID NO: 32 (encoded by the polynucleotide sequence of SEQ ID NO: 31; UNIPROT:C0XL11). Any aspect described herein related to sequence identity, hybridization, amino acid modifications (e.g., substitutions, deletions, and/or insertions), fragments or subsequences thereof is embraced for the lactaldehyde dehydrogenases above.

In some aspects, the lactaldehyde dehydrogenase has at least 20%, e.g., at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or at least 100% of the lactaldehyde dehydrogenase activity of the mature polypeptide sequence of SEQ ID NO: 6, 8, or 26 under the same conditions.

The lactaldehyde dehydrogenase may also be identified and obtained from other sources including microorganisms isolated from nature (e.g., soil, composts, water, silage, etc.) or DNA samples obtained directly from natural materials (e.g., soil, composts, water, silage, etc.) as described supra.

### Lactaldehyde Reductases and Polynucleotides Encoding Lactaldehyde Reductases

In some aspects of the recombinant host cells and methods of use thereof, the host cells have lactaldehyde reductase activity. The lactaldehyde reductase can be any lactaldehyde reductase that is suitable for the host cells and their methods of use described herein, such as a naturally occurring lactaldehyde reductase or a variant thereof that retains lactaldehyde reductase activity. In one aspect, the lactaldehyde reductase is present in the cytosol of the host cells. In some aspects, the host cells comprises one or more (e.g., two, several) heterologous polynucleotides that encode a lactaldehyde reductase.

In some aspects, the host cells comprising the one or more (e.g., two, several) heterologous polynucleotides that encode a lactaldehyde reductase have an increased level of lactaldehyde reductase activity compared to the host cells without the one or more polynucleotides that encode a lactaldehyde reductase, when cultivated under the same conditions. In some aspects, the host cells comprising the one or more heterologous polynucleotides that encode a lactaldehyde reductase have an increased level of lactaldehyde reductase activity of at least 5%, e.g., at least 10%, at least 15%, at least 20%, at least 25%, at least 50%, at least 100%, at least 150%, at least 200%, at least 300%, or at 500% compared to the host cells without the one or more polynucleotides that encode a lactaldehyde reductase, when cultivated under the same conditions.

In some aspects, the host cell comprises a heterologous polynucleotide that encodes a lactaldehyde reductase. In one aspect, the heterologous polynucleotide that encodes a lactaldehyde reductase is selected from: (a) a polynucleotide that encodes a lactaldehyde reductase having at least 65% sequence identity to SEQ ID NO: 10, 12, 28, or the mature polypeptide sequence thereof; (b) a polynucleotide that hybridizes under low stringency conditions with SEQ ID NO: 9, 11, 27, the mature polypeptide coding sequence thereof, or the full-length complementary strand of the foregoing; and (c) a polynucleotide that has at least 65% sequence identity to SEQ ID NO: 9, 11, 27, or the mature polypeptide coding sequence thereof. As can be appreciated by one of skill in the art, in some instances the heterologous polynucleotide that encodes a lactaldehyde reductase may qualify under more than one of the selections (a), (b) and (c) noted above.

In one aspect, the heterologous polynucleotide encodes a lactaldehyde reductase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 10, 12, 28, or the mature polypeptide sequence thereof. In one aspect, the lactaldehyde reductase sequence differs by no more than ten amino acids, e.g., by no more than five amino acids, by no more than four amino acids, by no more than three amino acids, by no more than two amino acids, or by one amino acid from SEQ ID NO: 10, 12, 28, or the mature polypeptide sequence thereof.

In one aspect, the lactaldehyde reductase comprises or consists of the amino acid sequence of SEQ ID NO: 10, 12, or 28; the mature polypeptide sequence of SEQ ID NO: 10, 12, or 28; an allelic variant thereof, or a fragment of the foregoing, having lactaldehyde reductase activity. In another aspect, the lactaldehyde reductase comprises or consists of the amino acid sequence of SEQ ID NO: 10, 12, or 28. In another aspect, the lactaldehyde reductase comprises or consists of the mature polypeptide sequence of SEQ ID NO: 10, 12, or 28. In another aspect, the lactaldehyde reductase comprises or consists of amino acids 1 to 383 of SEQ ID NO: 10, amino acids 1 to 382 of SEQ ID NO: 12, or amino acids 1 to 389 of SEQ ID NO: 28.

In one aspect, the heterologous polynucleotide encodes a lactaldehyde reductase having an amino acid substitution, deletion, and/or insertion of one or more (e.g., two, several) amino acids of SEQ ID NO: 10, 12, 28, or the mature polypeptide sequence thereof, as described supra. In some aspects, the total number of amino acid substitutions, deletions and/or insertions of SEQ ID NO: 10, 12, 28, or the mature polypeptide sequence thereof, is not more than 10, e.g., not more than 9, 8, 7, 6, 5, 4, 3, 2, or 1.

In one aspect, the heterologous polynucleotide that encodes a lactaldehyde reductase hybridizes under at least low stringency conditions, e.g., medium stringency conditions, medium-high stringency conditions, high stringency conditions, or very high stringency conditions with SEQ ID NO: 9, 11, 27, the mature polypeptide coding sequence thereof, or the full-length complementary strand of the foregoing (see, e.g., J. Sambrook, E. F. Fritsch, and T. Maniatus, 1989, supra).

In one aspect, the heterologous polynucleotide that encodes a lactaldehyde reductase has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 9, 11, 27, or the mature polypeptide coding sequence thereof.

In one aspect, the heterologous polynucleotide that encodes a lactaldehyde reductase comprises SEQ ID NO: 9, 11, 27, or the mature polypeptide coding sequence thereof. In one aspect, the heterologous polynucleotide that encodes the lactaldehyde reductase comprises SEQ ID NO: 9, 11, or 27. In one aspect, the heterologous polynucleotide that encodes the lactaldehyde reductase comprises the mature polypeptide coding sequence of SEQ ID NO: 9, 11, or 27. In one aspect, the mature polypeptide coding sequence is nucleotides 1 to 1152 of SEQ ID NO: 9, nucleotides 1 to 1149 of SEQ ID NO: 11, or nucleotides 1 to 1170 of SEQ ID NO: 27. In one aspect, the heterologous polynucleotide comprises a subsequence of SEQ ID NO: 9, 11, 27, or the mature polypeptide coding sequence thereof, wherein the subsequence encodes a polypeptide having lactaldehyde reductase activity. In one aspect, the number of nucleotides residues in the subsequence is at least 75%, e.g., at least 80%, 85%, 90%, or 95% of the number of nucleotide residues in SEQ ID NO: 9, 11, or 27.

In one aspect, the heterologous polynucleotide encodes a fragment of SEQ ID NO: 10, 12, 28, or the mature polypeptide sequence thereof, wherein the fragment has lactaldehyde reductase activity. In one aspect, the number of amino acid residues in the fragment is at least 75%, e.g., at least 80%, 85%, 90%, or 95% of the number of amino acid residues in SEQ ID NO: 10, 12, or 28.

The lactaldehyde reductase may also be an allelic variant or artificial variant of a lactaldehyde reductase.

The lactaldehyde reductase can also include fused polypeptides or cleavable fusion polypeptides, as described supra.

Techniques used to isolate or clone a polynucleotide encoding a lactaldehyde reductase are described supra.

The polynucleotide sequence of SEQ ID NO: 9, 11, 27, or a subsequence thereof; as well as the amino acid sequence of SEQ ID NO: 10, 12, 28, or a fragment thereof; may be used to design nucleic acid probes to identify and clone DNA encoding a lactaldehyde reductase from strains of different genera or species, as described supra. A genomic DNA or cDNA library prepared from such other organisms may be screened for DNA that hybridizes with the probes described above and encodes a lactaldehyde reductase, as described supra.

In one aspect, the nucleic acid probe is SEQ ID NO: 9, 11, or 27. In another aspect, the nucleic acid probe is the mature polypeptide coding sequence of SEQ ID NO: 9, 11, or 27. In another aspect, the nucleic acid probe is a polynucleotide sequence that encodes SEQ ID NO: 10, 12, 28, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

Polynucleotides encoding the lactaldehyde reductase may be obtained from microorganisms of any genus. In one aspect, the lactaldehyde reductase may be a bacterial, a yeast, or a filamentous fungal lactaldehyde reductase obtained from the microorganisms described herein. In another aspect, the lactaldehyde reductase is a Lactobacillus lactaldehyde reductase. In another aspect, the lactaldehyde reductase is an Escherichia lactaldehyde reductase, such as the Escherichia coli lactaldehyde reductase of SEQ ID NO: 10. In another aspect, the lactaldehyde reductase is a Citrobacter lactaldehyde reductase, such as the Citrobacter koseri lactaldehyde reductase of SEQ ID NO: 12.

Other lactaldehyde reductase candidates that can be used with the host cells and methods of use described herein include, but are not limited to, the Salmonella enterica lactaldehyde reductase of SEQ ID NO: 54 (encoded by the polynucleotide sequence of SEQ ID NO: 53; UNIPROT:E7XFN0) and the Clostridium butyricum lactaldehyde reductase of SEQ ID NO: 56 (encoded by the polynucleotide sequence of SEQ ID NO: 55; UNIPROT:C4ILU0). Any aspect described herein related to sequence identity, hybridization, amino acid modifications (e.g., substitutions, deletions, and/or insertions), fragments or subsequences thereof is embraced for the lactaldehyde reductases above.

In some aspects, the lactaldehyde reductase has at least 20%, e.g., at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or at least 100% of the lactaldehyde reductase activity of the mature polypeptide sequence of SEQ ID NO: 10, 12, or 28 under the same conditions.

The lactaldehyde reductase may also be identified and obtained from other sources including microorganisms isolated from nature (e.g., soil, composts, water, silage, etc.) or DNA samples obtained directly from natural materials (e.g., soil, composts, water, silage, etc.) as described supra.

### Propanediol Dehydratases and Polynucleotides Encoding Propanediol Dehydratases

In some aspects of the recombinant host cells and methods of use thereof, the host cells have propanediol dehydratase activity. The propanediol dehydratase can be any propanediol dehydratase that is suitable for the host cells and their methods of use described herein, such as a naturally occurring propanediol dehydratase or a variant thereof that retains propanediol dehydratase activity. In one aspect, the propanediol dehydratase is present in the cytosol of the host cells. In some aspects, the host cells comprise one or more (e.g., two, several) heterologous polynucleotides that encode a propanediol dehydratase. In some aspects, the propanediol dehydratase is a protein complex comprising a first propanediol dehydratase subunit and the second propanediol dehydratase subunit wherein the subunits comprise different amino acid sequences.

In some aspects, the host cells comprise one or more (e.g., two, several) heterologous polynucleotides encoding a propanediol dehydratase. In some aspects, the host cells comprising the one or more heterologous polynucleotides that encode a propanediol dehydratase have an increased level of propanediol dehydratase activity compared to the host cells without the one or more polynucleotides that encode a propanediol dehydratase, when cultivated under the same conditions. In some aspects, the host cells comprising the one or more heterologous polynucleotides that encode a propanediol dehydratase have an increased level of propanediol dehydratase activity of at least 5%, e.g., at least 10%, at least 15%, at least 20%, at least 25%, at least 50%, at least 100%, at least 150%, at least 200%, at least 300%, or at 500% compared to the host cells without the one or more polynucleotides that encode a propanediol dehydratase, when cultivated under the same conditions.

In one aspect, host cells comprise a first heterologous polynucleotide encoding a first propanediol dehydratase subunit and a second heterologous polynucleotide encoding a second propanediol dehydratase subunit, wherein the first propanediol dehydratase subunit and second propanediol dehydratase subunit form a protein complex having propanediol dehydratase activity.

In one aspect, the heterologous polynucleotide encoding the first propanediol dehydratase subunit and the heterologous polynucleotide encoding the second propanediol dehydratase subunit are contained in a single heterologous polynucleotide. In another aspect, the heterologous polynucleotide encoding the first propanediol dehydratase subunit and the heterologous polynucleotide encoding the second propanediol dehydratase subunit are each contained in separate unlinked heterologous polynucleotides. An expanded discussion of nucleic acid constructs and expression vectors related to propanediol dehydratases and other polypeptides is described herein.

In one aspect, the heterologous polynucleotide that encodes the first propanediol dehydratase subunit is selected from: (a) a polynucleotide that encodes a propanediol dehydratase subunit having at least 65% sequence identity to SEQ ID NO: 14, 18, or the mature polypeptide sequence thereof; (b) a polynucleotide that hybridizes under low stringency conditions with SEQ ID NO: 13, 17, the mature polypeptide coding sequence thereof, or the full-length complementary strand of the foregoing; and (c) a polynucleotide that has at least 65% sequence identity to SEQ ID NO: 13, 17, or the mature polypeptide coding sequence thereof; and

the heterologous polynucleotide that encodes the second propanediol dehydratase subunit is selected from: (a) a polynucleotide that encodes a propanediol dehydratase subunit having at least 65% sequence identity to SEQ ID NO: 16, 20, or the mature polypeptide sequence thereof; (b) a polynucleotide that hybridizes under low stringency conditions with SEQ ID NO: 15, 19, the mature polypeptide coding sequence thereof, or the full-length complementary strand of the foregoing; and (c) a polynucleotide that has at least 65% sequence identity to SEQ ID NO: 15, 19, or the mature polypeptide coding sequence thereof.

In one aspect, the first propanediol dehydratase subunit has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 14, 18, or the mature polypeptide sequence thereof; and the second propanediol dehydratase subunit has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 16, 20, or the mature polypeptide sequence thereof. In one aspect, the first propanediol dehydratase subunit differs by no more than ten amino acids, e.g., by no more than five amino acids, by no more than four amino acids, by no more than three amino acids, by no more than two amino acids, or by one amino acid from SEQ ID NO: 14, 18, or the mature polypeptide sequence thereof; and the second propanediol dehydratase subunit differs by no more than ten amino acids, e.g., by no more than five amino acids, by no more than four amino acids, by no more than three amino acids, by no more than two amino acids, or by one amino acid from SEQ ID NO: 16, 20, or the mature polypeptide sequence thereof.

In one aspect, the first propanediol dehydratase subunit comprises or consists of the amino acid sequence of SEQ ID NO: 14, 18, the mature polypeptide sequence of SEQ ID NO: 14, 18, an allelic variant thereof, or a fragment of the foregoing; and the second propanediol dehydratase subunit comprises or consists of the amino acid sequence of SEQ ID NO: 16, 20, the mature polypeptide sequence of SEQ ID NO: 16, 20, an allelic variant thereof, or a fragment of the foregoing. In another aspect, the first propanediol dehydratase subunit comprises or consists of the amino acid sequence of SEQ ID NO: 14 or 18; and the second propanediol dehydratase subunit comprises or consists of the amino acid sequence of SEQ ID NO: 16 or 20. In another aspect, the first propanediol dehydratase subunit comprises or consists of the mature polypeptide sequence of SEQ ID NO: 14 or 18; and the second propanediol dehydratase subunit comprises or consists of the mature polypeptide sequence of SEQ ID NO: 16 or 20. In another aspect, the first propanediol dehydratase subunit comprises or consists of amino acids 1 to 224 of SEQ ID NO: 14 or amino acids 1 to 236 of SEQ ID NO: 18; and the second propanediol dehydratase subunit comprises or consists of amino acids 1 to 173 of SEQ ID NO: 16 or amino acids 1 to 171 of SEQ ID NO: 20.

In one aspect, the first propanediol dehydratase subunit comprises a substitution, deletion, and/or insertion of one or more (e.g., two, several) amino acids of SEQ ID NO: 14, 18, or the mature polypeptide sequence thereof; and/or the second propanediol dehydratase subunit comprises a substitution, deletion, and/or insertion of one or more amino acids of SEQ ID NO: 16, 20, or the mature polypeptide sequence thereof, as described supra. In some aspects, the total number of amino acid substitutions, deletions and/or insertions of SEQ ID NO: 14, 18, or the mature polypeptide sequence thereof is not more than 10, e.g., not more than 9, 8, 7, 6, 5, 4, 3, 2, or 1. In some aspects, the total number of amino acid substitutions, deletions and/or insertions of SEQ ID NO: 16, 20, or the mature polypeptide sequence thereof, is not more than 10, e.g., not more than 9, 8, 7, 6, 5, 4, 3, 2, or 1.

In one aspect, the heterologous polynucleotide that encodes the first propanediol dehydratase subunit hybridizes under at least low stringency conditions, e.g., medium stringency conditions, medium-high stringency conditions, high stringency conditions, or very high stringency conditions with SEQ ID NO: 13, 17, the mature polypeptide coding sequence thereof, or the full-length complementary strand of the foregoing; and the heterologous polynucleotide that encodes the second propanediol dehydratase subunit hybridizes under at least low stringency conditions, e.g., medium stringency conditions, medium-high stringency conditions, high stringency conditions, or very high stringency conditions with SEQ ID NO: 15, 19, the mature polypeptide coding sequence thereof, or the full-length complementary strand of the foregoing (see, e.g., J. Sambrook, E. F. Fritsch, and T. Maniatus, 1989, supra).

In one aspect, the heterologous polynucleotide that encodes the first propanediol dehydratase subunit has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 13, 17, or the mature polypeptide coding sequence thereof; and the heterologous polynucleotide that encodes the second propanediol dehydratase subunit has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 15, 19, or the mature polypeptide coding sequence thereof.

In one aspect, the heterologous polynucleotide that encodes the first propanediol dehydratase subunit comprises SEQ ID NO: 13, 17, or the mature polypeptide coding sequence thereof; and the heterologous polynucleotide that encodes the second propanediol dehydratase subunit comprises SEQ ID NO: 15, 19, or the mature polypeptide coding sequence thereof. In one aspect, the heterologous polynucleotide that encodes the first propanediol dehydratase subunit comprises SEQ ID NO: 13, 17; and the heterologous polynucleotide that encodes the second propanediol dehydratase subunit comprises SEQ ID NO: 15, 19. In one aspect, the heterologous polynucleotide that encodes the first propanediol dehydratase subunit comprises the mature polypeptide coding sequence of SEQ ID NO: 13, 17; and the heterologous polynucleotide that encodes the second propanediol dehydratase subunit comprises the mature polypeptide coding sequence of SEQ ID NO: 15, 19. In one aspect, the mature polypeptide coding sequence is nucleotides 1 to 675 of SEQ ID NO: 13, nucleotides 1 to 711 of SEQ ID NO: 17, nucleotides 1 to 522 of SEQ ID NO: 15, and nucleotides 1 to 516 of SEQ ID NO: 19.

In one aspect, the heterologous polynucleotide that encodes the first propanediol dehydratase subunit comprises a subsequence of SEQ ID NO: 13, 17, or the mature polypeptide coding sequence thereof; and/or the heterologous polynucleotide that encodes the second propanediol dehydratase subunit is encoded by a subsequence of SEQ ID NO: 15, 19, or the mature polypeptide coding sequence thereof; wherein the first propanediol dehydratase subunit together with the second propanediol dehydratase subunit form a protein complex having propanediol dehydratase activity. In one aspect, the number of nucleotides residues in the subsequence is at least 75%, e.g., at least 80%, 85%, 90%, or 95% of the number of nucleotide residues in SEQ ID NO: 13, 15, 17, or 19.

In one aspect, the heterologous polynucleotide that encodes the first propanediol dehydratase subunit encodes a fragment of SEQ ID NO: 14, 18, or the mature polypeptide sequence thereof; and/or the heterologous polynucleotide that encodes the second propanediol dehydratase subunit encodes a fragment of SEQ ID NO: 16, 20, or the mature polypeptide sequence thereof; wherein the first and second propanediol dehydratase subunits together form a protein complex having propanediol dehydratase activity. In one aspect, the number of amino acid residues in the fragment is at least 75%, e.g., at least 80%, 85%, 90%, or 95% of the number of amino acid residues in SEQ ID NO: 14, 16, 18, or 20.

The propanediol dehydratase (or subunits thereof) may also be an allelic variant or artificial variant of a propanediol dehydratase.

The propanediol dehydratase (or subunits thereof) can also include fused polypeptides or cleavable fusion polypeptides, as described supra.

Techniques used to isolate or clone a polynucleotide encoding a propanediol dehydratase (or subunits thereof) are described supra.

The polynucleotide sequence of SEQ ID NO: 13, 15, 17, or 19, or a subsequence thereof; as well as the amino acid sequence of SEQ ID NO: 14, 16, 18, or 20, or a fragment thereof; may be used to design nucleic acid probes to identify and clone DNA encoding a propanediol dehydratase from strains of different genera or species, as described supra. A genomic DNA or cDNA library prepared from such other organisms may be screened for DNA that hybridizes with the probes described above and encodes a propanediol dehydratase, as described supra.

In one aspect, the nucleic acid probe is SEQ ID NO: 13, 15, 17, or 19. In another aspect, the nucleic acid probe is the mature polypeptide coding sequence of SEQ ID NO: 13, 15, 17, or 19. In another aspect, the nucleic acid probe is a polynucleotide sequence that encodes SEQ ID NO: 14, 16, 18, or 20, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

Polynucleotides encoding the propanediol dehydratase and subunits thereof, may be obtained from microorganisms of any genus. In one aspect, the propanediol dehydratase may be a bacterial, a yeast, or a filamentous fungal propanediol dehydratase obtained from the microorganisms described herein. In another aspect, the propanediol dehydratase is a Klebsiella propanediol dehydratase, such as the Klebsiella oxytoca propanediol dehydratase complex comprising of the mature polypeptide sequence of SEQ ID NO: 14 and the mature polypeptide sequence of SEQ ID NO: 16. In another aspect, the propanediol dehydratase is a Lactobacillus propanediol dehydratase, such as the Lactobacillus reuteri propanediol dehydratase complex comprising of the mature polypeptide sequence of SEQ ID NO: 18 and the mature polypeptide sequence of SEQ ID NO: 20.

Other propanediol dehydratase candidates that can be used with the host cells and methods of use described herein include, but are not limited to, the Lactobacillus dioliverans propanediol dehydratase complex comprising the subunits of SEQ ID NO: 58 (encoded by the polynucleotide sequence of SEQ ID NO: 57; InterPro:IPR003208) and SEQ ID NO: 60 (encoded by the polynucleotide sequence of SEQ ID NO: 59; InterPro:IPR003207). Any aspect described herein related to sequence identity, hybridization, amino acid modifications (e.g., substitutions, deletions, and/or insertions), fragments or subsequences thereof is embraced for the propanediol dehydratase above.

In some aspects, the propanediol dehydratase has at least 20%, e.g., at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or at least 100% of the propanediol dehydratase activity of a protein complex comprising a first subunit having the mature polypeptide sequence of SEQ ID NO: 14, and a second subunit having the mature polypeptide sequence of SEQ ID NO: 16; or a protein complex comprising a first subunit having the mature polypeptide sequence of SEQ ID NO: 18, and a second subunit having the mature polypeptide sequence of SEQ ID NO: 20 under the same conditions.

The propanediol dehydratase may also be identified and obtained from other sources including microorganisms isolated from nature (e.g., soil, composts, water, silage, etc.) or DNA samples obtained directly from natural materials (e.g., soil, composts, water, silage, etc.) as described supra.

n-Propanol Dehydrogenases and Polynucleotides Encoding n-Propanol Dehydrogenases

In some aspects of the recombinant host cells and methods of use thereof, the host cells have n-propanol dehydrogenase activity. The n-propanol dehydrogenase can be any n-propanol dehydrogenase that is suitable for the host cells and their methods of use described herein, such as a naturally occurring n-propanol dehydrogenase or a variant thereof that retains n-propanol dehydrogenase activity. In one aspect, the n-propanol dehydrogenase is present in the cytosol of the host cells. In some aspects, the host cells comprise one or more (e.g., two, several) heterologous polynucleotides that encode a n-propanol dehydrogenase.

In some aspects, the host cells comprising the one or more heterologous polynucleotides that encode an n-propanol dehydrogenase have an increased level of n-propanol dehydrogenase activity compared to the host cells without the one or more polynucleotides that encode an n-propanol dehydrogenase, when cultivated under the same conditions. In some aspects, the host cells comprising the one or more heterologous polynucleotides that encode an n-propanol dehydrogenase have an increased level of n-propanol dehydrogenase activity of at least 5%, e.g., at least 10%, at least 15%, at least 20%, at least 25%, at least 50%, at least 100%, at least 150%, at least 200%, at least 300%, or at 500% compared to host cell without the one or more polynucleotides that encode an n-propanol dehydrogenase, when cultivated under the same conditions.

In some aspects, the host cell comprises a heterologous polynucleotide that encodes an n-propanol dehydrogenase. In one aspect, the heterologous polynucleotide that encodes an n-propanol dehydrogenase is selected from: (a) a polynucleotide that encodes an n-propanol dehydrogenase having at least 65% sequence identity to SEQ ID NO: 22, 24 or the mature polypeptide sequence thereof; (b) a polynucleotide that hybridizes under low stringency conditions with SEQ ID NO: 21, 23, the mature polypeptide coding sequence thereof, or the full-length complementary strand of the foregoing; and (c) a polynucleotide that has at least 65% sequence identity to SEQ ID NO: 21, 23, or the mature polypeptide coding sequence thereof. As can be appreciated by one of skill in the art, in some instances the heterologous polynucleotide that encodes an n-propanol may qualify under more than one of the selections (a), (b) and (c) noted above.

In one aspect, the heterologous polynucleotide encodes an n-propanol dehydrogenase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 22, 24, or the mature polypeptide sequence thereof. In one aspect, the n-propanol dehydrogenase sequence differs by no more than ten amino acids, e.g., by no more than five amino acids, by no more than four amino acids, by no more than three amino acids, by no more than two amino acids, or by one amino acid from SEQ ID NO: 22, 24, or the mature polypeptide sequence thereof.

In one aspect, the n-propanol dehydrogenase comprises or consists of the amino acid sequence of SEQ ID NO: 22 or 24, the mature polypeptide sequence of SEQ ID NO: 22 or 24, an allelic variant thereof, or a fragment of the foregoing having n-propanol dehydrogenase activity. In another aspect, the n-propanol dehydrogenase comprises or consists of the amino acid sequence of SEQ ID NO: 22 or 24. In another aspect, the n-propanol dehydrogenase comprises or consists of the mature polypeptide sequence of SEQ ID NO: 22 or 24. In another aspect, the n-propanol dehydrogenase comprises or consists of amino acids 1 to 336 of SEQ ID NO: 22 or amino acids 1 to 376 of SEQ ID NO: 24.

In one aspect, the heterologous polynucleotide encodes an n-propanol dehydrogenase having an amino acid substitution, deletion, and/or insertion of one or more (e.g., two, several) amino acids of SEQ ID NO: 22, 24, or the mature polypeptide sequence thereof, as described supra. In some aspects, the total number of amino acid substitutions, deletions and/or insertions of SEQ ID NO: 22, 24, or the mature polypeptide sequence thereof, is not more than 10, e.g., not more than 9, 8, 7, 6, 5, 4, 3, 2, or 1.

In one aspect, the heterologous polynucleotide that encodes an n-propanol dehydrogenase hybridizes under at least low stringency conditions, e.g., medium stringency conditions, medium-high stringency conditions, high stringency conditions, or very high stringency conditions with SEQ ID NO: 21, 23, the mature polypeptide coding sequence thereof, or the full-length complementary strand of the foregoing (see, e.g., J. Sambrook, E. F. Fritsch, and T. Maniatus, 1989, supra).

In one aspect, the heterologous polynucleotide that encodes an n-propanol dehydrogenase has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 21, 23, or the mature polypeptide coding sequence thereof.

In one aspect, the heterologous polynucleotide that encodes an n-propanol dehydrogenase comprises SEQ ID NO: 21, 23, or the mature polypeptide coding sequence thereof. In one aspect, the heterologous polynucleotide that encodes the n-propanol dehydrogenase comprises SEQ ID NO: 21 or 23. In one aspect, the heterologous polynucleotide that encodes the n-propanol dehydrogenase comprises the mature polypeptide coding sequence of SEQ ID NO: 21 or 23. In one aspect, the mature polypeptide coding sequence is nucleotides 1 to 1011 of SEQ ID NO: 21 or nucleotides 1 to 1131 of SEQ ID NO: 23. In one aspect, the heterologous polynucleotide comprises a subsequence of SEQ ID NO: 21 or 23, wherein the subsequence encodes a polypeptide having n-propanol dehydrogenase activity. In one aspect, the number of nucleotides residues in the subsequence is at least 75%, e.g., at least 80%, 85%, 90%, or 95% of the number of nucleotide residues in SEQ ID NO: 21 or 23.

In one aspect, the heterologous polynucleotide encodes a fragment of SEQ ID NO: 22, 24, or the mature polypeptide sequence thereof, wherein the fragment has n-propanol dehydrogenase activity. In one aspect, the number of amino acid residues in the fragment is at least 75%, e.g., at least 80%, 85%, 90%, or 95% of the number of amino acid residues in SEQ ID NO: 22 or 24.

The n-propanol dehydrogenase may also be an allelic variant or artificial variant of a n-propanol dehydrogenase.

The n-propanol dehydrogenase can also include fused polypeptides or cleavable fusion polypeptides, as described supra.

Techniques used to isolate or clone a polynucleotide encoding a n-propanol dehydrogenase are described supra.

The polynucleotide sequence of SEQ ID NO: 21 or 23, or a subsequence thereof; as well as the amino acid sequence of SEQ ID NO: 22 or 24, or a fragment thereof; may be used to design nucleic acid probes to identify and clone DNA encoding a n-propanol dehydrogenase from strains of different genera or species, as described supra. A genomic DNA or cDNA library prepared from such other organisms may be screened for DNA that hybridizes with the probes described above and encodes a n-propanol dehydrogenase, as described supra.

In one aspect, the nucleic acid probe is SEQ ID NO: 21 or 23. In another aspect, the nucleic acid probe is the mature polypeptide coding sequence of SEQ ID NO: 21 or 23. In another aspect, the nucleic acid probe is a polynucleotide sequence that encodes SEQ ID NO: 22 or 24, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

Polynucleotides encoding the n-propanol dehydrogenase may be obtained from microorganisms of any genus. In one aspect, the n-propanol dehydrogenase may be a bacterial, a yeast, or a filamentous fungal n-propanol dehydrogenase obtained from the microorganisms described herein. In another aspect, the n-propanol dehydrogenase is a Lactobacillus n-propanol dehydrogenase. In another aspect, the n-propanol dehydrogenase is an Escherichia n-propanol dehydrogenase, such as the Escherichia coli n-propanol dehydrogenase of SEQ ID NO: 22. In another aspect, the n-propanol dehydrogenase is a Propionibacterium n-propanol dehydrogenase, such as the Propionibacterium freudenreichii n-propanol dehydrogenase of SEQ ID NO: 24.

Other n-propanol dehydrogenase candidates that can be used with the host cells and methods of use described herein include, but are not limited to, the Lactobacillus reuteri n-propanol dehydrogenases of SEQ ID NO: 62 (encoded by the polynucleotide sequence of SEQ ID NO: 61; InterPro:IPR001670). Any aspect described herein related to sequence identity, hybridization, amino acid modifications (e.g., substitutions, deletions, and/or insertions), fragments or subsequences thereof is embraced for the n-propanol dehydrogenases above.

In some aspects, the n-propanol dehydrogenase has at least 20%, e.g., at least 40%, at least 50%, at least 60%, at least 70%, at least 80%, at least 90%, at least 95%, at least 96%, at least 97%, at least 98%, at least 99%, or 100% of the n-propanol dehydrogenase activity of the mature polypeptide sequence of SEQ ID NO: 22 or 24 under the same conditions.

The n-propanol dehydrogenase may also be identified and obtained from other sources including microorganisms isolated from nature (e.g., soil, composts, water, silage, etc.) or DNA samples obtained directly from natural materials (e.g., soil, composts, water, silage, etc.) as described supra.

### Thiolases and Polynucleotides Encoding Thiolases

In some aspects of the recombinant host cells and methods of use thereof, the host cells have thiolase activity. The thiolase can be any thiolase that is suitable for the host cells and their methods of use described herein, such as a naturally occurring thiolase or a variant thereof that retains thiolase activity. In one aspect, the thiolase is present in the cytosol of the host cells. In some aspects, the host cells comprise one or more (e.g., two, several) heterologous polynucleotides that encode a thiolase.

In some aspects, the host cells comprising the one or more heterologous polynucleotides that encode a thiolase have an increased level of thiolase activity compared to the host cells without the one or more polynucleotides that encode a thiolase, when cultivated under the same conditions. In some aspects, the host cells comprising the one or more heterologous polynucleotides that encode a thiolase have an increased level of thiolase activity of at least 5%, e.g., at least 10%, at least 15%, at least 20%, at least 25%, at least 50%, at least 100%, at least 150%, at least 200%, at least 300%, or at 500% compared to the host cells without the one or more polynucleotides that encode a thiolase, when cultivated under the same conditions.

In some aspects, the host cell comprises a heterologous polynucleotide that encodes a thiolase. In one aspect, the heterologous polynucleotide that encodes a thiolase is selected from: (a) a polynucleotide that encodes a thiolase having at least 65% sequence identity to SEQ ID NO: 38, or the mature polypeptide sequence thereof; (b) a polynucleotide that hybridizes under low stringency conditions with SEQ ID NO: 37, the mature polypeptide coding sequence thereof, or the full-length complementary strand of the foregoing; and (c) a polynucleotide that has at least 65% sequence identity to SEQ ID NO: 37, or the mature polypeptide coding sequence thereof. As can be appreciated by one of skill in the art, in some instances the heterologous polynucleotide that encodes a thiolase may qualify under more than one of the selections (a), (b) and (c) noted above.

In one aspect, the heterologous polynucleotide encodes a thiolase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 38, or the mature polypeptide sequence thereof. In one aspect, the thiolase sequence differs by no more than ten amino acids, e.g., by no more than five amino acids, by no more than four amino acids, by no more than three amino acids, by no more than two amino acids, or by one amino acid from SEQ ID NO: 38, or the mature polypeptide sequence thereof.

In one aspect, the thiolase comprises or consists of the amino acid sequence of SEQ ID NO: 38, the mature polypeptide sequence of SEQ ID NO: 38, an allelic variant thereof, or a fragment of the foregoing having thiolase activity. In another aspect, the thiolase comprises or consists of the amino acid sequence of SEQ ID NO: 38. In another aspect, the thiolase comprises or consists of the mature polypeptide sequence of SEQ ID NO: 38. In another aspect, the thiolase comprises or consists of amino acids 1 to 392 of SEQ ID NO: 38.

In one aspect, the heterologous polynucleotide encodes a thiolase having an amino acid substitution, deletion, and/or insertion of one or more (e.g., two, several) amino acids of SEQ ID NO: 38, or the mature polypeptide sequence thereof, as described supra. In some aspects, the total number of amino acid substitutions, deletions and/or insertions of SEQ ID NO: 38, or the mature polypeptide sequence thereof, is not more than 10, e.g., not more than 9, 8, 7, 6, 5, 4, 3, 2, or 1.

In one aspect, the heterologous polynucleotide that encodes a thiolase hybridizes under at least low stringency conditions, e.g., medium stringency conditions, medium-high stringency conditions, high stringency conditions, or very high stringency conditions with SEQ ID NO: 37, the mature polypeptide coding sequence thereof, or the full-length complementary strand of the foregoing (see, e.g., J. Sambrook, E. F. Fritsch, and T. Maniatus, 1989, supra).

In one aspect, the heterologous polynucleotide that encodes a thiolase has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 37, or the mature polypeptide coding sequence thereof.

In one aspect, the heterologous polynucleotide that encodes a thiolase comprises SEQ ID NO: 37, or the mature polypeptide coding sequence thereof. In one aspect, the heterologous polynucleotide that encodes the thiolase comprises SEQ ID NO: 37. In one aspect, the heterologous polynucleotide that encodes the thiolase comprises the mature polypeptide coding sequence of SEQ ID NO: 37. In one aspect, the mature polypeptide coding sequence is nucleotides 1 to 1179 of SEQ ID NO: 37. In one aspect, the heterologous polynucleotide comprises a subsequence of SEQ ID NO: 37, or the mature polypeptide coding sequence thereof, wherein the subsequence encodes a polypeptide having thiolase activity. In one aspect, the number of nucleotides residues in the subsequence is at least 75%, e.g., at least 80%, 85%, 90%, or 95% of the number of nucleotide residues in SEQ ID NO: 37.

In one aspect, the heterologous polynucleotide encodes a fragment of SEQ ID NO: 38, or the mature polypeptide sequence thereof, wherein the fragment has thiolase activity. In one aspect, the number of amino acid residues in the fragment is at least 75%, e.g., at least 80%, 85%, 90%, or 95% of the number of amino acid residues in SEQ ID NO: 38.

The thiolase may also be an allelic variant or artificial variant of a thiolase.

The thiolase can also include fused polypeptides or cleavable fusion polypeptides, as described supra.

Techniques used to isolate or clone a polynucleotide encoding a thiolase are described supra.

The polynucleotide sequence of SEQ ID NO: 37, or a subsequence thereof; as well as the amino acid sequence of SEQ ID NO: 38, or a fragment thereof; may be used to design nucleic acid probes to identify and clone DNA encoding a thiolase from strains of different genera or species, as described supra. A genomic DNA or cDNA library prepared from such other organisms may be screened for DNA that hybridizes with the probes described above and encodes a thiolase, as described supra.

In one aspect, the nucleic acid probe is SEQ ID NO: 37. In another aspect, the nucleic acid probe is the mature polypeptide coding sequence of SEQ ID NO: 37. In another aspect, the nucleic acid probe is a polynucleotide sequence that encodes SEQ ID NO: 38, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

Polynucleotides encoding the thiolase may be obtained from microorganisms of any genus. In one aspect, the thiolase may be a bacterial, a yeast, or a filamentous fungal thiolase obtained from the microorganisms described herein. In another aspect, the thiolase is a Lactobacillus thiolase. In another aspect, the thiolase is a Clostridium thiolase, such as the Clostridium acetobutylicum thiolase of SEQ ID NO: 38.

Other thiolase candidates that can be used with the host cells and methods of use described herein include, but are not limited to, the a E. coli thiolase (NP_{—}416728, Martin et al., Nat. Biotechnology 21:796-802 (2003)), and a S. cerevisiae thiolase (NP_{—}015297, Hiser et al., J. Biol. Chem. 269:31383-31389 (1994)), a C. pasteurianum thiolase (e.g., protein ID ABAI8857.1), a C. beijerinckii thiolase (e.g., protein ID EAP59904.1 or EAP59331.1), a Clostridium perfringens thiolase (e.g., protein ID ABG86544.I, ABG83108.1), a Clostridium diflicile thiolase (e.g., protein ID CAJ67900.1 or ZP_{—}01231975.1), a Thermoanaerobacterium thermosaccharolyticum thiolase (e.g., protein ID CAB07500.1), a Thermoanaerobacter tengcongensis thiolase (e.g., A.L\.M23825.1), a Carboxydothermus hydrogenoformans thiolase (e.g., protein ID ABB13995.1), a Desulfotomaculum reducens MI-I thiolase (e.g., protein ID EAR45123.1), or a Candida tropicalis thiolase (e.g., protein ID BAA02716.1 or BAA02715.1). Any aspect described herein related to sequence identity, hybridization, amino acid modifications (e.g., substitutions, deletions, and/or insertions), fragments or subsequences thereof is embraced for the thiolases above.

In some aspects, the thiolase has at least 20%, e.g., at least 40%, at least 50%, at least 60%, at least 70%, at least 80%, at least 90%, at least 95%, at least 96%, at least 97%, at least 98%, at least 99%, or 100% of the thiolase activity of the mature polypeptide of SEQ ID NO: 38 under the same conditions.

The thiolase may also be identified and obtained from other sources including microorganisms isolated from nature (e.g., soil, composts, water, silage, etc.) or DNA samples obtained directly from natural materials (e.g., soil, composts, water, silage, etc.) as described supra.

### CoA-Transferases and Polynucleotides Encoding CoA-Transferases

In some aspects of the recombinant host cells and methods of use thereof, the host cells have CoA-transferase activity. The CoA-transferase can be any CoA-transferase that is suitable for the host cells and their methods of use described herein, such as a naturally occurring CoA-transferase or a variant thereof that retains CoA-transferase activity. In some aspects, the CoA-transferase is an acetoacetyl-CoA:acetate/butyrate CoA transferase. In some aspects, the CoA-transferase is an acetoacetyl-CoA hydrolase. In some aspects, the CoA-transferase is an acetoacetyl-CoA transferase that converts acetoacetyl-CoA and acetate to acetoacetate and acetyl-CoA. In some aspects, the CoA-transferase is a succinyl-CoA:acetoacetate transferase. In some aspects, the CoA-transferase is present in the cytosol of the host cells. In some aspects, the CoA-transferase is a protein complex comprising a first CoA-transferase subunit and the second CoA-transferase subunit wherein the subunits comprise different amino acid sequences.

In some aspects, the host cells comprise one or more (e.g., two, several) heterologous polynucleotides encoding a CoA-transferase transferase. In some aspects, the host cell comprising the one or more heterologous polynucleotides that encode a CoA-transferase has an increased level of CoA-transferase activity compared to the host cell without the one or more polynucleotides that encode a CoA-transferase, when cultivated under the same conditions. In some aspects, the host cells comprising the one or more heterologous polynucleotides that encode a CoA-transferase have an increased level of CoA-transferase activity of at least 5%, e.g., at least 10%, at least 15%, at least 20%, at least 25%, at least 50%, at least 100%, at least 150%, at least 200%, at least 300%, or at 500% compared to the host cells without the one or more polynucleotides that encode the CoA-transferase, when cultivated under the same conditions.

In some aspects, the host cells comprise one or more (e.g., two, several) heterologous polynucleotides encoding a succinyl-CoA:acetoacetate transferase. In some aspects, the host cells comprise one or more heterologous polynucleotides encoding a succinyl-CoA:acetoacetate transferase. In one aspect, host cells comprise a first heterologous polynucleotide encoding a first succinyl-CoA:acetoacetate transferase subunit and a second heterologous polynucleotide encoding a second succinyl-CoA:acetoacetate transferase subunit, wherein the first succinyl-CoA:acetoacetate transferase subunit and second succinyl-CoA:acetoacetate transferase subunit form a protein complex having succinyl-CoA:acetoacetate transferase activity.

In one aspect, the heterologous polynucleotide encoding the first succinyl-CoA:acetoacetate transferase subunit and the heterologous polynucleotide encoding the second succinyl-CoA:acetoacetate transferase subunit are contained in a single heterologous polynucleotide. In another aspect, the heterologous polynucleotide encoding the first succinyl-CoA:acetoacetate transferase subunit and the heterologous polynucleotide encoding the second succinyl-CoA:acetoacetate transferase subunit are each contained in separate unlinked heterologous polynucleotides. An expanded discussion of nucleic acid constructs and expression vectors related to succinyl-CoA:acetoacetate transferases and other polypeptides is described herein.

In one aspect, the heterologous polynucleotide that encodes the first succinyl-CoA:acetoacetate transferase subunit is selected from: (a) a polynucleotide that encodes a succinyl-CoA:acetoacetate transferase subunit having at least 65% sequence identity to SEQ ID NO: 40, 44, or the mature polypeptide sequence thereof; (b) a polynucleotide that hybridizes under low stringency conditions with SEQ ID NO: 39, 43, the mature polypeptide coding sequence thereof, or the full-length complementary strand of the foregoing; and (c) a polynucleotide that has at least 65% sequence identity to SEQ ID NO: 39, 43, or the mature polypeptide coding sequence thereof; and

the heterologous polynucleotide that encodes the second succinyl-CoA:acetoacetate transferase subunit is selected from: (a) a polynucleotide that encodes a succinyl-CoA:acetoacetate transferase subunit having at least 65% sequence identity to SEQ ID NO: 42, 46, or the mature polypeptide sequence thereof; (b) a polynucleotide that hybridizes under low stringency conditions with SEQ ID NO: 41, 45, the mature polypeptide coding sequence thereof, or the full-length complementary strand of the foregoing; and (c) a polynucleotide that has at least 65% sequence identity to SEQ ID NO: 41, 45, or the mature polypeptide coding sequence thereof. As can be appreciated by one of skill in the art, in some instances the heterologous polynucleotide that encodes the first succinyl-CoA:acetoacetate transferase or the second succinyl-CoA:acetoacetate transferase may qualify under more than one of the respective selections (a), (b) and (c) noted above.

In one aspect, the first succinyl-CoA:acetoacetate transferase subunit has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 40, 44, or the mature polypeptide sequence thereof; and the second succinyl-CoA:acetoacetate transferase subunit has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 42, 46, or the mature polypeptide sequence thereof. In one aspect, the first succinyl-CoA:acetoacetate transferase subunit sequence differs by no more than ten amino acids, e.g., by no more than five amino acids, by no more than four amino acids, by no more than three amino acids, by no more than two amino acids, or by one amino acid from SEQ ID NO: 40, 44, or the mature polypeptide sequence thereof; and the second succinyl-CoA:acetoacetate transferase subunit sequence differs by no more than ten amino acids, e.g., by no more than five amino acids, by no more than four amino acids, by no more than three amino acids, by no more than two amino acids, or by one amino acid from SEQ ID NO: 42, 46, or the mature polypeptide sequence thereof.

In one aspect, the first succinyl-CoA:acetoacetate transferase subunit comprises or consists of the amino acid sequence of SEQ ID NO: 40 or 44, the mature polypeptide sequence of SEQ ID NO: 40 or 44, an allelic variant thereof, or a fragment of the foregoing; and the second succinyl-CoA:acetoacetate transferase subunit comprises or consists of the amino acid sequence of SEQ ID NO: 42 or 46, the mature polypeptide sequence of SEQ ID NO: 42 or 46, an allelic variant thereof, or a fragment of the foregoing. In another aspect, the first succinyl-CoA:acetoacetate transferase subunit comprises or consists of the amino acid sequence of SEQ ID NO: 40 or 44; and the second succinyl-CoA:acetoacetate transferase subunit comprises or consists of the amino acid sequence of SEQ ID NO: 42 or 46. In another aspect, the first succinyl-CoA:acetoacetate transferase subunit comprises or consists of the mature polypeptide sequence of SEQ ID NO: 40 or 44; and the second succinyl-CoA:acetoacetate transferase subunit comprises or consists of the mature polypeptide sequence of SEQ ID NO: 42 or 46. In another aspect, the first succinyl-CoA:acetoacetate transferase subunit comprises or consists of amino acids 1 to 233 of SEQ ID NO: 40, or amino acids 1 to 237 of SEQ ID NO: 44; and the second succinyl-CoA:acetoacetate transferase subunit comprises or consists of amino acids 1 to 216 of SEQ ID NO: 42, or amino acids 1 to 218 of SEQ ID NO: 46.

In one aspect, the first succinyl-CoA:acetoacetate transferase subunit comprises a substitution, deletion, and/or insertion of one or more (e.g., two, several) amino acids of SEQ ID NO: 40, 44, or the mature polypeptide sequence thereof; and/or the second succinyl-CoA:acetoacetate transferase subunit comprises a substitution, deletion, and/or insertion of one or more amino acids of SEQ ID NO: 42, 46, or the mature polypeptide sequence thereof, as described supra. In some aspects, the total number of amino acid substitutions, deletions and/or insertions of SEQ ID NO: 40, 44, or the mature polypeptide sequence thereof is not more than 10, e.g., not more than 9, 8, 7, 6, 5, 4, 3, 2, or 1. In some aspects, the total number of amino acid substitutions, deletions and/or insertions of SEQ ID NO: 42, 46, or the mature polypeptide sequence thereof, is not more than 10, e.g., not more than 9, 8, 7, 6, 5, 4, 3, 2, or 1.

In one aspect, the heterologous polynucleotide that encodes the first succinyl-CoA:acetoacetate transferase subunit hybridizes under at least low stringency conditions, e.g., medium stringency conditions, medium-high stringency conditions, high stringency conditions, or very high stringency conditions with SEQ ID NO: 39, 43, the mature polypeptide coding sequence thereof, or the full-length complementary strand of the foregoing; and the heterologous polynucleotide that encodes the second succinyl-CoA:acetoacetate transferase subunit hybridizes under at least low stringency conditions, e.g., medium stringency conditions, medium-high stringency conditions, high stringency conditions, or very high stringency conditions with SEQ ID NO: 41, 45, the mature polypeptide coding sequence thereof, or the full-length complementary strand of the foregoing (see, e.g., J. Sambrook, E. F. Fritsch, and T. Maniatus, 1989, supra).

In one aspect, the heterologous polynucleotide that encodes the first succinyl-CoA:acetoacetate transferase subunit has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 39, 43, or the mature polypeptide coding sequence thereof; and the heterologous polynucleotide that encodes the second succinyl-CoA:acetoacetate transferase subunit has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 41, 45, or the mature polypeptide coding sequence thereof.

In one aspect, the heterologous polynucleotide that encodes the first succinyl-CoA:acetoacetate transferase subunit comprises SEQ ID NO: 39, 43, or the mature polypeptide coding sequence thereof; and the heterologous polynucleotide that encodes the second succinyl-CoA:acetoacetate transferase subunit comprises SEQ ID NO: 41, 45, or the mature polypeptide coding sequence thereof. In one aspect, the heterologous polynucleotide that encodes the first succinyl-CoA:acetoacetate transferase subunit comprises SEQ ID NO: 39, 43; and the heterologous polynucleotide that encodes the second succinyl-CoA:acetoacetate transferase subunit comprises SEQ ID NO: 41, 45. In one aspect, the heterologous polynucleotide that encodes the first succinyl-CoA:acetoacetate transferase subunit comprises the mature polypeptide coding sequence of SEQ ID NO: 39, 43; and the heterologous polynucleotide that encodes the second succinyl-CoA:acetoacetate transferase subunit comprises the mature polypeptide coding sequence of SEQ ID NO: 41, 45. In one aspect, the mature polypeptide coding sequence is nucleotides 1 to 702 of SEQ ID NO: 39, and nucleotides 1 to 714 of SEQ ID NO: 43.

In one aspect, the heterologous polynucleotide that encodes the first succinyl-CoA:acetoacetate transferase subunit comprises a subsequence of SEQ ID NO: 39 or 43; and/or the heterologous polynucleotide that encodes the second polypeptide subunit comprises a subsequence of SEQ ID NO: 41 or 45; wherein the first and second succinyl-CoA:acetoacetate transferase subunits together form a protein complex having succinyl-CoA:acetoacetate transferase activity. In one aspect, the number of nucleotides residues in the subsequence is at least 75%, e.g., at least 80%, 85%, 90%, or 95% of the number of nucleotide residues in SEQ ID NO: 39, 43, 41, or 45.

In one aspect, the heterologous polynucleotide that encodes the first succinyl-CoA:acetoacetate transferase subunit encodes a fragment of SEQ ID NO: 40, 44, or the mature polypeptide sequence thereof; and/or the heterologous polynucleotide that encodes the second succinyl-CoA:acetoacetate transferase subunit encodes a fragment of SEQ ID NO: 42, 46, or the mature polypeptide sequence thereof; wherein the first and second succinyl-CoA:acetoacetate transferase subunits together form a protein complex having succinyl-CoA:acetoacetate transferase activity. In one aspect, the number of amino acid residues in the fragment is at least 75%, e.g., at least 80%, 85%, 90%, or 95% of the number of amino acid residues in SEQ ID NO: 40, 44, 42, or 46.

The CoA-transferase (or subunits thereof) may also be an allelic variant or artificial variant of a CoA-transferase.

The CoA-transferase (or subunits thereof) can also include fused polypeptides or cleavable fusion polypeptides, as described supra.

Techniques used to isolate or clone a polynucleotide encoding a CoA-transferase (or subunits thereof) are described supra.

The polynucleotide sequence of SEQ ID NO: 39, 43, 41, 45, or a subsequence thereof; as well as the amino acid sequence of SEQ ID NO: 40, 44, 42, 46, or a fragment thereof; may be used to design nucleic acid probes to identify and clone DNA encoding a succinyl-CoA:acetoacetate transferase from strains of different genera or species, as described supra. A genomic DNA or cDNA library prepared from such other organisms may be screened for DNA that hybridizes with the probes described above and encodes a succinyl-CoA:acetoacetate transferase, as described supra.

In one aspect, the nucleic acid probe is SEQ ID NO: 39, 43, 41, or 45. In another aspect, the nucleic acid probe is the mature polypeptide coding sequence of SEQ ID NO: 39, 43, 41, or 45. In another aspect, the nucleic acid probe is a polynucleotide sequence that encodes SEQ ID NO: 40, 44, 42, or 46, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

Polynucleotides encoding the CoA-transferase and subunits thereof, may be obtained from microorganisms of any genus. In one aspect, the CoA-transferase may be a bacterial, a yeast, or a filamentous fungal CoA-transferase obtained from the microorganisms described herein. In one aspect, the CoA-transferase is a Lactobacillus CoA-transferase. In another aspect, the CoA-transferase is a Bacillus CoA-transferase, such as a Bacillus succinyl-CoA:acetoacetate transferase, e.g., the Bacillus subtilis succinyl-CoA:acetoacetate transferase complex comprising of the mature polypeptide sequence of SEQ ID NO: 40 and the mature polypeptide sequence of SEQ ID NO: 42, or the Bacillus mojavensis succinyl-CoA:acetoacetate transferase complex comprising of the mature polypeptide sequence of SEQ ID NO: 44 and the mature polypeptide sequence of SEQ ID NO: 46.

Other succinyl-CoA:acetoacetate transferase candidates that can be used with the host cells and methods of use described herein include, but are not limited to, a Helicobacter pylori succinyl-CoA:acetoacetate transferase (YP_{—}627417, YP_{—}627418, Corthesy-Theulaz, et al., J Biol Chem 272:25659-25667 (1997)), and a Homo sapiens succinyl-CoA:acetoacetate transferase (NP_{—}000427, NP071403, Fukao, T., et al., Genomics 68:144-151 (2000); Tanaka, H., et al., Mol Hum Reprod 8:16-23 (2002)). Any aspect described herein related to sequence identity, hybridization, amino acid modifications (e.g., substitutions, deletions, and/or insertions), fragments or subsequences thereof is embraced for the succinyl-CoA:acetoacetate transferase candidates above.

Other acetoacetyl-CoA:acetate/butyrate CoA transferase candidates that can be used with the host cells and methods of use described herein include, but are not limited to, an E. coli acetoacetyl-CoA:acetate CoA transferase (NP 416726.1, NP_{—}416725.1; Hanai et al., Appl Environ Microbiol 73:7814-7818 (2007)), a Clostridium acetobutylicum acetoacetyl-CoA:acetate CoA transferase (NP_{—}149326.1, NP_{—}149327.1; Jojima et al., Appl Microbiol Biotechnol 77:1219-1224 (2008)), and a Clostridium saccharoperbutylacetonicum acetoacetyl-CoA:acetate CoA transferase (AAP42564.1, AAP42565.1; Kosaka et al., Biosci. Biotechnol Biochem. 71:58-68 (2007)). Any aspect described herein related to sequence identity, hybridization, amino acid modifications (e.g., substitutions, deletions, and/or insertions), fragments or subsequences thereof is embraced for the acetoacetyl-CoA:acetate/butyrate CoA transferase candidates above.

Other acetoacetyl-CoA hydrolase candidates that can be used with the host cells and methods of use described herein include, but are not limited to, acyl-CoA hydrolases, 3-hydroxyisobutyryl-CoA hydrolases, acetyl-CoA hydrolases, and dicarboxylic acid thioesterases, such as a Rattus norvegicus 3-hydroxyisobutyryl-CoA hydrolase (Q5XIE6.2; Shimomura et al., J Biol. Chem. 269:14248-14253 (1994)), a Homo sapiens 3-hydroxyisobutyryl-CoA hydrolase (Q6NVY1.2; Shimomura et al., supra), a Rattus norvegicus acetyl-CoA hydrolase (NP 570103.1; Robinson et al., Res. Commun. 71:959-965 (1976)), a Saccharomyces cerevisiae acetyl-CoA hydrolase (NP_{—}009538; Buu et al., J. Biol. Chem. 278: 17203-17209 (2003)), a Homo sapiens dicarboxylic acid thioesterase (CAA15502; Westin et al., J Biol. Chem. 280:38125-38132 (2005)), and an Escherichia coli dicarboxylic acid thioesterase (Naggert et al., J Biol. Chem. 266: 11044-11050 (1991)). Any aspect described herein related to sequence identity, hybridization, amino acid modifications (e.g., substitutions, deletions, and/or insertions), fragments or subsequences thereof is embraced for the acetoacetyl-CoA hydrolase candidates above.

In some aspects, the CoA-transferase has at least 20%, e.g., at least 40%, at least 50%, at least 60%, at least 70%, at least 80%, at least 90%, at least 95%, at least 96%, at least 97%, at least 98%, at least 99%, or 100% of the CoA-transferase activity of a protein complex comprising a first subunit having the mature polypeptide sequence of SEQ ID NO: 40, and a first subunit having the mature polypeptide sequence of SEQ ID NO: 42; or a protein complex comprising a first subunit having the mature polypeptide sequence of SEQ ID NO: 44, and a first subunit having the mature polypeptide sequence of SEQ ID NO: 46 under the same conditions.

The CoA-transferase may also be identified and obtained from other sources including microorganisms isolated from nature (e.g., soil, composts, water, silage, etc.) or DNA samples obtained directly from natural materials (e.g., soil, composts, water, silage, etc.) as described supra.

### Acetoacetate Decarboxylases and Polynucleotides Encoding Acetoacetate Decarboxylases

In some aspects of the recombinant host cells and methods of use thereof, the host cells have acetoacetate decarboxylase activity. The acetoacetate decarboxylase can be any acetoacetate decarboxylase that is suitable for the host cells and their methods of use described herein, such as a naturally occurring acetoacetate decarboxylase or a variant thereof that retains acetoacetate decarboxylase activity. In one aspect, the acetoacetate decarboxylase is present in the cytosol of the host cells. In some aspects, the host cells comprises one or more (e.g., two, several) heterologous polynucleotides that encode an acetoacetate decarboxylase.

In some aspects, the host cells comprising the one or more (e.g., two, several) heterologous polynucleotides that encode an acetoacetate decarboxylase have an increased level of acetoacetate decarboxylase activity compared to the host cells without the one or more polynucleotides that encode an acetoacetate decarboxylase, when cultivated under the same conditions. In some aspects, the host cells comprising the one or more heterologous polynucleotides that encode an acetoacetate decarboxylase have an increased level of acetoacetate decarboxylase activity of at least 5%, e.g., at least 10%, at least 15%, at least 20%, at least 25%, at least 50%, at least 100%, at least 150%, at least 200%, at least 300%, or at 500% compared to the host cells without the one or more polynucleotides that encode an acetoacetate decarboxylase, when cultivated under the same conditions.

In some aspects, the host cell comprises a heterologous polynucleotide that encodes an acetoacetate decarboxylase. In one aspect, the heterologous polynucleotide that encodes an acetoacetate decarboxylase is selected from: (a) a polynucleotide that encodes a acetoacetate decarboxylase having at least 65% sequence identity to SEQ ID NO: 48, or the mature polypeptide sequence thereof; (b) a polynucleotide that hybridizes under low stringency conditions with SEQ ID NO: 47, the mature polypeptide coding sequence thereof, or the full-length complementary strand of the foregoing; and (c) a polynucleotide that has at least 65% sequence identity to SEQ ID NO: 47, or the mature polypeptide coding sequence thereof. As can be appreciated by one of skill in the art, in some instances the heterologous polynucleotide that encodes an acetoacetate decarboxylase may qualify under more than one of the selections (a), (b) and (c) noted above.

In one aspect, the heterologous polynucleotide encodes an acetoacetate decarboxylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 48, or the mature polypeptide sequence thereof. In one aspect, the acetoacetate decarboxylase sequence differs by no more than ten amino acids, e.g., by no more than five amino acids, by no more than four amino acids, by no more than three amino acids, by no more than two amino acids, or by one amino acid from SEQ ID NO: 48, or the mature polypeptide sequence thereof.

In one aspect, the acetoacetate decarboxylase comprises or consists of the amino acid sequence of SEQ ID NO: 48, the mature polypeptide sequence of SEQ ID NO: 48, an allelic variant thereof, or a fragment of the foregoing, having acetoacetate decarboxylase activity. In another aspect, the acetoacetate decarboxylase comprises or consists of the amino acid sequence of SEQ ID NO: 48. In another aspect, the acetoacetate decarboxylase comprises or consists of the mature polypeptide sequence of SEQ ID NO: 48. In another aspect, the acetoacetate decarboxylase comprises or consists of amino acids 1 to 246 of SEQ ID NO: 48.

In one aspect, the heterologous polynucleotide encodes an acetoacetate decarboxylase having an amino acid substitution, deletion, and/or insertion of one or more (e.g., two, several) amino acids of SEQ ID NO: 48, or the mature polypeptide sequence thereof, as described supra. In some aspects, the total number of amino acid substitutions, deletions and/or insertions of SEQ ID NO: 48, or the mature polypeptide sequence thereof, is not more than 10, e.g., not more than 9, 8, 7, 6, 5, 4, 3, 2, or 1.

In one aspect, the heterologous polynucleotide that encodes an acetoacetate decarboxylase hybridizes under at least low stringency conditions, e.g., medium stringency conditions, medium-high stringency conditions, high stringency conditions, or very high stringency conditions with SEQ ID NO: 47, the mature polypeptide coding sequence thereof, or the full-length complementary strand of the foregoing (see, e.g., J. Sambrook, E. F. Fritsch, and T. Maniatus, 1989, supra).

In one aspect, the heterologous polynucleotide that encodes an acetoacetate decarboxylase has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 47, or the mature polypeptide coding sequence thereof.

In one aspect, the heterologous polynucleotide that encodes an acetoacetate decarboxylase comprises SEQ ID NO: 47, or the mature polypeptide coding sequence thereof. In one aspect, the heterologous polynucleotide that encodes the acetoacetate decarboxylase comprises SEQ ID NO: 47. In one aspect, the heterologous polynucleotide that encodes the acetoacetate decarboxylase comprises the mature polypeptide coding sequence of SEQ ID NO: 47. In one aspect, the mature polypeptide coding sequence is nucleotides 1 to 741 of SEQ ID NO: 47. In one aspect, the heterologous polynucleotide comprises a subsequence of SEQ ID NO: 47, or the mature polypeptide coding sequence thereof, wherein the subsequence encodes a polypeptide having acetoacetate decarboxylase activity. In one aspect, the number of nucleotides residues in the subsequence is at least 75%, e.g., at least 80%, 85%, 90%, or 95% of the number of nucleotide residues in SEQ ID NO: 47.

In one aspect, the heterologous polynucleotide encodes a fragment of SEQ ID NO: 48, or the mature polypeptide sequence thereof, wherein the fragment has acetoacetate decarboxylase activity. In one aspect, the number of amino acid residues in the fragment is at least 75%, e.g., at least 80%, 85%, 90%, or 95% of the number of amino acid residues in SEQ ID NO: 48.

The acetoacetate decarboxylase may also be an allelic variant or artificial variant of an acetoacetate decarboxylase.

The acetoacetate decarboxylase can also include fused polypeptides or cleavable fusion polypeptides, as described supra.

Techniques used to isolate or clone a polynucleotide encoding an acetoacetate decarboxylase are described supra.

The polynucleotide sequence of SEQ ID NO: 47, or a subsequence thereof; as well as the amino acid sequence of SEQ ID NO: 48, or a fragment thereof; may be used to design nucleic acid probes to identify and clone DNA encoding an acetoacetate decarboxylase from strains of different genera or species, as described supra. A genomic DNA or cDNA library prepared from such other organisms may be screened for DNA that hybridizes with the probes described above and encodes an acetoacetate decarboxylase, as described supra.

In one aspect, the nucleic acid probe is SEQ ID NO: 47. In another aspect, the nucleic acid probe is the mature polypeptide coding sequence of SEQ ID NO: 47. In another aspect, the nucleic acid probe is a polynucleotide sequence that encodes SEQ ID NO: 48, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

Polynucleotides encoding the acetoacetate decarboxylase may be obtained from microorganisms of any genus. In one aspect, the acetoacetate decarboxylase may be a bacterial, a yeast, or a filamentous fungal acetoacetate decarboxylase obtained from the microorganisms described herein. In one aspect, the acetoacetate decarboxylase is a Lactobacillus acetoacetate decarboxylase. In another aspect, the acetoacetate decarboxylase is a Clostridium acetoacetate decarboxylase, such as the Clostridium beijerinckii acetoacetate decarboxylase of SEQ ID NO: 48.

Other acetoacetate decarboxylase candidates that can be used with the host cells and methods of use described herein include, but are not limited to, a C. acetobutylicum acetoacetate decarboxylase (NP_{—}149328.1, Petersen and Bennett, Appl. Environ. Microbiol 56:3491-3498 (1990)) and a Clostridium saccharoperbutylacetonicum acetoacetate decarboxylase (AAP42566.1, Kosaka, et al., Biosci. Biotechnol Biochem. 71:58-68 (2007)). Any aspect described herein related to sequence identity, hybridization, amino acid modifications (e.g., substitutions, deletions, and/or insertions), fragments or subsequences thereof is embraced for the acetoacetate decarboxylases above.

In some aspects, the acetoacetate decarboxylase has at least 20%, e.g., at least 40%, at least 50%, at least 60%, at least 70%, at least 80%, at least 90%, at least 95%, at least 96%, at least 97%, at least 98%, at least 99%, or 100% of the acetoacetate decarboxylase activity of the mature polypeptide of SEQ ID NO: 48 under the same conditions.

The acetoacetate decarboxylase may also be identified and obtained from other sources including microorganisms isolated from nature (e.g., soil, composts, water, silage, etc.) or DNA samples obtained directly from natural materials (e.g., soil, composts, water, silage, etc.) as described supra.

### Isopropanol Dehydrogenases and Polynucleotides Encoding Isopropanol Dehydrogenases

In some aspects of the recombinant host cells and methods of use thereof, the host cells have isopropanol dehydrogenase activity. The isopropanol dehydrogenase can be any isopropanol dehydrogenase that is suitable for the host cells and their methods of use described herein, such as a naturally occurring isopropanol dehydrogenase or a variant thereof that retains isopropanol dehydrogenase activity. In one aspect, the isopropanol dehydrogenase is present in the cytosol of the host cells. In some aspects, the host cells comprises one or more (e.g., two, several) heterologous polynucleotides that encode an isopropanol dehydrogenase.

In some aspects, the host cells comprising the one or more (e.g., two, several) heterologous polynucleotides that encode an isopropanol dehydrogenase have an increased level of isopropanol dehydrogenase activity compared to the host cells without the one or more polynucleotides that encode an isopropanol dehydrogenase, when cultivated under the same conditions. In some aspects, the host cells comprising the one or more heterologous polynucleotides that encode an isopropanol dehydrogenase have an increased level of isopropanol dehydrogenase activity of at least 5%, e.g., at least 10%, at least 15%, at least 20%, at least 25%, at least 50%, at least 100%, at least 150%, at least 200%, at least 300%, or at 500% compared to the host cells without the one or more polynucleotides that encode an isopropanol dehydrogenase, when cultivated under the same conditions.

In some aspects, the host cell comprises a heterologous polynucleotide that encodes an isopropanol dehydrogenase. In one aspect, the heterologous polynucleotide that encodes an isopropanol dehydrogenase is selected from: (a) a polynucleotide that encodes an isopropanol dehydrogenase having at least 65% sequence identity to SEQ ID NO: 50, 52, or the mature polypeptide sequence thereof; (b) a polynucleotide that hybridizes under low stringency conditions with SEQ ID NO: 49, 51, the mature polypeptide coding sequence thereof, or the full-length complementary strand of the foregoing; and (c) a polynucleotide that has at least 65% sequence identity to SEQ ID NO: 49, 51, or the mature polypeptide coding sequence thereof. As can be appreciated by one of skill in the art, in some instances the heterologous polynucleotide that encodes an isopropanol dehydrogenase may qualify under more than one of the selections (a), (b) and (c) noted above.

In one aspect, the heterologous polynucleotide encodes an isopropanol dehydrogenase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 50, 52, or the mature polypeptide sequence thereof. In one aspect, the isopropanol dehydrogenase sequence differs by no more than ten amino acids, e.g., by no more than five amino acids, by no more than four amino acids, by no more than three amino acids, by no more than two amino acids, or by one amino acid from SEQ ID NO: 50, 52, or the mature polypeptide sequence thereof.

In one aspect, the isopropanol dehydrogenase comprises or consists of the amino acid sequence of SEQ ID NO: 50 or 52, the mature polypeptide sequence of SEQ ID NO: 50 or 52, an allelic variant thereof, or a fragment of the foregoing having isopropanol dehydrogenase activity. In another aspect, the isopropanol dehydrogenase comprises or consists of the amino acid sequence of SEQ ID NO: 50 or 52. In another aspect, the isopropanol dehydrogenase comprises or consists of the mature polypeptide sequence of SEQ ID NO: 50 or 52. In another aspect, the isopropanol dehydrogenase comprises or consists of amino acids 1 to 351 of SEQ ID NO: 50, or amino acids 1 to 352 of SEQ ID NO: 52.

In one aspect, the heterologous polynucleotide encodes an isopropanol dehydrogenase having an amino acid substitution, deletion, and/or insertion of one or more (e.g., two, several) amino acids of SEQ ID NO: 50, 52, or the mature polypeptide sequence thereof, as described supra. In some aspects, the total number of amino acid substitutions, deletions and/or insertions of SEQ ID NO: 50, 52, or the mature polypeptide sequence thereof, is not more than 10, e.g., not more than 9, 8, 7, 6, 5, 4, 3, 2, or 1.

In one aspect, the heterologous polynucleotide that encodes an isopropanol dehydrogenase hybridizes under at least low stringency conditions, e.g., medium stringency conditions, medium-high stringency conditions, high stringency conditions, or very high stringency conditions with SEQ ID NO: 49, 51, the mature polypeptide coding sequence thereof, or the full-length complementary strand of the foregoing (see, e.g., J. Sambrook, E. F. Fritsch, and T. Maniatus, 1989, supra).

In one aspect, the heterologous polynucleotide that encodes an isopropanol dehydrogenase has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 49, 51, or the mature polypeptide coding sequence thereof.

In one aspect, the heterologous polynucleotide that encodes the isopropanol dehydrogenase comprises SEQ ID NO: 49, 51, or the mature polypeptide coding sequence thereof. In one aspect, the heterologous polynucleotide that encodes the isopropanol dehydrogenase comprises SEQ ID NO: 49, 51. In one aspect, the heterologous polynucleotide that encodes the isopropanol dehydrogenase comprises the mature polypeptide coding sequence of SEQ ID NO: 49, 51. In one aspect, the mature polypeptide coding sequence is nucleotides 1 to 1056 of SEQ ID NO: 49, or nucleotides 1 to 1059 of SEQ ID NO: 51. In one aspect, the heterologous polynucleotide comprises a subsequence of SEQ ID NO: 49, 51, or the mature polypeptide coding sequence thereof, wherein the subsequence encodes a polypeptide having isopropanol dehydrogenase activity. In one aspect, the number of nucleotides residues in the subsequence is at least 75%, e.g., at least 80%, 85%, 90%, or 95% of the number of nucleotide residues in SEQ ID NO: 49 or 51.

In one aspect, the heterologous polynucleotide encodes a fragment of SEQ ID NO: 50, 52, or the mature polypeptide sequence thereof, wherein the fragment has isopropanol dehydrogenase activity. In one aspect, the number of amino acid residues in the fragment is at least 75%, e.g., at least 80%, 85%, 90%, or 95% of the number of amino acid residues in SEQ ID NO: 50 or 52.

The isopropanol dehydrogenase may also be an allelic variant or artificial variant of a isopropanol dehydrogenase.

The isopropanol dehydrogenase can also include fused polypeptides or cleavable fusion polypeptides, as described supra.

Techniques used to isolate or clone a polynucleotide encoding an isopropanol dehydrogenase are described supra.

The polynucleotide sequence of SEQ ID NO: 49, 51, or a subsequence thereof; as well as the amino acid sequence of SEQ ID NO: 50, 52, or a fragment thereof; may be used to design nucleic acid probes to identify and clone DNA encoding an isopropanol dehydrogenase from strains of different genera or species, as described supra. A genomic DNA or cDNA library prepared from such other organisms may be screened for DNA that hybridizes with the probes described above and encodes an isopropanol dehydrogenase, as described supra.

In one aspect, the nucleic acid probe is SEQ ID NO: 49 or 51. In another aspect, the nucleic acid probe is the mature polypeptide coding sequence of SEQ ID NO: 49, 51. In another aspect, the nucleic acid probe is a polynucleotide sequence that encodes SEQ ID NO: 50, 52, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

Polynucleotides encoding the isopropanol dehydrogenase may be obtained from microorganisms of any genus. In one aspect, the isopropanol dehydrogenase may be a bacterial, a yeast, or a filamentous fungal isopropanol dehydrogenase obtained from the microorganisms described herein. In another aspect, the isopropanol dehydrogenase is a Lactobacillus isopropanol dehydrogenase. In another aspect, the isopropanol dehydrogenase is a Clostridium isopropanol dehydrogenase, such as the Clostridium beijerinckii isopropanol dehydrogenase of SEQ ID NO: 50. In another aspect, the isopropanol dehydrogenase is a Thermoanaerobacter isopropanol dehydrogenase, such as the Thermoanaerobacter ethanolicus isopropanol dehydrogenase of SEQ ID NO: 52.

Other isopropanol dehydrogenase candidates that can be used with the host cells and methods of use described herein include, but are not limited to, a Thermoanaerobacter brockii isopropanol dehydrogenase (P14941.1, Hanai et al., Appl. Environ. Microbiol. 73:7814-7818 (2007); Peretz et al., Anaerobe 3:259-270 (1997)), a Ralstonia eutropha n-propanol dehydrogenase (formerly Alcaligenes eutrophus) (YP_{—}299391.1, Steinbuchel and Schlegel et al., Eur. J. Biochem. 141:555-564 (1984)), a Burkholderia sp. AIU 652 isopropanol dehydrogenase, and a Phytomonas species isopropanol dehydrogenase (AAP39869.1, Uttaro and Opperdoes et al., Mol. Biochem. Parasitol. 85:213-219 (1997)). Any aspect described herein related to sequence identity, hybridization, amino acid modifications (e.g., substitutions, deletions, and/or insertions), fragments or subsequences thereof is embraced for the isopropanol dehydrogenases above.

In some aspects, the isopropanol dehydrogenase has at least 20%, e.g., at least 40%, at least 50%, at least 60%, at least 70%, at least 80%, at least 90%, at least 95%, at least 96%, at least 97%, at least 98%, at least 99%, or 100% of the isopropanol dehydrogenase activity of the mature polypeptide of SEQ ID NO: 50 or 52 under the same conditions.

The isopropanol dehydrogenase may also be identified and obtained from other sources including microorganisms isolated from nature (e.g., soil, composts, water, silage, etc.) or DNA samples obtained directly from natural materials (e.g., soil, composts, water, silage, etc.) as described supra.

### Expression Vectors and Nucleic Acid Constructs

The recombinant host cells and methods utilize expression vectors comprising one or more (e.g., two, several) heterologous polynucleotides encoding a lactate dehydrogenase, lactaldehyde dehydrogenase, lactaldehyde reductase, propanediol dehydratase, and/or n-propanol dehydrogenase linked to one or more control sequences that direct expression in a suitable host cell under conditions compatible with the control sequence(s). Such expression vectors may be used in any of the host cells and methods describe herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

The various nucleotide and control sequences may be joined together to produce a recombinant expression vector that may include one or more (e.g., two, several) convenient restriction sites to allow for insertion or substitution of the polynucleotide encoding the polypeptide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

In one aspect, each polynucleotide encoding a lactate dehydrogenase, lactaldehyde dehydrogenase, lactaldehyde reductase, propanediol dehydratase, and/or n-propanol dehydrogenase described herein is contained on an independent vector. In one aspect, at least two of the polynucleotides are contained on a single vector. In one aspect, at least three of the polynucleotides are contained on a single vector. In one aspect, at least four of the polynucleotides are contained on a single vector. In one aspect, all the polynucleotides encoding the lactate dehydrogenase, lactaldehyde dehydrogenase, lactaldehyde reductase, propanediol dehydratase, and n-propanol dehydrogenase are contained on a single vector. Polynucleotides encoding heteromeric subunits of a protein complex (e.g., propandiol dehydratase) may be contained in a single heterologous polynucleotide on a single vector or alternatively contained in separate heterologous polynucleotides on separate vectors.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The expression vector may contain any suitable promoter sequence that is recognized by a host cell for expression of a polynucleotide encoding any polypeptide described herein (e.g., a lactate dehydrogenase, lactaldehyde dehydrogenase, lactaldehyde reductase, propanediol dehydratase, or n-propanol dehydrogenase). The promoter sequence contains transcriptional control sequences that mediate the expression of the polypeptide. The promoter may be any polynucleotide that shows transcriptional activity in the host cell of choice including mutant, truncated, and hybrid promoters, and may be obtained from genes encoding extracellular or intracellular polypeptides either homologous or heterologous to the host cell.

Each polynucleotide described herein may be operably linked to a promoter that is foreign to the polynucleotide. For example, in one aspect, the heterologous polynucleotide encoding a lactate dehydrogenase or subunit thereof is operably linked to a promoter that is foreign to the polynucleotide. In another aspect, the heterologous polynucleotide encoding a lactaldehyde dehydrogenase or subunit thereof is operably linked to promoter foreign to the polynucleotide. In another aspect, the heterologous polynucleotide encoding a lactaldehyde reductase or subunit thereof is operably linked to promoter foreign to the polynucleotide. In another aspect, the heterologous polynucleotide encoding a propanediol dehydratase or subunit thereof is operably linked to promoter foreign to the polynucleotide. In another aspect, the heterologous polynucleotide encoding a n-propanol dehydrogenase or subunit thereof is operably linked to promoter foreign to the polynucleotide.

As described supra, polynucleotides encoding heteromeric subunits of a protein complex may be contained in a single heterologous polynucleotide (e.g., a single plasmid), or alternatively contained in separate heterologous polynucleotides (e.g., on separate plasmids). For example, in one aspect, the heterologous polynucleotide encoding a first subunit, and the heterologous polynucleotide encoding a second subunit are contained in a single heterologous polynucleotide operably linked to a promoter that is foreign to both the heterologous polynucleotide encoding the first subunit and the heterologous polynucleotide encoding the second subunit. In one aspect, the heterologous polynucleotide encoding a first subunit, and the heterologous polynucleotide encoding a second subunit are each contained in separate unlinked heterologous polynucleotides, wherein the heterologous polynucleotide encoding the first subunit is operably linked to a foreign promoter, and the heterologous polynucleotide encoding the second subunit is operably linked to a foreign promoter. The promoters in the foregoing may be the same or different.

Examples of suitable promoters for directing the transcription of the nucleic acid constructs in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (gIaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from a gene encoding a neutral alpha-amylase in Aspergilli in which the untranslated leader has been replaced by an untranslated leader from a gene encoding triose phosphate isomerase in Aspergilli; non-limiting examples include modified promoters from the gene encoding neutral alpha-amylase in Aspergillus niger in which the untranslated leader has been replaced by an untranslated leader from the gene encoding triose phosphate isomerase in Aspergillus nidulans or Aspergillus oryzae); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

Examples of suitable promoters for directing the transcription of the nucleic acid constructs in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American, 242: 74-94; and in Sambrook et al., 1989, supra.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the host cell of choice may be used.

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase (gpd). Other useful terminators for yeast host cells are described by Romanos et al., 1992, supra.

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the host cell of choice may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of a host cell of choice may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present at the N-terminus of a polypeptide, the propeptide sequence is positioned next to the N-terminus of a polypeptide and the signal peptide sequence is positioned next to the N-terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

The vectors may contain one or more (e.g., two, several) selectable markers that permit easy selection of transformed, transfected, transduced, or the like cells. A selectable marker is a gene the product of which provides for biocide or viral resistance, resistance to heavy metals, prototrophy to auxotrophs, and the like.

Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

Examples of bacterial selectable markers are the dal genes from Bacillus subtilis or Bacillus licheniformis, or markers that confer antibiotic resistance such as ampicillin, chloramphenicol, kanamycin, or tetracycline resistance.

The vectors may contain one or more (e.g., two, several) elements that permit integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

More than one copy of a polynucleotide described herein may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors described herein are well known to one skilled in the art (see, e.g., Sambrook et al., 1989, supra).

### Host Cells

Recombinant host cells (e.g., yeast host cells or bacterial host cells) may comprise one or more (e.g., two, several) polynucleotides described herein which may be operably linked to one or more control sequences that direct the expression of one or more of the described polypeptides for the recombinant production of n-propanol. The host cell may comprise any one or combination of a plurality of the polynucleotides described. For example, in one aspect, the recombinant host cell comprises a heterologous polynucleotide encoding a lactate dehydrogenase, a lactaldehyde dehydrogenase, a lactaldehyde reductase, a propanediol dehydratase, or a n-propanol dehydrogenase described herein; wherein the host cell produces (or is capable of producing) a greater amount of n-propanol compared to the host cell without the heterologous polynucleotide when cultivated under the same conditions.

In one aspect, the recombinant host cell comprises one or more (e.g., two, several) heterologous polynucleotides encoding a lactate dehydrogenase described herein. In one aspect, the recombinant host cell comprises one or more heterologous polynucleotides encoding a lactaldehyde dehydrogenase described herein. In one aspect, the recombinant host cell comprises one or more heterologous polynucleotides encoding a lactaldehyde reductase described herein. In one aspect, the recombinant host cell comprises one or more heterologous polynucleotides encoding a propanediol dehydratase described herein. In one aspect, the recombinant host cell comprises one or more heterologous polynucleotides encoding a n-propanol dehydrogenase described herein.

In one aspect, the recombinant host cell comprises one or more (e.g., two, several) heterologous polynucleotides encoding a lactate dehydrogenase described herein, and one or more heterologous polynucleotides encoding a lactaldehyde dehydrogenase described herein. In one aspect, the recombinant host cell comprises one or more heterologous polynucleotides encoding a lactate dehydrogenase described herein, and one or more heterologous polynucleotides encoding a lactaldehyde reductase described herein. In one aspect, the recombinant host cell comprises one or more heterologous polynucleotides encoding a lactate dehydrogenase described herein, and one or more heterologous polynucleotides encoding a propanediol dehydratase described herein. In one aspect, the recombinant host cell comprises one or more heterologous polynucleotides encoding a lactate dehydrogenase described herein, and one or more heterologous polynucleotides encoding a n-propanol dehydrogenase described herein. In one aspect, the recombinant host cell comprises one or more heterologous polynucleotides encoding a lactaldehyde dehydrogenase described herein, and one or more heterologous polynucleotides encoding a lactaldehyde reductase described herein. In one aspect, the recombinant host cell comprises one or more heterologous polynucleotides encoding a lactaldehyde dehydrogenase described herein, and one or more heterologous polynucleotides encoding a propanediol dehydratase described herein. In one aspect, the recombinant host cell comprises one or more heterologous polynucleotides encoding a lactaldehyde dehydrogenase described herein, and one or more heterologous polynucleotides encoding a n-propanol dehydrogenase described herein. In one aspect, the recombinant host cell comprises one or more heterologous polynucleotides encoding a lactaldehyde reductase described herein, and one or more heterologous polynucleotides encoding a propanediol dehydratase described herein. In one aspect, the recombinant host cell comprises one or more heterologous polynucleotides encoding a lactaldehyde reductase described herein, and one or more heterologous polynucleotides encoding a n-propanol dehydrogenase described herein. In one aspect, the recombinant host cell comprises one or more heterologous polynucleotides encoding a propanediol dehydratase described herein, and one or more heterologous polynucleotides encoding a n-propanol dehydrogenase described herein.

In one aspect, the recombinant host cell comprises one or more (e.g., two, several) heterologous polynucleotides encoding a lactate dehydrogenase described herein, one or more heterologous polynucleotides encoding a lactaldehyde dehydrogenase described herein, and one or more heterologous polynucleotides encoding a lactaldehyde reductase described herein. In one aspect, the recombinant host cell comprises one or more heterologous polynucleotides encoding a lactate dehydrogenase described herein, one or more heterologous polynucleotides encoding a lactaldehyde dehydrogenase described herein, and one or more heterologous polynucleotides encoding a propanediol dehydratase described herein. In one aspect, the recombinant host cell comprises one or more heterologous polynucleotides encoding a lactate dehydrogenase described herein, one or more heterologous polynucleotides encoding a lactaldehyde reductase described herein, and one or more heterologous polynucleotides encoding a propanediol dehydratase described herein. In one aspect, the recombinant host cell comprises one or more heterologous polynucleotides encoding a lactaldehyde dehydrogenase described herein, one or more heterologous polynucleotides encoding a lactaldehyde reductase described herein, and one or more heterologous polynucleotides encoding a propanediol dehydratase described herein. In one aspect, the recombinant host cell comprises one or more heterologous polynucleotides encoding a lactate dehydrogenase described herein, one or more heterologous polynucleotides encoding a lactaldehyde dehydrogenase described herein, and one or more heterologous polynucleotides encoding a n-propanol dehydrogenase described herein. In one aspect, the recombinant host cell comprises one or more heterologous polynucleotides encoding a lactate dehydrogenase described herein, one or more heterologous polynucleotides encoding a lactaldehyde reductase described herein, and one or more heterologous polynucleotides encoding a n-propanol dehydrogenase described herein. In one aspect, the recombinant host cell comprises one or more heterologous polynucleotides encoding a lactaldehyde dehydrogenase described herein, one or more heterologous polynucleotides encoding a lactaldehyde reductase described herein, and one or more heterologous polynucleotides encoding a n-propanol dehydrogenase described herein. In one aspect, the recombinant host cell comprises one or more heterologous polynucleotides encoding a lactate dehydrogenase described herein, one or more heterologous polynucleotides encoding a propanediol dehydratase described herein, and one or more heterologous polynucleotides encoding a n-propanol dehydrogenase described herein. In one aspect, the recombinant host cell comprises one or more heterologous polynucleotides encoding a lactaldehyde dehydrogenase described herein, one or more heterologous polynucleotides encoding a propanediol dehydratase described herein, and one or more heterologous polynucleotides encoding a n-propanol dehydrogenase described herein. In one aspect, the recombinant host cell comprises one or more heterologous polynucleotides encoding a lactaldehyde reductase described herein, one or more heterologous polynucleotides encoding a propanediol dehydratase described herein, and one or more heterologous polynucleotides encoding a n-propanol dehydrogenase described herein.

In one aspect, the recombinant host cell comprises one or more (e.g., two, several) heterologous polynucleotides encoding a lactate dehydrogenase described herein, one or more heterologous polynucleotides encoding a lactaldehyde dehydrogenase described herein, one or more heterologous polynucleotides encoding a lactaldehyde reductase described herein, and one or more heterologous polynucleotides encoding a propanediol dehydratase described herein. In one aspect, the recombinant host cell comprises one or more heterologous polynucleotides encoding a lactate dehydrogenase described herein, one or more heterologous polynucleotides encoding a lactaldehyde dehydrogenase described herein, one or more heterologous polynucleotides encoding a lactaldehyde reductase described herein, and one or more heterologous polynucleotides encoding a n-propanol dehydrogenase described herein. In one aspect, the recombinant host cell comprises one or more heterologous polynucleotides encoding a lactate dehydrogenase described herein, one or more heterologous polynucleotides encoding a lactaldehyde dehydrogenase described herein, one or more heterologous polynucleotides encoding a propanediol dehydratase described herein, and one or more heterologous polynucleotides encoding a n-propanol dehydrogenase described herein. In one aspect, the recombinant host cell comprises one or more heterologous polynucleotides encoding a lactate dehydrogenase described herein, one or more heterologous polynucleotides encoding a lactaldehyde reductase described herein, one or more heterologous polynucleotides encoding a propanediol dehydratase described herein, and one or more heterologous polynucleotides encoding a n-propanol dehydrogenase described herein. In one aspect, the recombinant host cell comprises one or more heterologous polynucleotides encoding a lactaldehyde dehydrogenase described herein, one or more heterologous polynucleotides encoding a lactaldehyde reductase described herein, one or more heterologous polynucleotides encoding a propanediol dehydratase described herein, and one or more heterologous polynucleotides encoding a n-propanol dehydrogenase described herein.

In one aspect, the recombinant host cell comprises one or more (e.g., two, several) heterologous polynucleotides encoding a lactate dehydrogenase described herein, one or more heterologous polynucleotides encoding a lactaldehyde dehydrogenase described herein, one or more heterologous polynucleotides encoding a lactaldehyde reductase described herein, one or more heterologous polynucleotides encoding a propanediol dehydratase described herein, and one or more heterologous polynucleotides encoding a n-propanol dehydrogenase described herein.

In some of these aspects, the recombinant host cell lacks an endogenous lactate dehydrogenase, lacks an endogenous lactaldehyde dehydrogenase, lacks an endogenous lactaldehyde reductase, lacks an endogenous propanediol dehydratase, and/or lacks an endogenous n-propanol dehydrogenase.

For example, in one aspect, a recombinant host cell (e.g., a lactobacillus host cell) comprises one or more (e.g., two, several) heterologous polynucleotides selected from:

(1) a heterologous polynucleotide that encodes a lactate dehydrogenase, wherein the polynucleotide is selected from: (a) a polynucleotide that encodes a lactate dehydrogenase having at least 65% sequence identity to SEQ ID NO: 2, 4, 34, 36, or the mature polypeptide sequence thereof; (b) a polynucleotide that hybridizes under low stringency conditions with SEQ ID NO: 1, 3, 33, 35, the mature polypeptide coding sequence thereof, or the full-length complementary strand of the foregoing; and (c) a polynucleotide that has at least 65% sequence identity to SEQ ID NO: 1, 3, 33, 35, or the mature polypeptide coding sequence thereof;

(2) a heterologous polynucleotide that encodes a lactaldehyde dehydrogenase, wherein the polynucleotide is selected from: (a) a polynucleotide that encodes a lactaldehyde dehydrogenase having at least 65% sequence identity to SEQ ID NO: 6, 8, 26, 30, 32, or the mature polypeptide sequence thereof; (b) a polynucleotide that hybridizes under low stringency conditions with SEQ ID NO: 5, 7, 25, 29, 31, the mature polypeptide coding sequence thereof, or the full-length complementary strand of the foregoing; and (c) a polynucleotide that has at least 65% sequence identity to SEQ ID NO: 5, 7, 25, 29, 31, or the mature polypeptide coding sequence thereof;

(3) a heterologous polynucleotide that encodes a lactaldehyde reductase, wherein the polynucleotide is selected from: (a) a polynucleotide that encodes a lactaldehyde reductase having at least 65% sequence identity to SEQ ID NO: 10, 12, 28, 54, 56, or the mature polypeptide sequence thereof; (b) a polynucleotide that hybridizes under low stringency conditions with SEQ ID NO: 9, 11, 27, 53, 55, the mature polypeptide coding sequence thereof, or the full-length complementary strand of the foregoing; and (c) a polynucleotide that has at least 65% sequence identity to SEQ ID NO: 9, 11, 27, 53, 55, or the mature polypeptide coding sequence thereof;

(4) a heterologous polynucleotide that encodes a first propanediol dehydratase subunit, and a heterologous polynucleotide that encodes a second propanediol dehydratase subunit, wherein the polynucleotide that encodes the first subunit is selected from: (a) a polynucleotide that encodes a propanediol dehydratase subunit having at least 65% sequence identity to SEQ ID NO: 14, 18, 58, or the mature polypeptide sequence thereof; (b) a polynucleotide that hybridizes under low stringency conditions with SEQ ID NO: 13, 17, 57, the mature polypeptide coding sequence thereof, or the full-length complementary strand of the foregoing; and (c) a polynucleotide that has at least 65% sequence identity to SEQ ID NO: 13, 17, 57, or the mature polypeptide coding sequence thereof; and wherein the polynucleotide that encodes the second subunit is selected from: (a) a polynucleotide that encodes a propanediol dehydratase subunit having at least 65% sequence identity to SEQ ID NO: 16, 20, 60, or the mature polypeptide sequence thereof; (b) a polynucleotide that hybridizes under low stringency conditions with SEQ ID NO: 15, 19, 59, the mature polypeptide coding sequence thereof, or the full-length complementary strand of the foregoing; and (c) a polynucleotide that has at least 65% sequence identity to SEQ ID NO: 15, 19, 59, or the mature polypeptide coding sequence thereof; and

(5) a heterologous polynucleotide that encodes an n-propanol dehydrogenase, wherein the polynucleotide is selected from: (a) a polynucleotide that encodes an n-propanol dehydrogenase having at least 65% sequence identity to SEQ ID NO: 22, 24, 62, or the mature polypeptide sequence thereof; (b) a polynucleotide that hybridizes under low stringency conditions with SEQ ID NO: 21, 23, 61, the mature polypeptide coding sequence thereof, or the full-length complementary strand of the foregoing; and (c) a polynucleotide that has at least 65% sequence identity to SEQ ID NO: 21, 23, 61, or the mature polypeptide coding sequence thereof;

wherein the host cell produces (or is capable of producing) a greater amount of n-propanol compared to the host cell without the one or more polynucleotides, when cultivated under the same conditions.

As can be appreciated by one of skill in the art, in some instances the heterologous polynucleotides that encode the polypeptides noted above may qualify under more than one of the respective selections (a), (b) and (c).

Any one of the host cells described above may further comprise one or more (e.g., two, several) heterologous polynucleotides encoding a polypeptide of the isopropanol pathway as depicted in FIG. 2 for the coproduction of n-propanol and isopropanol. For example, in one aspect, the recombinant host cell further comprises one or more (e.g., two, several) heterologous polynucleotides encoding a thiolase, a CoA-transferase, an acetoacetate decarboxylase, or an isopropanol dehydrogenase described herein; wherein the host cell produces (or is capable of producing) a greater amount of isopropanol compared to the host cell without the heterologous polynucleotide when cultivated under the same conditions.

In one aspect, the recombinant host cell further comprises one or more (e.g., two, several) heterologous polynucleotides encoding a thiolase described herein. In one aspect, the recombinant host cell further comprises one or more heterologous polynucleotides encoding a CoA-transferase described herein. In one aspect, the recombinant host cell further comprises one or more heterologous polynucleotides encoding an acetoacetate decarboxylase described herein. In one aspect, the recombinant host cell further comprises one or more heterologous polynucleotides encoding an isopropanol dehydrogenase described herein.

In one aspect, the recombinant host cell further comprises one or more (e.g., two, several) heterologous polynucleotides encoding a thiolase described herein, and one or more heterologous polynucleotides encoding a CoA-transferase described herein. In one aspect, the recombinant host cell further comprises one or more heterologous polynucleotides encoding a thiolase described herein, and one or more heterologous polynucleotides encoding an acetoacetate decarboxylase described herein. In one aspect, the recombinant host cell further comprises one or more heterologous polynucleotides encoding a thiolase described herein, and one or more heterologous polynucleotides encoding an isopropanol dehydrogenase described herein.

In one aspect, the recombinant host cell further comprises one or more (e.g., two, several) heterologous polynucleotides encoding a thiolase described herein, one or more heterologous polynucleotides encoding a CoA-transferase described herein, and one or more heterologous polynucleotides encoding an acetoacetate decarboxylase described herein. In one aspect, the recombinant host cell further comprises one or more heterologous polynucleotides encoding a thiolase described herein, one or more heterologous polynucleotides encoding a CoA-transferase described herein, and one or more heterologous polynucleotides encoding an isopropanol dehydrogenase described herein. In one aspect, the recombinant host cell further comprises one or more heterologous polynucleotides encoding a thiolase described herein, one or more heterologous polynucleotides encoding an acetoacetate decarboxylase described herein, and one or more heterologous polynucleotides encoding an isopropanol dehydrogenase described herein. In one aspect, the recombinant host cell further comprises one or more heterologous polynucleotides encoding a CoA-transferase described herein, one or more heterologous polynucleotides encoding an acetoacetate decarboxylase described herein, and one or more heterologous polynucleotides encoding an isopropanol dehydrogenase described herein.

In one aspect, the recombinant host cell further comprises one or more (e.g., two, several) heterologous polynucleotides encoding a thiolase described herein, one or more heterologous polynucleotides encoding a CoA-transferase described herein, one or more heterologous polynucleotides encoding an acetoacetate decarboxylase described herein, and one or more heterologous polynucleotides encoding an isopropanol dehydrogenase described herein.

A construct or vector (or multiple constructs or vectors) comprising the one or more (e.g., two, several) polynucleotides is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source. The aspects described below apply to the host cells, per se, as well as methods using the host cells.

The host cell may be any eukaryotic cell capable of the recombinant production of a polypeptide described herein and/or any cell capable of the recombinant production of n-propanol. The host cell may also be any suitable eukaryote, such as a mammalian, insect, plant, or fungal cell.

The host cell may be a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes described herein, yeast shall be defined as described in Biology and Activities of Yeast (Skinner, F. A., Passmore, S. M., and Davenport, R. R., eds, Soc. App. Bacteriol. Symposium Series No. 9, 1980).

The yeast host cell may be a Candida, Hansenula, Kluyveromyces, Pichia, Saccharomyces, Schizosaccharomyces, Yarrowia, or Issatchenkia cell, such as a Candida sonorensis, Candida methanosorbosa, Candida ethanolica, Kluyveromyces lactis, Kluyveromyces marxianus, Pichia fermentans, Pichia galeiformis, Pichia membranifaciens, Pichia deserticola, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Saccharomyces bulderi, Yarrowia lipolytica or Issatchenkia orientalis cell.

In some aspects, the yeast host cell is derived from a cell that has been genetically modified to produce high lactic acid titers, exhibit increased tolerance to acidic pH, and/or display increased ability to ferment pentose sugars. Exemplary genetically modified yeast cells are described in WO 00/71738, WO 03/049525, WO 03/102201, 03/102152, WO 02/42471, WO 2007/032792, WO 2007/106524, WO 2007/117282, the content of which is hereby incorporated by reference with respect to said cells. Any yeast cell described in the foregoing applications is contemplated to be further modified by introducing one or more (e.g., two, several) polynucleotide(s) described herein to produce n-propanol (e.g., one or more heterologous polynucleotides encoding a lactatate dehydrogenase, one or more heterologous polynucleotides encoding a lactaldehyde dehydrogenase, one or more heterologous polynucleotides encoding a lactaldehyde reductase, one or more heterologous polynucleotides encoding a propanediol dehydratase, and/or one or more heterologous polynucleotides encoding a n-propanol dehydrogenase).

In some aspects, the yeast is of a crabtree-positive phenotype or a crabtree-negative phenotype. Crabtree-negative organisms are characterized by the ability to be induced into an increased fermentative state. Both naturally occurring organisms and genetically modified organisms can be characterized as Crabtree-negative. The Crabtree effect is defined as oxygen consumption inhibition in a microorganism when the microorganism is cultured under aerobic conditions in the presence of a high concentration of glucose (e.g. >5 mM glucose). Crabtree-positive organisms continue to ferment (rather than respire) irrespective of oxygen availability in the presence of glucose, while Crabtree-negative organisms do not exhibit glucose-mediated inhibition of oxygen consumption. This characteristic is useful for organic product synthesis, since it permits cells to be grown at high substrate concentrations but to retain the beneficial energetic effects of oxidative phosphorylation. In one aspect, the yeast has a crabtree-negative phenotype.

In some aspects, the yeast host cell has reduced pyruvate decarboxylase (PDC) activity compared to the wild-type strain in order to divert sugar metabolism from ethanol production to lactic acid production, as described in the gene disruption section below. In some aspects, the yeast host cell been genetically modified to disrupt an endogenous polynucleotide encoding a pyruvate decarboxylase (PDC). Disruption of the pdc genes can be accomplished in a variety of ways, including, for example, methods analogous to those described in WO 99/114335, WO 02/42471, WO 03/049525, WO 03/102152 and WO 03/102201. Other methods of disrupting PDC activity are described in Porro, “Development of metabolically engineered Saccharomyces cerevisiae cells for the production of lactic acid”, Biotechnol. Prog. 1995 May-June; 11(3): 294-8; Porro et al., “Replacement of a metabolic pathway for large-scale production of lactic acid from engineered yeasts”, App. Environ. Microbiol. 1999 September:65(9):4211-5; Bianchi et al., “Efficient homolactic fermentation by Kluyveromyces lactis strains defective in pyruvate utilization and transformed with the heterologous LDH gene”, App. Environ. Microbiol. 2001 December; 67(12)5621-5; and WO 99/14335. In some aspects, the pyruvate decarboxylase (PDC) gene is disrupted at the locus of the PDC gene by insertion of one or more heterologous polynucleotides that encodes a lactatate dehydrogenase supra, as described in WO 03/102201.

In some aspects, the yeast host cell has reduced L- or D-lactate:ferricytochrome c oxidoreductase activity compared to that of a wild-type stain in order to reduce the conversion of lactate back into pyruvate. In some aspects, the yeast host cell been genetically modified to disrupt an endogenous polynucleotide encoding a L- or D-lactate:ferricytochrome c oxidoreductase. Disruption of the L- or D-lactate:ferricytochrome c oxidoreductase genes can be accomplished in a variety of ways, including, for example, methods analogous to those described in WO 2007/117282. In some aspects, the yeast host cell been genetically modified to disrupt an endogenous polynucleotide encoding a L-lactate:ferricytochrome c oxidoreductase gene. In some aspects, the disrupted L-lactate:ferricytochrome c oxidoreductase gene comprises SEQ ID NO: 1 of WO 2007/117282 (the L-lactate:ferricyochrome c oxidoreductase gene CYB2 of a wild-type K. marxianus strain), SEQ ID NO: 79 of WO 2007/117282 (the CYB2A gene of a wild-type I. orientalis strain), or SEQ ID NO: 81 of WO 2007/117282 (the CYB2B gene of a wild-type I. orientalis strain); and/or encodes for an enzyme having an amino acid sequence identified as SEQ ID NO: 2, SEQ ID NO: 80, or SEQ ID NO: 82 of WO 2007/117282. In some aspects, the yeast host cell been genetically modified to disrupt an endogenous polynucleotide encoding a D-lactate:ferricytochrome c oxidoreductase. In some aspects, the disrupted D-lactate:ferricytochrome c oxidoreductase gene comprises SEQ ID NO: 83 of WO 2007/117282 (the D-lactate:ferricyochrome c oxidoreductase (DLD1) gene of a wild-type K. nwrxianus strain), the DLD1 gene of S. cerevisiae described in WO 2007/117282, or the DLDI1 gene of I. orientalis described in WO 2007/117282; and/or encodes for an enzyme having an amino acid sequence identified as SEQ ID NO: 84 of WO 2007/117282, an enzyme having the amino acid sequence of a protein encoded by the S. cerevisiae DLD1 gene of WO 2007/117282, or an enzyme having the amino acid sequence of a protein encoded by the I. orientalis DLD1 gene described in WO 2007/117282. In some aspects, the L-lactate:ferricytochrome c oxidoreductase activity of the yeast host cell, D-lactate:ferricytochrome c oxidoreductase of the yeast host cell, or total lactate:ferricytochrome c oxidoreductase activity of the yeast host cell is decreased by at least 50%, e.g., at least 60%, at least 70%, at least 80%, at least 90%, or at least 95% compared to the yeast host cell without the disruption under the same conditions.

In some aspects, the yeast host cell has been genetically modified to disrupt a native metabolic pathway for the production of glycerol. In some aspects, the yeast host cell has reduced glycerol-3-phosphate dehydrogenase (GPD) activity and/or reduced glycerol-3-phosphatase (GPP) activity. In some aspects, the yeast host cell has been genetically modified to disrupt an endogenous polynucleotide encoding a glycerol-3-phosphate dehydrogenase (GPD) and/or to disrupt an endogenous polynucleotide encoding a glycerol-3-phosphatase (GPP). In some aspects, the glycerol-3-phosphate dehydrogenase (GPD) activity of the yeast host cell is decreased by at least 50%, e.g., at least 60%, at least 70%, at least 80%, at least 90%, or at least 95% compared to the yeast host cell without the disruption under the same conditions. In some aspects, the glycerol-3-phosphate dehydrogenase (GPD) activity of the yeast host cell is decreased by at least 50%, e.g., at least 60%, at least 70%, at least 80%, at least 90%, or at least 95% compared to the yeast host cell without the disruption under the same conditions. In some aspects, the amount of glycerol produced by the yeast host cell is decreased by at least 50%, e.g., at least 60%, at least 70%, at least 80%, at least 90%, or at least 95% compared to the host cell without disruption of the glycerol-3-phosphate dehydrogenase gene and/or glycerol-3-phosphatase gene under the same conditions. Disruption of the glycerol-3-phosphate dehydrogenase (GPD) gene and the glycerol-3-phosphatase (GPP) gene can be accomplished in a variety of ways, including, for example, methods analogous to those described in WO 2007/106524.

In some aspects, wherein the yeast host cell has an alternate pathway to glycerol production through dihydroxyacetone (e.g., S. pombe), the cell been genetically modified to reduce glycerol production by reducing dihydroxyacetone phosphate phosphatase activity and/or reducing glycerol dehydrogenase activity. In some aspects, the yeast host cell has been genetically modified to disrupt or delete a native dihydroxyacetone phosphate phosphatase gene and/or to disrupt an endogenous polynucleotide encoding a glycerol dehydrogenase. In some aspects, the dihydroxyacetone phosphate phosphatase activity of the yeast host cell is decreased by at least 50%, e.g., at least 60%, at least 70%, at least 80%, at least 90%, or at least 95% compared to the yeast host cell without the disruption under the same conditions. In some aspects, the glycerol dehydrogenase activity of the yeast host cell is decreased by at least 50%, e.g., at least 60%, at least 70%, at least 80%, at least 90%, or at least 95% compared to the yeast host cell without the disruption under the same conditions. In some aspects, the amount of glycerol produced by the yeast host cell is decreased by at least 50%, e.g., at least 60%, at least 70%, at least 80%, at least 90%, or at least 95% compared to a host cell without disruption of the dihydroxyacetone phosphate phosphatase gene and/or glycerol dehydrogenase under the same conditions. Disruption of the dihydroxyacetone phosphate phosphatase gene and the glycerol dehydrogenase gene can be accomplished in a variety of ways, including, for example, methods analogous to those described in WO 2007/106524.

In some aspects, the yeast host cell comprises (1) one or more heterologous polynucleotides encoding a xylose isomerase gene, (2) a disruption of a native gene that produces an enzyme that catalyzes the conversion of xylose to xylitol, (3) a disruption of a functional xylitol dehydrogenase gene and/or (4) a modification that cause the cell to overexpress a functional xylulokinase. Methods for introducing such modifications into yeast cells are described, for example, in WO 04/099381, the content of which is incorporated herein by reference with respect to the methods and host cells thereof. In some aspects, the yeast host cell can metabolize sugars other than glucose or other monosaccharide hexoses, in particular pentoses including the non-limiting examples of xylose and L-arabinose.

The host cell may be fungal host cell, such as a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocaffimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus aculeatus, Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023 and Yelton et al., 1984, Proc. Natl. Acad. Sci. USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

The host cell may be any prokaryotic cell capable of the recombinant production of a polypeptide described herein and/or any cell capable of the recombinant production of n-propanol. The prokaryotic host cell may be any gram-positive or gram-negative bacterium. Gram-positive bacteria include, but not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The bacterial host cell may also be any Lactobacillus cell including, but not limited to, L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aquaticus, L. arizonensis, L. aviarius, L. bavaricus, L. bifermentans, L. bobalius, L. brevis, L. buchneri, L. bulgaricus, L. cacaonum, L. camelliae, L. capillatus, L. carni, L. casei, L. catenaformis, L. cellobiosus, L. ceti, L. coleohominis, L. collinoides, L. composti, L. concavus, L. confusus, L. coryniformis, L. crispatus, L. crustorum, L. curvatus, L. cypricasei, L. delbrueckii, L. dextrinicus, L. diolivorans, L. divergens, L. durianis, L. equi, L. equicursoris, L. equigenerosi, L. fabifermentans, L. farciminis, L. farraginis, L. ferintoshensis, L. fermentum, L. fornicalis, L. fructivorans, L. fructosus, L. frumenti, L. fuchuensis, L. gallinarum, L. gasseri, L. gastricus, L. ghanensis, L. graminis, L. halotolerans, L. hammesii, L. hamsteri, L. harbinensis, L. hayakitensis, L. helveticus, L. heterohiochii, L. hilgardii, L. homohiochii, L. hordei, L. iners, L. ingluviei, L. intestinalis, L. jensenii, L. johnsonii, L. kalixensis, L. kandleri, L. kefiranofaciens, L. kefiranofaciens, L. kefirgranum, L. kefiri, L. kimchii, L. kisonensis, L. kitasatonis, L. kunkeei, L. lactis, L. leichmannii, L. lindneri, L. malefermentans, L. mali, L. maltaromicus, L. manihotivorans, L. mindensis, L. minor, L. minutus, L. mucosae, L. murinus, L. nagelii, L. namurensis, L. nantensis, L. nodensis, L. oeni, L. oligofermentans, L. oris, L. otakiensis, L. panis, L. pantheris, L. parabrevis, L. parabuchneri, L. paracasei, L. paracollinoides, L. parafarraginis, L. parakefiri, L. paralimentarius, L. paraplantarum, L. pentosus, L. perolens, L. piscicola, L. plantarum, L. pobuzihii, L. pontis, L. psittaci, L. rapi, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. senmaizukei, L. sharpeae, L. siliginis, L. similis, L. sobrius, L. spicheri, L. sucicola, L. suebicus, L. sunkii, L. suntoryeus, L. taiwanensis, L. thailandensis, L. thermotolerans, L. trichodes, L. tucceti, L. uli, L. ultunensis, L. uvarum, L. vaccinostercus, L. vaginalis, L. versmoldensis, L. viridescens, L. vitulinus, L. xylosus, L. yamanashiensis, L. zeae, and L. zymae. In one aspect, the bacterial host cell is L. plantarum, L. fructivorans, or L. reuteri.

In one aspect, the host cell is a member of a genus selected from Escherichia (e.g., Escherichia coli), Lactobacillus (e.g., Lactobacillus plantarum, Lactobacillus fructivorans, or Lactobacillus reuteri), and Propionibacterium (e.g., Propionibacterium freudenreichii). In one aspect, the host cell is Lactobacillus plantarum. In one aspect, the host cell is Lactobacillus reuteri.

The introduction of DNA into a Bacillus cell may, for instance, be effected by protoplast transformation (see, e.g., Chang and Cohen, 1979, Mol. Gen. Genet. 168: 111-115), by using competent cells (see, e.g., Young and Spizizen, 1961, J. Bacteriol. 81: 823-829, or Dubnau and Davidoff-Abelson, 1971, J. Mol. Biol. 56: 209-221), by electroporation (see, e.g., Shigekawa and Dower, 1988, Biotechniques 6: 742-751), or by conjugation (see, e.g., Koehler and Thorne, 1987, J. Bacteriol. 169: 5271-5278). The introduction of DNA into an E. coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios 68: 189-207, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

In some aspects, the host cell comprises one or more (e.g., two, several) polynucleotides of the n-propanol pathway described herein, wherein the host cell secretes (and/or is capable of secreting) an increased level of n-propanol compared to the host cell without the one or more polynucleotides when cultivated under the same conditions. In some aspects, the host cell secretes and/or is capable of secreting an increased level of n-propanol of at least 5%, e.g., at least 10%, at least 15%, at least 20%, at least 25%, at least 50%, at least 100%, at least 150%, at least 200%, at least 300%, or at 500% compared to the host cell without the one or more polynucleotides, when cultivated under the same conditions. Examples of suitable cultivation conditions are described below and will be readily apparent to one of skill in the art based on the teachings herein.

In some aspects, the host cell further comprises one or more (e.g., two, several) polynucleotides of the isopropanol pathway described herein, wherein the host cell secretes (and/or is capable of secreting) an increased level of isopropanol compared to the host cell without the one or more polynucleotides when cultivated under the same conditions. In some aspects, the host cell secretes and/or is capable of secreting an increased level of n-propanol of at least 5%, e.g., at least 10%, at least 15%, at least 20%, at least 25%, at least 50%, at least 100%, at least 150%, at least 200%, at least 300%, or at 500% compared to the host cell without the one or more polynucleotides, when cultivated under the same conditions.

In any of these aspects, the host cell produces (and/or is capable of producing) n-propanol and/or isopropanol at a yield of at least than 10%, e.g., at least than 20%, at least than 30%, at least than 40%, at least than 50%, at least than 60%, at least than 70%, at least than 80%, or at least than 90%, of theoretical.

In any of these aspects, the recombinant host has a n-propanol and/or an isopropanol volumetric productivity greater than about 0.1 g/L per hour, e.g., greater than about 0.2 g/L per hour, 0.5 g/L per hour, 0.6 g/L per hour, 0.7 g/L per hour, 0.8 g/L per hour, 0.9 g/L per hour, 1.0 g/L per hour, 1.1 g/L per hour, 1.2 g/L per hour, 1.3 g/L per hour, 1.5 g/L per hour, 1.75 g/L per hour, 2.0 g/L per hour, 2.25 g/L per hour, 2.5 g/L per hour, or 3.0 g/L per hour; or between about 0.1 g/L per hour and about 2.0 g/L per hour, e.g., between about 0.3 g/L per hour and about 1.7 g/L per hour, about 0.5 g/L per hour and about 1.5 g/L per hour, about 0.7 g/L per hour and about 1.3 g/L per hour, about 0.8 g/L per hour and about 1.2 g/L per hour, or about 0.9 g/L per hour and about 1.1 g/L per hour.

The recombinant host cells may be cultivated in a nutrient medium suitable for production of the lactate dehydrogenase, lactaldehyde dehydrogenase, lactaldehyde reductase, propanediol dehydratase, or n-propanol dehydrogenase using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the desired polypeptide to be expressed and/or isolated. The cultivation takes place in a suitable nutrient medium comprising carbon and nitrogen sources and inorganic salts, using procedures known in the art. Suitable media are available from commercial suppliers, may be prepared according to published compositions (e.g., in catalogues of the American Type Culture Collection), or may be prepared from commercially available ingredients.

The lactate dehydrogenase, lactaldehyde dehydrogenase, lactaldehyde reductase, propanediol dehydratase, and n-propanol dehydrogenase, and activities thereof, can be detected using methods known in the art. These detection methods may include use of specific antibodies, formation of an enzyme product, or disappearance of an enzyme substrate. See, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999); and Hanai et al., Appl. Environ. Microbiol. 73:7814-7818 (2007)).

### Gene Disruptions

The host cell may also comprise one or more (e.g., two, several) gene disruptions, e.g., to divert sugar metabolism from undesired products to n-propanol. Disruptions of a particular gene of interest, such as a pyruvate decarboxylase, can be generated by methods known in the art, e.g., by directed homologous recombination (see Methods in Yeast Genetics (1997 edition), Adams, Gottschling, Kaiser, and Stems, Cold Spring Harbor Press (1998)); or by RNAi or antisense technology.

In some aspects, the host cell has an absence or decrease of pyruvate decarboxylase (PDC) activity. In some aspects, the host cell comprises a disruption to an endogenous polynucleotide encoding a pyruvate decarboxylase (PDC). In some aspects, the polynucleotide encoding the pyruvate decarboxylase (PDC) is disrupted at the locus of the pyruvate decarboxylase gene by insertion of one or more heterologous polynucleotides that encode a polypeptide described herein (e.g., a polynucleotide that encodes a lactatate dehydrogenase supra). In some aspects, the host cell has no pyruvate decarboxylase activity. In some aspects, the pyruvate decarboxylase activity of the host cell is decreased by at least 50%, e.g., at least 60%, at least 70%, at least 80%, at least 90%, or at least 95% compared to the host cell without the disruption of the polynucleotide encoding the pyruvate carboxylase under the same conditions. In some aspects, the host cell has no pyruvate decarboxylase encoded by the disrupted polynucleotide. In some aspects, the pyruvate decarboxylase encoded by the disrupted polynucleotide is decreased by at least 50%, e.g., at least 60%, at least 70%, at least 80%, at least 90%, or at least 95% compared to the host cell without the disruption under the same conditions. In some aspects, the host cell has no corresponding mRNA from the disrupted polynucleotide. In some aspects, the corresponding mRNA from the disrupted polynucleotide is decreased by at least 50%, e.g., at least 60%, at least 70%, at least 80%, at least 90%, or at least 95% compared to the host cell without the disruption under the same conditions. In some aspects, the amount of ethanol produced by the host cell is decreased by at least 50%, e.g., at least 60%, at least 70%, at least 80%, at least 90%, or at least 95% compared to the host cell without disruption of the polynucleotide encoding the pyruvate decarboxylase under the same conditions. In some aspect, the amount of n-propanol produced is at least 5%, e.g., at least 10%, at least 15%, at least 20%, at least 25%, at least 30%, at least 50%, or at least 100% greater compared to the host cell without the disruption to the polynucleotide encoding the pyruvate decarboxylase under the same conditions.

In some aspects, the host cell has an absence or decrease of propionaldehyde dehydrogenase activity. Decreasing priopionaldehyde dehydrogenase activity may avoid metabolism of the downstream metabolite proionaldehyde to propionyl-CoA. The host cell may lack an endogenous polynucleotide encoding a propionaldehyde dehydrogenase, or comprise a disruption to an endogenous polynucleotide encoding a propionaldehyde dehydrogenase. In some aspects, the host cell comprises a disruption to the coding sequence of the pduP gene (SEQ ID NO: 67), which encodes the propionaldehyde dehydrogenase of SEQ ID NO: 68. In some aspects, the host cell comprises a disruption to the endogenous gene that encodes a propionaldehyde dehydrogenase having at least 60%, e.g., at least 70%, at least 75%, at least 80%, at least 85%, at least 90%, at least 95%, at least 97%, at least 98%, at least 99%, or 100% sequence identity to SEQ ID NO: 68, or the mature polypeptide sequence thereof. In some aspects, the host cell comprises a disruption to an endogenous propionaldehyde dehydrogenase gene, wherein the coding sequence of the gene has at least 60%, e.g., at least 70%, at least 75%, at least 80%, at least 85%, at least 90%, at least 95%, at least 97%, at least 98%, at least 99%, or 100% sequence identity to SEQ ID NO: 67, or the mature polypeptide coding sequence thereof. In some aspects, the coding sequence of the endogenous gene comprises or consists of SEQ ID NO: 67.

In some aspects, the host cell has an absence or decrease of alcohol dehydrogenase activity. Decreasing alcohol dehydrogenase activity may improve the redox potential for conversion of lactate to lactaldehyde. The host cell may lack an endogenous polynucleotide encoding an alcohol dehydrogenase, or comprise a disruption to an endogenous polynucleotide encoding an alcohol dehydrogenase. In some aspects, the host cell comprises a disruption to the endogenous adhE gene having the coding sequence of SEQ ID NO: 65, which encodes the bifunctional alcohol dehydrogenase of SEQ ID NO: 66. In some aspects, the host cell comprises a disruption to an endogenous gene that encodes an acetate kinase having at least 60%, e.g., at least 70%, at least 75%, at least 80%, at least 85%, at least 90%, at least 95%, at least 97%, at least 98%, at least 99%, or 100% sequence identity to SEQ ID NO: 66, or the mature polypeptide sequence thereof. In some aspects, the host cell comprises a disruption to the endogenous alcohol dehydrogenase gene, wherein the coding sequence of the gene has at least 60%, e.g., at least 70%, at least 75%, at least 80%, at least 85%, at least 90%, at least 95%, at least 97%, at least 98%, at least 99%, or 100% sequence identity to SEQ ID NO: 65, or the mature polypeptide coding sequence thereof. In some aspects, the coding sequence of the endogenous gene comprises or consists of SEQ ID NO: 65.

In some aspects, the polynucleotide encoding the propionaldehyde dehydrogenase or alcohol dehydrogenase is disrupted at the locus of the propionaldehyde dehydrogenase or alcohol dehydrogenase gene by insertion of one or more heterologous polynucleotides that encode a polypeptide described herein (e.g., a polynucleotide that encodes a lactate dehydrogenase supra). In some aspects, the host cell has no propionaldehyde dehydrogenase or alcohol dehydrogenase activity. In some aspects, the propionaldehyde dehydrogenase or alcohol dehydrogenase activity of the host cell is decreased by at least 50%, e.g., at least 60%, at least 70%, at least 80%, at least 90%, or at least 95% compared to the host cell without the disruption of the polynucleotide encoding the propionaldehyde dehydrogenase or alcohol dehydrogenase under the same conditions. In some aspects, the host cell has no propionaldehyde dehydrogenase or alcohol dehydrogenase encoded by the disrupted polynucleotide. In some aspects, the propionaldehyde dehydrogenase or alcohol dehydrogenase encoded by the disrupted polynucleotide is decreased by at least 50%, e.g., at least 60%, at least 70%, at least 80%, at least 90%, or at least 95% compared to the host cell without the disruption under the same conditions. In some aspects, the host cell has no corresponding mRNA from the disrupted polynucleotide. In some aspects, the corresponding mRNA from the disrupted polynucleotide is decreased by at least 50%, e.g., at least 60%, at least 70%, at least 80%, at least 90%, or at least 95% compared to the host cell without the disruption under the same conditions. In some aspects, the amount of propionyl-CoA or propionic acid produced by the host cell is decreased by at least 50%, e.g., at least 60%, at least 70%, at least 80%, at least 90%, or at least 95% compared to the host cell without disruption of the polynucleotide encoding the propionaldehyde dehydrogenase under the same conditions. In some aspect, the amount of n-propanol produced is at least 5%, e.g., at least 10%, at least 15%, at least 20%, at least 25%, at least 30%, at least 50%, or at least 100% greater compared to the host cell without the disruption to the polynucleotide encoding the propionaldehyde dehydrogenase under the same conditions.

### Methods

The recombinant host cells described herein may be used for the production of n-propanol or for the coproduction of n-propanol with isopropanol. In one aspect is a method of producing n-propanol, comprising: (a) cultivating any one of the recombinant host cells described herein (e.g., any host cell with lactate dehydrogenase activity, lactaldehyde dehydrogenase activity, lactaldehyde reductase activity, propanediol dehydratase activity and n-propanol dehydrogenase activity) in a medium under suitable conditions to produce the n-propanol; and (b) recovering the n-propanol. In one aspect is a method of producing n-propanol, comprising: (a) cultivating in a medium any one of the recombinant host cells described herein, wherein the host cell comprises one or more (e.g., two, several) heterologous polynucleotides encoding a lactate dehydrogenase described herein, a lactaldehyde dehydrogenase described herein, a lactaldehyde reductase described herein, a propanediol dehydratase described herein, and/or a n-propanol dehydrogenase described herein, under suitable conditions to produce the n-propanol; and (b) recovering the n-propanol.

In one aspect is a method of coproducing n-propanol and isopropanol, comprising: (a) cultivating any one of the recombinant host cells described herein (e.g., any eukaryotic or bacterial host cell with lactate dehydrogenase activity, lactaldehyde dehydrogenase activity, lactaldehyde reductase activity, propanediol dehydratase activity, n-propanol dehydrogenase activity, thiolase activity, CoA-transferase activity, acetoacetate decarboxylase activity, and isopropanol dehydrogenase activity) in a medium under suitable conditions to produce the n-propanol and isopropanol; and (b) recovering the n-propanol and isopropanol. In one aspect is a method of coproducing n-propanol and isopropanol, comprising: (a) cultivating in a medium any one of the recombinant host cells described herein, wherein the host cell comprises (i) one or more (e.g., two, several) heterologous polynucleotides encoding a lactate dehydrogenase described herein, a lactaldehyde dehydrogenase described herein, a lactaldehyde reductase described herein, a propanediol dehydratase described herein, and/or a n-propanol dehydrogenase described herein, and (ii) one or more heterologous polynucleotides encoding a thiolase described herein, a CoA-transferase described herein, an acetoacetate decarboxylase described herein, and an isopropanol dehydrogenase described herein, under suitable conditions to produce the n-propanol and isopropanol; and (b) recovering the n-propanol and isopropanol.

Methods for the production of propanol may be performed in a fermentable medium comprising any one or more (e.g., two, several) sugars, such as glucose, fructose, sucrose, cellobiose, xylose, xylulose, arabinose, mannose, galactose, and/or soluble oligosaccharides. In some instances, the fermentation medium is derived from a natural source, such as sugar cane, starch, or cellulose, and may be the result of pretreating the source by enzymatic hydrolysis (saccharification).

In addition to the appropriate carbon sources from one or more (e.g., two, several) sugar(s), the fermentable medium may contain other nutrients or stimulators known to those skilled in the art, such as macronutrients (e.g., nitrogen sources) and micronutrients (e.g., vitamins, mineral salts, and metallic cofactors). In some aspects, the carbon source can be preferentially supplied with at least one nitrogen source, such as yeast extract, N₂ or peptone (e.g., Bacto™ Peptone). Nonlimiting examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. Examples of mineral salts and metallic cofactors include, but are not limited to Na, P, K, Mg, S, Ca, Fe, Zn, Mn, and Cu.

Suitable conditions used for the methods of propanol production may be determined by one skilled in the art in light of the teachings herein. In some aspects of the methods, the host cells are cultivated for about 12 hours to about 216 hours, such as about 24 hours to about 144 hours, or about 36 hours to about 96 hours. The temperature is typically between about 26° C. to about 60° C., e.g., about 34° C. to about 50° C., and at a pH of about 3.0 to about 8.0, such as about 3.0 to about 7.0, about 3.0 to about 6.0, about 3.0 to about 5.0, about 3.5 to about 4.5, about 4.0 to about 8.0, about 4.0 to about 7.0, about 4.0 to about 6.0, about 4.0 to about 5.0, about 5.0 to about 8.0, about 5.0 to about 7.0, or about 5.0 to about 6.0 or less than about 6.0, less than about 5.5, less than about 5.0, less than about 4.5, less than about 4.0, less than about 3.5, less than about 3.0, or less than about 2.5. In some aspects of the methods, the resulting intracellular pH of the host cell is about 2.0 to about 8.0, such as about 2.0 to about 7.0, about 2.0 to about 6.0, about 2.0 to about 5.0, about 1.5 to about 4.5, about 3.0 to about 8.0, such as about 3.0 to about 7.0, about 3.0 to about 6.0, about 3.0 to about 5.0, about 3.5 to about 4.5, about 4.0 to about 8.0, about 4.0 to about 7.0, about 4.0 to about 6.0, about 4.0 to about 5.0, about 5.0 to about 8.0, about 5.0 to about 7.0, or about 5.0 to about 6.0, or less than about 6.0, less than about 5.5, less than about 5.0, less than about 4.5, less than about 4.0, less than about 3.5, less than about 3.0, or less than about 2.5. Cultivation may be performed under anaerobic, microaerobic, or aerobic conditions, as appropriate. In some aspects, the cultivation is performed under anaerobic conditions.

Cultivation may be performed under anaerobic, substantially anaerobic (microaerobic), or aerobic conditions, as appropriate. Briefly, anaerobic refers to an environment devoid of oxygen, substantially anaerobic (microaerobic) refers to an environment in which the concentration of oxygen is less than air, and aerobic refers to an environment wherein the oxygen concentration is approximately equal to or greater than that of the air. Substantially anaerobic conditions include, for example, a culture, batch fermentation or continuous fermentation such that the dissolved oxygen concentration in the medium remains less than 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases. In some embodiments, the cultivation is performed under anaerobic conditions or substantially anaerobic conditions.

The methods of described herein can employ any suitable fermentation operation mode. For example, a batch mode fermentation may be used with a close system where culture media and host microorganism, set at the beginning of fermentation, have no additional input except for the reagents certain reagents, e.g., for pH control, foam control or others required for process sustenance. The process described herein can also be employed in Fed-batch or continuous mode.

The methods described herein may be practiced in several bioreactor configurations, such as stirred tank, bubble column, airlift reactor and others known to those skilled in the art.

The methods may be performed in free cell culture or in immobilized cell culture as appropriate. Any material support for immobilized cell culture may be used, such as alginates, fibrous bed, or argyle materials such as chrysotile, montmorillonite KSF and montmorillonite K-10.

In one aspect of the methods, the n-propanol, isopropanol, or combined n-propanol and isopropanol, is produced at a titer greater than about 10 g/L, e.g., greater than about 25 g/L, 50 g/L, 75 g/L, 100 g/L, 125 g/L, 150 g/L, 160 g/L, 170 g/L, 180 g/L, 190 g/L, 200 g/L, 210 g/L, 225 g/L, 250 g/L, 275 g/L, 300 g/L, 325 g/L, 350 g/L, 400 g/L, or 500 g/L; or between about 10 g/L and about 500 g/L, e.g., between about 50 g/L and about 350 g/L, about 100 g/L and about 300 g/L, about 150 g/L and about 250 g/L, about 175 g/L and about 225 g/L, or about 190 g/L and about 210 g/L. In one aspect of the methods, the n-propanol is produced at a titer greater than about 0.01 gram per gram of carbohydrate, e.g., greater than about 0.02, 0.05, 0.75, 0.1, 0.2, 0.3, 0.4, 0.5, 0.6, 0.7, 0.8, 0.9, or 1.0 gram per gram of carbohydrate.

In one aspect of the methods, the amount of produced n-propanol, isopropanol, or combined n-propanol, is at least 5%, e.g., at least 10%, at least 15%, at least 20%, at least 25%, at least 30%, at least 50%, or at least 100% greater compared to cultivating the host cell without the one or more (e.g., two, several) polynucleotide that encode the lactate dehydrogenase, lactaldehyde dehydrogenase, lactaldehyde reductase, propanediol dehydratase and/or n-propanol dehydrogenase under the same conditions.

The recombinant product (e.g., n-propanol or isopropanol) can be optionally recovered from the fermentation medium using any procedure known in the art including, but not limited to, chromatography (e.g., size exclusion chromatography, adsorption chromatography, ion exchange chromatography), electrophoretic procedures, differential solubility, osmosis, distillation, extraction (e.g., liquid-liquid extraction), pervaporation, extractive filtration, membrane filtration, membrane separation, reverse, or ultrafiltration. In one aspect, the n-propanol and isopropanol is separated from other fermented material and purified by conventional methods of distillation. Accordingly, in one aspect, the method further comprises purifying the recovered n-propanol, or combined n-propanol and isopropanol, by distillation.

The recombinant n-propanol and isopropanol may also be purified by the chemical conversion of impurities (contaminants) to products more easily removed from propanol by the procedures described above (e.g., chromatography, electrophoretic procedures, differential solubility, distillation, or extraction) and/or by direct chemical conversion of one or more (e.g., two, several) of the impurities to n-propanol or isopropanol. For example, in one aspect, the method further comprises purifying the recovered n-propanol by converting propanal contaminant to n-propanol, or converting acetone contaminant to isopropanol. Conversion of propanal to n-propanol or acetone to isopropanol may be accomplished using any suitable reducing agent known in the art (e.g., lithium aluminium hydride (LiAlH₄), a sodium species (such as sodium amalgam or sodium borohydride (NaBH₄)), tin species (such as tin(II) chloride), hydrazine, zinc-mercury amalgam (Zn(Hg)), diisobutylaluminum hydride (DIBAH), oxalic acid (C₂H₂O₄), formic acid (HCOOH), Ascorbic acid, iron species (such as iron(II) sulfate), and the like).

In some aspects of the methods, the recombinant propanol preparation before and/or after being optionally purified is substantially pure. With respect to the methods of producing n-propanol and coproducing n-propanol with isopropanol, “substantially pure” intends a recovered preparation that contains no more than 15% impurity, wherein impurity intends compounds other than propanol but does not include either propanol isomer. A substantially pure preparation may contain mixtures of both n-propanol and isopropanol. In one variation, a substantially pure preparation is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

The n-propanol and isopropanol produced by any of the methods described herein may be converted to propylene. Propylene can be produced by the chemical dehydration of n-propanol and/or isopropanol using acidic catalysts known in the art, such as acidic alumina, zeolites, and other metallic oxides; acidic organic-sulfonic acid resins; mineral acids such as phosphoric and sulfuric acids; and Lewis acids such as boron trifluoride and aluminum compounds (March, Jerry. Advanced Organic Chemistry. New York: John Wiley and Sons, 1992). Suitable temperatures for dehydration of n-propanol and/or isopropanol to propylene typically range from about 180° C. to about 600° C., e.g., 300° C. to about 500° C., or 350° C. to about 450° C.

The dehydration reaction of n-propanol and/or iso-propanol is typically conduced in an adiabatic or isothermal reactor, which can also be a fixed or a fluidized bed reactor; and can be optimized using residence time ranging from about 0.1 to about 60 seconds, e.g., from about 1 to about 30 seconds. Non-converted alcohol can be recycled to the dehydration reactor.

In one aspect is a method of producing propylene, comprising: (a) cultivating a recombinant host cell described herein in a medium under suitable conditions to produce n-propanol; (b) recovering the n-propanol; (c) dehydrating the n-propanol under suitable conditions to produce propylene; and (d) recovering the propylene. In another aspect is a method of producing propylene, comprising: (a) cultivating a recombinant host cell described herein in a medium under suitable conditions to produce n-propanol and isopropanol; (b) recovering the n-propanol and isopropanol; (c) dehydrating the n-propanol and isopropanol under suitable conditions to produce propylene; and (d) recovering the propylene.

In some aspects of producing propylene, the medium is a fermentable medium. In another aspect, the medium is a fermentable medium comprising sugarcane juice (e.g., non-sterilized sugarcane juice). In one aspect, the amount of n-propanol produced prior to dehydrating the n-propanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L. The recovered n-propanol and isopropanol prior to dehydration may or may not be substantially pure, as described supra. In one aspect, dehydrating the n-propanol and isopropanol under suitable conditions to produce propylene comprises contacting or treating the n-propanol and isopropanol with an acid catalyst, as known in the art.

Contaminants that may be generated during dehydration may be removed through purification using techniques known in the art. For example, propylene can be washed with water or a caustic solution to remove acidic compounds like carbon dioxide and/or fed into beds to absorb polar compounds like water or for the removal of, e.g., carbon monoxide. Alternatively, a distillation column can be used to separate higher hydrocarbons such as propane, butane, butylene and higher compounds. The separation of propylene from contaminants like ethylene may be carried out by methods known in the art, such as cryogenic distillation.

Suitable assays to test for the production of n-propanol, isopropanol, and propylene for the methods of production and host cells described herein can be performed using methods known in the art. For example, final n-propanol and isopropanol product and intermediates (e.g., propanal and acetone), as well as other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of n-propanol and isopropanol in the fermentation broth can also be tested with the culture supernatant. Byproducts and residual sugar in the fermentation medium (e.g., glucose) can be quantified by HPLC using, for example, a refractive index detector for glucose and alcohols, and a UV detector for organic acids (Lin et al., Biotechnol. Bioeng. 90:775-779 (2005)), or using other suitable assay and detection methods well known in the art.

The propylene produced from n-propanol and isopropanol may be further converted to polypropylene or polypropylene copolymers by polymerization processes known in the art. Suitable temperatures typically range from about 105° C. to about 300° C. for bulk polymerization, or from about 50° C. to about 100° C. for polymerization in suspension. Alternatively, polypropylene can be produced in a gas phase reactor in the presence of a polymerization catalyst such as Ziegler-Natta or metalocene catalysts with temperatures ranging from about 60° C. to about 80° C.

The following examples are provided by way of illustration and are not intended to be limiting of the invention.

### EXAMPLES

Chemicals used as buffers and substrates were commercial products of at least reagent grade.

### Host Strains

Lactobacillus reuteri SJ10655 (O4ZXV)

A strain described as Lactobacillus reuteri DSM20016 was obtained from a public strain collection. This strain was subcultured in MRS medium, and an aliquot frozen as SJ10468. SJ10468 was inoculated into MRS medium, propagated without shaking for one day at 37° C., and spread on MRS agar plates to obtain single colonies. After two days of growth at 37° C., a single colony was reisolated on a MRS agar plate, the plate incubated at 37° C. for three days, and the cell growth on the plate was scraped off and stored in the strain collection as SJ10655 (alternative name: O4ZXV).

The same cell growth was used to inoculate a 10 ml MRS culture, which was incubated without shaking at 37° C. for 3 days, whereafter cells were harvested by centrifugation and genomic DNA was prepared using a QIAamp DNA Blood Kit (Qiagen, Hilden, Germany) and sent for genome sequencing.

The genome sequence revealed that the isolate SJ10655 (O4ZXV) has a genome essentially identical to that of JCM1112, rather than to that of the closely related strain DSM20016. JCM1112 and DSM20016 are derived from the same original isolate, L. reuteri F275 (Morita et al. DNA research, 2008, 15, 151-161.)

Lactobacillus reuteri SJ11294

The Lactobacillus reuteri strain SJ11294 is a mutant of SJ10655 with improved transformability (EcFc to provide when available U.S. Ser. No. 61/648,958, filed May 18, 2012).

Lactobacillus reuteri SJ 11360

The Lactobacillus reuteri strain SJ11360 is the Lactobacillus reuteri strain SJ11294 stransformed with pSJ10600 (see WO2012/058603) using the transformation procedure described below.

Lactobacillus reuteri TRGU1014

The Lactobacillus reuteri strain TRGU1014 is the Lactobacillus reuteri strain TRGU1013 (see Example 22) transformed with pSJ10600 (see WO2012/058603) using the transformation procedure described herein.

Escherichia coli TG1

TG1 is a commonly used cloning strain and was obtained from a commercial supplier; it has the following genotype: F′[traD36 laclq Δ(lacZ) M15 proA+B+] glnV (supE) thi-1 Δ(mcrB-hsdSM)5 (rK-mK-McrB-) thi Δ(lac-proAB).

### Media

LB plates were composed of 37 g LB agar (Sigma cat no. L3027) and double distilled water to 1 L.

LBPGS plates were composed of 37 g LB agar (Sigma cat no. L3027), 0.5% starch (Merck cat. no. 101252), 0.01 M K₂PO₄, 0.4% glucose, and double distilled water to 1 L.

TY bouillon medium was composed of 20 g tryptone (Difco cat no. 211699), 5 g yeast extract (Difco cat no. 212750), 7*10⁻³ g ferrochloride, 1*10⁻³ g manganese(II)-chloride, 1.5*10⁻³ g magnesium sulfate, and double distilled water to 1 L.

Minimal medium (MM) was composed of 20 g glucose, 1.1 g KH₂PO₄, 8.9 g K₂HPO₄; 1.0 g (NH₄)₂SO₄; 0.5 g Na-citrate; 5.0 g MgSO₄.7H₂O; 4.8 mg MnSO₄.H₂O; 2 mg thiamine; 0.4 mg/L biotin; 0.135 g FeCl₃.6H₂O; 10 mg ZnCl₂.4H₂O; 10 mg CaCl₂.6H₂O; 10 mg Na₂MoO₄.2H₂O; 9.5 mg CuSO₄.5H₂O; 2.5 mg H₃BO₃; and double distilled water to 1 L, pH adjusted to 7 with HCl.

MRS medium was obtained from Difco™, as either Difco™ Lactobacilli MRS Agar or Difco™ Lactobacilli MRS Broth, having the following compositions—Difco™ Lactobacilli MRS Agar: Proteose Peptone No. 3 (10.0 g), Beef Extract (10.0 g), Yeast Extract (5.0 g), Dextrose (20.0 g), Polysorbate 80 (1.0 g), Ammonium Citrate (2.0 g), Sodium Acetate (5.0 g), Magnesium Sulfate (0.1 g), Manganese Sulfate (0.05 g), Dipotassium Phosphate (2.0 g), Agar (15.0 g) and water to 1 L. Difco™ Lactobacilli MRS Broth: Consists of the same ingredients without the agar.

LC (Lactobacillus carrying) medium was composed of Trypticase (10 g), Tryptose (3 g), Yeast extract (5 g), KH₂PO₄ (3 g), Tween 80 (1 ml), sodium-acetate (1 g), ammonium citrate (1.5 g), Cystein-HCl (0.2 g), MgSO₄.7H₂O (12 mg), FeSO₄.7H₂O (0.68 mg), MnSO₄.2H₂O (25 mg), and double distilled water to 1 L, pH adjusted to 7.0. Stearile glucose was added after autoclaving to 1% (5 ml of a 20% glucose stock solution/100 ml medium).

Transformation Protocol for Lactobacillus reuteri

Unless noted otherwise, plasmid DNA was introduced into Lactobacillus reuteri by electroporation. The Lactobacillus reuteri strains were prepared for electroporation as follows: The strain was inoculated from a frozen stock culture into LCM medium, and incubated without shaking at 37° C. overnight. A 5 ml aliquot was transferred into 500 ml LCM and incubated at 37° C. without shaking until OD₆₀₀ reached approximately 0.8. The cells were harvested by centrifugation as above, resuspended and washed 2 times in 50 ml of ion-exchanged stearile water at room temperature, and harvested by centrifugation. The cells were finally gently resuspended in 2.5 ml of 30% PEG1500, and 50 microliter aliquots were quickly frozen in an alcohol/dry ice bath, and stored at −80° C. until use.

For electroporation of Lb. reuteri, the frozen cells were thawed on ice, and 2 microliter of a DNA suspension in TE buffer was added. 40 microliters of the mixture was transferred to an ice-cold 2 mm electroporation cuvette, kept on ice for 1-3 minutes, and electroporation carried out in a BioRad Gene Pulser™ with a setting of 1.5 kV; 25 microFarad; 400 Ohms. 500 microliter of LCM was added, and the mixture incubated without shaking for 2 hours at 37° C. before plating. Cells were plated on MRS agar plates, supplemented with the required antibiotics, and incubated in an anaerobic chamber (Oxoid; equipped with Anaerogen sachet).

### Example 1

### Cloning of a C. acetobuylicum Thiolase Gene and Construction of Vector pTRGU51

The 1176 bp coding sequence (CDS) of a thiolase gene identified in C. acetobutylicum was optimized for expression in E. coli and synthetically constructed into pTRGU51. The DNA fragment containing the codon optimized CDS was designed with a ribosomal binding site (RBS, sequence 5′-GAAGGAGATATACC-3′) immediately prior to the start codon. The resulting sequence was then submitted to and synthesized by Geneart AG (Regenburg, Germany) and delivered in the pMA backbone vector containing the β-lactamase encoding gene blaTEM-1. When synthesized, the CDS and RBS fragment was flanked by restriction sites to facilitate subsequent cloning steps. The entire synthetic fragment cloned into the pMA vector was EcoRI-RBS-CDS-STOP-BamHI-XbaI, resulting in pTRGU51.

The wild-type nucleotide sequence (WT) and deduced amino acid sequence of the C. acetobutylicum thiolase gene correspond to SEQ ID NO: 37 and 38, respectively. The coding sequence is 1179 bp including the stop codon and the encoded predicted protein is 392 amino acids. Using the SignalP program (Nielsen et al., 1997, Protein Engineering 10: 1-6), no signal peptide in the sequence was predicted. Based on this program, the predicted mature protein contains 392 amino acids with a predicted molecular mass of 41.4 kDa and an isoelectric pH of 7.08.

### Example 2

### Cloning of a B. subtilis Succinyl-CoA:Acetoacetate Transferase Gene and Construction of Vectors pTRGU58 and pTRGU59

The 699 bp coding sequence (CDS) of the scoA subunit of the B. subtilis succinyl-CoA:acetoacetate transferase gene and the 648 bp coding sequence of the scoB subunit of the B. subtilis succinyl-CoA:acetoacetate transferase gene were optimized for expression in E. coli and synthetically constructed into pTRGU58 and pTRGU59, respectively. Each DNA fragment containing a codon optimized CDS was designed with a ribosomal binding site and synthesized by Geneart AG as described in Example 1, with modified restriction sites as noted below. The entire synthetic fragment containing scoA cloned into the pMA vector was EcoRI-BamHI-RBS-scoA-STOP-NotI-XbaI, resulting in pTRGU58. The entire synthetic fragment containing scoB cloned into the pMA vector was EcoRI-NotI-RBS-scoB-STOP-HindIII-XbaI, resulting vector is named pTRGU59.

The wild-type nucleotide sequence (WT) and deduced amino acid sequence of the B. subtilis scoA subunit of the succinyl-CoA:acetoacetate transferase correspond to SEQ ID NO: 39 and 40, respectively. The coding sequence is 702 bp including the stop codon and the encoded predicted protein is 233 amino acids. Using the SignalP program (Nielsen et al., supra), no signal peptide in the sequence was predicted. Based on this program, the predicted mature protein contains 233 amino acids with a predicted molecular mass of 25.1 kDa and an isoelectric pH of 6.50.

The wild-type nucleotide sequence (WT) and deduced amino acid sequence of the B. subtilis scoB subunit of the succinyl-CoA:acetoacetate transferase gene correspond to SEQ ID NO: 41 and 42, respectively. The coding sequence is 651 bp including the stop codon and the encoded predicted protein is 216 amino acids. Using the SignalP program (Nielsen et al., supra), no signal peptide in the sequence was predicted. Based on this program, the predicted mature protein contains 216 amino acids with a predicted molecular mass of 23.4 kDa and an isoelectric pH of 5.07.

### Example 3

### Cloning of a B. mojavensis Succinyl-CoA:Acetoacetate Transferase Gene and Construction of Vectors pTRGU60 and pTRGU61

The genomic sequence of Bacillus mojavensis was obtained by sequencing with a Genome Analyzer II (Illumine, San Diego, Calif., USA) using standard techniques known in the art. 8,327,624 reads of 72 base-pairs were assembled with Velvet version 0.7.31 in to 73 contigs and 3,913,09 base-pairs. Glimmer3 was used for gene finding resulting in 4,092 putative genes. The coding sequence (CDS) of the scoA and scoB subunits of the B. mojavensis succinyl-CoA:acetoacetate transferase gene were found via homology to the known scoA and scoB genes from Bacillus subtilis.

The 711 bp coding sequence (CDS) of the scoA subunit of the B. mojavensis succinyl-CoA:acetoacetate transferase gene and the 654 bp coding sequence of the scoB subunit of the B. mojavensis succinyl-CoA:acetoacetate transferase gene were optimized for expression in E. coli and synthetically constructed into pTRGU60 and pTRGU61, respectively. Each DNA fragment containing a codon optimized CDS was designed with a ribosomal binding site and synthesized by Geneart AG as described in Example 1, with modified restriction sites as noted below. The entire synthetic fragment containing scoA cloned into the pMA vector was EcoRI-BamHI-RBS-scoA-STOP-NotI-XbaI, resulting in pTRGU60. The entire synthetic fragment containing scoB cloned into the pMA vector was EcoRI-NotI-RBS-scoB-STOP-HindIII-XbaI, resulting in pTRGU61.

The wild-type nucleotide sequence (WT) and deduced amino acid sequence of the B. mojavensis scoA subunit of the succinyl-CoA:acetoacetate transferase gene correspond to SEQ ID NO: 43 and 44, respectively. The coding sequence is 714 bp including the stop codon and the encoded predicted protein is 237 amino acids. Using the SignalP program (Nielsen et al., supra), no signal peptide in the sequence was predicted. Based on this program, the predicted mature protein contains 237 amino acids with a predicted molecular mass of 25.5 kDa and an isoelectric pH of 5.82.

The wild-type nucleotide sequence (WT) and deduced amino acid sequence of the B. mojavensis scoB subunit of the succinyl-CoA:acetoacetate transferase gene correspond to SEQ ID NO: 45 and 46, respectively. The coding sequence is 657 bp including the stop codon and the encoded predicted protein is 218 amino acids. Using the SignalP program (Nielsen et al., supra), no signal peptide in the sequence was predicted. Based on this program, the predicted mature protein contains 218 amino acids with a predicted molecular mass of 23.7 kDa and an isoelectric pH of 5.40.

### Example 4

### Cloning of a C. beijerinckii Acetoacetate Decarboxylase Gene and Construction of Vector pTRGU62

The 738 bp coding sequence (CDS) of the acetoacetate decarboxylase gene of C. beijerinckii was optimized for expression in E. coli and synthetically constructed into pTRGU62. The DNA fragment containing a codon optimized CDS was designed with a ribosomal binding site and synthesized by Geneart AG as described in Example 1, with modified restriction sites as noted below. The entire synthetic fragment cloned into the pMA vector was EcoRI-HindIII-RBS-CDS-STOP-AscI-XbaI, resulting in pTRGU62.

The wild-type nucleotide sequence (WT) and deduced amino acid sequence of the C. beijerinckii acetoacetate decarboxylase gene correspond to SEQ ID NO: 47 and 48, respectively. The coding sequence is 741 bp including the stop codon and the encoded predicted protein is 246 amino acids. Using the SignalP program (Nielsen et al., supra), no signal peptide in the sequence was predicted. Based on this program, the predicted mature protein contains 246 amino acids with a predicted molecular mass of 27.5 kDa and an isoelectric pH of 6.18.

### Example 5

### Cloning of a C. beijerinckii Isopropanol Dehydrogenase Gene and Construction of Vector pTRGU53

The 1053 bp coding sequence (CDS) of the isopropanol dehydrogenase gene of C. beijerinckii was optimized for expression in E. coli and synthetically constructed into pTRGU53. The DNA fragment containing a codon optimized CDS was designed with a ribosomal binding site and synthesized by Geneart AG as described in Example 1, with modified restriction sites as noted below. The entire synthetic fragment cloned into the pMA vector was EcoRI-AscI-RBS-CDS-STOP-XbaI, resulting in pTRGU53.

The wild-type nucleotide sequence (WT) and deduced amino acid sequence of the C. beijerinckii isopropanol dehydrogenase gene correspond to SEQ ID NO: 49 and 50, respectively. The coding sequence is 1056 bp including the stop codon and the encoded predicted protein is 351 amino acids. Using the SignalP program (Nielsen et al., supra), no signal peptide in the sequence was predicted. Based on this program, the predicted mature protein contains 351 amino acids with a predicted molecular mass of 37.8 kDa and an isoelectric pH of 6.64.

### Example 6

### Cloning of a T. ethanolicus Isopropanol Dehydrogenase Gene and Construction of Vector pTRGU54

The 1056 bp coding sequence (CDS) of the isopropanol dehydrogenase gene of T. ethanolicus was optimized for expression in E. coli and synthetically constructed into pTRGU54. The DNA fragment containing a codon optimized CDS was designed with a ribosomal binding site and synthesized by Geneart AG as described in Example 1, with modified restriction sites as noted below. The entire synthetic fragment cloned into the pMA vector was EcoRI-AscI-RBS-CDS-STOP-XbaI, resulting in pTRGU54.

The wild-type nucleotide sequence (WT) and deduced amino acid sequence of the T. ethanolicus isopropanol dehydrogenase gene corresponds to SEQ ID NO: 51 and 52, respectively. The coding sequence is 1059 bp including the stop codon and the encoded predicted protein is 352 amino acids. Using the SignalP program (Nielsen et al., supra), no signal peptide in the sequence was predicted. Based on this program, the predicted mature protein contains 352 amino acids with a predicted molecular mass of 37.7 kDa and an isoelectric pH of 6.23.

### Example 7

### Construction and Transformation of Empty Expression Vector pTRGU88

A 2349 bp fragment containing the LacI^{q} repressor, the trc promoter, and a multiple cloning site (MCS) was amplified from pTrc99A (E. Amann and J. Brosius, 1985, Gene 40(2-3), 183-190) using primers pTrcBgIIItop and pTrcScaIbot shown below.

Primer pTrcBglIItop:
(SEQ ID NO: 63)
5′-GAAGATCTATGGTGCAAAACCTTTCGCGG-3′
Primer pTrcScaIbot:
(SEQ ID NO: 64)
5′-AAAAGTACTCAACCAAGTCATTCTGAG-3′

The PCR reaction was contained 12.5 pmol primer pTrcBgIIItop, 12.5 pmol primer pTrcScaIbot and 0.625 units of Platinum Pfx DNA polymerase (Invitrogen, UK). The amplification reaction was programmed for 25 cycles each at 95° C. for 2 minutes; 95° C. for 30 seconds, 42° C. for 30 seconds, and 72° C. for 2 minute; then one cycle at 72° C. for 3 minutes. The resulting PCR product was purified with a PCR Purification Kit (Qiagen, Hilden, Germany) according to manufacturer's instructions and digested overnight at 37° C. with 5 units each of BgIII (New England Biolabs, Ipswich, Mass., USA) and ScaI (New England Biolabs) (restriction sites are underlined in the above primers). The digested fragment was then purified with a PCR Purification Kit (Qiagen) according to manufacturer's instructions.

Plasmid pACYC177 (Y. K. Mok, et al., 1988, Nucleic Acids Res. 16(1), 356) containing a p15A origin of replication was digested at 37° C. with 5 units ScaI (New England Biolabs) and 10 units BamHI (New England Biolabs) for two hours. 10 units of calf intestine phosphatase (CIP) (New England Biolabs) were added to the digest and incubation was continued for an additional hour, resulting in a 3256 bp fragment and a 685 bp fragment. The digest mixture was run on a 1% agarose gel and the 3256 bp fragment was excised from the gel and purified using a QIAquick Gel Extraction Kit (Qiagen) according to the manufacturer's instructions.

The purified 2349 bp PCR/restriction fragment was ligated into the 3256 bp restriction fragment using a Rapid Ligation Kit (F. Hoffmann-La Roche Ltd, Basel Switzerland) according to the manufacturer's instructions, resulting in pMIBa2. Plasmid pMIBa2 was digested with PstI using the standard buffer 3 and BSA as suggested by New England Biolabs, resulting in a 1078 bp PstI fragment containing the first 547 bp of blaTEM-1 (including the blaTEM-1 promoter and RBS) and a 4524 bp fragment containing the p15A origin of replication, the LacI^{q} repressor, the trc promoter, a multiple cloning site (MCS), and aminoglycoside 3′-phosphotransferase gene.

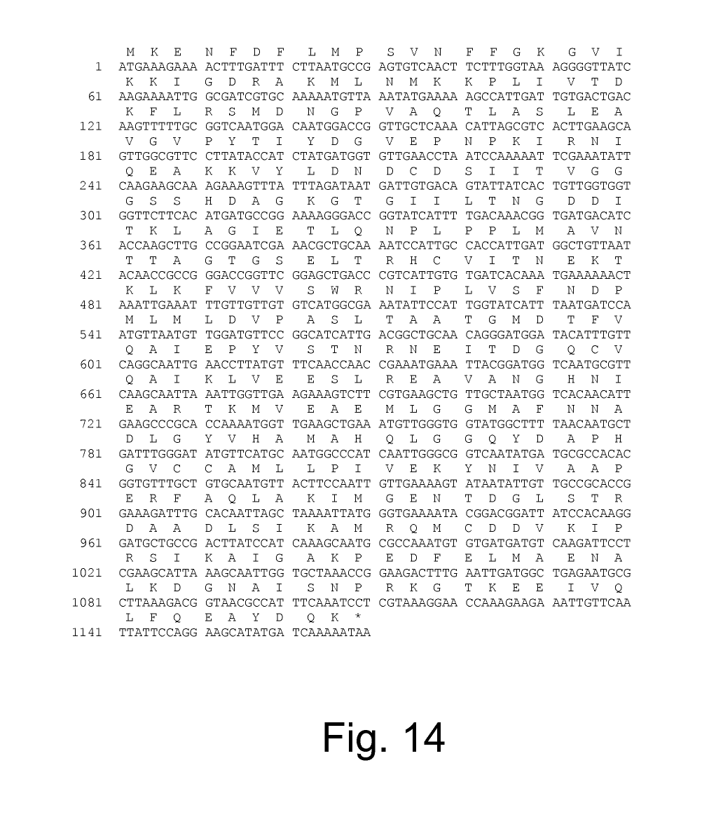

The 4524 bp fragment was ligated overnight at 16° C. using T4 DNA ligase in T4 DNA ligase buffer containing 10 mM ATP (F. Hoffmann-La Roche Ltd). A 1 μL aliquot of the ligation mixture was transformed into E. coli SJ2 cells using electroporation. Transformants were plated onto LBPGS plates containing 20 μg/mL kanamycin and incubated at 37° C. overnight. Selected colonies were then streaked on LB plates with 200 μg/mL ampicillin and on LB plates with 20 μg/mL kanamycin. Eight transformants that were ampicillin sensitive and kanamycin resistant were isolated and streak purified on LB plates with 20 μg/mL kanamycin. Each of eight colonies was inoculated in liquid TY bouillon medium and incubated overnight at 37° C. The plasmid from each colony was isolated using a Qiaprep®Spin Miniprep Kit (Qiagen) then double digested with EcoRI and MluI. Each plasmid resulted in a correct restriction pattern of 1041 bp and 3483 bp when analyzed using the electrophoresis system “FlashGel® System” from Lonza (Basel, Switzerland). The liquid overnight culture of one transformant designated E. coli TRGU88 was stored in 30% glycerol at −80° C. The corresponding plasmid pTRGU88 (FIG. 14) was isolated from E. coli TRGU88 with a Qiaprep® Spin Miniprep Kit (Qiagen) using the manufacturer's instructions and stored at −20° C.

### Example 8

### Construction and Transformation of pTRGU170 Expressing a C. acetobuylicum Thiolase Gene, a B. subtilis Succinyl-CoA:Acetoacetate Transferase Gene, and a C. beijerinckii Acetoacetate Decarboxylase Gene

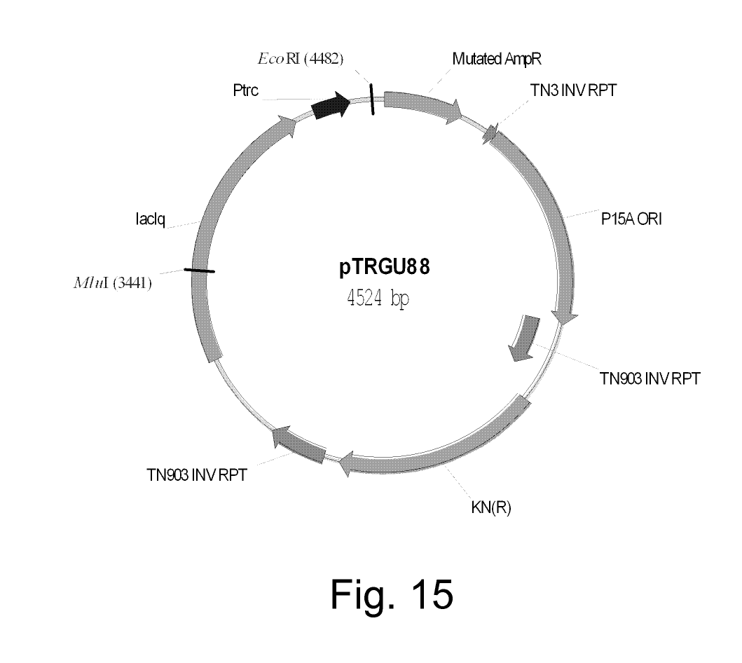

Plasmids pTRGU88 and pTRGU51 were digested individually with EcoRI and XbaI, and dephosphorylated with CIP using standard methods as outlined herein. The resulting 1205 bp fragment of pTRGU51 and 4497 bp fragment of pTRGU88 were each purified using gel electrophoresis then ligated as outlined herein. A 1 μl aliquot of the ligation mixture was transformed into E. coli TOP10 cells (InVitrogen, UK) using electroporation. 10 μl of the transformation mixture was plated onto LB plates containing 20 μg/mL kanamycin. Each of four resulting colonies was inoculated in liquid TY bouillon medium and cultivated overnight at 37° C. Plasmids from each colony were isolated, digested with EcoRI and XbaI, and the resulting fragments size separated on a 1% agarose gel as described herein. The insert sequences were the correct size and verified by sequencing. One transformant designated E. coli TRGU130 was stored at −80° C. in 30% glycerol. The corresponding plasmid pTRGU130 (FIG. 15) was isolated from E. coli TRGU130 as described herein and stored at −20° C. Plasmid pTRGU130 was restricted with BamHI and XbaI, and dephosphorylated with CIP using the standard methods as outlined herein. The resulting 5696 bp DNA fragment was purified on a 1% agarose gel as described herein.

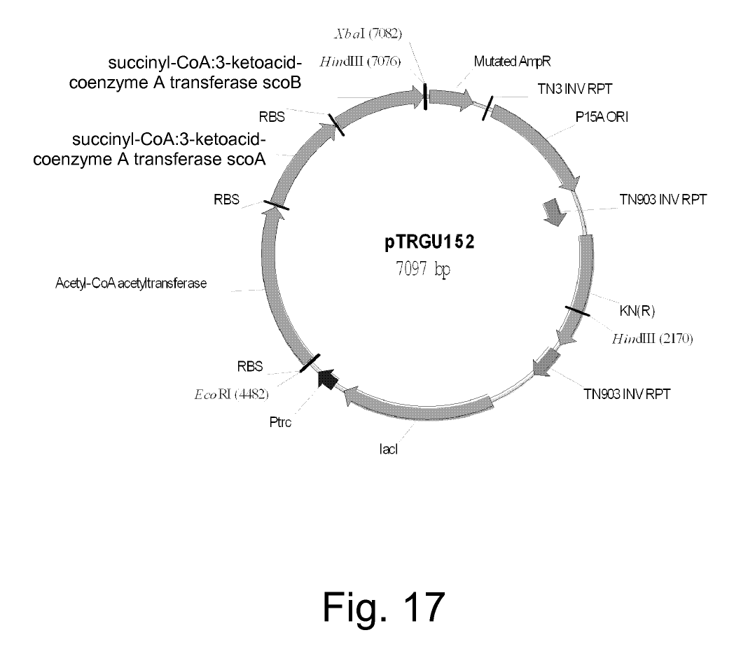

Plasmids pTRGU58, pTRGU59, and pTRGU88 were digested individually with EcoRI/NotI, NotI/XbaI, and EcoRI/XbaI, respectively, and dephosphorylated with CIP using the standard methods as outlined herein. The resulting scoA fragment of pTRGU58, scoB fragment of pTRGU59, and 4497 bp fragment of pTRGU88 were each purified using gel electrophoresis as outlined herein. A three fragment ligation of the resulting fragments was carried out using standard methods with T4 DNA ligase and T4 DNA ligase buffer containing 10 mM ATP (F. Hoffmann-La Roche Ltd), and then transformed, isolated, and stored, as outlined herein, resulting in pTRGU136 wherein the combined scoA and scoB sequence (scoAB) was flanked by the restriction sites BamHI and XbaI. The scoAB fragment was then excised from pTRGU136 with BamHI and XbaI, purified, and ligated with the purified BamHI-XbaI 5696 bp DNA fragment of pTRGU130 above using standard methods described herein. The resulting ligation product was transformed, isolated, and stored, as outlined herein, resulting in pTRGU152 (FIG. 17) containing the C. acetobutylicum thiolase gene and B. subtilis succinyl-CoA:acetoacetate transferase gene.

Plasmids pTRGU88 and pTRGU62 were digested individually with EcoRI and XbaI, and dephosphorylated with CIP using standard methods as outlined herein. The resulting fragment of pTRGU62 containing the C. beijerinckii acetoacetate decarboxylase gene and the 4497 bp fragment of pTRGU88 were each purified using gel electrophoresis, ligated, transformed and stored, as outlined herein, resulting in pTRGU140. Plasmid pTRGU140 was then digested with HindIII and XbaI and the resulting 769 bp fragment was purified using gel electrophoresis as outlined herein.

Plasmid pTRGU152 was digested in parallel with 1) EcoRI and XbaI, resulting in a XbaI-EcoRI fragment (4497 bp) and 2) EcoRI and HindIII, resulting in a XbaI-HindIII fragment (4497 bp). Each fragment was individually purified on a 1% agarose gel as described herein and ligated together in a 3-fragment ligation with the 769 bp HindIII-XbaI fragment above using T4 DNA ligase under standard conditions as recommended by the manufacturer (F. Hoffmann-La Roche Ltd). A 1 μl aliquot of the ligation mixture was transformed into E. coli TOP10 cells and plated onto LB plates containing 20 μg/mL kanamycin as described herein. Colony TRGU170 was stored in 30% glycerol at −80° C. after streak purification on LB plates containing 20 μg/mL kanamycin. The corresponding plasmid pTRGU170 was isolated from TRGU170 and the desired gene inserts verified as described above.

### Example 9

### Construction and Transformation of pTRGU178 Expressing a C. acetobuylicum Thiolase Gene, a B. mojavensis Succinyl-CoA:Acetoacetate Transferase Gene, and a C. beijerinckii Acetoacetate Decarboxylase Gene

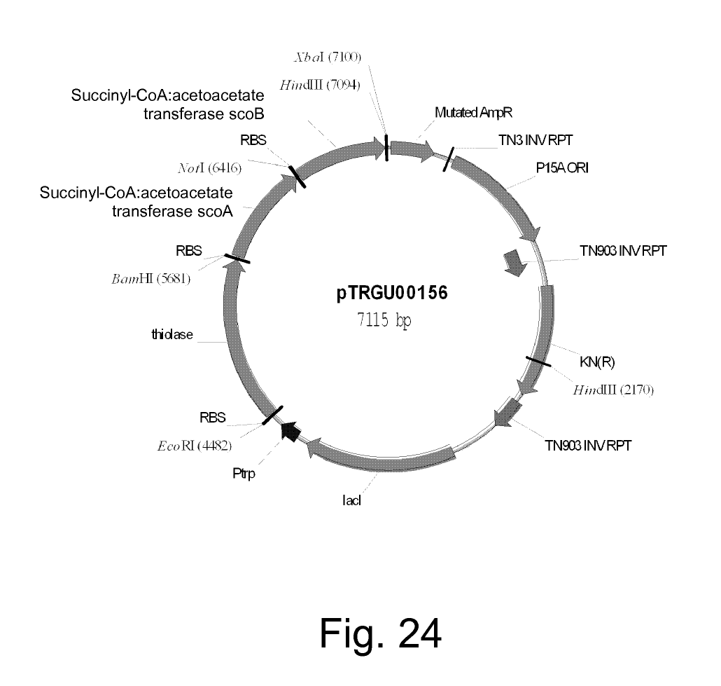

Plasmid pTRGU178 was constructed in a similar manner as described for pTRGU170 in Example 8 except that the B. mojavensis succinyl-CoA:acetoacetate transferase subunit genes scoA and scoB were isolated from pTRGU60 and pTRGU61, respectively. The scoA and scoB genes were cloned into pTRGU88, transformed, isolated, and stored, as outlined in detail in Example 8 creating vector pTRGU138. A fragment containing the scoAB genes was then excised from pTRGU138 with BamHI and XbaI, purified, ligated with the purified BamHI-XbaI fragment of pTRGU130, transformed, isolated, and stored, as outlined herein, resulting in the vector pTRGU156 (FIG. 24) containing a thiolase gene and scoAB.

The C. beijerinckii acetoacetate decarboxylase 769 bp fragment from pTRGU140 was ligated via a 3-fragment ligation with a XbaI-EcoRI pTRGU156 fragment and a XbaI-HindIII pTRGU156 fragment in a similar manner as described in Example 8, followed by transformation. One transformant, E. coli TRGU178, was selected and streak purified on LB plates containing 20 μg/mL kanamycin and stored in 30% glycerol at −80° C. The corresponding plasmid pTRGU178 was isolated from E. coli TRGU178 and the desired gene inserts verified as described above.

### Example 10

### Construction and Transformation of pTRGU196 Expressing a C. acetobuylicum Thiolase Gene, a B. subtilis Succinyl-CoA:Acetoacetate Transferase Gene, a C. beijerinckii Acetoacetate Decarboxylase Gene, and a C. beijerinckii Isopropanol Dehydrogenase Gene

Plasmids pTRGU170 and pTRGU53 were digested with AscI and XbaI in standard buffer 4 (NEB) following the manufacturer's instructions. A 7853 bp fragment of pTRGU170 and a 1077 bp fragment of pTRGU53 were purified on a 1% agarose gel as described herein. The two fragments were ligated together overnight at 16° C. using T4 DNA ligase in T4 DNA ligase buffer containing 10 mM ATP (F. Hoffmann-La Roche Ltd). A 1 μl aliquot of the ligation mixture was transformed into E. coli TOP10 cells and plated onto LB plates containing 20 μg/mL kanamycin as described herein. Colony TRGU196 was stored in 30% glycerol at −80° C. as described herein. The corresponding plasmid pTRGU196 was isolated from TRGU196 and the desired gene inserts verified as described above.

### Example 11

### Construction and Transformation of pTRGU198 Expressing a C. acetobuylicum Thiolase Gene, a B. subtilis Succinyl-CoA:Acetoacetate Transferase Gene, a C. beijerinckii Acetoacetate Decarboxylase Gene, and a T. ethanolicus Isopropanol Dehydrogenase Gene

Plasmids pTRGU170 and pTRGU54 were digested with AscI and XbaI in standard buffer 4 (NEB) following the manufacturer's instructions. A 7853 bp fragment of pTRGU170 and a 1080 bp fragment of pTRGU54 were purified on a 1% agarose gel as described herein. The two fragments were ligated overnight at 16° C. using T4 DNA ligase in T4 DNA ligase buffer containing 10 mM ATP (F. Hoffmann-La Roche Ltd). A 1 μl aliquot of the ligation mixture was transformed into E. coli TOP10 cells and plated onto LB plates containing 20 μg/mL kanamycin as described herein. Colony TRGU198 was stored in 30% glycerol at −80° C. as described herein. The corresponding plasmid pTRGU198 was isolated from TRGU198 and the desired gene inserts verified as described above.

### Example 12

### Construction and Transformation of pTRGU200 Expressing a C. acetobuylicum Thiolase Gene, a B. mojavensis Succinyl-CoA:Acetoacetate Transferase Gene, a C. beijerinckii Acetoacetate Decarboxylase Gene, and a C. beijerinckii Isopropanol Dehydrogenase Gene

Plasmids pTRGU178 and pTRGU53 were digested with AscI and XbaI in standard buffer 4 (NEB) following the manufacturer's instructions. A 7871 bp fragment of pTRGU178 and a 1077 bp fragment of pTRGU53 were purified on a 1% agarose gel as described herein. The two fragments were ligated overnight at 16° C. using T4 DNA ligase in T4 DNA ligase buffer containing 10 mM ATP (F. Hoffmann-La Roche Ltd). A 1 μl aliquot of the ligation mixture was transformed into E. coli TOP10 cells and plated onto LB plates containing 20 μg/mL kanamycin as described herein. One transformant, E. coli TRGU200, was stored in 30% glycerol at −80° C. as described herein. The corresponding plasmid pTRGU200 was isolated from E. coli TRGU200 and the desired gene inserts verified as described above.

### Example 13

### Construction and Transformation of pTRGU202 Expressing a C. acetobuylicum Thiolase Gene, a B. mojavensis Succinyl-CoA:Acetoacetate Transferase Gene, a C. beijerinckii Acetoacetate Decarboxylase Gene, and a T. ethanolicus Isopropanol Dehydrogenase Gene

Plasmids pTRGU178 and pTRGU54 were digested with AscI and XbaI in standard buffer 4 (NEB) following the manufacturer's instructions. A 7871 bp fragment of pTRGU178 and a 1080 bp fragment of pTRGU54 were purified on a 1% agarose gel as described herein. The two fragments were ligated together overnight at 16° C. using T4 DNA ligase in T4 DNA ligase buffer containing 10 mM ATP (F. Hoffmann-La Roche Ltd). A 1 μL aliquot of the ligation mixture was transformed into E. coli TOP10 cells and plated onto LB plates containing 20 μg/mL kanamycin as described herein. One transformant, E. coli TRGU202, was stored in 30% glycerol at −80° C. as described herein. The corresponding plasmid pTRGU202 was isolated from E. coli TRGU202 and the desired gene inserts verified as described above.

### Example 14

### Production of Acetone and Isopropanol in Shake Flask Cultures

Seven E. coli strains (TRGU170, TRGU178, TRGU196, TRGU198, TRGU200, TRGU202, and TRGU88 as a negative control) were grown individually overnight with shaking (250 rpm) at 37° C. in 10 mL LB medium containing 10 μg/mL kanamycin and 1 mM isopropyl-beta-thio galactopyranoside. A 2 mL sample from each medium was withdrawn after 24 hours. Each sample was centrifuged at 15000×g using a table centrifuge and the supernatant was analyzed using gas chromatography. Acetone, n-propanol and isopropanol in fermentation broths were detectable by GC-FID. Samples were diluted 1+1 with 0.05% tetrahydrofuran in methanol and analyzed. GC parameters are listed in Table 1.

The analysis results showed a major acetone peak detected in supernatants from E. coli TRGU170 and E. coli TRGU178 (each lacking a heterologous isopropanol dehydrogenase gene) with a retention time of 2.012 minutes (compared with the acetone retention time in the standards at 2.012 min). As shown in Table 2, the acetone in the supernatant of E. coli TRGU170 and E. coli TRGU178 was estimated to be at least 7 to 8-fold higher than E. coli TRGU88. Isopropanol was detected in TRGU196, TRGU198, TRGU200, and TRGU202 (each containing a heterologous thiolase gene, a heterologous succinyl-CoA:acetoacetate transferase gene, a heterologous acetoacetate decarboxylase gene, and a heterologous isopropanol dehydrogenase gene) with retention time between 3.253 and 3.258 (compared with the isopropanol retention time in the standards at 3.240 min). Acetone levels in E. coli TRGU196, TRGU198, TRGU200, and TRGU202 were at similar levels observed in the negative control TRGU88.

TABLE 1

| | | Approx. |
|---|---|---|
| | | Retention |
| | | time |
| Parameter | | (min) |
| GC column | DB-WAX 30 m-0.25 mm i.d-0.50 μm | |
| | film part-no 122-7033 from J&W Scientific | |
| Carrier gas | Hydrogen | |
| Temp. gradient | 0-4.5 min: 50° C. | |
| | 4.5-9.93 min: 50-240° C. linear gradient | |
| Detection | FID | |
| Internal | Tetrahydrofuran | 2.4 |
| standard | | |
| External | Acetone (Analytical grade) | 2.0 |
| standards | n-propanol (Analytical grade) | 5.4 |
| | isopropanol (HPLC grade) | 3.3 |

TABLE 2

| | Metabolite detected by gas | Amount produced |
|---|---|---|
| Medium/Strain | chromatography | [mg/L] |
| LB medium | Trace of acetone | Not quantifiable |
| TRGU88 | Trace of acetone | Not quantifiable |
| TRGU170 | Acetone | 10 |
| TRGU178 | Acetone | 10 |
| TRGU196 | Isopropanol | 80 |
| TRGU198 | Trace of isopropanol | Not quantifiable |
| TRGU200 | Isopropanol | 70 |
| TRGU202 | Isopropanol | 70 |

### Example 15

### Production of Isopropanol with Lactobacillus plantarum (Prophetic)

A plasmid is constructed containing expression vectors for a thiolase gene, a succinyl-CoA:acetoacetate transferase gene, a acetoacetate decarboxylase gene, and a isopropanol dehydrogenase gene, codon optimized for expression in Lactobacillus plantarum as described herein. The plasmid is then transformed into Lactobacillus plantarum cells and plated onto LB plates containing 20 μg/mL kanamycin as described herein. Selected transformants are grown overnight with shaking at 37° C. in 10 mL LB medium, and the corresponding plasmid is isolated and the desired gene inserts verified as described above. Transformants containing the desired plasmid are stored in 30% glycerol at −80° C.

Several transformants and a negative control are grown individually overnight with shaking at 37° C. in 10 mL LB medium containing 10 μg/mL kanamycin and 1 mM isopropyl-beta-thio galactopyranoside. A 2 mL sample from each medium is withdrawn after 24 hours. Each sample is centrifuged at 15000×g using a table centrifuge and the supernatant is analyzed using gas chromatography as described herein. Acetone, n-propanol and isopropanol in fermentation broths are detected by GC-FID. Samples are diluted 1+1 with 0.05% tetrahydrofuran in methanol and analyzed for isopropanol production as described above.

### Example 16

### n-Propanol Tolerance in Lactobacillus reuteri

Lactobacillus reuteri was shown to be resistant to n-propanol under the conditions described below.

To prepare the inoculum for the tank fermentation, a preculture of a strain of Lactobacillus reuteri was performed as described above. 50 mL of this culture was used to inoculate a fermentor containing 1950 mL of a medium prepared as described in the following:

Medium Composition:

Concentrated sugar cane juice (BRIX 53) adjusted to final BRIX of 5 with tap water was used as the base component. To this diluted sugar cane juice, yeast extract (Bacto) was added in the amount of 10 g/L and antifoam (Pluronic/Dowfax 63N) was added in the amount of 1 mL/L. This mixture was transferred to a labscale fermentor (3 liter vessel) and autoclaved for 30 minutes at 121-123° C. After autoclavation, temperature was adjusted to 37° C. and 80 mL (corresponding to 40 ml/L) of n-propanol was added to the tank by sterile filtration.

Following inoculation, the temperature was held at about 37° C. and the pH maintained at either pH 6.5 or pH 3.8 (e.g., by the addition of 10%(w/w) NH₄OH). A small inflow (0.1 liter per minute) of N₂ ensured that the culture was anaerobic during agitation at 400 rpm. OD₆₅₀ measurements were taken throughout the fermentation to monitor cell growth.

Lactobacillus reuteri was capable of growth at both pH 6.5 and pH 3.8 in 4% n-propanol. At pH 3.8, the growth rate was somewhat delayed, but achieved the same maximum OD after about 40 hours of fermentation. A gas chromatography-mass spectrometry (GCMS) based analysis of a fermentation sample taken after 112 hours of fermentation showed that of the initial amount of n-propanol, the pH 6.5 and pH 3.8 contained 79.8% and 93.1%, respectively. It was determined that the n-propanol used for the experiment initially contained approximately 4% isopropanol in addition to 96% n-propanol.

### Example 17

### n-Propanol Produced in Wt Lactobacillus reuteri

Wild-type Lactobacillus reuteri was shown to produce n-propanol under the conditions described below.

A preculture of wt Lactobacillus reuteri for the tank fermentations was grown for two days at 37° C. in MRS-medium without aeration or shaking. A 50 mL sample of this culture was used to inoculate a fermenter containing 1950 mL of the following medium:

Medium Composition:

Concentrated sugar cane juice (BRIX 53) adjusted to final BRIX of 5 with tap water was used as the base component. To this diluted sugar cane juice, yeast extract (Bacto) was added in the amount of 10 g/L and antifoam (Pluronic/Dowfax 63N) was added in the amount of 1 mL/L. This mixture was transferred to a labscale fermenter (3 liter vessel) and autoclaved for 30 minutes at 121-123° C.

Following inoculation, the pH was kept constant at 6.5 by the addition of 10% (w/w) NH₄OH, and the temperature was kept at 37° C. The culture was kept anaerobic by a small flow of pumped N₂ (0.1 liter per minute) and the agitation rate was 400 rpm.

A gas chromatography-mass spectrometry (GCMS) based analysis of a fermentation sample taken after 48 hours of fermentation indicated that the culture contained approximately 40 μL/L n-propanol.

In another experiment performed with the same strain as above and under the same conditions but with pH being kept constant at pH 3.8 instead of pH 6.5, a sample taken after 48 hours of fermentation showed that the culture contained approximately 40 μL/L n-propanol.

### Example 18

### Production of n-Propanol from 1,2-Propanediol in Wt Lactobacillus reuteri

The conversion of 1,2-propanediol into n-propanol was demonstrated in wild type Lactobacillus reuteri.

A preculture of wt Lactobacillus reuteri for the tank fermentations was grown for two days at 37° C. in MRS-medium without aeration or shaking. A 50 mL sample of this culture was used to inoculate a fermenter containing 1950 mL of the following medium containing 1,2 propanediol:

1,2-Propanediol Medium:

Concentrated sugar cane juice (BRIX 53) adjusted to final BRIX of 5 with tap water was used as the base component. To this diluted sugar cane juice, yeast extract (Bacto) was added in the amount of 10 g/L and antifoam (Pluronic/Dowfax 63N) was added in the amount of 1 mL/L. This mixture was transferred to a labscale fermentor (3 liter vessel) and autoclaved for 30 minutes at 121-123° C. After autoclavation, the temperature was adjusted to 37° C. and 20 g (corresponding to 10 g/L) of 1,2 propanediol was added to the tank by sterile filtration.

Following inoculation, the temperature was maintained at about 37° C. with pH of approximately pH 6.5 by the addition of 10%(w/w) NH₄OH. A small inflow (0.1 liter per minute) of N₂ ensured that the culture was anaerobic during agitation at 400 rpm.

Fermentation samples were taken and analyzed by gas chromatography-mass spectrometry (GCMS) based on the following procedure:

Sample Preparation:

The fermentation samples were centrifuged and the supernatants were used for the analysis of volatile components. Equal volumes of sample supernatant and an internal standard consisting of 5 mL/L 2-methyl-1-propanol in 0.25 M HCl (aqueous) were mixed, producing the solution that was applied to the GCMS.

GC Settings:

0.1 μL of the above solution was injected on to the GCMS (Agilent 7890A, Agilent Technologies, Santa Clara, Calif., USA) using a split ratio of 200:1. The inlet temperature was 200° C. and the column was a DB200 column (length 30 m/inner diameter 250 μm/film thickness 0.5 μm) purchased from Agilent Technologies. The carrier gas was helium and the initial flow rate was 0.489 mL/min (held constant for 1 minute) ramped up to 0.90 mL/min (ramp rate: 0.2907 mL/min/min) and further up to a final flow rate of 1.0 mL/min (ramp rate: 0.6 mL/min/min), which was held for 1 minute. The temperature program for the GCMS was as follows: Initial temperature was 50° C. (kept constant for 2.3 minutes), ramped up to 175° C. (ramp: 120° C./min) held for 2 minutes. In some instances the temperature differed from the above temperature program due to the steep temperature gradient and capabilities of the GCMS.

MS Settings:

MS analysis was performed based on the selected ion monitoring (SIM) mode. The ions (m/z) used for quantifications were as follows (retention times stated in parentheses): 2-methyl-1-propanol (internal standard), 43 (2.89 minutes); isopropanol, 43 (2.43 minutes); n-propanol, 31 (2.62 minutes); propanal, 58 (2.66 minutes); propionic acid, 74 (3.27 minutes). While the retention times occasionally overlapped, the ions were chosen so that the interference of co-eluting compounds was minimized or eliminated entirely in the actual quantification.

A GCMS based analysis of a fermentation sample taken after 112 hours of fermentation showed almost quantitative conversion to n-propanol and propionic acid (7.30 mL/L n-propanol, 1.55 mL/L propionic acid and 60 μL/L propanal). A sample taken prior to inoculation, showed no signs of propionic acid and propanal, and an n-propanol concentration of 50 μL/L.

In a control experiment performed under the same conditions but without 1,2-propandiol addition, the GCMS analysis showed that there was no propionic acid and no propanal in the sample taken after 112 hours, and that n-propanol concentration was 20 μL/L. In a sample taken after 23 hours of cultivation, the GCMS analysis showed that there were 40 μL/L n-propanol in the culture and no propionic acid or propanal. The sample taken prior to inoculation was negative for both n-propanol, propanal and propionic acid.

### Example 19

### Preparation of n-Propanol Producing Bacteria and Production of n-Propanol in a Bacterial Host (Prophetic)

Each selected n-propanol pathway gene encoding a lactate dehydrogenase, lactaldehyde dehydrogenase, lactaldehyde reductase, propanediol dehydratase, or n-propanol dehydrogenase described (e.g., SEQ ID NO: 1, 3, 5, 7, 9, 11, 13, 15, 17, 19, 21, 23, 25, 27, 29, 31, 33, 35, 53, 55, 57, 59, or 61) is individually codon-optimized for expression an E. coli host or a Lactobacillus host (e.g., Lactobacillus reuteri) and cloned in a similar manner to the techniques described in Examples 1-6. Desired combinations of the resulting vectors are then digested and cloned into an operon with pTRGU88, followed by transformation into E. coli or Lactobacillus sp. and colony selection in a similar manner to the techniques described in Examples 8-13. The colonies are then incubated, harvested by centrifugation, and plasmids isolated, digested and verified, as described above.

For fermentation, the resulting transformed hosts and Trc99A (negative control) are individually grown overnight with shaking following techniques similar to those in Examples 14-18 (e.g., 250 rpm in 10 mL LB medium containing 100 μg/mL ampicillin and 1 mM isopropyl-beta-thio galactopyranoside (IPTG)). In additional experiments, the pH of the medium adjusted to various acidity, such as 6.5 or 3.8 (e.g., by the addition of 10% (w/w) NH₄OH) as described in Example 16.

Samples of each fermentation run are withdrawn after various incubation times (e.g., 72 hours, 100 hours), diluted, and analyzed for the presence of n-propanol, isopropanol, propanal, propionic acid using GC-FID using the parameters described in Example 14, or gas chromatography-mass spectrometry (GCMS) as described in Example 18.

The expression of the n-propanol pathway genes during fermentations is corroborated using methods well known in the art for determining expression, including for example, Northern blots, PCR amplification of mRNA, immunoblotting, and the like. Enzymatic activities of the expressed polynucleotides are confirmed using assays specific for the individual polypeptides as described herein. Strains having a functional n-propanol pathway, but with rate-limiting expression of any heterologous polynucleotide are further augmented by optimization of the pathway, e.g., by introduction of additional gene copies and/or by alternative promoters known in the art.

Experimental results demonstrating n-propanol production from 1,2-propanediol in wt Lactobacillus reuteri (Example 18) suggest that, in some instances, the heterologous expression of every gene shown in the n-propanol pathway of FIG. 1 may not be required for n-propanol production. For example, heterologous expression of some endogenous genes, e.g., genes converting lactate to 1,2 propanediol via lactaldehyde in L. buchneri may not be required. Accordingly, minimized pathways containing fewer heterologous genes (e.g., only a heterologous polynucleotide encoding a lactate dehydrogenase, a heterologous polynucleotide encoding a lactaldehyde dehydrogenase, a heterologous polynucleotide encoding a lactaldehyde reductase, or combinations thereof for L. reuteri) are designed, transformed, fermented, and analyzed for n-propanol production as described above.

### Example 20

### Preparation of n-Propanol/Isopropanol Coproducing Bacteria and Coproduction of n-Propanol/Isopropanol in a Bacterial Host (Prophetic)

An expression vector containing the n-propanol pathway genes above in Example 19 and an expression vector containing the isopropanol pathway genes above (e.g., as described in Example 1-14) are simultaneously transformed into an E. coli host or a Lactobacillus host (e.g., Lactobacillus reuteri) via electroporation followed by colony selection as described above. The colonies are then incubated, harvested by centrifugation, and plasmids isolated, digested and verified, as described above.

For fermentation, the resulting transformed hosts and negative controls are individually grown overnight with shaking following techniques similar to those in Examples 14-18 (e.g., 250 rpm in 10 mL LB medium containing 100 μg/mL ampicillin and 1 mM isopropyl-beta-thio galactopyranoside (IPTG)). In additional experiments, the pH of the medium adjusted to various acidity, such as 6.5 or 3.8 (e.g., by the addition of 10% (w/w) NH₄OH) as described in Example 16.

Samples of each fermentation run are withdrawn after various incubation times (e.g., 72 hours, 100 hours), diluted, and analyzed for the presence of n-propanol, isopropanol, propanal, propionic acid using GC-FID using the parameters described in Example 14, or gas chromatography-mass spectrometry (GCMS) as described in Example 18.

The expression of the n-propanol pathway genes and isopropanol pathway genes during fermentations is corroborated using methods well known in the art for determining expression, and enzymatic activities of the expressed polynucleotides are confirmed using assays specific for the individual polypeptides as described herein. Strains having a functional n-propanol and isopropanol pathways, but with rate-limiting expression of any heterologous polynucleotide are further augmented by optimization of the pathway, e.g., by introduction of additional gene copies and/or by alternative promoters known in the art.

### Example 21

### Preparation of n-Propanol Producing Yeast and Production of n-Propanol in a Yeast Host (Prophetic)

Expression vectors containing the n-propanol pathway polynucleotides encoding a lactatate dehydrogenase, lactaldehyde dehydrogenase, lactaldehyde reductase, propanediol dehydratase, or n-propanol dehydrogenase described (e.g., SEQ IDs: SEQ ID NO: 1, 3, 5, 7, 9, 11, 13, 15, 17, 19, 21, 23, 25, 27, 29, 31, 33, 35, 53, 55, 57, 59, or 61) are synthesized using standard molecular biology protocols (see, e.g., Methods in Enzymology, Volume 194, Guide to Yeast Genetics and Molecular and Cell Biology (Part A, 2004, Christin Guthrie and Gerald R. Fink (Eds.), Elsevier Academic Press, San Diego, Calif.)). Each selected non-native gene is individually codon-optimized for improved expression in the selected yeast host cell and designed to contain one or more upstream promoter, terminator, and/or selection marker sequences, as described herein. Selected gene sequences native to the yeast host cell (e.g., a native lactatate dehydrogenase, lactaldehyde dehydrogenase, lactaldehyde reductase, propanediol dehydratase, or n-propanol dehydrogenase) may be designed for improved expression by incorporating one or more alternative control sequences (e.g., a suitable promoter describe herein) into the an expression vector containing the desired native gene, as known in the art.

Desired combinations of the expression vectors above (e.g., one, two, or several different pathway genes described herein) are then inserted into target insertion sites of the yeast host cell genomic sequence using standard molecular biology transformation protocols (see, e.g., Methods in Yeast Genetics (1997 edition), Adams, Gottschling, Kaiser, and Stems, Cold Spring Harbor Press (1998)). A target insertion site may be any suitable site, such as a site having minimal or no adverse effect on propanol production, or at a site intended for gene disruption that may improve propanol production (such as the loci of an endogenous polynucleotide encoding a pyruvate decarboxylase (PDC), a glycerol dehydrogenase, a dihydroxyacetone phosphate phosphatase, a glycerol-3-phosphatase (GPP), a glycerol-3-phosphate dehydrogenase (GPD), an L- or D-lactate:ferricytochrome c oxidoreductase, or an endogenous polynucleotide that encodes a polypeptide that catalyzes the conversion of xylose to xylitol).

Transformants are then plated on media suitable for selected host and the selection marker(s) used. Single colonies are then replated, and the genomic DNA is isolated, as known in the art. Integration of each gene is then confirmed by PCR using appropriate primer sequences.

The transformants and negative controls are then cultivated in separate baffled shake flasks containing suitable media described herein (e.g., containing nitrogen sources, vitamins, mineral salts, and/or metallic cofactors, supplemented with glucose) at pH, temperature, and agitation levels suitable for the selected yeast host cell based techniques known in the art and the parameters described herein.

Samples of each fermentation run are withdrawn after various incubation times (e.g., 72 hours, 100 hours), diluted, and analyzed for the presence of n-propanol, isopropanol, propanal, ethanol, and propanoic acid using gas chromatography-mass spectrometry (GCMS), e.g., based on the parameters described in Example 18.

The expression of the n-propanol pathway genes during fermentations is corroborated using methods well known in the art for determining expression, including for example, Northern blots, PCR amplification of mRNA, immunoblotting, and the like. Enzymatic activities of the expressed polynucleotides are confirmed using assays specific for the individual polypeptides as described herein. Strains having a functional n-propanol pathway, but with rate-limiting expression of any heterologous polynucleotide are further augmented by optimization of the pathway, e.g., by introduction of additional gene copies and/or by alternative promoters known in the art or described herein.

As one in the art will appreciate, in some instances (e.g., depending on the selection of host) the heterologous expression of every gene shown in the n-propanol pathway of FIG. 1 may not be required for n-propanol production given that a host cell may have endogenous enzymatic activity from one or more pathway genes. Accordingly, minimized pathways containing fewer heterolougous genes, (e.g., a heterologous polynucleotide encoding a lactate dehydrogenase, a heterologous polynucleotide encoding lactaldehyde dehydrogenase, a heterologous polynucleotide encoding lactaldehyde reductase, or combinations thereof) are constructed as described above to test for n-propanol production.

### Example 22

### Preparation of n-Propanol/Isopropanol Coproducing Yeast and Coproduction of n-Propanol/Isopropanol in a Yeat Host (Prophetic)

For the n-propanol pathway, expression vectors containing polynucleotides encoding a lactatate dehydrogenase, lactaldehyde dehydrogenase, lactaldehyde reductase, propanediol dehydratase, or n-propanol dehydrogenase (e.g., SEQ ID NO: 1, 3, 5, 7, 9, 11, 13, 15, 17, 19, 21, 23, 25, 27, 29, 31, 33, 35, 53, 55, 57, 59, or 61) are designed with suitable control sequences, codon-optimized (as appropriate for non-native sequences), and synthesized as described above in Example 21. The desired combinations of the expression vectors (e.g., one, two, or several different n-propanol pathway genes described herein) are inserted into selected target insertion sites of the yeast host cell genome (supra) using standard molecular biology transformation protocols as described above.

For the isopropanol pathway, expression vectors containing polynucleotides encoding a thiolase, CoA-transferase, acetoacetate decarboxylase, or isopropanol dehydrogenase (e.g., SEQ ID NO: 37, 39, 41, 43, 45, 47, 49, or 51) are designed with suitable control sequences, codon-optimized (as appropriate for non-native sequences), and synthesized as described above. The desired combinations of the expression vectors (e.g., one, two, or several different isopropanol pathway genes described herein) are inserted into target insertion sites of the yeast host cell genome using standard molecular biology transformation protocols as described above.

Transformants are then plated on media suitable for selected host and the selection marker(s) used for both pathways. Single colonies are then replated, and the genomic DNA is isolated. Integration of each gene is then confirmed by PCR using appropriate primer sequences.

The transformants and negative controls are then cultivated in separate baffled shake flasks containing suitable media described herein (e.g., containing nitrogen sources, vitamins, mineral salts, and/or metallic cofactors, supplemented with glucose) at pH, temperature, and agitation levels suitable for the selected yeast host cell based techniques known in the art and the parameters described herein.

Samples of each fermentation run are withdrawn after various incubation times (e.g., 72 hours, 100 hours), diluted, and analyzed for the presence of n-propanol, isopropanol, propanal, ethanol, and propanoic acid using gas chromatography-mass spectrometry (GCMS), e.g., based on the parameters described in Example 18.

The expression of the n-propanol pathway genes and isopropanol pathway genes during fermentations is corroborated using methods well known in the art for determining expression, and enzymatic activities of the expressed polynucleotides are confirmed using assays specific for the individual polypeptides as described herein. Strains having a functional n-propanol and isopropanol pathways, but with rate-limiting expression of any heterologous polynucleotide are further augmented by optimization of the pathway, e.g., by introduction of additional gene copies and/or by alternative promoters known in the art.

### Example 23

### Production of n-Propanol from Lactaldehyde in Lactobacillus reuteri

The conversion of lactaldehyde into n-propanol was demonstrated in Lactobacillus reuteri strain SJ 11294.

Strain SJ11294 was inoculated in 10 ml MRS medium and incubated anaerobically overnight at 37° C. Two×0.95 ml of culture was then harvested in 2 ml Eppendorf tubes and washed with MRS medium pH adjusted to 4.0 with DL-lactic acid. After wash and removal of the supernatant, 2×0.95 ml of fresh MRS medium pH adjusted to 4.0 with lactic acid was added. The cultures were incubated at 37° C. for 2 hours, wherein 0.05 ml of a 2.442% (w/v) lactaldehyde solution was added to one of the cultures, while no addition was made to the other. The cultures were incubated overnight at 37° C., followed by centrifugation and supernatant analysis for n-propanol content (Table 3).

Upon addition of lactaldehyde to the growth medium it was found that lactaldehyde was converted to n-propanol. A C-recovery for conversion of lactaldehyde to 1-propanol was corrected for background production of n-propanol and estimated to approximately 70%.

TABLE 3

| | | 1-propanol | C-recovery |
|---|---|---|---|
| Strain | Medium | (g/L) | (1-propanol/lactaldehyde) |
| L. reuteri SJ11294 | — | 0.03 | |
| L. reuteri SJ11294 | 1.221 g/L of | 0.72 | 0.70 |
| | lactaldehyde | | |
| BLANK | — | nd | |
| BLANK | 1.221 g/L of | nd | |
| | lactaldehyde | | |

### Example 24

### Construction of a Lactobacillus reuteri Strain with Disruption to adhE and pduP

To improve the redox potential for conversion of lactate to lactaldehyde, a disruption was made to the coding sequence of the adhE gene (SEQ ID NO: 65) which encodes the bifunctional alcohol dehydrogenase of SEQ ID NO: 66. This alcohol dehydrogenase consumes the needed NADH in the central L. reuteri energy metabolism through the conversion of acetyl-CoA to ethanol. Additionally, to avoid metabolism of the downstream metabolite propionaldehyde to propionyl-CoA, a disruption was made to the coding sequence of the pduP gene (SEQ ID NO: 67) which encodes the propionaldehyde dehydrogenase of SEQ ID NO: 68.

A mutant of L. reuteri JCM1112 in which both adhE and pduP were disrupted, resulting in strain L. reuteri TRGU1013, was constructed as described below.

Introduction of pJP042 into host strain L. reuteri JCM1112 (O4ZXV) and isolation of strain TRGU768

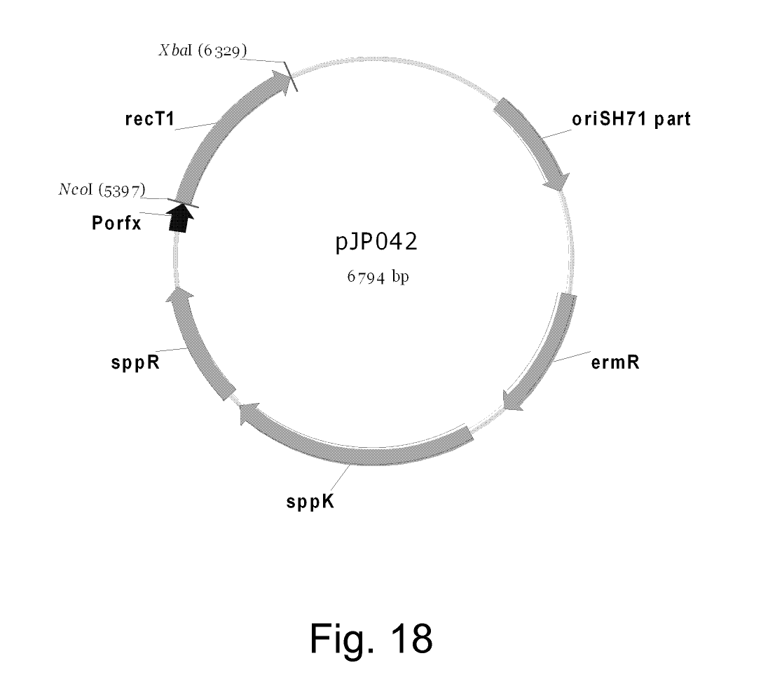

MRS medium containing 5 μg/ml erythromycin was inoculated with a L. reuteri MM4 strain harboring pJP042 (Pijkeren and Britton Nuc. Acids Res. 2012, 1-13; FIG. 18) and incubated overnight at 37° C. The strain was subcultured in 10 ml MRS containing 5 μg/ml erythromycin to OD₆₀₀=0.1. The culture was incubated at 37° C. for approximately 4 hours to OD₆₀₀=0.8 and centrifuged at 8000×g for 5 minutes. The supernatant was discarded and the cells were resuspended in 10 ml SET buffer (0.1 M NaCl, 1 mM EDTA, 10 mM Tris-Cl). The suspension was centrifuged at 8000×g for 5 minutes and the supernatant was discarded. The cells were then resuspended in 1 ml lysis buffer (6.7% saccharose, 50 mM Tris-Cl pH 8, 0.1 mM EDTA). Lysozyme was added to 10 mg/ml and the mixture was incubated at 37° C. for 1 hour. The lysate was then centrifuged at 8000×g for 5 minutes. The plasmid pJP042 DNA was isolated from the supernatant using a PureYield™ MiniPrep kit (Promega, USA) following the directions of the manufacturer.

JCM1112 cells were made competent from an overnight culture in MRS containing 5 μg/ml erythromycin by subculturing in 40 ml MRS containing 5 μg/ml erythromycin to OD=0.1 and harvested at OD=0.8. The cells were kept on ice and washed carefully twice with 40 ml ice cold Wash Buffer (0.5M sucrose, 10% (V/V) glycerol), and resuspended in 400 μl Wash Buffer.

5 μl of isolated pJP042 (supra) was added to 100 μl freshly prepared competent cells (supra) and electroporated in a BioRad Gene Pulser™ with a setting of 2.5 kV, 25 microFarad and 400 Ohms. To this was added 1 ml MRS medium and the cells incubated at 37° C. for 3 hours. The electroporated cells were incubated anaerobically overnight at 37° C. on MRS agar plates (MRS medium containing 15 g/I Bacto Agar) containing 5 μg/ml erythromycin for selection of pJP042 transformants. Erythromycin resistant colonies were checked for presence of pJP042 with colony PCR using primers flanking the recT1. Out of 11 transformants, 2 were isolated and confirmed to harbor pJP042. One of these strains was stored as TRGU768 in 10% glycerol at −80° C.

Disruption of adhE Via Homologous Recombination and Isolation of Strain TRGU980

To disrupt the adhE gene by recombineering, the four oligonucleotides below were designed using PyRec 3.1 (obtained from Robert Britton, Microbial Genomics Laboratory, Michigan State University, Mich., USA).

o524:
(SEQ ID NO: 69)
5′-CAAGAAACAA GTTGAAAAGA AAGAATTAAC TGCTGAAGAA
AAGCTTTAAA ACGCCCAAAA GCTAGTTGAC GATTTAATGA
CTAAGAGTCA-3′
o525:
(SEQ ID NO: 70)
5′-AGGGTGTTGGAGTAATGCGGT-3′
o526:
(SEQ ID NO: 71)
5′-AGAAAGAATTAACTGCTGAAGAAAAGCTTT-3′
o527:
(SEQ ID NO: 72)
5′-TGAATGATAGTGATTATGACGTTAAAGATC-3′

The four oligonucleotides were designed to construct and screen for mutants with an in-frame stop codon and a HindIII restriction site. Sequence o524 was used for the recombineering and incorporation of the nucleotides GCTTT, which in the complementary direction implements a stop codon in the reading frame and thus results in disruption of gene translation. Sequences o525, o526, and o527 were used in a PCR screen of all colonies screened. A 578 bp amplicon indicates that the mutations had been incorporated, whereas a single 1031 bp amplicon indicates that the o526 sequence did not anneal due to the mismatch between the oligo and the wild type sequence.

An overnight culture of TRGU768 was subcultured in 40 ml MRS medium containing 5 μg/ml erythromycin to OD₆₀₀ 0.1. After approximately 2 hours incubation at 37° C., OD₆₀₀ reached approximately 0.55 and recT1 expression was induced by addition of induction peptide (8 μl; 50 μg/ml) MAGNSSNFIHKIKQIFTHR (SEQ ID NO: 73). The incubation at 37° C. was prolonged for 20 minutes. Competent cells were then prepared by centrifugation and washing of the cells twice in 40 ml ice-cold Wash-Buffer (0.5M sucrose, 10% (v/v) glycerol). Finally the cells were resuspended in 800 ul Wash Buffer. 100 μl of the resuspended cells was used for each transformation. The cells were then transformed by electroporation with 5 μl o524 (20 μg/μl) as described in the procedure above. After two hours incubation in 1 ml MRS medium at 37° C. the cells were incubated anaerobically overnight on MRS agar plates.

288 colonies were screened with PCR using o525, o526, and o527. All PCR reactions resulted in a single 1031 bp amplicon, indicating that the cells were not mutated. After an additional 1 day of anaerobic incubation of the MRS agar plates with colonies, 7 additional colonies had appeared. These colonies were tested with PCR and resulted in one correct adhE mutant, TRGU956, as shown by the presence of a 578 bp amplicon.

As the mutagenic oligo o524 in the recombineering event is incorporated into one of the DNA strings of the chromosomal double helix, the colony identified could be a mixed genotype consisting of mutants and wildtype Lb. reuteri cells. Hence, 25 μl of an overnight culture of TRGU956 was streaked onto MRS agar plates to obtain single colonies. After overnight incubation, 16 colonies were tested by PCR with o525, o526, and o527 and all resulted in 1031 bp band but no 578 bp band was observed. Hence, the overnight culture appeared to consist primarily of the wildtype strain presumably because this strain grew faster than the adhE mutant. The colony on the plate from which the correct mutant was isolated was streaked out on an MRS agar plate to obtain single colonies. 96 colonies were then tested with colony PCR using o525, o526 and o527. 16 of these 96 colonies resulted in two amplicons of 578 bp and 1031 bp as estimated from agarose gel electrophoresis.

The 16 adhE mutants above were tested for presence of pJP042 by subculturing from an overnight culture without erythromycin into MRS containing 10 μg/ml erythromycin. One of the colonies grew overnight indicating presence of the erm^{R} gene and hence plasmid pJP042. This strain was designated TRGU980 and stored in 10% glycerol at −80° C. DNA sequencing showed that o524 sequence was not incorporated as expected but had implemented a short deletion internally in adhE. This internal gene deletion disrupted the reading frame and thus the adhE knock-out mutant had been established.

Disruption of pduP Via Homologous Recombination and Isolation of Strain TRGU1013

To disrupt the pduP gene by recombineering, the four oligonucleotides below were designed using PyRec 3.1 (obtained from Robert Britton, Microbial Genomics Laboratory, Michigan State University, Mich., USA).

o532:
(SEQ ID NO: 74)
5′-GTAGTAGCTG CAACGTTTGC ACTTGAAGAG CTGGCATTAT
CCTAAGCTTC GGCAAGAATT TTGCGTACAG CACTTTCAAT
ATCATTAATC-3′
o533:
(SEQ ID NO: 75)
5′-ACAACTAAATTATGAAGGCCTGTTGC-3′
o534:
(SEQ ID NO: 76)
5′-CGCAAAATTCTTGCCGAAGCTTAG-3′
o535:
(SEQ ID NO: 77)
5′-ATAATGCTTCTAAAAATCTATTTGATCGGC-3′

The four oligonucleotides were designed to construct and screen for mutants with an in-frame stop codon and a HindIII restriction site. Sequence o532 was used for the recombineering and incorporation of the nucleotides CTAAG, which in the complementary direction implements a stop codon in the reading frame and thus results in disruption of gene translation. Sequences o533, o534, and o535 were used in a PCR screen of all colonies screened. A 566 bp amplicon indicates that the mutations had been incorporated, whereas a single 1026 bp amplicon indicates that the o534 primer did not anneal due to the mismatch between oligo and the wild type sequence.

An overnight culture of TRGU980 (supra) was subcultured in 40 ml MRS medium containing 5 μg/ml erythromycin to OD₆₀₀ 0.1. After approximately 2 hours incubation at 37° C., OD₆₀₀ reached approximately 0.55 and recT1 expression was induced by addition of induction peptide (8 μl; 50 μg/ml) MAGNSSNFIHKIKQIFTHR (SEQ ID NO: 73). The incubation at 37° C. was prolonged for 20 minutes. Competent cells were then prepared by centrifugation and washing of the cells twice in 40 ml ice-cold Wash-Buffer (0.5M sucrose, 10% (v/v) glycerol). Finally the cells were resuspended in 800 ul Wash Buffer. 100 μl of the resuspended cells was used for each transformation. The cells were then transformed by electroporation with 5 μl o5324 (20 μg/μl) as described in the procedure above. After two hours incubation in 1 ml MRS medium at 37° C. the cells were incubated anaerobically overnight on MRS agar plates.

96 colonies were screened with PCR using o533, o534, and o535. Four colonies resulted in two bands each of correct sizes as estimated by agarose gel electrophoresis. Overnight cultures were streaked to single colonies on MRS agar plates and incubated anaerobically overnight at 37° C. 32 colonies from each were analyzed with colony PCR using o533 and o535, all of which resulted in the correct 1026 bp amplification products.

The amplification products were digested with HindIII and analyzed on agarose gel electrophoresis. The analyses showed that five amplification products were fully digested by HindIII indicating that the colonies were pduP-disrupted mutants with pure genotypes. Two of these mutants were inoculated in parallel in MRS medium and MRS medium containing 5 μg/ml erythromycin. After overnight incubation no growth was detected of either mutant strain in MRS medium containing erythromycin indicating that the mutants had lost the plasmid pJP042. One of these mutants, designated TRGU1013, was stored in 10% glycerol at −80° C. DNA sequencing of a PCR amplification product using o533 and o535 indicated that the correct mutation was incorporated in pduP, thereby verifying that TRGU1013 was an adhE pduP double mutant.

### Example 25

### Cloning of a L. brevis Lactaldehyde Dehydrogenase Gene and Construction of Vector pBKQ175

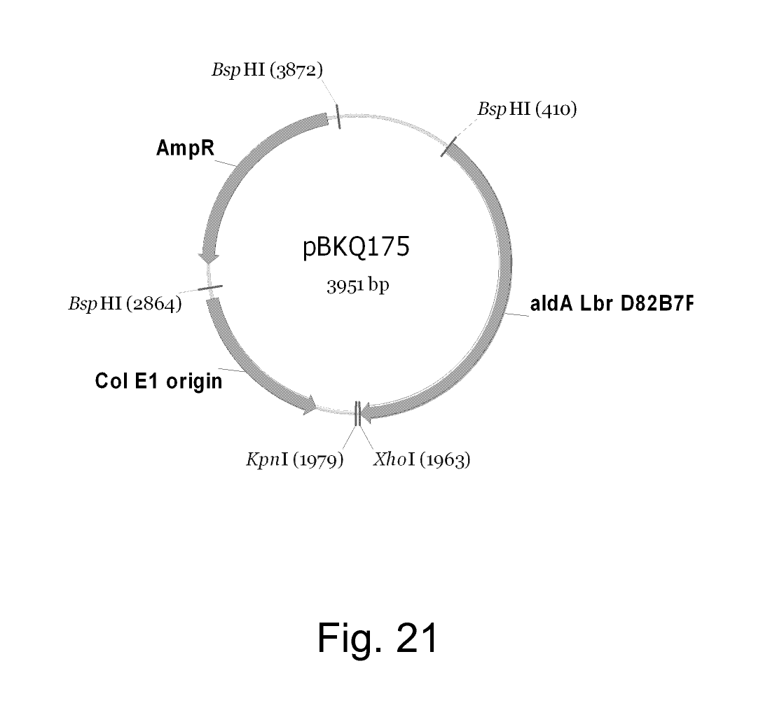

The 1548 bp coding sequence (CDS) of a lactaldehyde dehydrogenase gene aldA identified in L. brevis was synthetically constructed into pBKQ175. The DNA fragment containing the CDS was designed with a ribosomal binding site (RBS, sequence 5′-AAGGAGATTTTAGTC-3′) immediately prior to the start codon. The resulting sequence was then submitted to and synthesized by Geneart AG (Regenburg, Germany) and delivered in the pMA backbone vector containing the β-lactamase encoding gene blaTEM-1. When synthesized, the CDS and RBS fragment was flanked by restriction sites to facilitate subsequent cloning steps. The entire synthetic fragment cloned into the pMA vector was NcoI-EcoRI-BamHI-RBS-BspHI-CDS-STOP-XhoI-EcoRI-KpnI, resulting in pBKQ175 (FIG. 21).

The wild-type nucleotide sequence (WT) and deduced amino acid sequence of the L. brevis lactaldehyde dehydrogenase gene correspond to SEQ ID NO: 5 and 6, respectively. The coding sequence is 1551 bp including the stop codon and the encoded predicted protein is 516 amino acids. Using the SignalP program (Nielsen et al., 1997, Protein Engineering 10: 1-6), no signal peptide in the sequence was predicted. Based on this program, the predicted mature protein contains 516 amino acids with a predicted molecular mass of 56.4 kDa and an isoelectric pH of 5.11.

### Example 26

### Cloning of an E. coli Lactaldehyde Reductase Gene and Construction of Vector pBKQ186

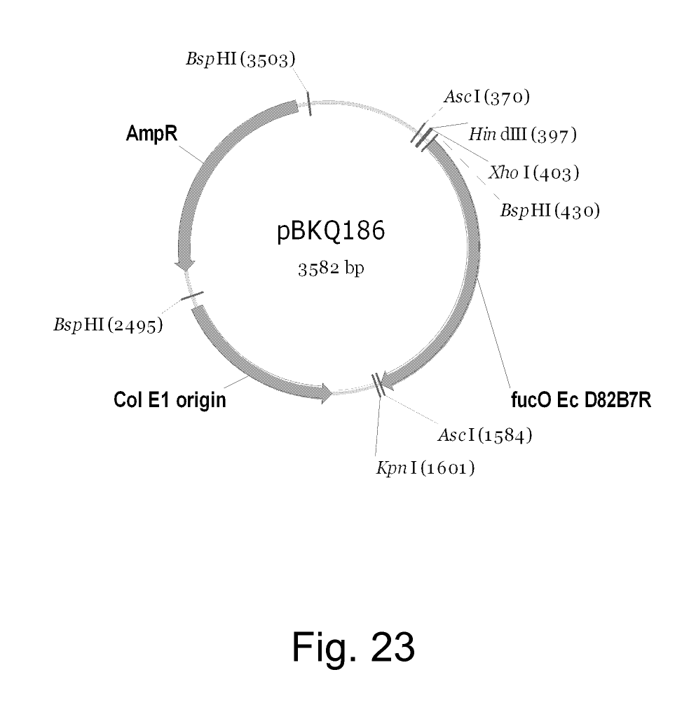

The 1149 bp coding sequence (CDS) of a lactaldehyde reductase gene fucO identified in E. coli was synthetically constructed into pBKQ186. The DNA fragment containing the CDS was designed with a ribosomal binding site (RBS, sequence 5′-AAGGAGATTTTAGTC-3′) immediately prior to the start codon. The resulting sequence was then submitted to and synthesized by Geneart AG (Regenburg, Germany) and delivered in the pMA backbone vector containing the β-lactamase encoding gene blaTEM-1. When synthesized, the CDS and RBS fragment was flanked by restriction sites to facilitate subsequent cloning steps. The entire synthetic fragment cloned into the pMA vector was NcoI-EcoRI-HindIII-XhoI-RBS-BspHI-CDS-STOP-AscI-EcoRI-KpnI, resulting in pBKQ186 (FIG. 23).

The wild-type nucleotide sequence (WT) and deduced amino acid sequence of the L. brevis lactaldehyde dehydrogenase gene correspond to SEQ ID NO: 9 and 10, respectively. The coding sequence is 1152 bp including the stop codon and the encoded predicted protein is 383 amino acids. Using the SignalP program (Nielsen et al., 1997, Protein Engineering 10: 1-6), no signal peptide in the sequence was predicted. Based on this program, the predicted mature protein contains 383 amino acids with a predicted molecular mass of 40.6 kDa and an isoelectric pH of 4.91.

### Example 27

### Construction and Transformation of pBKQ405 Expressing a L. brevis Lactaldehyde Dehydrogenase Gene and an E. coli Lactaldehyde Reductase Gene from a Synthetic Promoter

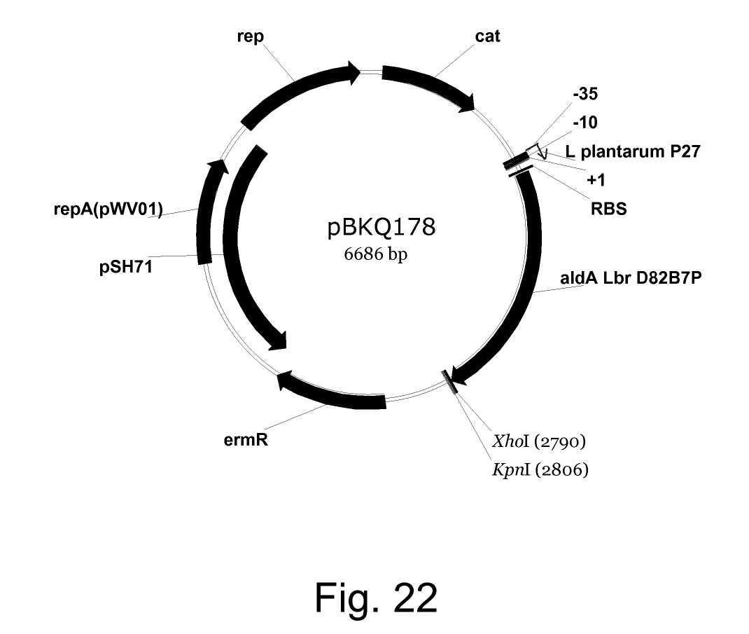

Plasmid pSJ10603 (see WO2012/058603) was digested with NcoI and KpnI, and a fragment of 5117 bp purified from 1% agarose gel using gel electrophoresis. Plasmid pBKQ175 (supra) was digested with BspHI and KpnI, and a 1569 bp fragment encoding the L. brevis lactaldehyde dehydrogenase from gene aldA was purified from 1% agarose gel using gel electrophoresis. The purified fragments were mixed, ligated, and the ligation mixture used to transform E. coli TG1 chemical competent cells, selecting for erythromycin resistance (200 microgram/ml) on LB plates at 37° C. A transformant containing the synthetic gene coding for the L. brevis lactaldehyde dehydrogenase driven by the P27 promoter was confirmed by restriction analysis and sequencing. The transformant was stored as E. coli BKQ178 and the plasmid designated as pBKQ178 (FIG. 22).

Promoter P27: 5′-CGGGGTTTAGTTGTTGACAGGGAGGCTTCGTTGTGATAAGATGGTAG-3′ (SEQ ID NO: 80; also descibed in Rud et al. Microbiology 2006, 152, 1011-1019)

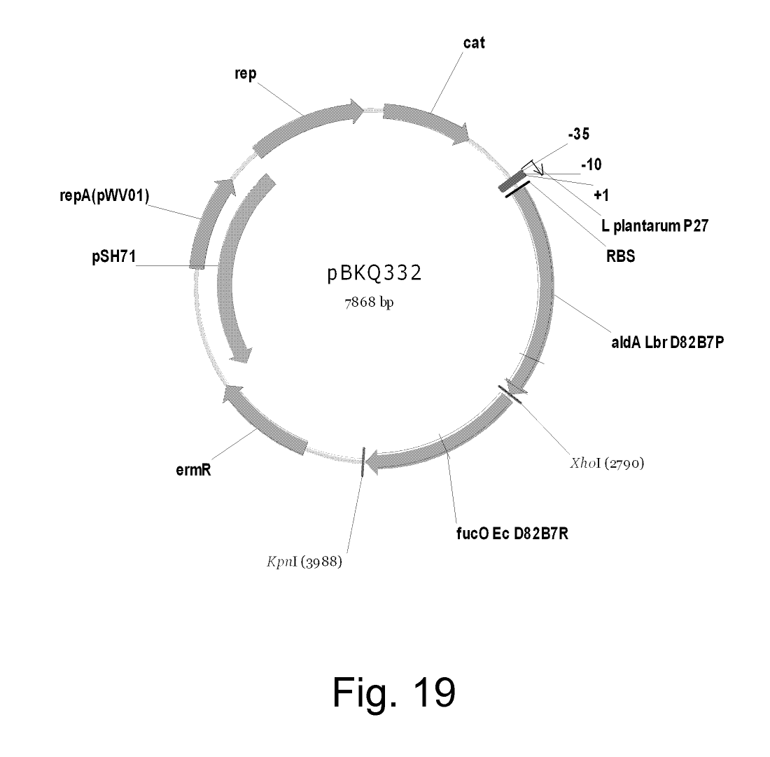

Plasmid pBKQ186 (supra) was digested with XhoI and KpnI, and the 1198 bp fragment encoding the E. coli lactaldehyde reductase from gene fucO was purified using gel electrophoresis. Plasmid pBKQ178 was digested with XhoI and KpnI and the 6670 bp fragment encoding pSH71 replication origin, erythromycin resistance gene, chloramphenicol resistance gene, and the L. brevis lactaldehyde dehydrogenase driven by the P27 promoter was purified using gel electrophoresis. The purified fragments were mixed, ligated, and the ligation mixture used to transform E. coli TG1 chemical competent cells, selecting for erythromycin resistance (200 microgram/ml) on LB plates at 37° C. A transformant containing both the synthetic gene coding for the L. brevis lactaldehyde dehydrogenase and the synthetic gene coding for the E. coli lactaldehyde reductase was confirmed by restriction analysis and sequencing. The transformant was stored as E. coli BKQ332 and the plasmid designated as pBKQ332 (FIG. 19).

Substitution of fucO in pBKQ332 with a version encoding the P11 promoter upstream to fucO was performed as follows: Plasmid pBKQ332 was digested with XhoI and KpnI resulting in a 6670 bp fragment, which was purified from 1% agarose gel using gel electrophoresis. A PCR fragment (1.3 kb) of fucO was obtained by amplification of primer pr037 and primer pr034 using plasmid pBKQ186 as template, thereby introducing the P11 promoter upstream to fucO.

P11 Promoter:
(SEQ ID NO: 81; descibed in Rud et al. Microbiology 2006, 152, 1011-1019)
5′-AGCGCTATAGTTGTTGACAGAATGGACATACTATGATATATTGTTGC-3′
Primer pr037:
(SEQ ID NO: 78)
5′-GTGCGAAGCT TGTCGACTCG AGAGCGCTAT AGTTGTTGAC AGAATGGACA
TACTATGATA TATTGTTGCT ATAGCGCGAG ACTATTACAA GGAGATTTTA G-3′
Primer pr034:
(SEQ ID NO: 79)
5-GTGCGAAGCT TCCGACTGGA AAGCGGGCAG TG -3′

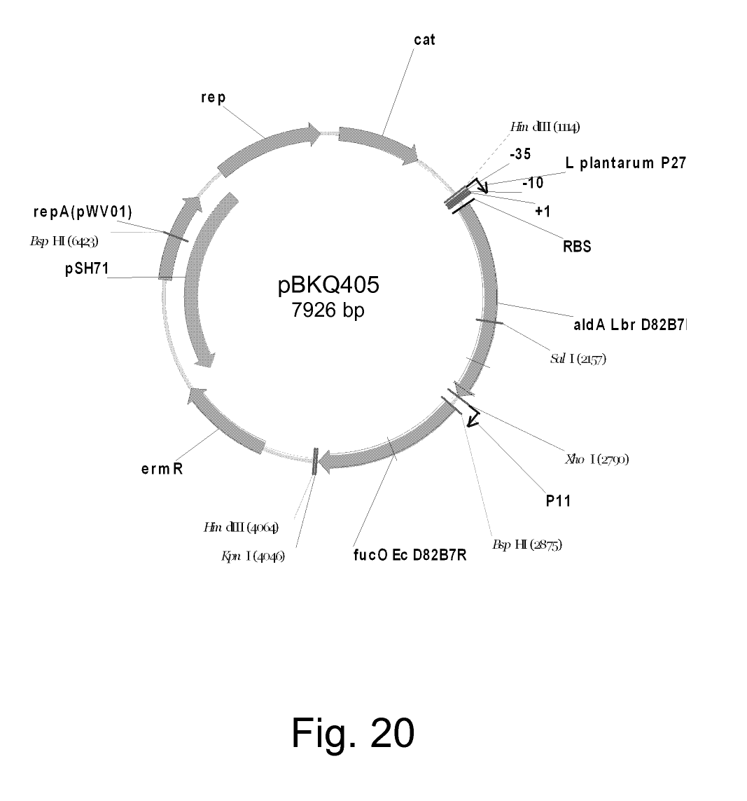

The PCR product was digested with XhoI and KpnI resulting in a 1256 bp fragment which was then purified from a 1% agarose gel using gel electrophoresis. The purified fragments of digested pBKQ332 and the digested PCR product were mixed, ligated, and the ligation mixture used to transform E. coli TG1 chemical competent cells selecting for erythromycin resistance (200 microgram/ml) on LB plates at 37° C. A transformant containing both the synthetic gene coding for the L. brevis lactaldehyde dehydrogenase and the synthetic gene coding for the E. coli lactaldehyde reductase was confirmed by restriction analysis and sequencing. The transformant was stored as E. coli BKQ405 and the plasmid designated as pBKQ405 (FIG. 20).

Plasmid pBKQ405 (supra) was introduced to the L. reuteri adhE pduP double mutant strain TRGU1013 (supra) using the electroporation procedure described above, resulting in strain BKQ498.

### Example 28

### Production of n-Propanol in Lactobacillus reuteri Containing a Lactaldehyde Dehydrogenase Gene

L. reuteri strains SJ11360, TRGU1014 and BKQ498 each were inoculated in 10 ml MRS medium (supplemented with 10 μg/ml erythromycin), followed by overnight anaerobic incubation at 37° C. 2×1 ml of cell cultures were harvested by centrifugation, and then washed in MRS-medium (pH adjusted to 4.0 with DL-lactic acid). The washed cells were inoculated into 10 ml of either: 1) MRS medium supplemented with 10 μg/ml, or 2) MRS medium supplemented with 10 μg/ml erythromycin and pH adjusted to 4.0 with DL-lactic acid. The cultures were then incubated for three days at 37° C. followed by n-propanol analysis. Results are shown in Table 4.

TABLE 4

| | | | | | 1-propanol |
|---|---|---|---|---|---|
| Strain | Background | Plasmid | n-propanol pathway genes | Medium pH | g/L |
| SJ11360 | SJ11294 (wt) | pSJ10600 | — | unchanged | 0.02 |
| | | | | 4.0 | 0.02 |
| TRGU1014 | TRGU1013 | pSJ10600 | — | unchanged | 0.03 |
| | (adhE-pduP-) | | | 4.0 | 0.02 |
| BKQ498 | TRGU1013 | pBKQ405 | P27-aldA-P11-fucO | unchanged | 0.04 |
| | (adhE-pduP-) | | | 4.0 | 0.09 |
| BLANK | — | — | — | unchanged | Nd |
| BLANK | — | — | — | 4.0 | Nd |

The presence of a lactaldehyde dehydrogenase from L. brevis encoded by aldA (SEQ ID NO: 5) and a lactaldehyde reductase from E. coli encoded by fucO (SEQ ID NO: 9) in L. reuteri BKQ498 resulted in increased level of n-propanol production (90 mg/L) compared to L. reuteri SJ11360 and TRGU1014 (20-30 mg/L) containing only an empty plasmid vector. As L. reuteri SJ11360 has the capacity to convert lactaldehyde into n-propanol without further genetic modifications (see Example 21), the observed increase in n-propanol production measured in the current experiment may be related to conversion of lactate to lactaldehyde through the lactaldehyde dehydrogenase encoded by aldA. Previous experiments have shown that the presence of aldA in wildtype strain L. reuteri SJ11294 is not sufficient to result in increased 1-propanol production in spite of a low pH (data not shown). The observed effect was found in the adhE disruption mutant L. reuteri TRGU1013 grown in pH adjusted medium (pH 4.0) with lactic acid. Thus, effective conversion of lactate to lactaldehyde in L. reuteri expressing a lactaldehyde dehydrogenase (aldA) may include a heterologous lactaldehyde dehydrogenase gene, improved redox potential (e.g., by knock-out of adhE), and lowered pH.

The present invention may be further described by the following numbered paragraphs:

[1] A recombinant host cell comprising lactate dehydrogenase activity, lactaldehyde dehydrogenase activity, lactaldehyde reductase activity, propanediol dehydratase activity, and n-propanol dehydrogenase activity, wherein the cell is capable of producing n-propanol.
[2] The recombinant host cell of paragraph [1], comprising:

one or more heterologous polynucleotides that encode a lactate dehydrogenase,

one or more heterologous polynucleotides that encode a lactaldehyde dehydrogenase,

one or more heterologous polynucleotides that encode a lactaldehyde reductase,

one or more heterologous polynucleotides that encode a propanediol dehydratase, or

one or more heterologous polynucleotides that encode an n-propanol dehydrogenase;

wherein the host cell is capable of producing a greater amount of n-propanol compared to the host cell without the heterologous polynucleotide(s) when cultivated under the same conditions.

[3] A recombinant host cell comprising:

one or more heterologous polynucleotides that encode a lactate dehydrogenase,

one or more heterologous polynucleotides that encode a lactaldehyde dehydrogenase,

one or more heterologous polynucleotides that encode a lactaldehyde reductase,

one or more heterologous polynucleotides that encode a propanediol dehydratase, or

one or more heterologous polynucleotides that encode an n-propanol dehydrogenase;

wherein the host cell is capable of producing a greater amount of n-propanol compared to the host cell without the heterologous polynucleotide(s) when cultivated under the same conditions.

[4] The recombinant host cell of any one of paragraphs [1]-[3], comprising:

one or more heterologous polynucleotides that encode a lactate dehydrogenase;

wherein the host cell is capable of producing a greater amount of n-propanol compared to the host cell without the one or more heterologous polynucleotides that encode the lactate dehydrogenase when cultivated under the same conditions.

[5] The recombinant host cell of any one of paragraphs [1]-[4], comprising:

one or more heterologous polynucleotides that encode a lactadehyde dehydrogenase;

wherein the host cell is capable of producing a greater amount of n-propanol compared to the host cell without the one or more heterologous polynucleotides that encode the lactadehyde dehydrogenase when cultivated under the same conditions.

[6] The recombinant host cell of any one of paragraphs [1]-[5], comprising:

one or more heterologous polynucleotides that encode a lactaldehyde reductase;

wherein the host cell is capable of producing a greater amount of n-propanol compared to the host cell without the one or more heterologous polynucleotides that encode the lactaldehyde reductase when cultivated under the same conditions.

[7] The recombinant host cell of any one of paragraphs [1]-[6], comprising:

one or more heterologous polynucleotides that encode a propanediol dehydratase;

wherein the host cell is capable of producing a greater amount of n-propanol compared to the host cell without the one or more heterologous polynucleotides that encode the propanediol dehydratase when cultivated under the same conditions.

[8] The recombinant host cell of any one of paragraphs [1]-[3], comprising:

one or more heterologous polynucleotides that encode a lactate dehydrogenase,

one or more heterologous polynucleotides that encode a lactaldehyde dehydrogenase,

one or more heterologous polynucleotides that encode a lactaldehyde reductase,

one or more heterologous polynucleotides that encode a propanediol dehydratase, and

one or more heterologous polynucleotides that encode n-propanol dehydrogenase;

wherein the host cell is capable of producing a greater amount of n-propanol compared to the host cell without the heterologous polynucleotides that encode the lactate dehydrogenase, lactaldehyde dehydrogenase, lactaldehyde reductase, propanediol dehydratase, and n-propanol dehydrogenase, when cultivated under the same conditions.

[9] The recombinant host cell of any one of paragraphs [2]-[8], wherein the host cell is capable of producing a greater amount of the n-propanol by at least 5%, e.g., at least 10%, at least 15%, at least 20%, at least 25%, at least 30%, at least 50%, or at least 100% compared to the host cell without the one or more heterologous polynucleotides that encodes the lactate dehydrogenase, when cultivated under the same conditions.
[10] The recombinant host cell of any one of paragraphs [2]-[9], wherein the host cell is capable of producing a greater amount of the n-propanol by at least 5%, e.g., at least 10%, at least 15%, at least 20%, at least 25%, at least 30%, at least 50%, or at least 100% compared to the host cell without the one or more heterologous polynucleotides that encodes the lactaldehyde dehydrogenase, when cultivated under the same conditions.
[11] The recombinant host cell of any one of paragraphs [2]-[10], wherein the host cell is capable of producing a greater amount of the n-propanol by at least 5%, e.g., at least 10%, at least 15%, at least 20%, at least 25%, at least 30%, at least 50%, or at least 100% compared to the host cell without the one or more heterologous polynucleotides that encodes the lactaldehyde reductase, when cultivated under the same conditions.
[12] The recombinant host cell of any one of paragraphs [2]-[11], wherein the host cell is capable of producing a greater amount of the n-propanol by at least 5%, e.g., at least 10%, at least 15%, at least 20%, at least 25%, at least 30%, at least 50%, or at least 100% compared to the host cell without the one or more heterologous polynucleotides that encodes the propanediol dehydratase, when cultivated under the same conditions.
[13] The recombinant host cell of any one of paragraphs [2]-[12], wherein the host cell is capable of producing a greater amount of the n-propanol by at least 5%, e.g., at least 10%, at least 15%, at least 20%, at least 25%, at least 30%, at least 50%, or at least 100% compared to the host cell without the one or more heterologous polynucleotides that encodes the n-propanol dehydrogenase, when cultivated under the same conditions.
[14] The recombinant host cell of any one of paragraphs [1]-[13], comprising a heterologous polynucleotide that encodes a lactate dehydrogenase, wherein the polynucleotide is selected from:

(a) a polynucleotide that encodes a lactate dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 2, 4, 34, 36, or the mature polypeptide sequence thereof;

(b) a polynucleotide that hybridizes under low stringency conditions, medium stringency conditions, medium-high stringency conditions, high stringency conditions, or very high stringency conditions with SEQ ID NO: 1, 3, 33, 35, the mature polypeptide coding sequence thereof, or the full-length complementary strand of the foregoing; and

(c) a polynucleotide that has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 1, 3, 33, 35, or the mature polypeptide coding sequence thereof.

[15] The recombinant host cell of paragraph [14], wherein the polynucleotide encodes a lactate dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 2 or the mature polypeptide sequence thereof.
[16] The recombinant host cell of paragraph [14], wherein the polynucleotide encodes a lactate dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4 or the mature polypeptide sequence thereof.
[17] The recombinant host cell of paragraph [14], wherein the polynucleotide encodes a lactate dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 34 or the mature polypeptide sequence thereof.
[18] The recombinant host cell of paragraph [14], wherein the polynucleotide encodes a lactate dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 36 or the mature polypeptide sequence thereof.
[19] The recombinant host cell of paragraph [14], wherein the polynucleotide hybridizes under low stringency conditions, medium stringency conditions, medium-high stringency conditions, high stringency conditions, or very high stringency conditions with SEQ ID NO: 1, the mature polypeptide coding sequence thereof, or the full-length complementary strand of the foregoing.
[20] The recombinant host cell of paragraph [14], wherein the polynucleotide hybridizes under low stringency conditions, medium stringency conditions, medium-high stringency conditions, high stringency conditions, or very high stringency conditions with SEQ ID NO: 3, the mature polypeptide coding sequence thereof, or the full-length complementary strand of the foregoing.
[21] The recombinant host cell of paragraph [14], wherein the polynucleotide hybridizes under low stringency conditions, medium stringency conditions, medium-high stringency conditions, high stringency conditions, or very high stringency conditions with SEQ ID NO: 33, the mature polypeptide coding sequence thereof, or the full-length complementary strand of the foregoing.
[22] The recombinant host cell of paragraph [14], wherein the polynucleotide hybridizes under low stringency conditions, medium stringency conditions, medium-high stringency conditions, high stringency conditions, or very high stringency conditions with SEQ ID NO: 35, the mature polypeptide coding sequence thereof, or the full-length complementary strand of the foregoing.
[23] The recombinant host cell of paragraph [14], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 1, or the mature polypeptide coding sequence thereof.
[24] The recombinant host cell of paragraph [14], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 3, or the mature polypeptide coding sequence thereof.
[25] The recombinant host cell of paragraph [14], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 33, or the mature polypeptide coding sequence thereof.
[26] The recombinant host cell of paragraph [14], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 35, or the mature polypeptide coding sequence thereof.
[27] The recombinant host cell of paragraph [14], wherein the polynucleotide encodes a lactate dehydrogenase variant of SEQ ID NO: 2 or the mature polypeptide sequence thereof, comprising a substitution, deletion, and/or insertion at not more than 10 positions, e.g., not more than 9, 8, 7, 6, 5, 4, 3, 2, or 1 position.
[28] The recombinant host cell of paragraph [14], wherein the polynucleotide encodes a lactate dehydrogenase that comprises or consists of SEQ ID NO: 2 or the mature polypeptide sequence thereof.
[29] The recombinant host cell of paragraph [14], wherein the polynucleotide encodes a lactate dehydrogenase that comprises or consists of SEQ ID NO: 2.
[30] The recombinant host cell of paragraph [14], wherein the polynucleotide encodes a lactate dehydrogenase variant of SEQ ID NO: 4 or the mature polypeptide sequence thereof, comprising a substitution, deletion, and/or insertion at not more than 10 positions, e.g., not more than 9, 8, 7, 6, 5, 4, 3, 2, or 1 position.
[31] The recombinant host cell of paragraph [14], wherein the polynucleotide encodes a lactate dehydrogenase that comprises or consists of SEQ ID NO: 4 or the mature polypeptide sequence thereof.
[32] The recombinant host cell of paragraph [14], wherein the polynucleotide encodes a lactate dehydrogenase that comprises or consists of SEQ ID NO: 4.
[33] The recombinant host cell of paragraph [14], wherein the polynucleotide encodes a lactate dehydrogenase variant of SEQ ID NO: 34 or the mature polypeptide sequence thereof, comprising a substitution, deletion, and/or insertion at not more than 10 positions, e.g., not more than 9, 8, 7, 6, 5, 4, 3, 2, or 1 position.
[34] The recombinant host cell of paragraph [14], wherein the polynucleotide encodes a lactate dehydrogenase that comprises or consists of SEQ ID NO: 34 or the mature polypeptide sequence thereof.
[35] The recombinant host cell of paragraph [14], wherein the polynucleotide encodes a lactate dehydrogenase that comprises or consists of SEQ ID NO: 34.
[36] The recombinant host cell of paragraph [14], wherein the polynucleotide encodes a lactate dehydrogenase variant of SEQ ID NO: 36 or the mature polypeptide sequence thereof, comprising a substitution, deletion, and/or insertion at not more than 10 positions, e.g., not more than 9, 8, 7, 6, 5, 4, 3, 2, or 1 position.
[37] The recombinant host cell of paragraph [14], wherein the polynucleotide encodes a lactate dehydrogenase that comprises or consists of SEQ ID NO: 36 or the mature polypeptide sequence thereof.
[38] The recombinant host cell of paragraph [14], wherein the polynucleotide encodes a lactate dehydrogenase that comprises or consists of SEQ ID NO: 36.
[39] The recombinant host cell of any one of paragraphs [2]-[38], wherein the heterologous polynucleotide that encodes a lactate dehydrogenase is operably linked to a promoter foreign to the polynucleotide.
[40] The recombinant host cell of any one of paragraphs [1]-[13], comprising a heterologous polynucleotide that encodes a lactaldehyde dehydrogenase, wherein the polynucleotide is selected from:

(a) a polynucleotide that encodes a lactaldehyde dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 6, 8, 26, 30, 32, or the mature polypeptide sequence thereof;

(b) a polynucleotide that hybridizes under low stringency conditions, medium stringency conditions, medium-high stringency conditions, high stringency conditions, or very high stringency conditions with SEQ ID NO: 5, 7, 25, 29, 31, the mature polypeptide coding sequence thereof, or the full-length complementary strand of the foregoing; and

(c) a polynucleotide that has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 5, 7, 25, 29, 31, or the mature polypeptide coding sequence thereof.

[41] The recombinant host cell of paragraph [40], wherein the polynucleotide encodes a lactaldehyde dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 6 or the mature polypeptide sequence thereof.
[42] The recombinant host cell of paragraph [40], wherein the polynucleotide encodes a lactaldehyde dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 8 or the mature polypeptide sequence thereof.
[43] The recombinant host cell of paragraph [40], wherein the polynucleotide encodes a lactaldehyde dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 26 or the mature polypeptide sequence thereof.
[44] The recombinant host cell of paragraph [40], wherein the polynucleotide encodes a lactaldehyde dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 30 or the mature polypeptide sequence thereof.
[45] The recombinant host cell of paragraph [40], wherein the polynucleotide encodes a lactaldehyde dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 32 or the mature polypeptide sequence thereof.
[46] The recombinant host cell of paragraph [40], wherein the polynucleotide hybridizes under low stringency conditions, medium stringency conditions, medium-high stringency conditions, high stringency conditions, or very high stringency conditions with SEQ ID NO: 5, the mature polypeptide coding sequence thereof, or the full-length complementary strand of the foregoing.
[47] The recombinant host cell of paragraph [40], wherein the polynucleotide hybridizes under low stringency conditions, medium stringency conditions, medium-high stringency conditions, high stringency conditions, or very high stringency conditions with SEQ ID NO: 7, the mature polypeptide coding sequence thereof, or the full-length complementary strand of the foregoing.
[48] The recombinant host cell of paragraph [40], wherein the polynucleotide hybridizes under low stringency conditions, medium stringency conditions, medium-high stringency conditions, high stringency conditions, or very high stringency conditions with SEQ ID NO: 25, the mature polypeptide coding sequence thereof, or the full-length complementary strand of the foregoing.
[49] The recombinant host cell of paragraph [40], wherein the polynucleotide hybridizes under low stringency conditions, medium stringency conditions, medium-high stringency conditions, high stringency conditions, or very high stringency conditions with SEQ ID NO: 29, the mature polypeptide coding sequence thereof, or the full-length complementary strand of the foregoing.
[50] The recombinant host cell of paragraph [40], wherein the polynucleotide hybridizes under low stringency conditions, medium stringency conditions, medium-high stringency conditions, high stringency conditions, or very high stringency conditions with SEQ ID NO: 31, the mature polypeptide coding sequence thereof, or the full-length complementary strand of the foregoing.
[51] The recombinant host cell of paragraph [40], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 5, or the mature polypeptide coding sequence thereof.
[52] The recombinant host cell of paragraph [40], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 7, or the mature polypeptide coding sequence thereof.
[53] The recombinant host cell of paragraph [40], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 25, or the mature polypeptide coding sequence thereof.
[54] The recombinant host cell of paragraph [40], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 29, or the mature polypeptide coding sequence thereof.
[55] The recombinant host cell of paragraph [40], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 31, or the mature polypeptide coding sequence thereof.
[56] The recombinant host cell of paragraph [40], wherein the polynucleotide encodes a lactaldehyde dehydrogenase variant of SEQ ID NO: 6 or the mature polypeptide sequence thereof, comprising a substitution, deletion, and/or insertion at not more than 10 positions, e.g., not more than 9, 8, 7, 6, 5, 4, 3, 2, or 1 position.
[57] The recombinant host cell of paragraph [40], wherein the polynucleotide encodes a lactaldehyde dehydrogenase that comprises or consists of SEQ ID NO: 6 or the mature polypeptide sequence thereof.
[58] The recombinant host cell of paragraph [40], wherein the polynucleotide encodes a lactaldehyde dehydrogenase that comprises or consists of SEQ ID NO: 6.
[59] The recombinant host cell of paragraph [40], wherein the polynucleotide encodes a lactaldehyde dehydrogenase variant of SEQ ID NO: 8 or the mature polypeptide sequence thereof, comprising a substitution, deletion, and/or insertion at not more than 10 positions, e.g., not more than 9, 8, 7, 6, 5, 4, 3, 2, or 1 position.
[60] The recombinant host cell of paragraph [40], wherein the polynucleotide encodes a lactaldehyde dehydrogenase that comprises or consists of SEQ ID NO: 8 or the mature polypeptide sequence thereof.
[61] The recombinant host cell of paragraph [40], wherein the polynucleotide encodes a lactaldehyde dehydrogenase that comprises or consists of SEQ ID NO: 8.
[62] The recombinant host cell of paragraph [40], wherein the polynucleotide encodes a lactaldehyde dehydrogenase variant of SEQ ID NO: 26 or the mature polypeptide sequence thereof, comprising a substitution, deletion, and/or insertion at not more than 10 positions, e.g., not more than 9, 8, 7, 6, 5, 4, 3, 2, or 1 position.
[63] The recombinant host cell of paragraph [40], wherein the polynucleotide encodes a lactaldehyde dehydrogenase that comprises or consists of SEQ ID NO: 26 or the mature polypeptide sequence thereof.
[64] The recombinant host cell of paragraph [40], wherein the polynucleotide encodes a lactaldehyde dehydrogenase that comprises or consists of SEQ ID NO: 26.
[65] The recombinant host cell of paragraph [40], wherein the polynucleotide encodes a lactaldehyde dehydrogenase variant of SEQ ID NO: 30 or the mature polypeptide sequence thereof, comprising a substitution, deletion, and/or insertion at not more than 10 positions, e.g., not more than 9, 8, 7, 6, 5, 4, 3, 2, or 1 position.
[66] The recombinant host cell of paragraph [40], wherein the polynucleotide encodes a lactaldehyde dehydrogenase that comprises or consists of SEQ ID NO: 30 or the mature polypeptide sequence thereof.
[67] The recombinant host cell of paragraph [40], wherein the polynucleotide encodes a lactaldehyde dehydrogenase that comprises or consists of SEQ ID NO: 30.
[68] The recombinant host cell of paragraph [40], wherein the polynucleotide encodes a lactaldehyde dehydrogenase variant of SEQ ID NO: 32 or the mature polypeptide sequence thereof, comprising a substitution, deletion, and/or insertion at not more than 10 positions, e.g., not more than 9, 8, 7, 6, 5, 4, 3, 2, or 1 position.
[69] The recombinant host cell of paragraph [40], wherein the polynucleotide encodes a lactaldehyde dehydrogenase that comprises or consists of SEQ ID NO: 32 or the mature polypeptide sequence thereof.
[70] The recombinant host cell of paragraph [40], wherein the polynucleotide encodes a lactaldehyde dehydrogenase that comprises or consists of SEQ ID NO: 32.
[71] The recombinant host cell of any one of paragraphs [2]-[70], wherein the heterologous polynucleotide that encodes a lactaldehyde dehydrogenase is operably linked to a promoter foreign to the polynucleotide.
[72] The recombinant host cell of any one of paragraphs [1]-[13], comprising a heterologous polynucleotide that encodes a lactaldehyde reductase, wherein the polynucleotide is selected from:

(a) a polynucleotide that encodes a lactaldehyde reductase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 10, 12, 28, 54, 56, or the mature polypeptide sequence thereof;

(b) a polynucleotide that hybridizes under low stringency conditions, medium stringency conditions, medium-high stringency conditions, high stringency conditions, or very high stringency conditions with SEQ ID NO: 9, 11, 27, 53, 55, the mature polypeptide coding sequence thereof, or the full-length complementary strand of the foregoing; and

(c) a polynucleotide that has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 9, 11, 27, 53, 55, or the mature polypeptide coding sequence thereof.

[73] The recombinant host cell of paragraph [72], wherein the polynucleotide encodes a lactaldehyde reductase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 10 or the mature polypeptide sequence thereof.
[74] The recombinant host cell of paragraph [72], wherein the polynucleotide encodes a lactaldehyde reductase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 12 or the mature polypeptide sequence thereof.
[75] The recombinant host cell of paragraph [72], wherein the polynucleotide encodes a lactaldehyde reductase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 28 or the mature polypeptide sequence thereof.
[76] The recombinant host cell of paragraph [72], wherein the polynucleotide encodes a lactaldehyde reductase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 54 or the mature polypeptide sequence thereof.
[77] The recombinant host cell of paragraph [72], wherein the polynucleotide encodes a lactaldehyde reductase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 56 or the mature polypeptide sequence thereof.
[78] The recombinant host cell of paragraph [72], wherein the polynucleotide hybridizes under low stringency conditions, medium stringency conditions, medium-high stringency conditions, high stringency conditions, or very high stringency conditions with SEQ ID NO: 9, the mature polypeptide coding sequence thereof, or the full-length complementary strand of the foregoing.
[79] The recombinant host cell of paragraph [72], wherein the polynucleotide hybridizes under low stringency conditions, medium stringency conditions, medium-high stringency conditions, high stringency conditions, or very high stringency conditions with SEQ ID NO: 11, the mature polypeptide coding sequence thereof, or the full-length complementary strand of the foregoing.
[80] The recombinant host cell of paragraph [72], wherein the polynucleotide hybridizes under low stringency conditions, medium stringency conditions, medium-high stringency conditions, high stringency conditions, or very high stringency conditions with SEQ ID NO: 27, the mature polypeptide coding sequence thereof, or the full-length complementary strand of the foregoing.
[81] The recombinant host cell of paragraph [72], wherein the polynucleotide hybridizes under low stringency conditions, medium stringency conditions, medium-high stringency conditions, high stringency conditions, or very high stringency conditions with SEQ ID NO: 53, the mature polypeptide coding sequence thereof, or the full-length complementary strand of the foregoing.
[82] The recombinant host cell of paragraph [72], wherein the polynucleotide hybridizes under low stringency conditions, medium stringency conditions, medium-high stringency conditions, high stringency conditions, or very high stringency conditions with SEQ ID NO: 55, the mature polypeptide coding sequence thereof, or the full-length complementary strand of the foregoing.
[83] The recombinant host cell of paragraph [72], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 9, or the mature polypeptide coding sequence thereof.
[84] The recombinant host cell of paragraph [72], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 11, or the mature polypeptide coding sequence thereof.
[85] The recombinant host cell of paragraph [72], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 27, or the mature polypeptide coding sequence thereof.
[86] The recombinant host cell of paragraph [72], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 53, or the mature polypeptide coding sequence thereof.
[87] The recombinant host cell of paragraph [72], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 55, or the mature polypeptide coding sequence thereof.
[88] The recombinant host cell of paragraph [72], wherein the polynucleotide encodes a lactaldehyde reductase variant of SEQ ID NO: 10 or the mature polypeptide sequence thereof, comprising a substitution, deletion, and/or insertion at not more than 10 positions, e.g., not more than 9, 8, 7, 6, 5, 4, 3, 2, or 1 position.
[89] The recombinant host cell of paragraph [72], wherein the polynucleotide encodes a lactaldehyde reductase that comprises or consists of SEQ ID NO: 10 or the mature polypeptide sequence thereof.
[90] The recombinant host cell of paragraph [72], wherein the polynucleotide encodes a lactaldehyde reductase that comprises or consists of SEQ ID NO: 10.
[91] The recombinant host cell of paragraph [72], wherein the polynucleotide encodes a lactaldehyde reductase variant of SEQ ID NO: 12 or the mature polypeptide sequence thereof, comprising a substitution, deletion, and/or insertion at not more than 10 positions, e.g., not more than 9, 8, 7, 6, 5, 4, 3, 2, or 1 position.
[92] The recombinant host cell of paragraph [72], wherein the polynucleotide encodes a lactaldehyde reductase that comprises or consists of SEQ ID NO: 12 or the mature polypeptide sequence thereof.
[93] The recombinant host cell of paragraph [72], wherein the polynucleotide encodes a lactaldehyde reductase that comprises or consists of SEQ ID NO: 12.
[94] The recombinant host cell of paragraph [72], wherein the polynucleotide encodes a lactaldehyde reductase variant of SEQ ID NO: 28 or the mature polypeptide sequence thereof, comprising a substitution, deletion, and/or insertion at not more than 10 positions, e.g., not more than 9, 8, 7, 6, 5, 4, 3, 2, or 1 position.
[95] The recombinant host cell of paragraph [72], wherein the polynucleotide encodes a lactaldehyde reductase that comprises or consists of SEQ ID NO: 28 or the mature polypeptide sequence thereof.
[96] The recombinant host cell of paragraph [72], wherein the polynucleotide encodes a lactaldehyde reductase that comprises or consists of SEQ ID NO: 28.
[97] The recombinant host cell of paragraph [72], wherein the polynucleotide encodes a lactaldehyde reductase variant of SEQ ID NO: 54 or the mature polypeptide sequence thereof, comprising a substitution, deletion, and/or insertion at not more than 10 positions, e.g., not more than 9, 8, 7, 6, 5, 4, 3, 2, or 1 position.
[98] The recombinant host cell of paragraph [72], wherein the polynucleotide encodes a lactaldehyde reductase that comprises or consists of SEQ ID NO: 54 or the mature polypeptide sequence thereof.
[99] The recombinant host cell of paragraph [72], wherein the polynucleotide encodes a lactaldehyde reductase that comprises or consists of SEQ ID NO: 54.
[100] The recombinant host cell of paragraph [72], wherein the polynucleotide encodes a lactaldehyde reductase variant of SEQ ID NO: 56 or the mature polypeptide sequence thereof, comprising a substitution, deletion, and/or insertion at not more than 10 positions, e.g., not more than 9, 8, 7, 6, 5, 4, 3, 2, or 1 position.
[101] The recombinant host cell of paragraph [72], wherein the polynucleotide encodes a lactaldehyde reductase that comprises or consists of SEQ ID NO: 56 or the mature polypeptide sequence thereof.
[102] The recombinant host cell of paragraph [72], wherein the polynucleotide encodes a lactaldehyde reductase that comprises or consists of SEQ ID NO: 56.
[103] The recombinant host cell of any one of paragraphs [2]-[102], wherein the heterologous polynucleotide that encodes a lactaldehyde reductase is operably linked to a promoter foreign to the polynucleotide.
[104] The recombinant host cell of any one of paragraphs [1]-[13], comprising a first heterologous polynucleotide that encodes a first propanediol dehydratase subunit and a second heterologous polynucleotide that encodes a second propanediol dehydratase subunit, wherein the first propanediol dehydratase subunit and second propanediol dehydratase subunit form a protein complex having propanediol dehydratase activity.
[105] The recombinant host cell of paragraph [104], wherein the first heterologous polynucleotide is selected from:

(a) a polynucleotide that encodes a first dehydratase subunit having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 14, 18, 58, or the mature polypeptide sequence thereof;

(b) a polynucleotide that hybridizes under low stringency conditions, medium stringency conditions, medium-high stringency conditions, high stringency conditions, or very high stringency conditions with SEQ ID NO: 13, 17, 57, the mature polypeptide coding sequence thereof, or the full-length complementary strand of the foregoing; and

(c) a polynucleotide that has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 13, 17, 57, or the mature polypeptide coding sequence thereof; and

wherein the second heterologous polynucleotide is selected from:

(a) a polynucleotide that encodes a second dehydratase subunit having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 16, 20, 60, or the mature polypeptide sequence thereof;

(b) a polynucleotide that hybridizes under low stringency conditions, medium stringency conditions, medium-high stringency conditions, high stringency conditions, or very high stringency conditions with SEQ ID NO: 15, 19, 59, the mature polypeptide coding sequence thereof, or the full-length complementary strand of the foregoing; and

(c) a polynucleotide that has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 15, 19, 59, or the mature polypeptide coding sequence thereof.

[106] The recombinant host cell of paragraph [104], wherein the first heterologous polynucleotide encodes a first dehydratase subunit having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 14, or the mature polypeptide sequence thereof; and

wherein the second heterologous polynucleotide encodes a second dehydratase subunit having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 16, or the mature polypeptide sequence thereof.

[107] The recombinant host cell of paragraph [104], wherein the first heterologous polynucleotide encodes a first dehydratase subunit having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 18, or the mature polypeptide sequence thereof; and

wherein the second heterologous polynucleotide encodes a second dehydratase subunit having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 20, or the mature polypeptide sequence thereof.

[108] The recombinant host cell of paragraph [104], wherein the first heterologous polynucleotide encodes a first dehydratase subunit having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 58, or the mature polypeptide sequence thereof; and

wherein the second heterologous polynucleotide encodes a second dehydratase subunit having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 60, or the mature polypeptide sequence thereof.

[109] The recombinant host cell of paragraph [104], wherein the first heterologous polynucleotide hybridizes under low stringency conditions, medium stringency conditions, medium-high stringency conditions, high stringency conditions, or very high stringency conditions with SEQ ID NO: 13, the mature polypeptide coding sequence thereof, or the full-length complementary strand of the foregoing; and

wherein the second heterologous polynucleotide hybridizes under low stringency conditions, medium stringency conditions, medium-high stringency conditions, high stringency conditions, or very high stringency conditions with SEQ ID NO: 15, the mature polypeptide coding sequence thereof, or the full-length complementary strand of the foregoing.

[110] The recombinant host cell of paragraph [104], wherein the first heterologous polynucleotide hybridizes under low stringency conditions, medium stringency conditions, medium-high stringency conditions, high stringency conditions, or very high stringency conditions with SEQ ID NO: 17, the mature polypeptide coding sequence thereof, or the full-length complementary strand of the foregoing; and

wherein the second heterologous polynucleotide hybridizes under low stringency conditions, medium stringency conditions, medium-high stringency conditions, high stringency conditions, or very high stringency conditions with SEQ ID NO: 19, the mature polypeptide coding sequence thereof, or the full-length complementary strand of the foregoing.

[111] The recombinant host cell of paragraph [104], wherein the first heterologous polynucleotide hybridizes under low stringency conditions, medium stringency conditions, medium-high stringency conditions, high stringency conditions, or very high stringency conditions with SEQ ID NO: 57, the mature polypeptide coding sequence thereof, or the full-length complementary strand of the foregoing; and

wherein the second heterologous polynucleotide hybridizes under low stringency conditions, medium stringency conditions, medium-high stringency conditions, high stringency conditions, or very high stringency conditions with SEQ ID NO: 59, the mature polypeptide coding sequence thereof, or the full-length complementary strand of the foregoing.

[112] The recombinant host cell of paragraph [104], wherein the first heterologous polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 13, or the mature polypeptide coding sequence thereof; and

wherein the second heterologous polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 15, or the mature polypeptide coding sequence thereof.

[113] The recombinant host cell of paragraph [104], wherein the first heterologous polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 17, or the mature polypeptide coding sequence thereof; and

wherein the second heterologous polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 19, or the mature polypeptide coding sequence thereof.

[114] The recombinant host cell of paragraph [104], wherein the first heterologous polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 57, or the mature polypeptide coding sequence thereof; and

wherein the second heterologous polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 59, or the mature polypeptide coding sequence thereof.

[115] The recombinant host cell of paragraph [104], wherein the first heterologous polynucleotide encodes a first dehydratase subunit variant of SEQ ID NO: 14 or the mature polypeptide sequence thereof, comprising a substitution, deletion, and/or insertion at not more than 10 positions, e.g., not more than 9, 8, 7, 6, 5, 4, 3, 2, or 1 position; and

wherein the second heterologous polynucleotide encodes a second dehydratase subunit variant of SEQ ID NO: 16 or the mature polypeptide sequence thereof, comprising a substitution, deletion, and/or insertion at not more than 10 positions, e.g., not more than 9, 8, 7, 6, 5, 4, 3, 2, or 1 position.

[116] The recombinant host cell of paragraph [104], wherein the first heterologous polynucleotide encodes a first dehydratase subunit that comprises or consists of SEQ ID NO: 14 or the mature polypeptide sequence thereof; and

wherein the second heterologous polynucleotide encodes a second dehydratase subunit that comprises or consists of SEQ ID NO: 16 or the mature polypeptide sequence thereof.

[117] The recombinant host cell of paragraph [104], wherein the first heterologous polynucleotide encodes a first dehydratase subunit that comprises or consists of SEQ ID NO: 14; and

wherein the second heterologous polynucleotide encodes a second dehydratase subunit that comprises or consists of SEQ ID NO: 16.

[118] The recombinant host cell of paragraph [104], wherein the first heterologous polynucleotide encodes a first dehydratase subunit variant of SEQ ID NO: 18 or the mature polypeptide sequence thereof, comprising a substitution, deletion, and/or insertion at not more than 10 positions, e.g., not more than 9, 8, 7, 6, 5, 4, 3, 2, or 1 position; and

wherein the second heterologous polynucleotide encodes a second dehydratase subunit variant of SEQ ID NO: 20 or the mature polypeptide sequence thereof, comprising a substitution, deletion, and/or insertion at not more than 10 positions, e.g., not more than 9, 8, 7, 6, 5, 4, 3, 2, or 1 position.

[119] The recombinant host cell of paragraph [104], wherein the first heterologous polynucleotide encodes a first dehydratase subunit that comprises or consists of SEQ ID NO: 18 or the mature polypeptide sequence thereof; and

wherein the second heterologous polynucleotide encodes a second dehydratase subunit that comprises or consists of SEQ ID NO: 20 or the mature polypeptide sequence thereof.

[120] The recombinant host cell of paragraph [104], wherein the first heterologous polynucleotide encodes a first dehydratase subunit that comprises or consists of SEQ ID NO: 18; and

wherein the second heterologous polynucleotide encodes a second dehydratase subunit that comprises or consists of SEQ ID NO: 20.

[121] The recombinant host cell of paragraph [104], wherein the first heterologous polynucleotide encodes a first dehydratase subunit variant of SEQ ID NO: 58 or the mature polypeptide sequence thereof, comprising a substitution, deletion, and/or insertion at not more than 10 positions, e.g., not more than 9, 8, 7, 6, 5, 4, 3, 2, or 1 position; and

wherein the second heterologous polynucleotide encodes a second dehydratase subunit variant of SEQ ID NO: 60 or the mature polypeptide sequence thereof, comprising a substitution, deletion, and/or insertion at not more than 10 positions, e.g., not more than 9, 8, 7, 6, 5, 4, 3, 2, or 1 position.

[122] The recombinant host cell of paragraph [104], wherein the first heterologous polynucleotide encodes a first dehydratase subunit that comprises or consists of SEQ ID NO: 58 or the mature polypeptide sequence thereof; and

wherein the second heterologous polynucleotide encodes a second dehydratase subunit that comprises or consists of SEQ ID NO: 60 or the mature polypeptide sequence thereof.

[123] The recombinant host cell of paragraph [104], wherein the first heterologous polynucleotide encodes a first dehydratase subunit that comprises or consists of SEQ ID NO: 58; and

wherein the second heterologous polynucleotide encodes a second dehydratase subunit that comprises or consists of SEQ ID NO: 60.

[124] The recombinant host cell of any one of paragraphs [104]-[123], wherein the first heterologous polynucleotide that encodes the first dehydratase subunit and the second heterologous polynucleotide encodes the second dehydratase subunit are contained in a single heterologous polynucleotide.
[125] The recombinant host cell of paragraph [124], wherein the single heterologous polynucleotide is operably linked to a promoter foreign to both the first heterologous polynucleotide that encodes the first dehydratase subunit and the second heterologous polynucleotide encodes the second dehydratase subunit.
[126] The recombinant host cell of any one of paragraphs [104]-[123], wherein the first heterologous polynucleotide that encodes the first dehydratase subunit and the second heterologous polynucleotide encodes the second dehydratase subunit are contained in a separate heterologous polynucleotides.
[127] The recombinant host cell of paragraph [126], wherein the first heterologous polynucleotide is operably linked to a foreign promoter, and the second heterologous polynucleotide is operably linked to a foreign promoter.
[128] The recombinant host cell of any one of paragraphs [1]-[13], comprising a heterologous polynucleotide that encodes an n-propanol dehydrogenase, wherein the polynucleotide is selected from:

(a) a polynucleotide that encodes an n-propanol dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 22, 24, 62, or the mature polypeptide sequence thereof;

(b) a polynucleotide that hybridizes under low stringency conditions, medium stringency conditions, medium-high stringency conditions, high stringency conditions, or very high stringency conditions with SEQ ID NO: 21, 23, 61, the mature polypeptide coding sequence thereof, or the full-length complementary strand of the foregoing; and

(c) a polynucleotide that has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 21, 23, 61, or the mature polypeptide coding sequence thereof.

[129] The recombinant host cell of paragraph [128], wherein the polynucleotide encodes an n-propanol dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 22 or the mature polypeptide sequence thereof.
[130] The recombinant host cell of paragraph [128], wherein the polynucleotide encodes an n-propanol dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 24 or the mature polypeptide sequence thereof.
[131] The recombinant host cell of paragraph [128], wherein the polynucleotide encodes an n-propanol dehydrogenase having at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 62 or the mature polypeptide sequence thereof.
[132] The recombinant host cell of paragraph [128], wherein the polynucleotide hybridizes under low stringency conditions, medium stringency conditions, medium-high stringency conditions, high stringency conditions, or very high stringency conditions with SEQ ID NO: 21, the mature polypeptide coding sequence thereof, or the full-length complementary strand of the foregoing.
[133] The recombinant host cell of paragraph [128], wherein the polynucleotide hybridizes under low stringency conditions, medium stringency conditions, medium-high stringency conditions, high stringency conditions, or very high stringency conditions with SEQ ID NO: 23, the mature polypeptide coding sequence thereof, or the full-length complementary strand of the foregoing.
[134] The recombinant host cell of paragraph [128], wherein the polynucleotide hybridizes under low stringency conditions, medium stringency conditions, medium-high stringency conditions, high stringency conditions, or very high stringency conditions with SEQ ID NO: 61, the mature polypeptide coding sequence thereof, or the full-length complementary strand of the foregoing.
[135] The recombinant host cell of paragraph [128], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 21, or the mature polypeptide coding sequence thereof.
[136] The recombinant host cell of paragraph [128], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 23, or the mature polypeptide coding sequence thereof.
[137] The recombinant host cell of paragraph [128], wherein the polynucleotide has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to SEQ ID NO: 61, or the mature polypeptide coding sequence thereof.
[138] The recombinant host cell of paragraph [128], wherein the polynucleotide encodes an n-propanol dehydrogenase variant of SEQ ID NO: 22 or the mature polypeptide sequence thereof, comprising a substitution, deletion, and/or insertion at not more than 10 positions, e.g., not more than 9, 8, 7, 6, 5, 4, 3, 2, or 1 position.
[139] The recombinant host cell of paragraph [128], wherein the polynucleotide encodes an n-propanol dehydrogenase that comprises or consists of SEQ ID NO: 22 or the mature polypeptide sequence thereof.
[140] The recombinant host cell of paragraph [128], wherein the polynucleotide encodes an n-propanol dehydrogenase that comprises or consists of SEQ ID NO: 22.
[141] The recombinant host cell of paragraph [128], wherein the polynucleotide encodes an n-propanol dehydrogenase variant of SEQ ID NO: 24 or the mature polypeptide sequence thereof, comprising a substitution, deletion, and/or insertion at not more than 10 positions, e.g., not more than 9, 8, 7, 6, 5, 4, 3, 2, or 1 position.
[142] The recombinant host cell of paragraph [128], wherein the polynucleotide encodes an n-propanol dehydrogenase that comprises or consists of SEQ ID NO: 24 or the mature polypeptide sequence thereof.
[143] The recombinant host cell of paragraph [128], wherein the polynucleotide encodes an n-propanol dehydrogenase that comprises or consists of SEQ ID NO: 24.
[144] The recombinant host cell of paragraph [128], wherein the polynucleotide encodes an n-propanol dehydrogenase variant of SEQ ID NO: 62 or the mature polypeptide sequence thereof, comprising a substitution, deletion, and/or insertion at not more than 10 positions, e.g., not more than 9, 8, 7, 6, 5, 4, 3, 2, or 1 position.
[145] The recombinant host cell of paragraph [128], wherein the polynucleotide encodes an n-propanol dehydrogenase that comprises or consists of SEQ ID NO: 62 or the mature polypeptide sequence thereof.
[146] The recombinant host cell of paragraph [128], wherein the polynucleotide encodes an n-propanol dehydrogenase that comprises or consists of SEQ ID NO: 62.
[147] The recombinant host cell of any one of paragraphs [2]-[146], wherein the heterologous polynucleotide that encodes an n-propanol dehydrogenase is operably linked to a promoter foreign to the polynucleotide.
[148] The recombinant host cell of any one of paragraphs [1]-[147], further comprising thiolase activity, CoA-transferase activity, acetoacetate decarboxylase activity, and isopropanol dehydrogenase activity, wherein the cell is capable of producing isopropanol.
[149] The recombinant host cell of any one of paragraphs [1]-[148], comprising:

one or more heterologous polynucleotides that encode a thiolase,

one or more heterologous polynucleotides that encode a CoA-transferase,

one or more heterologous polynucleotides that encode a acetoacetate decarboxylase, or

one or more heterologous polynucleotides that encode an isopropanol dehydrogenase;

wherein the host cell is capable of producing a greater amount of isopropanol compared to the host cell without the heterologous polynucleotide(s) when cultivated under the same conditions.

[150] The recombinant host cell of paragraph [149], wherein the CoA-transferase is a succinyl-CoA:acetoacetate transferase.
[151] The recombinant host cell of any one of paragraph [1]-[150], wherein the cell is capable of n-propanol volumetric productivity greater than about 0.1 g/L per hour, e.g., greater than about 0.2 g/L per hour, 0.5 g/L per hour, 0.6 g/L per hour, 0.7 g/L per hour, 0.8 g/L per hour, 0.9 g/L per hour, 1.0 g/L per hour, 1.1 g/L per hour, 1.2 g/L per hour, 1.3 g/L per hour, 1.5 g/L per hour, 1.75 g/L per hour, 2.0 g/L per hour, 2.25 g/L per hour, 2.5 g/L per hour, or 3.0 g/L per hour; or between about 0.1 g/L per hour and about 2.0 g/L per hour, e.g., between about 0.3 g/L per hour and about 1.7 g/L per hour, about 0.5 g/L per hour and about 1.5 g/L per hour, about 0.7 g/L per hour and about 1.3 g/L per hour, about 0.8 g/L per hour and about 1.2 g/L per hour, or about 0.9 g/L per hour and about 1.1 g/L per hour.
[152] The recombinant host cell of any one of paragraphs [1]-[151], wherein the cell has decreased propionaldehyde dehydrogenase activity compared to that of a wild-type strain under the same conditions.
[153] The recombinant host cell of any one of paragraphs [1]-[151], wherein the cell has an absence of propionaldehyde dehydrogenase activity.
[154] The recombinant host cell of any one of paragraphs [1]-[155], wherein the cell has a disruption to an endogenous polynucleotide that encodes a propionaldehyde dehydrogenase.
[155] The recombinant host cell of paragraph [154], wherein the cell has a disruption to an endogenous polynucleotide that encodes a propionaldehyde dehydrogenase having at least 60%, e.g., at least 70%, at least 75%, at least 80%, at least 85%, at least 90%, at least 95%, at least 97%, at least 98%, at least 99%, or 100% sequence identity to SEQ ID NO: 68.
[156] The recombinant host cell of paragraph [154] or [155], wherein the disruption inactivates the propionaldehyde dehydrogenase gene.
[157] The recombinant host cell of any one of paragraphs [154]-[156], wherein the propionaldehyde dehydrogenase activity of the host cell is decreased by at least 50%, e.g., at least 60%, at least 70%, at least 80%, at least 90%, or at least 95% compared to the host cell without the disruption of the polynucleotide that encodes the propionaldehyde dehydrogenase under the same conditions.
[158] The recombinant host cell of any one of paragraphs [1]-[157], wherein the cell has a disruption to an endogenous polynucleotide that encodes an alcohol dehydrogenase.
[159] The recombinant host cell of paragraph [158], wherein the cell has a disruption to an endogenous polynucleotide that encodes an alcohol dehydrogenase having at least 60%, e.g., at least 70%, at least 75%, at least 80%, at least 85%, at least 90%, at least 95%, at least 97%, at least 98%, at least 99%, or 100% sequence identity to SEQ ID NO: 66.
[160] The recombinant host cell of paragraph [158] or [159], wherein the disruption inactivates the alcohol dehydrogenase gene.
[161] The recombinant host cell of any one of paragraphs [158]-[160], wherein the alcohol dehydrogenase activity of the host cell is decreased by at least 50%, e.g., at least 60%, at least 70%, at least 80%, at least 90%, or at least 95% compared to the host cell without the disruption of the polynucleotide that encodes the alcohol dehydrogenase under the same conditions.
[162] The recombinant host cell of any one of paragraphs [1]-[161], wherein host cell is a bacterial host cell.
[163] The recombinant host cell of paragraph [162], wherein the host cell is a member of a genus selected from the group consisting of Bacillus, Clostridium, Enterococcus, Geobacillus, Lactobacillus, Lactococcus, Oceanobacillus, Propionibacterium, Staphylococcus, Streptococcus, Streptomyces, Campylobacter, Escherichia, Flavobacterium, Fusobacterium, Helicobacter, Ilyobacter, Neisseria, Pseudomonas, Salmonella, and Ureaplasma.
[164] The recombinant host cell of paragraph [162], wherein the host cell is a member of a genus selected from Escherichia (e.g., Escherichia coli), Lactobacillus (e.g., Lactobacillus plantarum, Lactobacillus fructivorans, or Lactobacillus reuteri), and Propionibacterium (e.g., Propionibacterium freudenreichii).
[165] The recombinant host cell of any one of paragraphs [1]-[161], wherein host cell is a eukaryotic host cell.
[166] The recombinant host cell of paragraph [165], wherein the host cell is a filamentous fungal host cell.
[167] The recombinant host cell of paragraph [165], wherein the host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Rhizopus, Schizophyllum, Talaromyces, Thermoascus, Thielavia, Tolypocladium, Trametes, or Trichoderma host cell.
[168] The recombinant host cell of paragraph [165], wherein the host cell is an Aspergillus host cell.
[169] The recombinant host cell of paragraph [165], wherein the host cell is an Aspergillus oryzae host cell.
[170] The recombinant host cell of paragraph [165], wherein the host cell is a yeast host cell.
[171] The recombinant host cell of paragraph [170], wherein the host cell is a Candida, Hansenula, Kluyveromyces, Pichia, Saccharomyces, Schizosaccharomyces, Yarrowia, or Issatchenkia host cell.
[172] The recombinant host cell of paragraph [170], wherein the host cell is an I. orientalis, P. galeiformis, Pichia sp. YB-4149 (NRRL designation), C. ethanolica, P. deserticola, P. membranifaciens or P. fermentans, or S. cerevisiae host cell.
[173] The recombinant host cell of paragraph [170], wherein the host cell is an I. orientalis or P. membranifaciens host cell.
[174] The recombinant host cell of any one of paragraphs [1]-[173], wherein the host cell has been genetically modified to produce less ethanol compared to that of a wild-type strain under the same conditions.
[175] The recombinant host cell of any one of paragraphs [1]-[174], wherein the host cell is unable to produce more than about 20 g/L, e.g., about 15 g/L, about 10 g/L, about 5 g/L, about 2.5 g/L, about 1 g/L, about 0.5 g/L, or about 0.1 g/L ethanol.
[176] The recombinant host cell of any one of paragraphs [1]-[174], wherein the host cell is unable to produce detectable amounts of ethanol.
[177] The recombinant host cell of any one of paragraphs [1]-[176], wherein the cell has decreased pyruvate decarboxylase activity compared to that of a wild-type strain under the same conditions.
[178] The recombinant host cell of any one of paragraphs [1]-[176], wherein the cell has an absence of pyruvate decarboxylase activity.
[179] The recombinant host cell of any one of paragraphs [1]-[178], wherein the cell has a disruption to an endogenous polynucleotide that encodes a pyruvate decarboxylase.
[180] The recombinant host cell of paragraph [179], wherein the pyruvate decarboxylase activity of the host cell is decreased by at least 50%, e.g., at least 60%, at least 70%, at least 80%, at least 90%, or at least 95% compared to the host cell without the disruption of the polynucleotide that encodes the pyruvate decarboxylase under the same conditions.
[181] The recombinant host cell of any one of paragraphs [1]-[180], comprising:

one or more heterologous polynucleotides that encode a xylose isomerase gene,

a disruption of an endogenous polynucleotide that encodes a polypeptide that catalyzes the conversion of xylose to xylitol,

a disruption of an endogenous polynucleotide that encodes a xylitol dehydrogenase; and/or

one or more heterologous polynucleotides that encode a xylulokinase.

[182] The recombinant host cell of any one of paragraphs [1]-[181], wherein the cell has decreased L- or D-lactate:ferricytochrome c oxidoreductase activity compared to that of a wild-type strain under the same conditions.
[183] The recombinant host cell of any one of paragraphs [1]-[181], wherein the cell has an absence of L- or D-lactate:ferricytochrome c oxidoreductase activity.
[184] The recombinant host cell of any one of paragraphs [1]-[183], wherein the cell has a disruption to an endogenous polynucleotide that encodes a L- or D-lactate:ferricytochrome c oxidoreductase.
[185] The recombinant host cell of paragraph [184], wherein the L- or D-lactate:ferricytochrome c oxidoreductase activity of the host cell is decreased by at least 50%, e.g., at least 60%, at least 70%, at least 80%, at least 90%, or at least 95% compared to the host cell without the disruption of the polynucleotide that encodes the L- or D-lactate:ferricytochrome c oxidoreductase under the same conditions.
[186] The recombinant host cell of any one of paragraphs [1]-[185], wherein the host cell has been genetically modified to produce less glycerol compared to that of a wild-type strain under the same conditions.
[187] The recombinant host cell of any one of paragraphs [1]-[186], wherein the host cell is unable to produce more than about 20 g/L, e.g., about 15 g/L, about 10 g/L, about 5 g/L, about 2.5 g/L, about 1 g/L, about 0.5 g/L, or about 0.1 g/L glycerol.
[188] The recombinant host cell of any one of paragraphs [1]-[186], wherein the host cell is unable to produce detectable amounts of glycerol.
[189] The recombinant host cell of any one of paragraphs [1]-[188], wherein the cell has decreased glycerol-3-phosphate dehydrogenase (GPD) activity compared to that of a wild-type strain under the same conditions.
[190] The recombinant host cell of any one of paragraphs [1]-[188], wherein the cell has an absence of glycerol-3-phosphate dehydrogenase (GPD) activity.
[191] The recombinant host cell of any one of paragraphs [1]-[190], wherein the cell has a disruption to an endogenous polynucleotide that encodes a glycerol-3-phosphate dehydrogenase (GPD).
[192] The recombinant host cell of paragraph [191], wherein the glycerol-3-phosphate dehydrogenase (GPD) activity of the host cell is decreased by at least 50%, e.g., at least 60%, at least 70%, at least 80%, at least 90%, or at least 95% compared to the host cell without the disruption of the polynucleotide that encodes the glycerol-3-phosphate dehydrogenase (GPD) under the same conditions.
[193] The recombinant host cell of any one of paragraphs [1]-[192], wherein the cell has decreased glycerol-3-phosphatase (GPP) activity compared to that of a wild-type strain under the same conditions.
[194] The recombinant host cell of any one of paragraphs [1]-[192], wherein the cell has an absence of glycerol-3-phosphatase (GPP) activity.
[195] The recombinant host cell of any one of paragraphs [1]-[194], wherein the cell has a disruption to an endogenous polynucleotide that encodes a glycerol-3-phosphatase (GPP).
[196] The recombinant host cell of paragraph [195], wherein the glycerol-3-phosphatase (GPP) activity of the host cell is decreased by at least 50%, e.g., at least 60%, at least 70%, at least 80%, at least 90%, or at least 95% compared to the host cell without the disruption of the polynucleotide that encodes the glycerol-3-phosphatase (GPP) under the same conditions.
[197] The recombinant host cell of any one of paragraphs [1]-[196], wherein the cell has decreased dihydroxyacetone phosphate phosphatase activity compared to that of a wild-type strain under the same conditions.
[198] The recombinant host cell of any one of paragraphs [1]-[196], wherein the cell has an absence of dihydroxyacetone phosphate phosphatase activity.
[199] The recombinant host cell of any one of paragraphs [1]-[198], wherein the cell has a disruption to an endogenous polynucleotide that encodes a dihydroxyacetone phosphate phosphatase.
[200] The recombinant host cell of paragraph [199], wherein the dihydroxyacetone phosphate phosphatase activity of the host cell is decreased by at least 50%, e.g., at least 60%, at least 70%, at least 80%, at least 90%, or at least 95% compared to the host cell without the disruption of the polynucleotide that encodes the dihydroxyacetone phosphate phosphatase under the same conditions.
[201] The recombinant host cell of any one of paragraphs [1]-[200], wherein the cell has decreased glycerol dehydrogenase activity compared to that of a wild-type strain under the same conditions.
[202] The recombinant host cell of any one of paragraphs [1]-[200], wherein the cell has an absence of glycerol dehydrogenase activity.
[203] The recombinant host cell of any one of paragraphs [1]-[202], wherein the cell has a disruption to an endogenous polynucleotide that encodes a glycerol dehydrogenase.
[204] The recombinant host cell of paragraph [203], wherein the glycerol dehydrogenase activity of the host cell is decreased by at least 50%, e.g., at least 60%, at least 70%, at least 80%, at least 90%, or at least 95% compared to the host cell without the disruption of the polynucleotide that encodes the glycerol dehydrogenase under the same conditions.
[205] A composition comprising the recombinant host cell of any one of paragraphs [1]-[204].
[206] The composition of paragraph [205], wherein the medium is a fermentable medium.
[207] The composition of paragraph [205] or [206], further comprising n-propanol.
[208] The composition of paragraph [207], wherein the n-propanol is at a titer greater than about 10 g/L, e.g., greater than about 25 g/L, 50 g/L, 75 g/L, 100 g/L, 125 g/L, 150 g/L, 160 g/L, 170 g/L, 180 g/L, 190 g/L, 200 g/L, 210 g/L, 225 g/L, 250 g/L, 275 g/L, 300 g/L, 325 g/L, 350 g/L, 400 g/L, or 500 g/L; or between about 10 g/L and about 500 g/L, e.g., between about 50 g/L and about 350 g/L, about 100 g/L and about 300 g/L, about 150 g/L and about 250 g/L, about 175 g/L and about 225 g/L, or about 190 g/L and about 210 g/L.
[209] The composition of paragraph any one of paragraphs [205]-[208], further comprising isopropanol.
[210] The composition of paragraph [209], wherein the isopropanol is at a titer greater than about 10 g/L, e.g., greater than about 25 g/L, 50 g/L, 75 g/L, 100 g/L, 125 g/L, 150 g/L, 160 g/L, 170 g/L, 180 g/L, 190 g/L, 200 g/L, 210 g/L, 225 g/L, 250 g/L, 275 g/L, 300 g/L, 325 g/L, 350 g/L, 400 g/L, or 500 g/L; or between about 10 g/L and about 500 g/L, e.g., between about 50 g/L and about 350 g/L, about 100 g/L and about 300 g/L, about 150 g/L and about 250 g/L, about 175 g/L and about 225 g/L, or about 190 g/L and about 210 g/L.
[211] A method of producing n-propanol, comprising:

(a) cultivating the recombinant host cell of any one of paragraphs [1]-[204] in a medium under suitable conditions to produce n-propanol; and

(b) recovering the n-propanol.

[212] The method of paragraph [211], wherein the medium is a fermentable medium.
[213] The method of paragraph [212], wherein the fermentable medium comprises sugarcane juice (e.g., non-sterilized sugarcane juice).
[214] The method of any one of paragraphs [211]-[213], wherein the produced n-propanol is at a titer greater than about 10 g/L, e.g., greater than about 25 g/L, 50 g/L, 75 g/L, 100 g/L, 125 g/L, 150 g/L, 160 g/L, 170 g/L, 180 g/L, 190 g/L, 200 g/L, 210 g/L, 225 g/L, 250 g/L, 275 g/L, 300 g/L, 325 g/L, 350 g/L, 400 g/L, or 500 g/L; or between about 10 g/L and about 500 g/L, e.g., between about 50 g/L and about 350 g/L, about 100 g/L and about 300 g/L, about 150 g/L and about 250 g/L, about 175 g/L and about 225 g/L, or about 190 g/L and about 210 g/L.
[215] The method of any one of paragraphs [211]-[214], further comprising purifying the recovered n-propanol by distillation.
[216] The method of any one of paragraphs [211]-[214], further comprising purifying the recovered n-propanol by converting propionaldehyde contaminant to n-propanol in the presence of a reducing agent.
[217] The method of any one of paragraph [211]-[216], wherein the recombinant host cell is cultivated at a pH of about 1.5 to about 7.0, e.g., about 1.5 to about 5.0, about 2.0 to about 4.0, or about 2.0 to about 4.5.
[218] A method of producing propylene, comprising:

(a) cultivating the recombinant host cell of any one of paragraphs [1]-[204] in a medium under suitable conditions to produce n-propanol;

(b) recovering the n-propanol;

(c) dehydrating the n-propanol under suitable conditions to produce propylene; and

(d) recovering the propylene.

[219] The method of paragraph [218], wherein the medium is a fermentable medium.
[220] The method of paragraph [219], wherein the fermentable medium comprises sugarcane juice (e.g., non-sterilized sugarcane juice).
[221] The method of any one of paragraphs [218]-[220], wherein the produced n-propanol is at a titer greater than about 10 g/L, e.g., greater than about 25 g/L, 50 g/L, 75 g/L, 100 g/L, 125 g/L, 150 g/L, 160 g/L, 170 g/L, 180 g/L, 190 g/L, 200 g/L, 210 g/L, 225 g/L, 250 g/L, 275 g/L, 300 g/L, 325 g/L, 350 g/L, 400 g/L, or 500 g/L; or between about 10 g/L and about 500 g/L, e.g., between about 50 g/L and about 350 g/L, about 100 g/L and about 300 g/L, about 150 g/L and about 250 g/L, about 175 g/L and about 225 g/L, or about 190 g/L and about 210 g/L.
[222] The method of any one of paragraphs [218]-[221], wherein dehydrating the n-propanol comprises treating the n-propanol with an acid catalyst.
[223] A method of coproducing n-propanol and isopropanol, comprising:

(a) cultivating the recombinant host cell of any one of paragraphs [148]-[204] in a medium under suitable conditions to produce n-propanol and isopropanol; and

(b) recovering the n-propanol and isopropanol.

[224] The method of paragraph [223], wherein the medium is a fermentable medium.
[225] The method of paragraph [224], wherein the fermentable medium comprises sugarcane juice (e.g., non-sterilized sugarcane juice).
[226] The method of any one of paragraphs [223]-[225], wherein the produced n-propanol and/or isopropanol is at a titer greater than about 10 g/L, e.g., greater than about 25 g/L, 50 g/L, 75 g/L, 100 g/L, 125 g/L, 150 g/L, 160 g/L, 170 g/L, 180 g/L, 190 g/L, 200 g/L, 210 g/L, 225 g/L, 250 g/L, 275 g/L, 300 g/L, 325 g/L, 350 g/L, 400 g/L, or 500 g/L; or between about 10 g/L and about 500 g/L, e.g., between about 50 g/L and about 350 g/L, about 100 g/L and about 300 g/L, about 150 g/L and about 250 g/L, about 175 g/L and about 225 g/L, or about 190 g/L and about 210 g/L.
[227] The method of any one of paragraphs [223]-[226], further comprising purifying the recovered n-propanol and isopropanol by distillation.
[228] The method of any one of paragraphs [223]-[226], further comprising purifying the recovered n-propanol by converting propionaldehyde contaminant to n-propanol and/or converting acetone contaminant to isopropanol in the presence of a reducing agent.
[229] The method of any one of paragraph [223]-[228], wherein the recombinant host cell is cultivated at a pH of about 1.5 to about 7.0, e.g., about 1.5 to about 5.0, about 2.0 to about 4.0, or about 2.0 to about 4.5.

Although the foregoing has been described in some detail by way of illustration and example for the purposes of clarity of understanding, it is apparent to those skilled in the art that any equivalent aspect or modification, may be practiced. Therefore, the description and examples should not be construed as limiting the scope of the invention.

1. A recombinant host cell comprising a heterologous polynucleotide that encodes a lactaldehyde dehydrogenase, wherein the host cell is capable of producing n-propanol.

2. The recombinant host cell of claim 1, further comprising a heterologous polynucleotide that encodes a lactaldehyde reductase.

3. The recombinant host cell of claim 1, further comprising
a heterologous polynucleotides that encodes a thiolase,
one or more heterologous polynucleotides that encode a CoA-transferase,
a heterologous polynucleotides that encodes a acetoacetate decarboxylase, or
a heterologous polynucleotides that encodes an isopropanol dehydrogenase;
wherein the host cell is capable of producing isopropanol.

4. The recombinant host cell of claim 1, wherein the cell has a disruption to an endogenous polynucleotide that encodes a propionaldehyde dehydrogenase.

5. The recombinant host cell of claim 4, wherein the cell has a disruption to an endogenous polynucleotide that encodes a propionaldehyde dehydrogenase having at least 60% sequence identity to SEQ ID NO: 68.

6. The recombinant host cell of claim 4, wherein the disruption inactivates the propionaldehyde dehydrogenase gene.

7. The recombinant host cell of claim 1, wherein the cell has a disruption to an endogenous polynucleotide that encodes an alcohol dehydrogenase.

8. The recombinant host cell of claim 7, wherein the cell has a disruption to an endogenous polynucleotide that encodes an alcohol dehydrogenase having at least 60% sequence identity to SEQ ID NO: 66.

9. The recombinant host cell of claim 7, wherein the disruption inactivates the alcohol dehydrogenase gene.

10. The recombinant host cell claim 1, wherein the heterologous polynucleotide that encodes a lactaldehyde dehydrogenase is selected from:
(a) a polynucleotide that encodes a lactaldehyde dehydrogenase having at least 65% sequence identity to SEQ ID NO: 6, 8, 26, 30, 32, or the mature polypeptide sequence thereof;
(b) a polynucleotide that hybridizes under medium stringency conditions with SEQ ID NO: 5, 7, 25, 29, 31, the mature polypeptide coding sequence thereof, or the full-length complementary strand of the foregoing; and
(c) a polynucleotide that has at least 65% sequence identity to SEQ ID NO: 5, 7, 25, 29, 31, or the mature polypeptide coding sequence thereof.

11. The recombinant host cell of claim 1, wherein the lactaldehyde dehydrogenase has at least 65% sequence identity to SEQ ID NO: 6, or the mature polypeptide sequence thereof.

12-13. (canceled)

14. The recombinant host cell of claim 1, wherein the lactaldehyde dehydrogenase has the amino acid sequence of SEQ ID NO: 6.

15. The recombinant host cell of claim 2, wherein the heterologous polynucleotide that encodes a lactaldehyde reductase is selected from:
(a) a polynucleotide that encodes a lactaldehyde reductase having at least 65% sequence identity to SEQ ID NO: 10, 12, 28, 54, 56, or the mature polypeptide sequence thereof;
(b) a polynucleotide that hybridizes under medium stringency conditions with SEQ ID NO: 9, 11, 27, 53, 55, the mature polypeptide coding sequence thereof, or the full-length complementary strand of the foregoing; and
(c) a polynucleotide that has at least 65% sequence identity to SEQ ID NO: 9, 11, 27, 53, 55, or the mature polypeptide coding sequence thereof.

16. The recombinant host cell of claim 2, wherein the lactaldehyde reductase has at least 65% sequence identity to SEQ ID NO: 10, or the mature polypeptide sequence thereof.

17-18. (canceled)

19. The recombinant host cell of claim 2, wherein the lactaldehyde reductase has the amino acid sequence of SEQ ID NO: 10.

20. The recombinant host cell of claim 1, wherein host cell is a bacterial host cell.

21. The recombinant host cell of claim 20, wherein the host cell is a member of a genus selected from Escherichia , Lactobacillus, and Propionibacterium.

22. The recombinant host cell of claim 20, wherein the host cell is Lactobacillus reuteri.

23. (canceled)

24. A method of producing n-propanol, comprising:
(a) cultivating the recombinant host cell of claim 1 in a medium under suitable conditions to produce n-propanol; and
(b) recovering the n-propanol.

25. A method of producing propylene, comprising:
(a) cultivating the recombinant host cell of claim 1 in a medium under suitable conditions to produce n-propanol;
(b) recovering the n-propanol;
(c) dehydrating the n-propanol under suitable conditions to produce propylene; and
(d) recovering the propylene.

26. The method of claim 24, wherein the medium comprises sugarcane juice.

27. The method of any one claim 24, wherein the recombinant host cell is cultivated at a pH of about 1.5 to about 5.0.
